SEQUENCE LISTING (154 sequences)

SEQ ID NO: 1 - protein, 12 aa, Artificial Sequence
  Synthetic peptide
HSIVYAIEAAIF

SEQ ID NO: 2 - protein, 15 aa, Artificial Sequence
  Synthetic peptide
RNVEHALMRIVLAIY

SEQ ID NO: 3 - protein, 27 aa, Artificial Sequence
  Synthetic peptide
SDVNEALHSIVYAIEAAIFALEAAERT

SEQ ID NO: 4 - protein, 27 aa, Artificial Sequence
  Synthetic peptide
RNVEHALMRIVLAIYLAEENLREAEES

SEQ ID NO: 5 - protein, 22 aa, Artificial Sequence
  Synthetic peptide
EVRELARELVRLAVEAAEEVQR

SEQ ID NO: 6 - protein, 22 aa, Artificial Sequence
  Synthetic peptide
EKREKARERVREAVERAEEVQR

SEQ ID NO: 7 - protein, 108 aa, Artificial Sequence
  Synthetic peptide
SDEEEARELIERAKEAAERAQEAAERTGDPRVRELARELKRLAQEAAEEVKRDPSSSDVNEALKLIVEAIEAAVDALEAAERTGDPEVRELARELVRLAVEAAEEVQR

SEQ ID NO: 8 - protein, 229 aa, Artificial Sequence
  Synthetic peptide
SSDEEEARELIERAKEAAERAQEAAERTGDPRVRELARELKRLAQEAAEEVKRDPSSSDVNEALKLIVEAIEAAVDALEAAERTGDPEVRELARELVRLAVEAAEEVQRNPSSSDVNEALHSIVYAIEAAIFALEAAERTGDPEVRELARELVRLAVEAAEEVQRNPSSRNVEHALMRIVLAIYLAEENLREAEESGDPEKREKARERVREAVERAEEVQRDPSGWLNH

SEQ ID NO: 9 - protein, 229 aa, Artificial Sequence
  Synthetic peptide
SSDEEEARELIERAKEAAERAQEAAERTGDPRVRELARELKRLAQEAAEEVKRDPSSSDVNEALKLIVEAIEAAVDALEAAERTGDPEVRELARELVRLAVEAAEEVQRNPSSSDVNEALLSIVIAIEAAVHALEAAERTGDPEVRELARELVRLAVEAAEEVQRNPSSREVEHALMKIVLAIYEAEESLREAEESGDPEKREKARERVREAVERAEEVQRDPSGWLNH

SEQ ID NO: 10 - protein, 229 aa, Artificial Sequence
  Synthetic peptide
SSDEEEARELIERAKEAAERAQEAAERTGDPRVRELARELKRLAQEAAEEVKRDPSSSDVNEALKLIVEAIEAAVDALEAAERTGDPEVRELARELVRLAVEAAEEVQRNPSSSDVNEALLTIVIAIEAAVNALEAAERTGDPEVRELARELVRLAVEAAEEVQRNPSSREVNIALWKIVLAIQEAVESLREAEESGDPEKREKARERVREAVERAEEVQRDPSGWLNH

SEQ ID NO: 11 - protein, 233 aa, Artificial Sequence
  Synthetic peptide
DIEKLCKKAEEEAKEAQEKADELRQRHPDSQAAEDAEDLANLAVAAVLTACLLAQEHPNADIAKLCIKAASEAAEAASKAAELAQRHPDSQAARDAIKLASQAARAVILAIMLAAENPNADIAKLCIKAASEAAEAASKAAELAQRHPDSQAARDAIKLASQAAEAVERAIWLAAENPNADIAKKCIKAASEAAEEASKAAEEAQRHPDSQKARDEIKEASQKAEEVKERCKS

SEQ ID NO: 12 - protein, 233 aa, Artificial Sequence
  Synthetic peptide
DIEKLCKKAEEEAKEAQEKADELRQRHPDSQAAEDAEDLANEAEAAVLAACSLAQEHPNADIAKLCIKAASEAAEAASKAAELAQRHPDSQAARDAIKLASQAARAVILAIMLAAENPNADIAKLCIKAASEAAEAASKAAELAQRHPDSQAARDAIKLASQAAEAVERAIWLAAENPNADIAKKCIKAASEAAEEASKAAEEAQRHPDSQKARDEIKEASQKAEEVKERCKS

SEQ ID NO: 13 - protein, 21 aa, Artificial Sequence
  Synthetic peptide
GELGRLVYLLDGPGYDPIHSD

SEQ ID NO: 14 - protein, 21 aa, Artificial Sequence
  Synthetic peptide
GELDELVYLLDGPGYDPIHSD

SEQ ID NO: 15 - protein, 21 aa, Artificial Sequence
  Synthetic peptide
GELGELVYLLDGPGYDPIHSD

SEQ ID NO: 16 - protein, 21 aa, Artificial Sequence
  Synthetic peptide
GELDRLVYLLDGPGYDPIHSD

SEQ ID NO: 17 - protein, 33 aa, Artificial Sequence
  Synthetic peptide
GELDELVYLLDGPGYDPIHSDVVTRGGSHLFNF

SEQ ID NO: 18 - protein, 7 aa, Artificial Sequence
  Synthetic peptide
LKGSSGG

SEQ ID NO: 19 - skipped

SEQ ID NO: 20 - protein, 15 aa, Artificial Sequence
  Synthetic peptide
DLANLAVAAVLTACL

SEQ ID NO: 21 - protein, 8 aa, Artificial Sequence
  Synthetic peptide
RAVILAIM

SEQ ID NO: 22 - protein, 8 aa, Artificial Sequence
  Synthetic peptide
RAIWLAAE

SEQ ID NO: 23 - protein, 27 aa, Artificial Sequence
  Synthetic peptide
QAAEDAEDLANLAVAAVLTACLLAQEH

SEQ ID NO: 24 - protein, 25 aa, Artificial Sequence
  Synthetic peptide
QAARDAIKLASQAARAVILAIMLAA

SEQ ID NO: 25 - protein, 26 aa, Artificial Sequence
  Synthetic peptide
QAARDAIKLASQAAEAVERAIWLAAE

SEQ ID NO: 26 - protein, 26 aa, Artificial Sequence
  Synthetic peptide
IEKLCKKAEEEAKEAQEKADELRQRH

SEQ ID NO: 27 - protein, 26 aa, Artificial Sequence
  Synthetic peptide
DIAKLCIKAASEAAEAASKAAELAQR

SEQ ID NO: 28 - protein, 26 aa, Artificial Sequence
  Synthetic peptide
DIAKLCIKAASEAAEAASKAAELAQR

SEQ ID NO: 29 - protein, 50 aa, Artificial Sequence
  Synthetic peptide
DIAKKCIKAASEAAEEASKAAEEAQRHPDSQKARDEIKEASQKAEEVKER

SEQ ID NO: 30 - protein, 194 aa, Artificial Sequence
  Synthetic peptide
MAKGSVVIVGRINLSGDTAYSQQTRGAAGTAATSATGRDKNQVDGEVQVLSTATQSFLATCVNGVCWTVYHGAGSKTLAGPKGPITQMYTNVDQDLVGWPAPPGARSMTPCTCGSSDLYLVTRHADVIPVRRRGDSRGSLLSPRPVSYLKGSSGGPLLCPSGHVVGIFRAAVCTRGVAKAVDFIPVESMETTMR

SEQ ID NO: 31 - protein, 197 aa, Artificial Sequence
  Synthetic peptide
MKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSSGGPLLCPAGHAVGIFRAAVSTRGVAKAVDFIPVESLETTMRSP

SEQ ID NO: 32 - protein, 197 aa, Artificial Sequence
  Synthetic peptide
MKKKGSVVIVGRINLSGDTAYSQQTRGLEGCQETSQTGRDKNQVEGEVQVVSTATQSFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSSGGPLLCPAGHAVGIFRAAVSTRGVAKAVDFIPVESLETTMRSP

SEQ ID NO: 33 - protein, 197 aa, Artificial Sequence
  Synthetic peptide
MKKKGSVVIVGRINLSGDTAYSQQTRGELGCQETSQTGRDKNQVEGEVQVVSTATQSFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSSGGPLLCPAGHAVGIFRAAVSTRGVAKAVDFIPVESLETTMRSP

SEQ ID NO: 34 - protein, 197 aa, Artificial Sequence
  Synthetic peptide
MKKKGSVVIVGRINLSGDTAYSQQTRGLLGCQETSQTGRDKNQVEGEVQVVSTATQSFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSSGGPLLCPAGHAVGIFRAAVSTRGVAKAVDFIPVESLETTMRSP

SEQ ID NO: 35 - protein, 197 aa, Artificial Sequence
  Synthetic peptide
MKKKGSVVIVGRINLSGDTAYSQQTRGLLGCIETSQTGRDKNQVEGEVQVVSTATQSFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSSGGPLLCPAGHAVGIFRAAVSTRGVAKAVDFIPVESLETTMRSP

SEQ ID NO: 36 - protein, 197 aa, Artificial Sequence
  Synthetic peptide
MKKKGSVVIVGRINLSGDTAYSQQTRGLLGCIITSQTGRDKNQVEGEVQVVSTATQSFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSSGGPLLCPAGHAVGIFRAAVSTRGVAKAVDFIPVESLETTMRSP

SEQ ID NO: 37 - protein, 197 aa, Artificial Sequence
  Synthetic peptide
MKKKGSVVIVGRINLSGDTAYSQQTRGLEGCIETSQTGRDKNQVEGEVQVVSTATQSFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSSGGPLLCPAGHAVGIFRAAVSTRGVAKAVDFIPVESLETTMRSP

SEQ ID NO: 38 - protein, 197 aa, Artificial Sequence
  Synthetic peptide
MKKKGSVVIVGRINLSGDTAYSQQTRGEEGCQETSQTGRDKNQVEGEVQVVSTATQSFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSSGGPLLCPAGHAVGIFRAAVSTRGVAKAVDFIPVESLETTMRSP

SEQ ID NO: 39 - protein, 731 aa, Artificial Sequence
  Synthetic peptide
MGELDELVYLLDGPGYDPIHSDGSGTGSGTGSGTGSGTGDVYRFAEPDSEENIIFEENMQPKAGIPIIKAGTVIKLIERLTYHMYADPNFVRTFLTTYRSFCKPQELLSLIIERFEIPEPEPTEADRIAIENGDQPLSAELKRFRKEYIQPVQLRVLNVCRHWVEHHFYDFERDAYLLQRMEEFIGTVRGKAMKKWVESITKIIQRKKIARDNGPGHNITFQSSPPTVEWHISRPGHIETFDLLTLHPIEIARQLTLLESDLYRAVQPSELVGSVWTKEDKEINSPNLLKMIRHTTNLTLWFEKCIVETENLEERVAVVSRIIEILQVFQELNNFNGVLEVVSAMNSSPVYRLDHTFEQIPSRQKKILEEAHELSEDHYKKYLAKLRSINPPCVPFFGIYLTNILKTEEGNPEVLKRHGKELINFSKRRKVAEILGEIQQYQNQPYCLRVESDIKRFFENLNPMGNSMEKEFTDYLFNKSLEIEPGSGTGSGMAKGSVVIVGRINLSGDTAYSQQTRGLLGIIITSLTGRDKNQVDGEVQVLSTATQSFLATCVNGVCWTVYHGAGSKTLAGPKGPITQMYTNVDQDLVGWPAPPGARSMTPCTCGSSDLYLVTRHADVIPVRRRGDSRGSLLSPRPVSYLKGSSGGPLLCPSGHVVGIFRAAVCTRGVAKAVDFIPVESMETTMRGSGTGSGGSGTGDYKDDDDKQHKLRKLNPPDESGPGCMSCKCVLS

SEQ ID NO: 40 - protein, 25 aa, Artificial Sequence
  Synthetic peptide
  MISC_FEATURE (1)..(1): fluorescein (FAM), connected by a flexible glycine and serine linker, fused to the N-terminus of ANR
GSGSGELDELVYLLDGPGYDPIHSD

SEQ ID NO: 41 - protein, 6 aa, Artificial Sequence
  Synthetic peptide
LLGIII

SEQ ID NO: 42 - protein, 6 aa, Artificial Sequence
  Synthetic peptide
EEGCQE

SEQ ID NO: 43 - protein, 6 aa, Artificial Sequence
  Synthetic peptide
EEGCQE

SEQ ID NO: 44 - protein, 6 aa, Artificial Sequence
  Synthetic peptide
LEGCIE

SEQ ID NO: 45 - protein, 6 aa, Artificial Sequence
  Synthetic peptide
LLGCII

SEQ ID NO: 46 - protein, 6 aa, Artificial Sequence
  Synthetic peptide
LLGCIE

SEQ ID NO: 47 - protein, 6 aa, Artificial Sequence
  Synthetic peptide
LLGCQE

SEQ ID NO: 48 - protein, 6 aa, Artificial Sequence
  Synthetic peptide
ELGCQE

SEQ ID NO: 49 - protein, 6 aa, Artificial Sequence
  Synthetic peptide
LEGCQE

SEQ ID NO: 50 - protein, 261 aa, Artificial Sequence
  Synthetic peptide
MHHHHHHGSGTGSGELGRLVYLLDGPGYDPIHSDGTGSSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPR

SEQ ID NO: 51 - protein, 387 aa, Artificial Sequence
  Synthetic peptide
MDKDCEMKRTTLDSPLGKLELSGCEQGLHEIKLLGKGTSAADAVEVPAPAAVLGGPEPLMQATAWLNAYFHQPEAIEEFPVPALHHPVFQQESFTRQVLWKLLKVVKFGEVISYQQLAALAGNPAATAAVKTALSGNPVPILIPCHRVVSSSGAVGGYEGGLAVKEWLLAHEGHRLGKPGLGGTGTAKGSVVIVGRINLSGDTAYSQQTRGLLGIIITSATGRDKNQVDGEVQVLSTATQSFLATCVNGVCWTVYHGAGSKTLAGPKGPITQMYTNVDQDLVGWPAPPGARSMTPCTCGSSDLYLVTRHADVIPVRRRGDSRGSLLSPRPVSYLKGSSGGPLLCPSGHVVGIFRAAVCTRGVAKAVDFIPVESMETTMRGSHHHHHH

SEQ ID NO: 52 - protein, 230 aa, Artificial Sequence
  Synthetic peptide
MAGGLNDIFEAQKIEWHEDTGGSSHHHHHHGSGSGSMAKGSVVIVGRINLSGDTAYSQQTRGLLGCIITSATGRDKNQVDGEVQVLSTATQSFLATCVNGVCWTVYHGAGSKTLAGPKGPITQMYTNVDQDLVGWPAPPGARSMTPCTCGSSDLYLVTRHADVIPVRRRGDSRGSLLSPRPVSYLKGSAGGPLLCPSGHVVGIFRAAVCTRGVAKAVDFIPVESMETTMR

SEQ ID NO: 53 - protein, 230 aa, Artificial Sequence
  Synthetic peptide
MAGGLNDIFEAQKIEWHEDTGGSSHHHHHHGSGSGSMAKGSVVIVGRINLSGDTAYSQQTRGLLGCIITSATGRDKNQVDGEVQVLSTATQSFLATCVNGVCWTVYHGAGSKTLAGPKGPITQMYTNVDQDLVGWPAPPGARSMTPCTCGSSDLYLVTRHADVIPVRRRGDSRGSLLSPRPVSYLKGSSGGPLLCPSGHVVGIFRAAVCTRGVAKAVDFIPVESMETTMR

SEQ ID NO: 54 - protein, 215 aa, Artificial Sequence
  Synthetic peptide
MAGGSSHHHHHHGSGSGSMKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSP

SEQ ID NO: 55 - protein, 215 aa, Artificial Sequence
  Synthetic peptide
MAGGSSHHHHHHGSGSGSMKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSSGGPLLCPAGHAVGIFRAAVSTRGVAKAVDFIPVESLETTMRSP

SEQ ID NO: 56 - protein, 214 aa, Artificial Sequence
  Synthetic peptide
MGGSSHHHHHHGSGSGSMKKKGSVVIVGRINLSGDTAYSQQTRGLEGCQETSQTGRDKNQVEGEVQVVSTATQSFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSSGGPLLCPAGHAVGIFRAAVSTRGVAKAVDFIPVESLETTMRSP

SEQ ID NO: 57 - protein, 519 aa, Artificial Sequence
  Synthetic peptide
MVGRNSAIAAGVCGALFIGYCIYFDRKRRSDPNFGSGGSMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKGSGSTGTSGSGSGTGSGSGTGMKKKGSVVIVGRINLSGDTAYSQQTRGLEGCQETSQTGRDKNQVEGEVQVVSTATQSFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSSGGPLLCPAGHAVGIFRAAVSTRGVAKAVDFIPVESLETTMRSPGSGTGSGTSGSTGTGSTGDYKDDDDK

SEQ ID NO: 58 - protein, 519 aa, Artificial Sequence
  Synthetic peptide
MVGRNSAIAAGVCGALFIGYCIYFDRKRRSDPNFGSGGSMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKGSGSTGTSGSGSGTGSGSGTGMKKKGSVVIVGRINLSGDTAYSQQTRGELGCQETSQTGRDKNQVEGEVQVVSTATQSFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSSGGPLLCPAGHAVGIFRAAVSTRGVAKAVDFIPVESLETTMRSPGSGTGSGTSGSTGTGSTGDYKDDDDK

SEQ ID NO: 59 - protein, 519 aa, Artificial Sequence
  Synthetic peptide
MVGRNSAIAAGVCGALFIGYCIYFDRKRRSDPNFGSGGSMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKGSGSTGTSGSGSGTGSGSGTGMKKKGSVVIVGRINLSGDTAYSQQTRGLLGCQETSQTGRDKNQVEGEVQVVSTATQSFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSSGGPLLCPAGHAVGIFRAAVSTRGVAKAVDFIPVESLETTMRSPGSGTGSGTSGSTGTGSTGDYKDDDDK

SEQ ID NO: 60 - protein, 519 aa, Artificial Sequence
  Synthetic peptide
MVGRNSAIAAGVCGALFIGYCIYFDRKRRSDPNFGSGGSMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKGSGSTGTSGSGSGTGSGSGTGMKKKGSVVIVGRINLSGDTAYSQQTRGLLGCIETSQTGRDKNQVEGEVQVVSTATQSFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSSGGPLLCPAGHAVGIFRAAVSTRGVAKAVDFIPVESLETTMRSPGSGTGSGTSGSTGTGSTGDYKDDDDK

SEQ ID NO: 61 - protein, 519 aa, Artificial Sequence
  Synthetic peptide
MVGRNSAIAAGVCGALFIGYCIYFDRKRRSDPNFGSGGSMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKGSGSTGTSGSGSGTGSGSGTGMKKKGSVVIVGRINLSGDTAYSQQTRGLLGCIITSQTGRDKNQVEGEVQVVSTATQSFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSSGGPLLCPAGHAVGIFRAAVSTRGVAKAVDFIPVESLETTMRSPGSGTGSGTSGSTGTGSTGDYKDDDDK

SEQ ID NO: 62 - protein, 519 aa, Artificial Sequence
  Synthetic peptide
MVGRNSAIAAGVCGALFIGYCIYFDRKRRSDPNFGSGGSMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKGSGSTGTSGSGSGTGSGSGTGMKKKGSVVIVGRINLSGDTAYSQQTRGLEGCIETSQTGRDKNQVEGEVQVVSTATQSFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSSGGPLLCPAGHAVGIFRAAVSTRGVAKAVDFIPVESLETTMRSPGSGTGSGTSGSTGTGSTGDYKDDDDK

SEQ ID NO: 63 - protein, 519 aa, Artificial Sequence
  Synthetic peptide
MVGRNSAIAAGVCGALFIGYCIYFDRKRRSDPNFGSGGSMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKGSGSTGTSGSGSGTGSGSGTGMKKKGSVVIVGRINLSGDTAYSQQTRGEEGCQETSQTGRDKNQVEGEVQVVSTATQSFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSSGGPLLCPAGHAVGIFRAAVSTRGVAKAVDFIPVESLETTMRSPGSGTGSGTSGSTGTGSTGDYKDDDDK

SEQ ID NO: 64 - protein, 445 aa, Artificial Sequence
  Synthetic peptide
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKGSGTGDYKDDDDKKKKGSVVIVGRINLSGDTAYAQQTRGLEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSP

SEQ ID NO: 65 - protein, 345 aa, Artificial Sequence
  Synthetic peptide
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGSGEQKLISEEDLGSGTGSGTGSGTGTTSGTGTGGSTGGELDELVYLLDGPGYDPIHSDGSGTGSGTGSGTGTTSGTGTGGSTGGELDELVYLLDGPGYDPIHSD

SEQ ID NO: 66 - protein, 362 aa, Artificial Sequence
  Synthetic peptide
MGCGCSSHPEDDGTGSGTGSMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKGSGSEQKLISEEDLGSGTGSGTGSGTGTTSGTGTGGSTGGELDELVYLLDGPGYDPIHSDGSGTGSGTGSGTGTTSGTGTGGSTGGELDELVYLLDGPGYDPIHSD

SEQ ID NO: 67 - protein, 366 aa, Artificial Sequence
  Synthetic peptide
MDPKKKRKVDPKKKRKVDPKKKRKVGSGSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLNSGSGEQKLISEEDLGSGTGSGTGSGTGTTSGTGTGGSTGGELDELVYLLDGPGYDPIHSDGSGTGSGTGSGTGTTSGTGTGGSTGGELDELVYLLDGPGYDPIHSD

SEQ ID NO: 68 - protein, 731 aa, Artificial Sequence; identical to SEQ ID NO: 39
  Synthetic peptide

SEQ ID NO: 69 - protein, 721 aa, Artificial Sequence
  Synthetic peptide
MAPPNLWAAQRYGRELRRMADEGEGSFKGSGTGSGTGSGTGSGTGDVYRFAEPDSEENIIFEENMQPKAGIPIIKAGTVIKLIERLTYHMYADPNFVRTFLTTYRSFCKPQELLSLIIERFEIPEPEPTEADRIAIENGDQPLSAELKRFRKEYIQPVQLRVLNVCRHWVEHHFYDFERDAYLLQRMEEFIGTVRGKAMKKWVESITKIIQRKKIARDNGPGHNITFQSSPPTVEWHISRPGHIETFDLLTLHPIEIARQLTLLESDLYRAVQPSELVGSVWTKEDKEINSPNLLKMIRHTTNLTLWFEKCIVETENLEERVAVVSRIIEILQVFQELNNFNGVLEVVSAMNSSPVYRLDHTFEQIPSRQKKILEEAHELSEDHYKKYLAKLRSINPPCVPFFGIYLTNILKTEEGNPEVLKRHGKELINFSKRRKVAEILGEIQQYQNQPYCLRVESDIKRFFENLNPMGNSMEKEFTDYLFNKSLEIEPGSGMAKGSVVIVGRINLSGDTAYSQQTRGLLGIIITSLTGRDKNQVDGEVQVLSTATQSFLATCVNGVCWTVYHGAGSKTLAGPKGPITQMYTNVDQDLVGWPAPPGARSMTPCTCGSSDLYLVTRHADVIPVRRRGDSRGSLLSPRPVSYLKGSSGGPLLCPSGHVVGIFRAAVCTRGVAKAVDFIPVESMETTMRDYKDDDDKQHKLRKLNPPDESGPGCMSCKCVLS

SEQ ID NO: 70 - protein, 1506 aa, Artificial Sequence
  Synthetic peptide
MDYKDDDDKDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSSNRELVVDFLSYKLSQKGYSWSQFSDVEENRTEAPEGTESEMETPSAINGNPSWHLADSPAVNGATGHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGNNGSGTASGTGSGTGSATGSGTVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVTGSGTAPPNLWAAQRYGRELRRMADEGEGSFK

SEQ ID NO: 71 - protein, 745 aa, Artificial Sequence
  Synthetic peptide
MKKKGSVVIVGRINLSGDTAYSQQTRGLEGCQETSQTGRDKNQVEGEVQVVSTATQSFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSSGGPLLCPAGHAVGIFRAAVSTRGVAKAVDFIPVESLETTMRSPGSGTGSGEQKLISEEDLEFSSAAGTSDALDDFDLDMLGSDALDDFDLDMLGSDALDDFDLDMLGSDALDDFDLDMLGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 72 - protein, 476 aa, Artificial Sequence
  Synthetic peptide
MPKKKRKVGSMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGSGGSGTGSGTGSGTGTTSGTGTGGSTGGELDELVYLLDGPGYDPIHSDGSGTGSGTGSGTGTTSGTGTGGSTGGELDELVYLLDGPGYDPIHSDGSGATNFSLLKQAGDVEENPGPSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 73 - protein, 1152 aa, Artificial Sequence
  Synthetic peptide
MKLLSSIEQACDICRLKKLKCSKEKPKCAKCLKNNWECRYSPKTKRSPLTRAHLTEVESRLERLEQLFLLIFPREDLDMILKMDSLQDIKALLGTPAAASTAGSGGMAKGSVVIVGRINLSGDTAYSQQTRGLEGCQETSQTGRDKNQVEGEVQVVSTATQSFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSPGSGATNFSLLKQAGDVEENPGPGALSGMGELDELVYLLDGPGYDPIHSDGVLSGSGTGSGTGSGTGTTSGTGTGGSTGEQKLISEEDLGSGSSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLNGSGSDALDDFDLDMLGSDALDDFDLDMLGSDALDDFDLDMLGSDALDDFDLDMLGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 74 - protein, 1092 aa, Artificial Sequence
  Synthetic peptide
MKLLSSIEQACDICRLKKLKCSKEKPKCAKCLKNNWECRYSPKTKRSPLTRAHLTEVESRLERLEQLFLLIFPREDLDMILKMDSLQDIKALLGTPAAASTLEGGGSAGSGGKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSPGSGATNFSLLKQAGDVEENPGPMSSDEEEARELIERAKEAAERAQEAAERTGDPRVRELARELKRLAQEAAEEVKRDPSSSDVNEALKLIVEAIEAAVDALEAAERTGDPEVRELARELVRLAVEAAEEVQRNPSSSDVNEALHSIVYAIEAAIFALEAAERTGDPEVRELARELVRLAVEAAEEVQRNPSSRNVEHALMRIVLAIYLAEENLREAEESGDPEKREKARERVREAVERAEEVQRDPSGWLNHEQKLISEEDLDALDDFDLDMLGSDALDDFDLDMLGSDALDDFDLDMLGSDALDDFDLDMLGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 75 - protein, 233 aa, Artificial Sequence
  Synthetic peptide
MSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 76 - protein, 183 aa, Artificial Sequence
  Synthetic peptide
GTACGTTCTCTATCACTGATAGTTTAAGAGCTATGCTGGAAACAGCATAGCAAGTTTAAATAAGGCTAGTCCGTTATCAACTTGAAAAAGTGGCACCGAGTCGGTGCGGGAGCACATGAGGATCACCCATGTGCGACTCCCACAGTCACTGGGGAGTCTTCCCTTTTTTTGTTTTTTATGTCT

SEQ ID NO: 77 - protein, 21 aa, Artificial Sequence
  Synthetic peptide
GTACGTTCTCTATCACTGATA

SEQ ID NO: 78 - protein, 309 aa, Artificial Sequence
  Synthetic peptide
MGHHHHHHHHHHGSLQDSEVNQEAKPEVKPEVKPETHINLKVSDGSSEIFFKIKKTTPLRRLMEAFAKRQGKEMDSLRFLYDGIRIQADQAPEDLDMEDNDIIEAHREQIGGMKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSP

SEQ ID NO: 79 - protein, 341 aa, Artificial Sequence
  Synthetic peptide
MGHHHHHHHHHHGSLQDSEVNQEAKPEVKPEVKPETHINLKVSDGSSEIFFKIKKTTPLRRLMEAFAKRQGKEMDSLRFLYDGIRIQADQAPEDLDMEDNDIIEAHREQIGGSSDEEEARELIERAKEAAERAQEAAERTGDPRVRELARELKRLAQEAAEEVKRDPSSSDVNEALKLIVEAIEAAVDALEAAERTGDPEVRELARELVRLAVEAAEEVQRNPSSSDVNEALHSIVYAIEAAIFALEAAERTGDPEVRELARELVRLAVEAAEEVQRNPSSRNVEHALMRIVLAIYLAEENLREAEESGDPEKREKARERVREAVERAEEVQRDPSGWLNH

SEQ ID NO: 80 - protein, 341 aa, Artificial Sequence
  Synthetic peptide
MGHHHHHHHHHHGSLQDSEVNQEAKPEVKPEVKPETHINLKVSDGSSEIFFKIKKTTPLRRLMEAFAKRQGKEMDSLRFLYDGIRIQADQAPEDLDMEDNDIIEAHREQIGGSSDEEEARELIERAKEAAERAQEAAERTGDPRVRELARELKRLAQEAAEEVKRDPSSSDVNEALKLIVEAIEAAVDALEAAERTGDPEVRELARELVRLAVEAAEEVQRNPSSSDVNEALLSIVIAIEAAVHALEAAERTGDPEVRELARELVRLAVEAAEEVQRNPSSREVEHALMKIVLAIYEAEESLREAEESGDPEKREKARERVREAVERAEEVQRDPSGWLNH

SEQ ID NO: 81 - protein, 233 aa, Artificial Sequence
  Synthetic peptide
MAGGLNDIFEAQKIEWHEDTGGSSHHHHHHGSGSGSMKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSP

SEQ ID NO: 82 - protein, 233 aa, Artificial Sequence
  Synthetic peptide
MAGGLNDIFEAQKIEWHEDTGGSSHHHHHHGSGSGSMKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSSGGPLLCPAGHAVGIFRAAVSTRGVAKAVDFIPVESLETTMRSP

SEQ ID NO: 83 - protein, 291 aa, Artificial Sequence
  Synthetic peptide
KDNSSTIEGRYPYDVPDYALQASGGGGSGGGGSGGGGSASHMDIEKLCKKAEEEAKEAQEKADELRQRHPDSQAAEDAEDLANEAEAAVLAACSLAQEHPNADIAKLCIKAASEAAEAASKAAELAQRHPDSQAARDAIKLASQAARAVILAIMLAAENPNADIAKLCIKAASEAAEAASKAAELAQRHPDSQAARDAIKLASQAAEAVERAIWLAAENPNADIAKKCIKAASEAAEEASKAAEEAQRHPDSQKARDEIKEASQKAEEVKERCKSLEGGGSEQKLISEEDL

SEQ ID NO: 84 - protein, 291 aa, Artificial Sequence
  Synthetic peptide
KDNSSTIEGRYPYDVPDYALQASGGGGSGGGGSGGGGSASHMDIEKLCKKAEEEAKEAQEKADELRQRHPDSQAAEDAEDLANLAVAAVLTACLLAQEHPNADIAKLCIKAASEAAEAASKAAELAQRHPDSQAARDAIKLASQAARAVILAIMLAAENPNADIAKLCIKAASEAAEAASKAAELAQRHPDSQAARDAIKLASQAAEAVERAIWLAAENPNADIAKKCIKAASEAAEEASKAAEEAQRHPDSQKARDEIKEASQKAEEVKERCKSLEGGGSEQKLISEEDL

SEQ ID NO: 85 - protein, 287 aa, Artificial Sequence
  Synthetic peptide
KDNSSTIEGRYPYDVPDYALQASGGGGSGGGGSGGGGSASHMSSDEEEARELIERAKEAAERAQEAAERTGDPRVRELARELKRLAQEAAEEVKRDPSSSDVNEALKLIVEAIEAAVDALEAAERTGDPEVRELARELVRLAVEAAEEVQRNPSSSDVNEALLTIVIAIEAAVNALEAAERTGDPEVRELARELVRLAVEAAEEVQRNPSSREVNIALWKIVLAIQEAVESLREAEESGDPEKREKARERVREAVERAEEVQRDPSGWLNHLEGGGSEQKLISEEDL

SEQ ID NO: 86 - protein, 287 aa, Artificial Sequence
  Synthetic peptide
KDNSSTIEGRYPYDVPDYALQASGGGGSGGGGSGGGGSASHMSSDEEEARELIERAKEAAERAQEAAERTGDPRVRELARELKRLAQEAAEEVKRDPSSSDVNEALKLIVEAIEAAVDALEAAERTGDPEVRELARELVRLAVEAAEEVQRNPSSSDVNEALLSIVIAIEAAVHALEAAERTGDPEVRELARELVRLAVEAAEEVQRNPSSREVEHALMKIVLAIYEAEESLREAEESGDPEKREKARERVREAVERAEEVQRDPSGWLNHLEGGGSEQKLISEEDL

SEQ ID NO: 87 - protein, 287 aa, Artificial Sequence
  Synthetic peptide
KDNSSTIEGRYPYDVPDYALQASGGGGSGGGGSGGGGSASHMSSDEEEARELIERAKEAAERAQEAAERTGDPRVRELARELKRLAQEAAEEVKRDPSSSDVNEALKLIVEAIEAAVDALEAAERTGDPEVRELARELVRLAVEAAEEVQRNPSSSDVNEALHSIVYAIEAAIFALEAAERTGDPEVRELARELVRLAVEAAEEVQRNPSSRNVEHALMRIVLAIYLAEENLREAEESGDPEKREKARERVREAVERAEEVQRDPSGWLNHLEGGGSEQKLISEEDL

SEQ ID NO: 88 - protein, 487 aa, Artificial Sequence
  Synthetic peptide
MSSDEEEARELIERAKEAAERAQEAAERTGDPRVRELARELKRLAQEAAEEVKRDPSSSDVNEALKLIVEAIEAAVDALEAAERTGDPEVRELARELVRLAVEAAEEVQRNPSSSDVNEALHSIVYAIEAAIFALEAAERTGDPEVRELARELVRLAVEAAEEVQRNPSSRNVEHALMRIVLAIYLAEENLREAEESGDPEKREKARERVREAVERAEEVQRDPSGWLNHEQKLISEEDLGSGTGSGTMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 89 - protein, 520 aa, Artificial Sequence
  Synthetic peptide
MVGRNSAIAAGVCGALFIGYCIYFDRKRRSDPNFSSDEEEARELIERAKEAAERAQEAAERTGDPRVRELARELKRLAQEAAEEVKRDPSSSDVNEALKLIVEAIEAAVDALEAAERTGDPEVRELARELVRLAVEAAEEVQRNPSSSDVNEALLSIVIAIEAAVHALEAAERTGDPEVRELARELVRLAVEAAEEVQRNPSSREVEHALMKIVLAIYEAEESLREAEESGDPEKREKARERVREAVERAEEVQRDPSGWLNHEQKLISEEDLGSGTGSGTMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 90 - protein, 625 aa, Artificial Sequence
  Synthetic peptide
MSSDEEEARELIERAKEAAERAQEAAERTGDPRVRELARELKRLAQEAAEEVKRDPSSSDVNEALKLIVEAIEAAVDALEAAERTGDPEVRELARELVRLAVEAAEEVQRNPSSSDVNEALLSIVIAIEAAVHALEAAERTGDPEVRELARELVRLAVEAAEEVQRNPSSREVEHALMKIVLAIYEAEESLREAEESGDPEKREKARERVREAVERAEEVQRDPSGWLNHEQKLISEEDLGSGTGSGTMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKGSGTGSGSGEPQQSFSEAQQQLCNTRQEVNELRKLLEEERDQRVAAENALSVAEEQIRRLEHSEWDSSRTPIIGSCGTQEQALLIDLTSNSCRRTRSGVGWKRVLRSLCHSRTRVPLLAAIYFLMIHVLLILCFTGHL

SEQ ID NO: 91 - protein, 511 aa, Artificial Sequence
  Synthetic peptide
MDPKKKRKVDPKKKRKVDPKKKRKVSSDEEEARELIERAKEAAERAQEAAERTGDPRVRELARELKRLAQEAAEEVKRDPSSSDVNEALKLIVEAIEAAVDALEAAERTGDPEVRELARELVRLAVEAAEEVQRNPSSSDVNEALLSIVIAIEAAVHALEAAERTGDPEVRELARELVRLAVEAAEEVQRNPSSREVEHALMKIVLAIYEAEESLREAEESGDPEKREKARERVREAVERAEEVQRDPSGWLNHEQKLISEEDLGSGTGSGTMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 92 - protein, 487 aa, Artificial Sequence
  Synthetic peptide
MSSDEEEARELIERAKEAAERAQEAAERTGDPRVRELARELKRLAQEAAEEVKRDPSSSDVNEALKLIVEAIEAAVDALEAAERTGDPEVRELARELVRLAVEAAEEVQRNPSSSDVNEALLSIVIAIEAAVHALEAAERTGDPEVRELARELVRLAVEAAEEVQRNPSSREVEHALMKIVLAIYEAEESLREAEESGDPEKREKARERVREAVERAEEVQRDPSGWLNHEQKLISEEDLGSGTGSGTMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 93 - protein, 445 aa, Artificial Sequence
  Synthetic peptide
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKGSGTGDYKDDDDKKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSP

SEQ ID NO: 94 - protein, 482 aa, Artificial Sequence
  Synthetic peptide
MVGRNSAIAAGVCGALFIGYCIYFDRKRRSDPNFGSGMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKGSGTGDYKDDDDKKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSP

SEQ ID NO: 95 - protein, 583 aa, Artificial Sequence
  Synthetic peptide
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKGSGTGDYKDDDDKKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSPGSGTGSGSGEPQQSFSEAQQQLCNTRQEVNELRKLLEEERDQRVAAENALSVAEEQIRRLEHSEWDSSRTPIIGSCGTQEQALLIDLTSNSCRRTRSGVGWKRVLRSLCHSRTRVPLLAAIYFLMIHVLLILCFTGHL

SEQ ID NO: 96 - protein, 473 aa, Artificial Sequence
  Synthetic peptide
MDPKKKRKVDPKKKRKVDPKKKRKVGSGMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKGSGTGDYKDDDDKKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSP

SEQ ID NO: 97 - protein, 464 aa, Artificial Sequence
  Synthetic peptide
MGCGCSSHPEDDGSGTGSGMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKGSGTGDYKDDDDKKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSP

SEQ ID NO: 98 - protein, 453 aa, Artificial Sequence
  Synthetic peptide
MKKKGSVVIVGRINLSGDTAYSQQTRGLEGCQETSQTGRDKNQVEGEVQVVSTATQSFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSSGGPLLCPAGHAVGIFRAAVSTRGVAKAVDFIPVESLETTMRSPGSGTGSGMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKGSGTGDYKDDDDK

SEQ ID NO: 99 - protein, 514 aa, Artificial Sequence
  Synthetic peptide
MDPKKKRKVDPKKKRKVDPKKKRKVGSGSSDEEEARELIERAKEAAERAQEAAERTGDPRVRELARELKRLAQEAAEEVKRDPSSSDVNEALKLIVEAIEAAVDALEAAERTGDPEVRELARELVRLAVEAAEEVQRNPSSSDVNEALHSIVYAIEAAIFALEAAERTGDPEVRELARELVRLAVEAAEEVQRNPSSRNVEHALMRIVLAIYLAEENLREAEESGDPEKREKARERVREAVERAEEVQRDPSGWLNHEQKLISEEDLGSGTGSGTMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 100 - protein, 364 aa, Artificial Sequence
  Synthetic peptide
MGELDELVYLLDGPGYDPIHSDGSGTGSGTGSGTGTTSGTGTGGSTGGELDELVYLLDGPGYDPIHSDGSGTGSGTGSGTGTTSGTGTGGSTGEQKLISEEDLGSGSSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLNRKHKEKMSKDGKKKKKKSKTKCVIM

SEQ ID NO: 101 - protein, 907 aa, Artificial Sequence
  Synthetic peptide
MVGRNSAIAAGVCGALFIGYCIYFDRKRRSDPNFMSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLNSGSGEQKLISEEDLGSGTGSGTGSGTGTTSGTGTGGSTGGELDELVYLLDGPGYDPIHSDGSGTGSGTGSGTGTTSGTGTGGSTGGELDELVYLLDGPGYDPIHSDGSGATNFSLLKQAGDVEENPGPMSSDEEEARELIERAKEAAERAQEAAERTGDPRVRELARELKRLAQEAAEEVKRDPSSSDVNEALKLIVEAIEAAVDALEAAERTGDPEVRELARELVRLAVEAAEEVQRNPSSSDVNEALHSIVYAIEAAIFALEAAERTGDPEVRELARELVRLAVEAAEEVQRNPSSRNVEHALMRIVLAIYLAEENLREAEESGDPEKREKARERVREAVERAEEVQRDPSGWLNHEQKLISEEDLGSGTGSGTMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKRKHKEKMSKDGKKKKKKSKTKCVIM

SEQ ID NO: 102 - protein, 520 aa, Artificial Sequence
  Synthetic peptide
MVGRNSAIAAGVCGALFIGYCIYFDRKRRSDPNFSSDEEEARELIERAKEAAERAQEAAERTGDPRVRELARELKRLAQEAAEEVKRDPSSSDVNEALKLIVEAIEAAVDALEAAERTGDPEVRELARELVRLAVEAAEEVQRNPSSSDVNEALHSIVYAIEAAIFALEAAERTGDPEVRELARELVRLAVEAAEEVQRNPSSRNVEHALMRIVLAIYLAEENLREAEESGDPEKREKARERVREAVERAEEVQRDPSGWLNHEQKLISEEDLGSGTGSGTMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 103 - protein, 505 aa, Artificial Sequence
  Synthetic peptide
MDIEKLCKKAEEEAKEAQEKADELRQRHPDSQAAEDAEDLANLAVAAVLTACLLAQEHPNADIAKLCIKAASEAAEAASKAAELAQRHPDSQAARDAIKLASQAARAVILAIMLAAENPNADIAKLCIKAASEAAEAASKAAELAQRHPDSQAARDAIKLASQAAEAVERAIWLAAENPNADIAKKCIKAASEAAEEASKAAEEAQRHPDSQKARDEIKEASQKAEEVKERCKSEQKLISEEDLGSGSSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLNRKHKEKMSKDGKKKKKKSKTKCVIM

SEQ ID NO: 104 - protein, 478 aa, Artificial Sequence
  Synthetic peptide
MKKKGSVVIVGRINLSGDTAYSQQTRGLEGCQETSQTGRDKNQVEGEVQVVSTATQSFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSSGGPLLCPAGHAVGIFRAAVSTRGVAKAVDFIPVESLETTMRSPGSGTGSGMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKGSGTGDYKDDDDKQHKLRKLNPPDESGPGCMSCKCVLS

SEQ ID NO: 105 - protein, 480 aa, Artificial Sequence
  Synthetic peptide
MDIEKLCKKAEEEAKEAQEKADELRQRHPDSQAAEDAEDLANLAVAAVLTACLLAQEHPNADIAKLCIKAASEAAEAASKAAELAQRHPDSQAARDAIKLASQAARAVILAIMLAAENPNADIAKLCIKAASEAAEAASKAAELAQRHPDSQAARDAIKLASQAAEAVERAIWLAAENPNADIAKKCIKAASEAAEEASKAAEEAQRHPDSQKARDEIKEASQKAEEVKERCKSEQKLISEEDLGSGSSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 106 - protein, 444 aa, Artificial Sequence
  Synthetic peptide
MSSDEEEARELIERAKEAAERAQEAAERTGDPRVRELARELKRLAQEAAEEVKRDPSSSDVNEALKLIVEAIEAAVDALEAAERTGDPEVRELARELVRLAVEAAEEVQRNPSSSDVNEALHSIVYAIEAAIFALEAAERTGDPEVRELARELVRLAVEAAEEVQRNPSSRNVEHALMRIVLAIYLAEENLREAEESGDPEKREKARERVREAVERAEEVQRDPSGWLNHEQKLISEEDLGSGTGSGTRLLYPVSKYQQDQIVKEDSVEAVGAQLKVYHQQYQDKSREYDQLYEEYTRTSQELQMKRTAIEAFNETIKIFEEQGQTQEKCSKEYLERFRREGNEKEMQRILLNSERLKSRIAEIHESRTKLEQQLRAQASDNREIDKRMNSLKPDLMQLRKIRDQYLVWLTQKGARQKKINEWLGIKNETEDQYALMEDEDDLP

SEQ ID NO: 107 - protein, 771 aa, Artificial Sequence
  Synthetic peptide
MSSDEEEARELIERAKEAAERAQEAAERTGDPRVRELARELKRLAQEAAEEVKRDPSSSDVNEALKLIVEAIEAAVDALEAAERTGDPEVRELARELVRLAVEAAEEVQRNPSSSDVNEALHSIVYAIEAAIFALEAAERTGDPEVRELARELVRLAVEAAEEVQRNPSSRNVEHALMRIVLAIYLAEENLREAEESGDPEKREKARERVREAVERAEEVQRDPSGWLNHEQKLISEEDLEFSSAAGTSDALDDFDLDMLGSDALDDFDLDMLGSDALDDFDLDMLGSDALDDFDLDMLGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 108 - protein, 326 aa, Artificial Sequence
  Synthetic peptide
MSSDEEEARELIERAKEAAERAQEAAERTGDPRVRELARELKRLAQEAAEEVKRDPSSSDVNEALKLIVEAIEAAVDALEAAERTGDPEVRELARELVRLAVEAAEEVQRNPSSSDVNEALHSIVYAIEAAIFALEAAERTGDPEVRELARELVRLAVEAAEEVQRNPSSRNVEHALMRIVLAIYLAEENLREAEESGDPEKREKARERVREAVERAEEVQRDPSGWLNHEQKLISEEDLEFSSAAGTSGGGGGMDAKSLTAWSRTLVTFKDVFVDFTREEWKLLDTAQQIVYRNVMLENYKNLVSLGYQLTKPDVILRLEKGEEP

SEQ ID NO: 109 - protein, 1855 aa, Artificial Sequence
  Synthetic peptide
MKKKGSVVIVGRINLSGDTAYSQQTRGLEGCQETSQTGRDKNQVEGEVQVVSTATQSFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSSGGPLLCPAGHAVGIFRAAVSTRGVAKAVDFIPVESLETTMRSPHMSSAAGATMSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLNSSAAGATMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAYPYDVPDYASLGSGSPKKKRKVEDPKKKRKVDGIGSGSNG

SEQ ID NO: 110 - DNA, 21 bp, Artificial Sequence
  Synthetic oligonucleotide
gcgggtggtcggtagtgagtc

SEQ ID NO: 111 - DNA, 23 bp, Artificial Sequence
  Synthetic oligonucleotide
gcagacgcgaggaaggagggcgc

SEQ ID NO: 112 - DNA, 25 bp, Artificial Sequence
  Synthetic oligonucleotide
gcctctgggaggtcctgtccggctc

SEQ ID NO: 113 - DNA, 21 bp, Artificial Sequence
  Synthetic oligonucleotide
gtacagcagaagcctttagaa

SEQ ID NO: 114 - DNA, 21 bp, Artificial Sequence
  Synthetic oligonucleotide
gtggcatgctcacttcaggtg

SEQ ID NO: 115 - DNA, 21 bp, Artificial Sequence
  Synthetic oligonucleotide
gaagcctcgctggggaacgcc

SEQ ID NO: 116 - DNA, 21 bp, Artificial Sequence
  Synthetic oligonucleotide
gtacgttctctatcactgata

SEQ ID NO: 117 - DNA, 183 bp, Artificial Sequence
  Synthetic oligonucleotide
gcgggtggtcggtagtgagtcgtttaagagctatgctggaaacagcatagcaagtttaaataaggctagtccgttatcaacttgaaaaagtggcaccgagtcggtgcgggagcacatgaggatcacccatgtgcgactcccacagtcactggggagtcttccctttttttgttttttatgtct

SEQ ID NO: 118 - DNA, 196 bp, Artificial Sequence
  Synthetic oligonucleotide
gcagacgcgaggaaggagggcgcgtttaagagctatgctggaaacagcatagcaagtttaaataaggctagtccgttatcaacttgaaaaagtggcaccgagtcggtgcgggagcacatgaggatcacccatgtgccacgagcgacatgaggatcacccatgtcgctcgtgttccctttttttgttttttatgtct

SEQ ID NO: 119 - DNA, 187 bp, Artificial Sequence
  Synthetic oligonucleotide
gcctctgggaggtcctgtccggctcgtttaagagctatgctggaaacagcatagcaagtttaaataaggctagtccgttatcaacttgaaaaagtggcaccgagtcggtgcgggagcacatgaggatcacccatgtgcgactcccacagtcactggggagtcttccctttttttgttttttatgtct

SEQ ID NO: 120 - protein, 630 aa, Artificial Sequence
  Synthetic peptide
MPKKKRKVGSMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGSGGSGDIEKLCKKAEEEAKEAQEKADELRQRHPDSQAAEDAEDLANLAVAAVLTACLLAQEHPNADIAKLCIKAASEAAEAASKAAELAQRHPDSQAARDAIKLASQAARAVILAIMLAAENPNADIAKLCIKAASEAAEAASKAAELAQRHPDSQAARDAIKLASQAAEAVERAIWLAAENPNADIAKKCIKAASEAAEEASKAAEEAQRHPDSQKARDEIKEASQKAEEVKERCKSEQKLISEEDLGSGATNFSLLKQAGDVEENPGPSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 121 - DNA, 146 bp, Artificial Sequence
  Synthetic oligonucleotide
gcgggtggtcggtagtgagtcgtttaagagctatgctggaaacagcatagcaagtttaaataaggctagtccgttatcaacttgaaaaagtggcaccgagtcggtgcctgaatgcctgcgagcatctttttttgttttttatgtct

SEQ ID NO: 122 - DNA, 148 bp, Artificial Sequence
  Synthetic oligonucleotide
gcagacgcgaggaaggagggcgcgtttaagagctatgctggaaacagcatagcaagtttaaataaggctagtccgttatcaacttgaaaaagtggcaccgagtcggtgcctgaatgcctgcgagcatctttttttgttttttatgtct

SEQ ID NO: 123 - DNA, 150 bp, Artificial Sequence
  Synthetic oligonucleotide
gcctctgggaggtcctgtccggctcgtttaagagctatgctggaaacagcatagcaagtttaaataaggctagtccgttatcaacttgaaaaagtggcaccgagtcggtgcctgaatgcctgcgagcatctttttttgttttttatgtct

SEQ ID NO: 124 - protein, 576 aa, Artificial Sequence
  Synthetic peptide
MPKKKRKVGSMKSIRCKNCNKLLFKADSFDHIEIRCPRCKRHIIMLNACEHPTEKHCGKREKITHSDETVRYGSGSGSGDIEKLCKKAEEEAKEAQEKADELRQRHPDSQAAEDAEDLANLAVAAVLTACLLAQEHPNADIAKLCIKAASEAAEAASKAAELAQRHPDSQAARDAIKLASQAARAVILAIMLAAENPNADIAKLCIKAASEAAEAASKAAELAQRHPDSQAARDAIKLASQAAEAVERAIWLAAENPNADIAKKCIKAASEAAEEASKAAEEAQRHPDSQKARDEIKEASQKAEEVKERCKSEQKLISEEDLGSGATNFSLLKQAGDVEENPGPSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 125 - DNA, 206 bp, Artificial Sequence
  Synthetic oligonucleotide
gtacagcagaagcctttagaagtttaagagctatgctggaaacagcatagcaagtttaaataaggctagtccgttatcaacttgaaaaagtggcaccgagtcggtgcgggagctaaggagtttatatggaaacccttagcctgctgcgtaaggagtttatatggaaacccttacgcagcagttccctttttttgttttttatgtct

SEQ ID NO: 126 - DNA, 206 bp, Artificial Sequence
  Synthetic oligonucleotide
gtggcatgctcacttcaggtggtttaagagctatgctggaaacagcatagcaagtttaaataaggctagtccgttatcaacttgaaaaagtggcaccgagtcggtgcgggagctaaggagtttatatggaaacccttagcctgctgcgtaaggagtttatatggaaacccttacgcagcagttccctttttttgttttttatgtct

SEQ ID NO: 127 - DNA, 206 bp, Artificial Sequence
  Synthetic oligonucleotide
gaagcctcgctggggaacgccgtttaagagctatgctggaaacagcatagcaagtttaaataaggctagtccgttatcaacttgaaaaagtggcaccgagtcggtgcgggagctaaggagtttatatggaaacccttagcctgctgcgtaaggagtttatatggaaacccttacgcagcagttccctttttttgttttttatgtct

SEQ ID NO: 128 - DNA, 206 bp, Artificial Sequence
  Synthetic oligonucleotide
gtacgttctctatcactgatagtttaagagctatgctggaaacagcatagcaagtttaaataaggctagtccgttatcaacttgaaaaagtggcaccgagtcggtgcgggagctaaggagtttatatggaaacccttagcctgctgcgtaaggagtttatatggaaacccttacgcagcagttccctttttttgttttttatgtct

SEQ ID NO: 129 - protein, 630 aa, Artificial Sequence
  Synthetic peptide
MPKKKRKVGSMSKTIVLSVGEATRTLTEIQSTADRQIFEEKVGPLVGRLRLTASLRQNGAKTAYRVNLKLDQADVVDSGLPKVRYTQVWSHDVTIVANSTEASRKSLYDLTKSLVATSQVEDLVVNLVPLGRGSGSGSSDEEEARELIERAKEAAERAQEAAERTGDPRVRELARELKRLAQEAAEEVKRDPSSSDVNEALKLIVEAIEAAVDALEAAERTGDPEVRELARELVRLAVEAAEEVQRNPSSSDVNEALHSIVYAIEAAIFALEAAERTGDPEVRELARELVRLAVEAAEEVQRNPSSRNVEHALMRIVLAIYLAEENLREAEESGDPEKREKARERVREAVERAEEVQRDPSGWLNHEQKLISEEDLGSGATNFSLLKQAGDVEENPGPSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 130 - DNA, 183 bp, Artificial Sequence
  Synthetic oligonucleotide
gtacgttctctatcactgatagtttaagagctatgctggaaacagcatagcaagtttaaataaggctagtccgttatcaacttgaaaaagtggcaccgagtcggtgcgggagcacatgaggatcacccatgtgcgactcccacagtcactggggagtcttccctttttttgttttttatgtct

SEQ ID NO: 131 - protein, 476 aa, Artificial Sequence; identical to SEQ ID NO: 72
  Synthetic peptide

SEQ ID NO: 132 - protein, 745 aa, Artificial Sequence; identical to SEQ ID NO: 71
  Synthetic peptide

SEQ ID NO: 133 - protein, 1387 aa, Artificial Sequence
  Synthetic peptide
MDYKDDDDKDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKV

SEQ ID NO: 134 - DNA, 183 bp, Artificial Sequence; identical to SEQ ID NO: 117
  Synthetic oligonucleotide

SEQ ID NO: 135 - DNA, 185 bp, Artificial Sequence
  Synthetic oligonucleotide
gcagacgcgaggaaggagggcgcgtttaagagctatgctggaaacagcatagcaagtttaaataaggctagtccgttatcaacttgaaaaagtggcaccgagtcggtgcgggagcacatgaggatcacccatgtgcgactcccacagtcactggggagtcttccctttttttgttttttatgtct

SEQ ID NO: 136 - DNA, 187 bp, Artificial Sequence; identical to SEQ ID NO: 119
  Synthetic oligonucleotide

SEQ ID NO: 137 - DNA, 183 bp, Artificial Sequence
  Synthetic oligonucleotide
gtacagcagaagcctttagaagtttaagagctatgctggaaacagcatagcaagtttaaataaggctagtccgttatcaacttgaaaaagtggcaccgagtcggtgcgggagcacatgaggatcacccatgtgcgactcccacagtcactggggagtcttccctttttttgttttttatgtct

SEQ ID NO: 138 - DNA, 183 bp, Artificial Sequence
  Synthetic oligonucleotide
gtggcatgctcacttcaggtggtttaagagctatgctggaaacagcatagcaagtttaaataaggctagtccgttatcaacttgaaaaagtggcaccgagtcggtgcgggagcacatgaggatcacccatgtgcgactcccacagtcactggggagtcttccctttttttgttttttatgtct

SEQ ID NO: 139 - DNA, 183 bp, Artificial Sequence
  Synthetic oligonucleotide
gaagcctcgctggggaacgccgtttaagagctatgctggaaacagcatagcaagtttaaataaggctagtccgttatcaacttgaaaaagtggcaccgagtcggtgcgggagcacatgaggatcacccatgtgcgactcccacagtcactggggagtcttccctttttttgttttttatgtct

SEQ ID NO: 140 - protein, 1793 aa, Artificial Sequence
  Synthetic peptide
MPKKKRKVGSMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGSGGSGTGEQKLISEEDLGGKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSPGSGATNFSLLKQAGDVEENPGPMSSDEEEARELIERAKEAAERAQEAAERTGDPRVRELARELKRLAQEAAEEVKRDPSSSDVNEALKLIVEAIEAAVDALEAAERTGDPEVRELARELVRLAVEAAEEVQRNPSSSDVNEALHSIVYAIEAAIFALEAAERTGDPEVRELARELVRLAVEAAEEVQRNPSSRNVEHALMRIVLAIYLAEENLREAEESGDPEKREKARERVREAVERAEEVQRDPSGWLNHEQKLISEEDLSGGGSGGSGSMDAKSLTAWSRTLVTFKDVFVDFTREEWKLLDTAQQIVYRNVMLENYKNLVSLGYQLTKPDVILRLEKGEEPWLVSGGGSGGSGSSPKKKRKVEASVQVKRVLEKSPGKLLVKMPFQASPGGKGEGGGATTSAQVMVIKRPGRKRKAEADPQAIPKKRGRKPGSVVAAAAAEAKKKAVKESSIRSVQETVLPIKKRKTRETVSIEVKEVVKPLLVSTLGEKSGKGLKTCKSPGRKSKESSPKGRSSSASSPPKKEHHHHHHHAESPKAPMPLLPPPPPPEPQSSEDPISPPEPQDLSSSICKEEKMPRAGSLESDGCPKEPAKTQPMVAAAATTTTTTTTTVAEKYKHRGEGERKDIVSSSMPRPNREEPVDSRTPVTERVSGSGATNFSLLKQAGDVEENPGPDIEKLCKKAEEEAKEAQEKADELRQRHPDSQAAEDAEDLANLAVAAVLTACLLAQEHPNADIAKLCIKAASEAAEAASKAAELAQRHPDSQAARDAIKLASQAARAVILAIMLAAENPNADIAKLCIKAASEAAEAASKAAELAQRHPDSQAARDAIKLASQAAEAVERAIWLAAENPNADIAKKCIKAASEAAEEASKAAEEAQRHPDSQKARDEIKEASQKAEEVKERCKSEQKLISEEDLEFSSAAGTSDALDDFDLDMLGSDALDDFDLDMLGSDALDDFDLDMLGSDALDDFDLDMLGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 141 - protein, 260 aa, Artificial Sequence
  Synthetic peptide
MGVADLIKKFESISKEEGDPPVATMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 142 - protein, 1529 aa, Artificial Sequence
  Synthetic peptide
  MISC_FEATURE (478)..(479): IRES
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKGSGTGEQKLISEEDLGGKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSPSAGGSAGGEKMSKDGKKKKKKSKTKCVIMMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGSGEQKLISEEDLGSGSSDEEEARELIERAKEAAERAQEAAERTGDPRVRELARELKRLAQEAAEEVKRDPSSSDVNEALKLIVEAIEAAVDALEAAERTGDPEVRELARELVRLAVEAAEEVQRNPSSSDVNEALHSIVYAIEAAIFALEAAERTGDPEVRELARELVRLAVEAAEEVQRNPSSRNVEHALMRIVLAIYLAEENLREAEESGDPEKREKARERVREAVERAEEVQRDPSGWLNHSAGGSAGGSAGGSAGGSGASGSGATNFSLLKQAGDVEENPGPSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLNSGSGEQKLISEEDLGSGTGSGTGSGTGTTSGTGTGGSTGMDIEKLCKKAEEEAKEAQEKADELRQRHPDSQAAEDAEDLANLAVAAVLTACLLAQEHPNADIAKLCIKAASEAAEAASKAAELAQRHPDSQAARDAIKLASQAARAVILAIMLAAENPNADIAKLCIKAASEAAEAASKAAELAQRHPDSQAARDAIKLASQAAEAVERAIWLAAENPNADIAKKCIKAASEAAEEASKAAEEAQRHPDSQKARDEIKEASQKAEEVKERCKSSAGGSAGGSAGGSAGGSAG

SEQ ID NO: 143 - protein, 1719 aa, Artificial Sequence
  Synthetic peptide
  MISC_FEATURE (238)..(239): IRES
MEQKLISEEDLGGKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSPSAGGSAGGEKMSKDGKKKKKKSKTKCVIMMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGSGEQKLISEEDLGSGSSDEEEARELIERAKEAAERAQEAAERTGDPRVRELARELKRLAQEAAEEVKRDPSSSDVNEALKLIVEAIEAAVDALEAAERTGDPEVRELARELVRLAVEAAEEVQRNPSSSDVNEALHSIVYAIEAAIFALEAAERTGDPEVRELARELVRLAVEAAEEVQRNPSSRNVEHALMRIVLAIYLAEENLREAEESGDPEKREKARERVREAVERAEEVQRDPSGWLNHSAGGSAGGSAGGSAGGSGASRQLSDADKLRKVICELLETERTYVKDLNCLMERYLKPLQKETFLTQDELDVLFGNLTEMVEFQVEFLKTLEDGVRLVPDLEKLEKVDQFKKVLFSLGGSFLYYADRFKLYSAFCASHTKVPKVLVKAKTDTAFKAFLDAQNPKQQHSSTLESYLIKPIQRILKYPLLLRELFALTDAESEEHYHLDVAIKTMNKVASHINEMQKIHEEGSGATNFSLLKQAGDVEENPGPSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLNSGSGEQKLISEEDLGSGTGSGTGSGTGTTSGTGTGGSTGMDIEKLCKKAEEEAKEAQEKADELRQRHPDSQAAEDAEDLANLAVAAVLTACLLAQEHPNADIAKLCIKAASEAAEAASKAAELAQRHPDSQAARDAIKLASQAARAVILAIMLAAENPNADIAKLCIKAASEAAEAASKAAELAQRHPDSQAARDAIKLASQAAEAVERAIWLAAENPNADIAKKCIKAASEAAEEASKAAEEAQRHPDSQKARDEIKEASQKAEEVKERCKSSAGGSAGGSAGGSAGGSAGTPPNWQQLVSREVLLGLKPCEIKRQEVINELFYTERAHVRTLKVLDQVFYQRVSREGILSPSELRKIFSNLEDILQLHIGLNEQMKAVRKRNETSVIDQIGEDLLTWFSGPGEEKLKHAAATFCSNQPFALEMIKSRQKKDSRFQTFVQDAESNPLCRRLQLKDIIPTQMQRLTKYPLLLDNIAKYTEWPTEREKVKKAADHCRQILNYVNQAVKEAENKQR

SEQ ID NO: 144 - protein, 1092 aa, Artificial Sequence; identical to SEQ ID NO: 74
  Synthetic peptide

SEQ ID NO: 145 - DNA, 20 bp, Artificial Sequence
  Synthetic oligonucleotide
gaagctgttggctgaaaagg

SEQ ID NO: 146 - DNA, 20 bp, Artificial Sequence
  Synthetic oligonucleotide
ctcactgacgttggcaaaga

SEQ ID NO: 147 - DNA, 20 bp, Artificial Sequence
  Synthetic oligonucleotide
acagtcagccgcatcttctt

SEQ ID NO: 148 - DNA, 20 bp, Artificial Sequence
  Synthetic oligonucleotide
acgaccaaatccgttgactc

SEQ ID NO: 149 - DNA, 20 bp, Artificial Sequence
  Synthetic oligonucleotide
agcagaagaacggcatcaag

SEQ ID NO: 150 - DNA, 20 bp, Artificial Sequence
  Synthetic oligonucleotide
ggggtgttctgctggtagtg

SEQ ID NO: 151 - DNA, 20 bp, Artificial Sequence
  Synthetic oligonucleotide
atggtgtcaatgaagccaaa

SEQ ID NO: 152 - DNA, 20 bp, Artificial Sequence
  Synthetic oligonucleotide
tgatgccaattacgaagcag

SEQ ID NO: 153 - protein, 228 aa, Artificial Sequence
  Synthetic peptide
SSDEEEARELIERAKEAAERAQEAAERTGDPRVRELARELKRLAQEAAEEVKRDPSSSDVNEALKLIVEAIEAAVRALEAAERTGDPEVRELARELVRLAVEAAEEVQRNPSSSDVNEALKLIVEAIEAAVRALEAAERTGDPEVRELARELVRLAVEAAEEVQRNPSSEEVNEALKKIVKAIQEAVESLREAEESGDPEKREKARERVREAVERAEEVQRDPSGWLE

SEQ ID NO: 154 - protein, 233 aa, Artificial Sequence
  Synthetic peptide
DIEKLCKKAESEAREARSKAEELRQRHPDSQAARDAQKLASQAEEAVKLACELAQEHPNADIAKLCIKAASEAAEAASKAAELAQRHPDSQAARDAIKLASQAAEAVKLACELAQEHPNADIAKLCIKAASEAAEAASKAAELAQRHPDSQAARDAIKLASQAAEAVKLACELAQEHPNADIAKKCIKAASEAAEEASKAAEEAQRHPDSQKARDEIKEASQKAEEVKERCER

(19) United States
(12) Patent Application Publication
(10) Pub. No.: US 2022/0025003 A1
(43) Pub. Date: Jan. 27, 2022

(54) REAGENTS AND METHODS FOR CONTROLLING PROTEIN FUNCTION AND INTERACTION

(71) Applicant: University of Washington, Seattle, WA (US)

(72) Inventors: David BAKER, Seattle, WA (US); Daniel CUNNINGHAM-BRYANT, Seattle, WA (US); Emily DIETER, Seattle, WA (US); Glenna FOIGHT, Seattle, WA (US); Per GREISEN, Seattle, WA (US); Dustin MALY, Seattle, WA (US); Keunwan PARK, Seattle, WA (US); Zhizhi WANG, Seattle, WA (US); Cindy WEI, Seattle, WA (US)

(21) Appl. No.: 17/297,606

(22) PCT Filed: Dec. 3, 2019
(86) PCT No.: PCT/US2019/064203

§ 371 Date: May 27, 2021

Related U.S. Application Data
(60) Provisional application No. 62/775,171, filed on Dec. 4, 2018.

(51) Int. Cl. C07K 14/47; C12N 9/22; C12N 9/50

(52) U.S. Cl. CPC C07K 14/4703; C12N 9/22; C12N 9/506; C07K 2319/81; C07K 2319/01; C07K 2319/70; C12Y 304/21098

(57) ABSTRACT

The present disclosure provides danoprevir/NS3a complex reader (DNCR) and grazoprevir/NS3a complex readers (GNCR) polypeptides, fusion proteins, and combinations and their use.

### CROSS REFERENCE

This application claims priority to U.S. Provisional Patent Application Ser. No. 62/775,171 filed Dec. 4, 2018, incorporated by reference herein in its entirety.

### STATEMENT OF GOVERNMENT RIGHTS

This invention was made with government support under Grant No. R01GM086858 awarded by the National Institutes of Health. The government has certain rights in the invention.

### BACKGROUND

Rationally manipulating protein localization can provide fundamental insights into cellular processes and is a powerful tool for engineering cellular behaviors. Techniques that allow temporal regulation of protein localization are particularly valuable for interrogating and programming dynamic cellular processes, with light and small molecules serving as the most widely used means of user-defined control.

### SUMMARY

In one aspect, the disclosure provides non-naturally occurring polypeptides comprising the general formula X1-X2-X3-X4-X5, wherein:

X1 optionally comprises first, second, third, and fourth helical domains;

X2 comprises a fifth helical domain comprising the amino acid sequence having at least 50%, 55%, 60%, 65%, 70%, 75%, 80%, 85%, 90%, 91%, 92%, 93%, 94%, 95%, 96%, 97%, 98%, 99%, or 100% identity to the full length of HSIVYAIEAAIF (SEQ ID NO:1), wherein 1, 2, 3, or all 4 of the following changes from SEQ ID NO:1 are not permissible: H1K, S2L, Y5E, and F12R

X3 comprises a sixth helical domain;

X4 comprises a seventh helical domain comprising the amino acid sequence having at least 50%, 55%, 60%, 65%, 70%, 75%, 80%, 85%, 90%, 91%, 92%, 93%, 94%, 95%, 96%, 97%, 98%, 99%, or 100% identity to the full length of RNVEHALMRIVLAIY (SEQ ID NO:2), wherein 1, 2, 3, or all 4 of the following changes from SEQ ID NO:2 are not permissible: R1E, H5E, M8K, and L12K; and

X5 comprises an eighth helical domain. In various embodiments, acceptable substitutions in X2 relative to SEQ ID NO:1 are selected from the group shown in Table 1 and Table 2; acceptable substitutions in X4 relative to SEQ ID NO:2 are selected from the group shown in Table 3 and Table 4; X2 comprises the amino acid sequence having at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identity to the full length of

(SEQ ID NO: 3)
SDVNEALHSIVYAIEAAIFALEAAERT;

X4 comprises the amino acid sequence having at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identity to the full length of

(SEQ ID NO: 4)
RNVEHALMRIVLAIYLAEENLREAEES;

X3 comprises the amino acid sequence having at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identity to the full length of EVRELARELVRLAVEAAEEVQR (SEQ ID NO:5); X5 comprises the amino acid sequence having at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identity to the full length of EKREKARERVREAVERAEEVQR (SEQ ID NO:6); and/or X1, when present, comprises the amino acid sequence having at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identity to the full length of:

(SEQ ID NO: 7)
SDEEEARELIERAKEAAERAQEAAERTGDPRVRELARELKRLAQEAAEEV
KRDPSSSDVNEALKLIVEAIEAAVDALEAAERTGDPEVRELARELVRLAV
EAAEEVQR

In another aspect, the disclosure provides non-naturally occurring polypeptide comprising the general formula X1-X2-X3-X4-X5-X6-X7, wherein:

X1 comprises first helical domain;

X2 comprises a second helical domain comprising the amino acid sequence having at least 50%, 55%, 60%, 65%, 70%, 75%, 80%, 85%, 90%, 91%, 92%, 93%, 94%, 95%, 96%, 97%, 98%, 99%, or 100% identity to the full length of

(SEQ ID NO: 20)
DLANLAVAAVLTACL,

wherein 1, 2, 3, 4, 5, 6, or all 7 of the following changes from SEQ ID NO:20 are not permissible: D1K, N4S, L5Q, A8E, L11K, T12L, and L15E;

X3 comprises a third helical domain;

X4 comprises a fourth helical domain comprising the amino acid sequence having at least 50%, 55%, 60%, 65%, 70%, 75%, 80%, 85%, 90%, 91%, 92%, 93%, 94%, 95%, 96%, 97%, 98%, 99%, or 100% identity to the full length of RAVILAIM (SEQ ID NO:21), wherein 1, 2, 3, or all 4 of the following changes from SEQ ID NO:21 are not permissible: R1E, I4K, I7C, and M8E;

X5 comprises a fifth helical domain;

X6 comprises a sixth helical domain comprising the amino acid sequence having at least 50%, 55%, 60%, 65%, 70%, 75%, 80%, 85%, 90%, 91%, 92%, 93%, 94%, 95%, 96%, 97%, 98%, 99%, or 100% identity to the full length of RAIWLAAE (SEQ ID NO:22), wherein 1, 2, 3, or all 4 of the following changes from SEQ ID NO:22 are not permissible: R1L, I3C, W4E, and A7Q; and

X7 comprises seventh and eighth helical domains. In various embodiments, acceptable substitutions in X2 relative to SEQ ID NO:20 are selected from those shown in Table 6 and Table 7; acceptable substitutions in X4 relative to SEQ ID NO:21 are selected from those shown in Table 8 and Table 9; acceptable substitutions in X6 relative to SEQ ID NO:22 are selected from those shown in Table 10 and Table 11; X2 comprises the amino acid sequence having at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identity to the full length of

(SEQ ID NO: 23)
QAAEDAEDLANLAVAAVLTACLLAQEH;

X4 comprises the amino acid sequence having at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identity to the full length of

(SEQ ID NO: 24)
QAARDAIKLASQAARAVILAIMLAA;

X6 comprises the amino acid sequence having at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identity to the full length of

(SEQ ID NO:25)
QAARDAIKLASQAAEAVERAIWLAAE;

X1 comprises the amino acid sequence having at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identity to the full length of

(SEQ ID NO: 26)
IEKLCKKAEEEAKEAQEKADELRQRH;

X3 comprises the amino acid sequence having at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identity to the full length of DIAKLCIKAASEAAEAASKAAELAQR (SEQ ID NO: 27); X5 comprises the amino acid sequence having at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identity to the full length of DIAKLCIKAASEAAEAASKAAELAQR (SEQ ID NO:28); and/or X7 comprises the amino acid sequence having at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identity to the full length of

(SEQ ID NO: 29)
DIAKKCIKAASEAAEEASKAAEEAQRHPDSQKARDEIKEASQKAEEVKER.

In a further aspect, the disclosure provides fusion protein comprising:

(a) the polypeptide of any embodiment or combination of embodiments of the disclosure; and

(b) a polypeptide localization domain at the N-terminus and/or the C-terminus of the fusion protein, and/or a protein having one or more interaction surfaces.

In one aspect, the disclosure provides recombinant fusion proteins, comprising a polypeptide of the general formula X1-B1-X2-B2-X3, wherein

(a) one of X1 and X3 is selected from the group consisting of
(i) a peptide comprising the amino acid sequence having at least 75%, 80%, 85%, 90%, 91%, 92%, 93%, 94%, 95%, 96%, 97%, 98%, 99%, or 100% identity to the full length of the amino acid sequence selected from GELGRLVYLLDGPGYDPIHSD (SEQ ID NO:13), GELDELVYLLDGPGYDPIHSD (SEQ ID NO:14), GELGELVYLLDGPGYDPIHSD (SEQ ID NO: 15), or GELDRLVYLLDGPGYDPIHSD (SEQ ID NO:16), or GELDELVYLLDGPGYDPIHSDVVTRGGSHLFNF (SEQ ID NO:17) (“ANR peptide”).
(ii) the DNCR polypeptide of any embodiment or combination of embodiments disclosed herein; and
(iii) the GNCR polypeptide of any embodiment or combination of embodiments disclosed herein;

(b) the other of X1 and X3 is an NS3a peptide (either catalytically active or dead), wherein if X1 or X3 is the ANR peptide, then NS3a is one of SEQ ID NOS:30-38;

(c) X2 is a protein having one or more interaction surfaces; and

(d) B1 and B2 are optional amino acid linkers.

In one embodiment, the NS3a peptide comprises the amino acid sequence having at least 80%, 75%, 90%, 91%, 92%, 93%, 94%, 95%, 96%, 97%, 98%, 99%, or 100% identity to the full length of the amino acid sequence selected from the group consisting of SEQ ID NOS:30-38, wherein the bolded amino acid residue is the catalytic position, wherein the bolded “S” residue represents catalytically active NS3a peptides, and wherein the bolded ‘S” residue can be substituted with an alanine (or other) residue to render the NS3a peptide catalytically dead.

In another aspect, the disclosure provides polypeptides comprising the amino acid sequence selected from the group consisting SEQ ID NO:31-38, wherein the bolded amino acid residue is the catalytic position, wherein the bolded “S” residue represents catalytically active NS3a peptides, and wherein the bolded ‘S” residue can be substituted with an alanine (or other) residue to render the NS3a peptide catalytically dead.

In a further aspect, the disclosure provides combinations, comprising:

(a) a first fusion protein comprising:
(i) a localization tag or a protein having one or more interaction surfaces; and
(ii) an NS3a peptide comprising the amino acid sequence having at least 80%, 85%, 90%, 91%, 92%, 93%, 94%, 95%, 96%, 97%, 98%, 99%, or 100% identity to the full length of the amino acid sequence selected from the group consisting of SEQ ID NOS:31-38, wherein the bolded amino acid residue is the catalytic position, wherein the bolded “S” residue represents catalytically active NS3a peptides, and wherein the bolded ‘S” residue can be substituted with an alanine (or other) residue to render the NS3a peptide catalytically dead; and

(b) one or more second fusion proteins comprising:
(i) a localization tag if the first fusion protein comprises a protein having one or more interaction surfaces; or a protein having one or more interaction surfaces if the first fusion protein comprises a localization tag; and
(ii) a polypeptide selected from the group consisting of selected from the group consisting of:
   (A) a polypeptide comprising the amino acid sequence having at least 50%, 55%, 60%, 65%, 70%, 75%, 80%, 85%, 90%, 91%, 92%, 93%, 94%, 95%, 96%, 97%, 98%, 99%, or 100% identity to the full length of the amino acid sequence selected from GELGRLVYLLDGPGYDPIHSD (SEQ ID NO:13), GELDELVYLLDGPGYDPIHSD (SEQ ID NO:14), GELGELVYLLDGPGYDPIHSD (SEQ ID NO:15), GELDRLVYLLDGPGYDPIHSD (SEQ ID NO:16), or GELDELVYLLDGPGYDPIHSDVVTRGGSHLFNF (SEQ ID NO:17);
   (B) the DNCR polypeptide of any embodiment or combination of embodiments disclosed herein; and
   (C) the GNCR polypeptide of any embodiment or combination of embodiments disclosed herein.

In various further aspects, the disclosure provides nucleic acids encoding the polypeptide, fusion protein, or the recombinant fusion protein of any embodiment or combination of embodiments disclosed herein; expression vectors comprising the nucleic acid operatively linked to a promoter sequence; host cells comprising the nucleic acids and/or expression vectors; and use of the polypeptide, fusion protein, recombinant fusion protein, combination, nucleic acid, expression vector, or host cell or any embodiment disclosed herein to carry out any methods, including but not limited to those disclosed herein.

### DESCRIPTION OF THE FIGURES

FIG. 1. Chemically-disrupted proximity (CDP). (A) Components of a CDP system based on the HCVp NS3a. (B) CDP-mediated intramolecular regulation. (C) CDP-mediated intermolecular regulation.

FIG. 2. An NS3a-based chemically-disruptable activator of RAS (CDAR). (A) Schematic depiction of NS3a-CDAR's activation of RAS/ERK signaling. (B) Dependence of the NS3a/ANR complex's center-of-mass (in Å) relative to SOScat's active site on N- and C-terminal linker length (NL and CL). (C) Standard deviation of the NS3a/ANR complex's center-of-mass (in Å) as a function of NL and CL. (D) The NS3a-CDAR construct used in cellular studies. (E) Phospho-ERK blot (bottom) and quantification (top) of cells expressing NS3a-CDAR and treated with DMSO, danoprevir, grazoprevir, or asunaprevir for 60 min (n=2). (F) Phospho-ERK blot (bottom) and quantification (top) of NS3a-CDAR-expressing cells treated with asunaprevir for the times indicated (n=3).

FIG. 3. CDP control of protein localization. (A) Schematic of the mitochondrial colocalization assay. (B) Representative images of cells expressing mitochondrially-localized NS3a(H1) (Tom20-mCherry-NS3a(H1)) and EGFP-ANR₂ treated with DMSO or asunaprevir (Asun) for 5 min. (C) Quantification of EGFP and mCherry colocalization in DMSO and Asun-treated cells. (D) Representative images of cells expressing membrane-localized ANR (myr-mCherry-ANR₂) and EGFP-NS3a(H1) treated with DMSO or Asun for 15 min. (E) Quantification of EGFP and mCherry colocalization in DMSO and Asun-treated cells. (F) Representative images of cells expressing nuclear-localized ANR (NLS₃-BFP-ANR₂) and EGFP-NS3a(H1) treated with DMSO or Asun. (G) Quantification of EGFP and BFP colocalization in cells treated with Asun for the times shown. Quantification details and statistical analyses provided in FIG. 16.

FIG. 4. Intermolecular disruption of transcriptional activation. (A) Schematic of chemically-disruptable Gal4(DBD)-NS3a(H1)/ANR-VPR transcriptional regulation. (B) Quantification of median mCherry fluorescence for the conditions shown (n=3). (C) Schematic of chemically-inducible/disruptable, dciCas9-mediated transcriptional regulation. (D) Quantification of median GFP fluorescence for the conditions shown (n=3).

FIG. 5. ANR peptide sequence. (A) Amino acid sequence (SEQ ID NO:14) of the ANR portion of the NS3a-based CDP system. ANR is based on the Cp5 peptide scaffold described in Kigler et al. J. Biol. Chem. 2012, 287, 39224-32. (B) Structure of the ANR probe(SEQ ID NO:40) used in fluorescence polarization assays. The probe contains fluorescein (FAM), connected by a flexible glycine and serine linker, fused to the N-terminus of ANR.

FIG. 6. Characterization of ANR's affinity for NS3a. (A) The IC₅₀ value of an ANR-GST fusion against NS3a activity in a FRET-based protease assay (Taliani et al Anal. Biochem. 1996 240, 60-67). The apparent IC₅₀ value of ANR is less than the concentration of NS3a protease used in the assay. (B) The 50% fractional binding (FB₅₀) value of FAM-ANR (FIG. 5B) for catalytically active NS3a (NS3a active) and a catalytically inactive S139A variant (NS3a inactive) determined using a fluorescence polarization binding assay. Values shown are the mean of n=3.

FIG. 7. Danoprevir competes with ANR for NS3a binding. (A) Danoprevir titration in a fluorescence polarization competition binding assay with FAM-ANR (n=3). (B) FB₅₀ values of danoprevir for active NS3a (NS3a active) and a catalytically inactive S139A variant (NS3a inactive) determined from the titration shown in (A). Danoprevir's apparent IC₅₀ is less than the concentration of NS3a active and inactive (75 nM) used in the binding assay. (C) Danoprevir inhibits the ability of immobilized NS3a inactive to pull down ANR-GST. Biotinylated NS3a inactive was immobilized on streptavidin-agarose beads and 5 μM ANR-GST was added with danoprevir (10 μM) or DMSO. Following incubation, beads were washed, and bound ANR-GST was eluted. Eluted samples were subjected to SDS-PAGE and immunoblotting with an anti-GST antibody.

FIG. 8. Computational design of NS3a-CDAR. (A) The NS3a-CDAR construct used in computational modeling with RosettaRemodel™. The C-terminus of ANR is fused to the N-terminus of SOScat through a flexible N-terminal linker (NL). The C-terminus of SOScat is fused to the N-terminus of NS3a through a flexible C-terminal linker (CL). Combinations of NL and CL lengths ranging from 5-29 residues and 1-13 residues, respectively, were evaluated computationally. (B) RosettaRemodel™ closure frequency of NS3a-CDAR designs. Closure frequencies of the NS3a-CDAR constructs were determined as a function of NL and CL lengths by RosettaRemodel™ and plotted as the number of successfully closed trajectories divided by 1000 for each of the linker length pairs. We assigned an arbitrary lower bound on the chain closure frequency at 10%. Pairs of linker lengths that give fewer chain closure events would likely not allow intramolecular formation of the NS3a/ANR complex.

FIG. 9. RosettaRemodel™-determined values for the mean center-of-mass distance, standard deviation (SD) of this mean, and closure frequency of exemplary NS3a-CDAR designs. Values obtained from RosettaRemodel™ (FIGS. 2B, 2C, 8) were determined as a function of NL and CL lengths. Linker lengths are represented as NL-CL, with the values shown referring to the number of residues in each linker. We reasoned that the ability of the NS3a/ANR complex to autoinhibit SOScat likely depends on its overlap with the RAS-binding site of SOScat. The mean center-of-mass distance describes the average computed distance between the center-of-mass of SOScat-bound RAS and the NS3a/ANR complex. Designs with the smallest mean center-of-mass distance have the highest relative degree of overlap between the NS3a/ANR complex and SOScat-bound RAS. We used the standard deviation (SD) of this mean to predict the energetic penalty for the NS3a/ANR complex not adopting the average position relative to SOScat. Designs with the smallest SD have the most tightly clustered NS3a/ANR complexes in output PDBs.

FIG. 10. Functional characterization of NS3a-CDAR. (A) Schematic representation of the NS3a-CDAR variants that were tested for RAS/ERK activation in cells. The top construct (BH3-NS3a-CDAR) contains a similar architecture as NS3a-CDAR but ANR has been replaced with a peptide (BH3 domain from the protein Bad) that has no detectable affinity for NS3a. The bottom construct (NS3a-CDAR) was used in all experiments shown in FIG. 2. The number of residues in each linker connecting domains are shown as L_{#}. (B) Phospho-ERK blot of HEK293 cells transfected with an empty vector (E. V.) or a plasmid containing NS3a-CDAR and treated with DMSO (−) or 10 μM asunaprevir (+) for 60 min. Anti-ERK (middle) and anti-FLAG (bottom) immunoblots are also shown. (C) Phospho-ERK blot of HEK293 cells transfected with a plasmid containing BH3-NS3a-CDAR and treated with DMSO or asunaprevir (10 μM) for 60 min. Anti-ERK (middle) and anti-FLAG (bottom) immunoblots are also shown.

FIG. 11. Effects of NS3a inhibitors in cells lacking NS3a-CDAR. Phospho-ERK (top), total ERK (middle), and FLAG (bottom) blots of HEK293 cells transfected with an empty pcDNA5 vector and treated with 10 μM grazoprevir, asunaprevir, or danoprevir or HEK293 cells transfected with the FLAG-tagged NS3a-CDAR construct and treated with 10 μM grazoprevir. Cells were treated with the specified drugs for 60 min.

FIG. 12. NS3a-CDAR is necessary for temporal activation of the RAS/ERK pathway. Phospho-ERK (top), total ERK (middle), and FLAG (bottom) blots of HEK293 cells transfected with an empty pcDNA5 vector and treated with 10 μM asunaprevir for the time points indicated.

FIG. 13. NS3a/NS3a* chimeras. (A) Crystal structure of ANR bound to NS3a (PDB: 4A1X). Previous work (Brass, V.; Berke, J. M.; Montserret, R.; Blum, H. E.; Penin, F.; Moradpour, D. Proc. Natl. Acad. Sci. U.S.A. 2008, 105, 14545-50) has demonstrated that NS3a interacts with membranes through an amphipathic helix (helix-α0) and that this helix is partially responsible for the insolubility of recombinant NS3a. A variant of NS3a optimized for solubility (NS3a*) has been previously reported (Wittekind, M. et al. U.S. Pat. No. 6,333,186. 2004). However, NS3a* fails to bind ANR effectively (FIG. 14). Regions of NS3a that appear to make critical contacts with ANR and that differ between NS3a and NS3a* are shown in red [helix-α0 (residues 27-32)] and cyan [Tyr-finger pocket (residues 21, 49, and 56)]. (B) Crystal structure of NS3a bound to Asunaprevir (PDB: 4WF8). (C) Table depicting all NS3a/NS3a* chimeras that were generated and tested in the mitochondrial colocalization assay (FIG. 2A). The sequences of two regions [helix-α0 (residues 27-32) (From top to bottom, SEQ ID NOS:41-49) and the Tyr-finger pocket (residues 21, 49, and 56)] that differ between NS3a and NS3a* are shown in the first two rows. Chimeras were generated by introducing the sequences shown for both regions into NS3a*. All sequences for NS3a, NS3a*, and NS3a*/NS3a chimeras are provided in the methods section. Chimera's are henceforth referred to as NS3a(H #).

FIG. 14. In vitro characterization of the solubility optimized NS3a variant NS3a*. (A) 50% fractional binding (FB₅₀) curves of NS3a and NS3a* for FAM-ANR determined using a fluorescence polarization assay. Values shown for each concentration of NS3a and NS3a* are the mean+/−sem of n=3. (B) FB₅₀ values of NS3a and NS3a* for FAM-ANR.

FIG. 15. Screening of NS3a chimeras in a mitochondrial colocalization assay. (A) Pearson's r-correlation coefficients of mCherry™ and GFP fluorescence determined by confocal fluorescence microscopy in NIH-3T3 cells. Cells were co-transfected with EGFP-ANR₂ and a mitochondrially localized mCherry™-NS3a-chimera (Tom20-mCherry™-NS3a(H #), sequences shown in FIG. 13C) and treated with 10 μM asunaprevir or DMSO for 30 minutes followed immediately by fixation and analysis by confocal fluorescence microscopy. Pearson's r-correlation coefficients were determined using ImageJ and unpaired two-sided student's t-tests were calculated using Graphpad™ Prism. (B) Cell counts and statistics for both drug and DMSO treated cells for each NS3a-chimera. Only cells expressing both mCherry and EGFP were imaged and analyzed.

FIG. 16. Cell numbers and statistics for the colocalization experiments quantified in FIG. 3. Cells expressing EGFP and mCherry were imaged and analyzed. Pearson's r-correlation coefficients were determined in ImageJ and unpaired two-sided student's t-tests were calculated using Graphpad Prism. (A) Number of cells analyzed per condition and statistics for mitochondrial colocalization (data shown in FIG. 3C). (B) Number of cells analyzed per condition and statistics for plasma membrane colocalization (data shown in FIG. 3E). (C) Number of cells analyzed per time point and statistics for nuclear colocalization (data shown in FIG. 3G)

FIG. 17. In vitro characterization of the NS3a(H1) chimera. (A) 50% fractional binding (FB₅₀) curves of NS3a and NS3a(H1) for FAM-ANR determined using a fluorescence polarization assay. Values shown for each concentration of NS3a and NS3a* are the mean+/−sem of n=3. (B) Mean FB₅₀ values of NS3a and NS3a(H1) for FAM-ANR.

FIG. 18. PROCISiR concept and design of a danoprevir/NS3a complex reader. a, In the PROCISiR system, HCV protease NS3a acts as a central control hub that can receive various small molecule drug inputs. Reader proteins that discriminate between different states of NS3a then translate these inputs into a variety of output types including reversibility, tunability, multi-state control, and input ratio-sensing. PROCISiR can be used under multiple regimes, including direction of one protein fused to NS3a to multiple reader-defined locations or temporally-controlled assembly of multiple reader components to NS3a immobilized at one location or one protein complex. b, Goal and process for designing drug/NS3a complex readers. c, Rosetta model for D5 (left) and binding of 1 μM NS3a with avidity to yeast-displayed D5 in the presence or absence of 10 μM danoprevir. A point mutant of the D5 interface, W177D, and the original DHR79 scaffold show no binding. Representative technical replicate values (n=3) and their means for one of two independent experiments are shown. d, A co-crystal structure of the DNCR2/danoprevir/NS3a complex aligned with the D5/danoprevir/NS3a model via NS3a. e, Residues within 4 Å of NS3a/danoprevir are highlighted on the surface of DNCR2. Residues at the interface in the D5 model are outlined in black.

FIG. 19. Design of a grazoprevir/NS3a complex reader and the combined application of all PROCISiR components. a, Rosetta™ model and binding of 1 μM NS3a with avidity to yeast-displayed G3 in the presence or absence of 10 μM grazoprevir. Point mutants at the G3 interface, M112E and A175Q, and the original DHR18 scaffold show no binding. Representative technical replicate values (n=3) and their means for one of two independent experiments are shown. b, Colocalization of DNCR2-EGFP with mCherry™-NS3a immobilized at the mitochondria after 1 hour treatment with 10 μM drug or DMSO. c, Colocalization of NS3a-mCherry™ with GNCR1-BFP-CAAX or Tom20-DNCR2-EGFP after treatment with danoprevir (5 μM), grazoprevir (5 μM), or DMSO. See FIG. 26a for image examples. d, Colocalization of NS3a-mCherry™ with ANR—BFP-CAAX or NLS-DNCR2-EGFP after treatment with danoprevir (5 μM), grazoprevir (5 μM), or DMSO. See FIG. 26b for image examples. The mean and standard deviation of the Pearson's r of red/blue or red/green pixel intensities is given for each condition in (b-d) with the distributions for multiple NIH3T3 cells.

FIG. 20. Temporal and proportional transcriptional control paradigms achievable with PROCISiR. a, Reversibility of CXCR4 induction from danoprevir-promoted recruitment of DNCR2-VPR to NS3a-dCas9. “OFF” conditions indicate replacement of danoprevir-containing media with DMSO- or grazoprevir-containing media at 24 hours. Values shown are quantified by RTqPCR relative to a DMSO-only control. Mean and standard deviation of three biological replicates from one experiment. b, Varying the proportion of grazoprevir competitor in the presence of a uniform titration of danoprevir inducer in cells expressing DNCR2-VPR and NS3a-dCas9 extends the linear range of the CXCR4 (left) or CD95 (right) expression response. DMSO-baseline subtracted immunofluorescence values are shown, with mean and standard deviation of three biological replicates from one experiment. c, Diagram of system in (e) used to modulate expression of CXCR4 and GFP in cells expressing an MS2 scRNA targeting CXCR4, a PP7 scRNA targeting a GFP reporter, GNCR1-MCP, DNCR2-PCP, and NS3a-VPR. d, Modeling of the fraction of NS3a bound to danoprevir or grazoprevir at the drug concentrations used in (e), as described in Supplementary Note 3. e, Expression of CXCR4 and GFP after co-treatment with varying concentrations of danoprevir and grazoprevir. Each box is the value from one experiment, with a replicate shown in FIG. 28. Single bars to the left of CXCR4 and below GFP show single-drug titrations (mean of 3 biological replicates from one experiment).

FIG. 21. Proportional control of signaling pathway activation. a, NS3a was immobilized at the plasma membrane via a CAAX, with (b) or without an mCherry™ fusion (c). Varying combinations of danoprevir and grazoprevir were used to control the proportions of DNCR2 and GNCR1 fusions colocalizing with NS3a at the membrane. b, Colocalization of EGFP-DNCR2 with NS3a (green) and BFP-GNCR1 with NS3a (blue) quantified by Pearson's R (left axis, normalized to DMSO and single drug conditions, mean and standard deviation of ≥14 cells per condition). NS3a:DNCR2 and NS3a:GNCR1 colocalization data are shown overlaid with the predicted fractions of NS3a:danoprevir and NS3a:grazoprevir at the given drug concentrations (right axis). See Supplementary Note 3 for explanation of modeling. c, EGFP-DNCR2-TIAM (Rac GEF) and BFP-GNCR1-LARG (Rho GEF) direct spreading of HeLa cells when treated with 100 nM danoprevir (top panels) and contraction when treated with 100 nM grazoprevir (bottom panels), respectively. Lifeact-mCherry™ signal is shown to illustrate changes to actin fibers. Time is relative to addition of drug.

FIG. 22. Design and characterization of danoprevir/NS3a complex reader libraries. a, Process of Rosetta™ re-design-informed design of a combinatorial D5 interface library. b, Enrichment ratios of the DNCR1 site saturation mutagenesis (SSM) library sorted for (positive sort, top) or against (negative sort, bottom) binding to 50 nM NS3a in the presence of 500 nM danoprevir Gray boxes with letters are the wild-type residue and other gray boxes are positions with <15 counts in the naïve library sequencing results. c, Sequence logos of the theoretical library for the second combinatorial library varying the DNCR1 interface (top), and the mutations found in the final enriched clones (bottom). Residue identities at the varied positions are indicated for the starting DNCR1 and final DNCR2. d, Progression of binding improvement from DHR79 to D5 to DNCR1 to DNCR2 as measured by the deviation from average enrichment ratio of the DNCR1 SSM values at each position. Gray shaded region indicates the range of enrichment ratios of all amino acids at each position, and vertical gray bars indicate positions at the interface.

FIG. 23. Analysis of the DNCR2/danoprevir/NS3a complex crystal structure and the specificities of drug/NS3a complex reader proteins. a, 1 μM NS3a with avidity binding to yeast displayed D5, DNCR1, or DNCR2. Representative technical replicate values (n=3) and their means for one of two independent experiments are shown. b, Binding of 1 nM NS3a to DNCR2 displayed on the surface of yeast in the presence of increasing concentrations of danoprevir. Three technical replicate values from one experiment are shown. c, An overlay of DNCR2 (blue) from the DNCR2/danoprevir/NS3a complex with the original DHR79 scaffold (orange) crystal structure (PDBID: 5CWP). 13 Regions where there are modest changes in the backbone conformation are circled with a dotted line, including missing density for helix 8 and an unraveled helix 7 N-terminus. d, NS3a/danoprevir (blue) from the DNCR2/danoprevir/NS3a complex aligns closely to a crystal structure of NS3a/danoprevir (yellow) alone (PDBID: 3M5L). 36 e, Size exclusion chromatograms of DNCR2, NS3a, or DNCR2/NS3a complexes in the presence or absence of danoprevir. Representative of three technical replicates. f, Crystal structure of DNCR2/danoprevir/NS3a aligned to structures of asunaprevir/NS3a (lavender, PDBID: 4WF8) or grazoprevir/NS3a (PDBID: 3SUD) with clashes between residues of DNCR2 and asunaprevir and grazoprevir highlighted.

FIG. 24. Grazoprevir/NS3a complex reader binding and improvement. a, Binding of 1 μM NS3a with avidity to yeast-displayed G3 or GNCR1 in the presence of grazoprevir, danoprevir, asunaprevir, or DMSO. Representative technical replicate values (n=3) and their means for one of two independent experiments are shown. b, Predicted mutational preferences of the G3 interface for binding to NS3a/grazoprevir, as defined by the frequencies of mutations found in Rosetta™ re-designs of the interface. c, Sequence logos of the theoretical library for the combinatorial library varying the G3 interface (top), and the mutations found in the final enriched library (bottom). Residue identities at the varied positions are indicated for the starting G3 and final GNCR1.

FIG. 25. Characterization of kinetics and affinity of DNCR2/danoprevir/NS3a complex in mammalian cells. a, Kinetics of DNCR2-EGFP association with myristoylated NS3amCherry™ after adding 5 μM danoprevir. Mean and standard deviation of the cytoplasmic EGFP fluorescence (normalized to first and last frame) of 18 NIH3T3 cells collected from 4 separate experiments. b, Schematic of danoprevir-mediated PI3K-Akt pathway activation through recruitment of an inter-Src homology 2 domain (iSH2) of the regulatory PI3K subunit p85/DNCR2 fusion (DNCR2-iSH2) to myristoylated NS3a-mCherry™ (left panel). Quantification of phospho-Akt (pSer473) Western blots performed with varying concentrations of danoprevir in COS-7 cells expressing DNCR2-iSH2 and myristoylated NS3a-mCherry™. Mean and standard deviation of 3 biological replicates from one experiment, fit with a log dose-response curve, are shown.

FIG. 26. Combination of reader pairs for inducible 2-location and colocalization control with NS3a. a, Colocalization of NS3a-mCherry™ with GNCR1-BFP-CAAX or Tom20-DNCR2-EGFP after treatment with danoprevir (5 μM), grazoprevir (5 μM), or DMSO. b, Colocalization of NS3amCherry™ with ANR—BFP-CAAX or NLS-DNCR2-EGFP after treatment with danoprevir (5 μM), grazoprevir (5 μM), or DMSO. See FIG. 19c,d for quantification of multiple cells.

FIG. 27. Additional PROCISiR combinations for 2-location control of NS3a. a,Colocalization of GNCR1-BFP or DNCR2-EGFP with NS3a-mCherry™-CAAX after treatment with danoprevir (5 μM), grazoprevir (5 μM), or DMSO. b, Colocalization of NS3a-mCherry™ with Tom20-BFP-ANR or DNCR2-EGFP-CAAX after treatment with danoprevir (5 μM), grazoprevir (5 μM), or DMSO. c,d, The mean and standard deviation of the Pearson's r of red/blue or red/green pixel intensities is given for each condition in (a,b), with the distributions for multiple NIH3T3 cells.

FIG. 28. Gene expression titration with Gal4/UAS system and 2-gene titration. a, Titration of mCherry™ expression from a UAS-minCMV promoter using a danoprevir-inducible Gal4-NS3a/DNCR2-VPR system (left). Median mCherry™ values are shown in the middle panel, with the histograms for one replicate shown on right to illustrate that the full population shifts to intermediate levels of gene expression. b, Expression of CXCR4 and GFP in cells expressing an MS2 scRNA targeting CXCR4, a PP7 scRNA targeting a GFP reporter, GNCR1-MCP, DNCR2-PCP, and NS3a-VPR after treatment with DMSO, danoprevir, or grazoprevir. Fold changes relative to DMSO are given for each 10 μM drug response for three biological replicates from one experiment. c, Expression of CXCR4 and GFP in cells expressing constructs in (b) after co-treatment with varying concentrations of danoprevir and grazoprevir. Replicate of FIG. 20e. d, CXCR4 immunofluorescence from titration of grazoprevir alone in the same system as (b). e, GFP fluorescence from titration of danoprevir alone in the same system as (b). (a,d,e) are fit to a one-site, specific binding Hill equation, and each point shows the mean and standard deviation of 3 biological replicates from one experiment, with background fluorescence levels from a DMSO-only condition subtracted.

FIG. 29. Switchable repression and overexpression and 3-gene control. Median immunofluorescence of CXCR4 (a,b) or CD95 (c,d) expression controlled by danoprevir-promoted recruitment of (a,c) DNCR2-VPR or (b,d) DNCR2-KRAB to NS3a-dCas9 in the absence or presence of guides targeting the CXCR4 (a,b) or CD95 (c,d) promoter region. Fold change (a,c) or inverse fold change (b,d) are given above each DMSO/danoprevir condition pair. e, Switching between repression and overexpression is achieved from endogenous promoters for CXCR4 (right panel) and CD95 (f) using dCas9 with MCP-NS3a, GNCR1-VPR, and DNCR2-KRAB-MeCP2 (left panel). Fold change or inverse fold change is shown for treatment with 100 nM grazoprevir or danoprevir, respectively. (a-f) Median immunofluorescence intensities are given in arbitrary units for data from 3 biological replicates from one experiment. g, Expression of GFP, CD95, and CXCR4 using a MS2 scRNA targeting a GFP reporter, a PP7 scRNA targeting CD95, and a com scRNA targeting CXCR4 with MCP-ANR, PP7-DNCR2, and com-GNCR1, respectively. Responses for 3 biological replicates from one experiment are given for each gene relative to untransfected cells.

FIG. 30. Drug-regulated control of subcellular protein localization with intermediate-affinity danoprevir/NS3a reader, DNCR1. a, Colocalization of DNCR1-EGFP with mitochondria-, Golgi-, nuclear-, or plasma membrane-localized NS3a-mCherry under DMSO (left panel) or 10 μM danoprevir (right panel) treatment. b, Colocalization of mCherry™-NS3a with mitochondria-, Golgi-, or nuclear-localized DNCR1-EGFP under DMSO (left panel) or 10 μM danoprevir (right panel) treatment. Each panel in (a,b) is representative of the majority population of n≥18 NIH3T3 cells. Quantification of colocalization of mCherry™-NS3a with (c) Golgi- or (d) mitochondria-localized DNCR1-EGFP after treatment with grazoprevir (10 μM), danoprevir (10 μM), asunaprevir (10 μM), or DMSO. The mean and standard deviation of the Pearson's r of red/green pixel intensities is given for each condition with the distributions for multiple NIH3T3 cells.

FIG. 31. Modeling of NS3a:danoprevir, NS3a:grazoprevir, and NS3a:asunaprevir occupancies. a, The fraction of NS3a bound to danoprevir (left axis) and the fraction of NS3a bound to grazoprevir (right axis) was computed for a constant concentration of 100 nM danoprevir, with increasing concentrations of grazoprevir. b, The fraction of NS3a bound to danoprevir (left axis) and the fraction of NS3a bound to asunaprevir (right axis) was computed for a constant concentration of 100 nM danoprevir, with increasing concentrations of asunaprevir. c, The fraction of NS3a bound to asunaprevir (left axis) and the fraction of NS3a bound to grazoprevir (right axis) was computed for a constant concentration of 100 nM asunaprevir, with increasing concentrations of grazoprevir. The vertical gray lines mark the asunaprevir or grazoprevir concentrations used for the experiments in FIG. 21.

FIG. 32. Alignment of exemplary DNCR polypeptide variants with starting scaffold DHR79, showing position of helices.

FIG. 33. Alignment of exemplary GNCR polypeptide variants with starting scaffold DHR18, showing position of helices.

### DETAILED DESCRIPTION

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural or singular number, respectively. Additionally, the words “herein,” “above” and “below” and words of similar import, when used in this application, shall refer to this application as a whole and not to any particular portions of this application.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

All embodiments of any aspect of the invention can be used in combination, unless the context clearly dictates otherwise.

In a first aspect, the disclosure provides non-naturally occurring polypeptide comprising the general formula X1-X2-X3-X4-X5, wherein:

X1 optionally comprises first, second, third, and fourth helical domains;

X2 comprises a fifth helical domain comprising the amino acid sequence having at least 50%, 55%, 60%, 65%, 70%, 75%, 80%, 85%, 90%, 91%, 92%, 93%, 94%, 95%, 96%, 97%, 98%, 99%, or 100% identity to the full length of HSIVYAIEAAIF (SEQ ID NO:1), wherein 1, 2, 3, or all 4 of the following changes from SEQ ID NO:1 are not permissible: H1K, S2L, Y5E, and F12R

X3 comprises a sixth helical domain;

X4 comprises a seventh helical domain comprising the amino acid sequence having at least 50%, 55%, 60%, 65%, 70%, 75%, 80%, 85%, 90%, 91%, 92%, 93%, 94%, 95%, 96%, 97%, 98%, 99%, or 100% identity to the full length of RNVEHALMRIVLAIY (SEQ ID NO:2), wherein 1, 2, 3, or all 4 of the following changes from SEQ ID NO:2 are not permissible: R1E, H5E, M8K, and L12K; and

X5 comprises an eighth helical domain.

The polypeptides of this aspect are danoprevir/NS3a complex reader (DNCR) polypeptides that selectively bind a danoprevir/NS3a complex over the apo NS3a protein, where NS3a is any variant of the HCV protease NS3/4a (any genotype and catalytically active or dead), as described in detail in the attached appendices. The functional part of DNCR is the interface with danoprevir/NS3a, which includes portions of helices 5 and 7. This interface could be grafted onto any protein backbone that supported the arrangement of these helices while retaining activity as a danoprevir/NS3a complex reader. There is flexibility in the amino acid sequence of these interface helices, with the general mutational trends permitted discussed in the examples that follow. The X1 helical domains are optional, in that the inventors have shown binding in the absence of the first four helical domains. As will be understood, 1, 2, 3, or all 4 helical domains may be present or absent. For example, only helical domain 4 may be present; only helical domains 3-4 may be present, only helical domains 2-4 may be present; helical domains 1-4 may be present, or none of helical domains 1-4 may be present.

As used herein, a “helical domain” is any sequence of amino acids that forms an alpha-helical secondary structure. In one embodiment, the helical domains do not include any proline residues. In another embodiment, the length of the 5^{th} and 7^{th} helical domains is at least 12 amino acids. In other embodiments, the length of each helical domain is at least 12 amino acids in length. In other exemplary embodiments, the length of each helical domain is independently between 12 and 35, 12-30, 15-30, 20-30, 22-28, 23-27, 24-26, or 25 amino acids in length.

In various embodiments:
X2 comprises a fifth helical domain comprising the amino acid sequence having at least 60% identity to the full length of HSIVYAIEAAIF (SEQ ID NO:1), wherein 1, 2, 3, or all 4 of the following changes from SEQ ID NO:1 are not permissible: H1K, S2L, Y5E, and F12R, and X4 comprises a seventh helical domain comprising the amino acid sequence having at least 60% identity to the full length of RNVEHALMRIVLAIY (SEQ ID NO:2), wherein 1, 2, 3, or all 4 of the following changes from SEQ ID NO:2 are not permissible: R1E, H5E, M8K, and L12K;
X2 comprises a fifth helical domain comprising the amino acid sequence having at least 70% identity to the full length of HSIVYAIEAAIF (SEQ ID NO:1), wherein 1, 2, 3, or all 4 of the following changes from SEQ ID NO:1 are not permissible: H1K, S2L, Y5E, and F12R, and X4 comprises a seventh helical domain comprising the amino acid sequence having at least 70% identity to the full length of RNVEHALMRIVLAIY (SEQ ID NO:2), wherein 1, 2, 3, or all 4 of the following changes from SEQ ID NO:2 are not permissible: R1E, H5E, M8K, and L12K;
X2 comprises a fifth helical domain comprising the amino acid sequence having at least 80% identity to the full length of HSIVYAIEAAIF (SEQ ID NO:1), wherein 1, 2, 3, or all 4 of the following changes from SEQ ID NO:1 are not permissible: H1K, S2L, Y5E, and F12R, and X4 comprises a seventh helical domain comprising the amino acid sequence having at least 80% identity to the full length of RNVEHALMRIVLAIY (SEQ ID NO:2), wherein 1, 2, 3, or all 4 of the following changes from SEQ ID NO:2 are not permissible: R1E, H5E, M8K, and L12K;
X2 comprises a fifth helical domain comprising the amino acid sequence having at least 85% identity to the full length of HSIVYAIEAAIF (SEQ ID NO:1), wherein 1, 2, 3, or all 4 of the following changes from SEQ ID NO:1 are not permissible: H1K, S2L, Y5E, and F12R, and X4 comprises a seventh helical domain comprising the amino acid sequence having at least 85% identity to the full length of RNVEHALMRIVLAIY (SEQ ID NO:2), wherein 1, 2, 3, or all 4 of the following changes from SEQ ID NO:2 are not permissible: R1E, H5E, M8K, and L12K;
X2 comprises a fifth helical domain comprising the amino acid sequence having at least 90% identity to the full length of HSIVYAIEAAIF (SEQ ID NO:1), wherein 1, 2, 3, or all 4 of the following changes from SEQ ID NO:1 are not permissible: H1K, S2L, Y5E, and F12R, and X4 comprises a seventh helical domain comprising the amino acid sequence having at least 90% identity to the full length of RNVEHALMRIVLAIY (SEQ ID NO:2), wherein 1, 2, 3, or all 4 of the following changes from SEQ ID NO:2 are not permissible: R1E, H5E, M8K, and L12K;
X2 comprises a fifth helical domain comprising the amino acid sequence having at least 95% identity to the full length of HSIVYAIEAAIF (SEQ ID NO:1), wherein 1, 2, 3, or all 4 of the following changes from SEQ ID NO:1 are not permissible: H1K, S2L, Y5E, and F12R, and X4 comprises a seventh helical domain comprising the amino acid sequence having at least 95% identity to the full length of RNVEHALMRIVLAIY (SEQ ID NO:2), wherein 1, 2, 3, or all 4 of the following changes from SEQ ID NO:2 are not permissible: R1E, H5E, M8K, and L12K; or
X2 comprises a fifth helical domain comprising the amino acid sequence having 100% identity to the full length of HSIVYAIEAAIF (SEQ ID NO:1), and X4 comprises a seventh helical domain comprising the amino acid sequence having 100% identity to the full length of RNVEHALMRIVLAIY (SEQ ID NO:2).

In one embodiment, acceptable substitutions in X2 relative to SEQ ID NO:1 are selected from the group consisting of those shown in Table 1.

TABLE 1

| | Residue at that | |
|---|---|---|
| Residue # in | position in | Allowed physiochemical |
| SEQ ID NO: 1 | SEQ ID NO: 1 | classes |
| 1 | H | Any |
| 2 | S | Any |
| 3 | I | Aliphatic, polar |
| 4 | V | Aliphatic, polar, aromatic |
| 5 | Y | Any |
| 6 | A | Any |
| 7 | I | Aliphatic, aromatic, small |
| 8 | E | Any |
| 9 | A | Small, polar |
| 10 | A | Small, aliphatic |
| 11 | I | Any |
| 12 | F | Any |

As used herein, aliphatic residues include Ile, Val, Leu, and Ala; polar residues include Lys, Arg, Glu, Asp, Gln, Ser, Thr, and Asn; aromatic residues include Trp, Tyr, Phe; and small residues include Gly, Ser, Cys, Ala, and Thr. In another embodiment, acceptable substitutions in X2 relative to SEQ TD NO:1 are selected from the group consisting of those shown in Table 2.

TABLE 2

| | Residue at that | |
|---|---|---|
| Residue # in | position in | |
| SEQ ID NO: 1 | SEQ ID NO: 1 | Substitutions |
| 1 | H | P, V, M, F, Y, W, Q, E, or L |
| 2 | S | P, T, A, I, F, H, Q, R, or L |
| 3 | I | T, V, M, F, or N |
| 4 | V | T, C, I, L, M, F, or R |
| 5 | Y | A, V, L, M, F, W, H, Q, R, K, or I |
| 6 | A | S, C, V, L, M, Y, D, E, or R |
| 7 | I | T, S, C, V, L, M, or F |
| 8 | E | C, I, Y, or Q |
| 9 | A | S, C, or D |
| 10 | A | T, S, V, or M |
| 11 | I | F, N, R, A, or V |
| 12 | F | V, M, Y, W, N, Q, D, E, or H |

In a further embodiment, acceptable substitutions in X4 relative to SEQ TD NO:2 are selected from the group consisting of those shown in Table 3.

TABLE 3

| | Residue at that | |
|---|---|---|
| Residue # in | position in | Allowed physiochemical |
| SEQ ID NO: 2 | SEQ ID NO: 2 | classes |
| 1 | R | Any |
| 2 | N | Any |
| 3 | V | Any |
| 4 | E | Any |
| 5 | H | Any |
| 6 | A | Any |
| 7 | L | small, aliphatic, polar |
| 8 | M | aliphatic, aromatic, polar |
| 9 | R | Any |
| 10 | I | aliphatic, aromatic, polar |
| 11 | V | small, aliphatic, aromatic |
| 12 | L | small, aliphatic |
| 13 | A | Any |
| 14 | I | aliphatic, polar |
| 15 | Y | small, aromatic, polar |

In another embodiment, acceptable substitutions in X4 relative to SEQ TD NO:2 are selected from the group consisting of those shown in Table 4.

TABLE 4

| | Residue at that | |
|---|---|---|
| Residue # in | position in | |
| SEQ ID NO: 2 | SEQ ID NO: 2 | Substitutions |
| 1 | R | T, S, C, V, L, H, N, or Q |
| 2 | N | P, T, S, A, V, I, L, M, F, Y, W, H, N, |
| | | Q, D, K, or E |
| 3 | V | T, I, M, F, Y, W, H, Q, D, or E |
| 4 | E | L, Y, Q, D, K, N, or H |
| 5 | H | P, T, S, A, V, I, L, Y, N, Q, R, or K |
| 6 | A | P, T, S, C, V, L, N, Q, D, E, R, or K |
| 7 | L | P, C, V, I, M, or H |
| 8 | M | T, I, L, N, K, W, or R |
| 9 | R | P, A, I, M, W, N, Q, E, or K |
| 10 | I | V, L, M, F, or N |
| 11 | V | S, C, A, I, L, M, or F |
| 12 | L | P, I, H, F, S, or C |
| 13 | A | T, S, V, W, Q, E, R, or K |
| 14 | I | F or N |
| 15 | Y | C, F, H, N, G, W, D, or E |

In one embodiment, X2 comprises the amino acid sequence having at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identity to the full length of

(SEQ ID NO: 3)
SDVNEALHSIVYAIEAAIFALEAAERT.

In another embodiment, X4 comprises the amino acid sequence having at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identity to the full length of

(SEQ ID NO: 4)
RNVEHALMRIVLAIYLAEENLREAEES.

In a further embodiment, X3 comprises the amino acid sequence having at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identity to the full length of EVRELARELVRLAVEAAEEVQR (SEQ ID NO:5). In another embodiment, X5 comprises the amino acid sequence having at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identity to the full length of EKREKARERVREAVERAEEVQR (SEQ ID NO:6). In one embodiment, X1, when present, comprises the amino acid sequence having at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identity to the full length of SEQ ID NO:7.

(SEQ ID NO: 7)
SDEEEARELIERAKEAAERAQEAAERTGDPRVRELARELKRLAQEAAEE
VKRDPSSSDVNEALKLIVEAIEAAVDALEAAERTGDPEVRELARELVRL
AVEAAEEVQR

In various embodiments:
X2 comprises the amino acid sequence having at least 60% identity to the full length of

(SEQ ID NO: 3)
SDVNEALHSIVYAIEAAIFALEAAERT,

X4 comprises the amino acid sequence having at least 60% identity to the full length of

(SEQ ID NO: 4)
RNVEHALMRIVLAIYLAEENLREAEES,

X3 comprises the amino acid sequence having at least 60% identity to the full length of EVRELARELVRLAVEAAEEVQR (SEQ ID NO:5), X5 comprises the amino acid sequence having at least 60% identity to the full length of EKREKARERVREAVERAEEVQR (SEQ ID NO:6), and X1, when present, comprises the amino acid sequence having at least 60% identity to the full length of SEQ ID NO:7;
X2 comprises the amino acid sequence having at least 70% identity to the full length of

(SEQ ID NO: 3)
SDVNEALHSIVYAIEAAIFALEAAERT,

X4 comprises the amino acid sequence having at least 70% identity to the full length of

(SEQ ID NO: 4)
RNVEHALMRIVLAIYLAEENLREAEES,

X3 comprises the amino acid sequence having at least 70% identity to the full length of EVRELARELVRLAVEAAEEVQR (SEQ ID NO:5), X5 comprises the amino acid sequence having at least 70% identity to the full length of EKREKARERVREAVERAEEVQR (SEQ ID NO:6), and X1, when present, comprises the amino acid sequence having at least 70% identity to the full length of SEQ ID NO:7;
X2 comprises the amino acid sequence having at least 80% identity to the full length of

(SEQ ID NO: 3)
SDVNEALHSIVYAIEAAIFALEAAERT,

X4 comprises the amino acid sequence having at least 80% identity to the full length of

(SEQ ID NO: 4)
RNVEHALMRIVLAIYLAEENLREAEES,

X3 comprises the amino acid sequence having at least 80% identity to the full length of EVRELARELVRLAVEAAEEVQR (SEQ ID NO:5), X5 comprises the amino acid sequence having at least 80% identity to the full length of EKREKARERVREAVERAEEVQR (SEQ ID NO:6), and X1, when present, comprises the amino acid sequence having at least 80% identity to the full length of SEQ ID NO:7;
X2 comprises the amino acid sequence having at least 80% identity to the full length of

(SEQ ID NO: 3)
SDVNEALHSIVYAIEAAIFALEAAERT,

X4 comprises the amino acid sequence having at least 80% identity to the full length of

(SEQ ID NO: 4)
RNVEHALMRIVLAIYLAEENLREAEES,

X3 comprises the amino acid sequence having at least 80% identity to the full length of EVRELARELVRLAVEAAEEVQR (SEQ ID NO:5), X5 comprises the amino acid sequence having at least 80% identity to the full length of EKREKARERVREAVERAEEVQR (SEQ ID NO:6), and X1, when present, comprises the amino acid sequence having at least 80% identity to the full length of SEQ ID NO:7;
X2 comprises the amino acid sequence having at least 90% identity to the full length of

(SEQ ID NO: 3)
SDVNEALHSIVYAIEAAIFALEAAERT,

X4 comprises the amino acid sequence having at least 90% identity to the full length of

(SEQ ID NO: 4)
RNVEHALMRIVLAIYLAEENLREAEES,

X3 comprises the amino acid sequence having at least 90% identity to the full length of EVRELARELVRLAVEAAEEVQR (SEQ ID NO:5), X5 comprises the amino acid sequence having at least 90% identity to the full length of EKREKARERVREAVERAEEVQR (SEQ ID NO:6), and X1, when present, comprises the amino acid sequence having at least 90% identity to the full length of SEQ ID NO:7;
X2 comprises the amino acid sequence having at least 95% identity to the full length of

(SEQ ID NO: 3)
SDVNEALHSIVYAIEAAIFALEAAERT,

X4 comprises the amino acid sequence having at least 95% identity to the full length of

(SEQ ID NO: 4)
RNVEHALMRIVLAIYLAEENLREAEES,

X3 comprises the amino acid sequence having at least 95% identity to the full length of EVRELARELVRLAVEAAEEVQR (SEQ ID NO:5), X5 comprises the amino acid sequence having at least 95% identity to the full length of EKREKARERVREAVERAEEVQR (SEQ ID NO:6), and X1, when present, comprises the amino acid sequence having at least 95% identity to the full length of SEQ ID NO:7; or
X2 comprises the amino acid sequence having at least 100% identity to the full length of

(SEQ ID NO: 3)
SDVNEALHSIVYAIEAAIFALEAAERT,

X4 comprises the amino acid sequence having 100% identity to the full length of

(SEQ ID NO: 4)
RNVEHALMRIVLAIYLAEENLREAEES,

X3 comprises the amino acid sequence having 100% identity to the full length of EVRELARELVRLAVEAAEEVQR (SEQ ID NO:5), X5 comprises the amino acid sequence having 100% identity to the full length of EKREKARERVREAVERAEEVQR (SEQ ID NO:6), and X1, when present, comprises the amino acid sequence having 100% identity to the full length of SEQ ID NO:7.

In various further embodiments, the polypeptide comprises the amino acid sequence having at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identity to the full length of SEQ ID NO:8, SEQ ID NO:9, or SEQ ID NO:10.

DNCR2
(SEQ ID NO: 8)
SSDEEEARELIERAKEAAERAQEAAERTGDPRVRELARELKRLAQEAAEE
VKRDPSSSDVNEALKLIVEAIEAAVDALEAAERTGDPEVRELARELVRLA
VEAAEEVQRNPSSSDVNEALHSIVYAIEAAIFALEAAERTGDPEVRELAR
ELVRLAVEAAEEVQRNPSSRNVEHALMRIVLAIYLAEENLREAEESGDPE
KREKARERVREAVERAEEVQRDPSGWLNH;
DNCR1
(SEQ ID NO: 9)
SSDEEEARELIERAKEAAERAQEAAERTGDPRVRELARELKRLAQEAAEE
VKRDPSSSDVNEALKLIVEAIEAAVDALEAAERTGDPEVRELARELVRLA
VEAAEEVQRNPSSSDVNEALLSIVIAIEAAVHALEAAERTGDPEVRELAR
ELVRLAVEAAEEVQRNPSSREVEHALMKIVLAIYEAEESLREAEESGDPE
KREKARERVREAVERAEEVQRDPSGWLNH;
or
D5
(SEQ ID NO: 10)
SSDEEEARELIERAKEAAERAQEAAERTGDPRVRELARELKRLAQEAAEE
VKRDPSSSDVNEALKLIVEAIEAAVDALEAAERTGDPEVRELARELVRLA
VEAAEEVQRNPSSSDVNEALLTIVIAIEAAVNALEAAERTGDPEVRELAR
ELVRLAVEAAEEVQRNPSSREVNIALWKIVLAIQEAVESLREAEESGDPE
KREKARERVREAVERAEEVQRDPSGWLNH.

As discussed in the examples that follow, the inventors have extensively characterized permitted variability in the sequence of the DNCR polypeptides disclosed herein. Exemplary substitutions are provided in Table 5 and based on experimental variation of DNCR1 (SEQ ID NO: 9) positions 117-191. Thus, in one embodiment, acceptable substitutions relative to SEQ ID NO:8-10 are selected from the group shown in Table 5.

TABLE 5
DNCR permitted interface variation

| Position | DNCR2 | | Allowed physicochemical |
|---|---|---|---|
| number* | residue | Exemplary substitutions | classes |
| 117 | N | S, I, L, Y, H, Q, D, E, or K | Any |
| 118 | E | I, H, D, R, or K | Any |
| 119 | A | S, C, V, I, L, Y, N, Q, D, or K | Any |
| 120 | L | P, I, or H | Aliphatic, polar |
| 121 | H | P, V, M, F, Y, W, Q, E, or L | Any |
| 122 | S | P, T, A, I, F, H, Q, R, or L | Any |
| 123 | I | T, V, M, F, or N | Aliphatic, polar |
| 124 | V | T, C, I, L, M, F, or R | Aliphatic, polar, aromatic |
| 125 | Y | A, V, L, M, F, W, H, Q, R, K, or I | Any |
| 126 | A | S, C, V, L, M, Y, D, E, or R | Any |
| 127 | I | T, S, C, V, L, M, or F | Aliphatic, aromatic, small |
| 128 | E | C, I, Y, or Q | Any |
| 129 | A | S, C, or D | Small, polar |
| 130 | A | T, S, V, or M | Small, aliphatic |
| 131 | I | F, N, R, A, or V | Any |
| 132 | F | V, M, Y, W, N, Q, D, E, or H | Any |
| 133 | A | P, T, S, V, or E | Any |
| 134 | L | P | Any |
| 135 | E | A, Q, or D | Any |
| 136 | A | S or F | Any |
| 137 | A | T, S, V, I, L, M, F, W, or E | Any |
| 138 | E | F or D | Any |
| 139 | R | S, C, L, or H | Any |
| 140 | T | P, S, A, I, H, or N | Any |
| 141 | G | C, N, Q, D, R, or K | Any |
| 142 | D | T, S, A, V, Y, H, N, or E | Any |
| 143 | P | T, S, A, V, Y, W, H, Q, D, E, or R | Any |
| 144 | E | T, A, V, I, L, M, F, Y, W, H, Q, D, or R | Any |
| 145 | V | I, L, or M | Any |
| 146 | R | T, S, C, A, V, L, M, F, Y, W, H, N, Q, or E | Any |
| 147 | E | T, A, V, I, L, Y, H, N, Q, D, or R | Any |
| 148 | L | P, C, V, I, M, or Q | Any |
| 149 | A | T, S, or D | Any |
| 150 | R | T, S, C, I, L, M, F, Y, W, H, Q, or E | Any |
| 151 | E | D | Any |
| 152 | L | P, A, or M | Any |
| 153 | V | C, I, L, F, or D | Any |
| 154 | R | P, T, S, C, A, V, I, L, M, F, W, H, or E | Any |
| 155 | L | T, A, V, I, R, or K | Any |
| 156 | A | T, S, or D | Any |
| 157 | V | I or F | Any |
| 158 | E | P, T, A, V, H, Q, D, R, or K | Any |
| 159 | A | T, S, C, V, L, M, F, W, H, N, Q, E, or K | Any |
| 160 | A | P, T, or S | Any |
| 161 | E | T, S, C, A, V, I, L, M, Y, H, N, Q, D, R, or K | Any |
| 162 | E | P, T, S, A, V, W, N, Q, D, or R | Any |
| 163 | V | P, T, S, A, L, N, Q, or D | Any |
| 164 | Q | T, S, C, H, N, D, R, or K | Any |
| 165 | R | T, S, C, A, V, I, L, Y, H, N, E, or K | Any |
| 166 | N | P, S, V, I, D, or K | Any |
| 167 | P | T, I, L, F, Q, or K | Any |
| 168 | S | P, T, C, A, Y, H, Q, R, or K | Any |
| 169 | S | P, A, V, Y, H, N, Q, R, or K | Any |
| 170 | R | T, S, C, V, L, H, N, or Q | Any |
| 171 | N | P, T, S, A, V, I, L, M, F, Y, W, H, N, Q, D, | Any |
| | | K, or E | |
| 172 | V | T, I, M, F, Y, W, H, Q, D, or E | Any |
| 173 | E | L, Y, Q, D, K, N, or H | Any |
| 174 | H | P, T, S, A, V, I, L, Y, N, Q, R, or K | Any |
| 175 | A | P, T, S, C, V, L, N, Q, D, E, R, or K | Any |
| 176 | L | P, C, V, I, M, or H | small, aliphatic, polar |
| 177 | M | T, I, L, N, K, W, or R | aliphatic, aromatic, polar |
| 178 | R | P, A, I, M, W, N, Q, E, or K | Any |
| 179 | I | V, L, M, F, or N | aliphatic, aromatic, polar |
| 180 | V | S, C, A, I, L, M, or F | small, aliphatic, aromatic |
| 181 | L | P, I, H, F, S, or C | small, aliphatic |
| 182 | A | T, S, V, W, Q, E, R, or K | Any |
| 183 | I | F or N | aliphatic, polar |
| 184 | Y | C, F, H, N, G, W, D, or E | small, aromatic, polar |
| 185 | L | C, I, E, M, F, W, Q, D, or K | Any |
| 186 | A | T, S, V, I, H, Q, E, R, or K | Any |
| 187 | E | D, K, A, S, or Y | small, aromatic, polar |
| 188 | E | V, I, Q, D, R, or K | aliphatic, polar |
| 189 | N | P, T, C, A, V, Y, H, E, R, K, or S | Any |
| 190 | L | P, A, V, I, M, H, or K | Any |
| 191 | R | S, C, I, H, or K | small, aliphatic, polar |

*Key residues at the DNCR2 interface were defined as residues 121-132 (in helix 5) and 170-184 (in helix 7); see sequence alignments below that show position of helices. All residues outside these ranges can be replaced by any sequence that supports the positions of these helical domains.
**Exemplary substitutions are based on experimental variation of DNCR1 positions 117-191.

In another aspect, the disclosure provides non-naturally occurring polypeptide comprising the general formula X1-X2-X3-X4-X5-X6-X7, wherein:

X1 comprises first helical domain;

X2 comprises a second helical domain comprising the amino acid sequence having at least 50%, 55%, 60%, 65%, 70%, 75%, 80%, 85%, 90%, 91%, 92%, 93%, 94%, 95%, 96%, 97, 98% 99%, or 100% identity to the full length of

(SEQ ID NO: 20)
DLANLAVAAVLTACL,

wherein 1, 2, 3, 4, 5, 6, or all 7 of the following changes from SEQ ID NO:20 are not permissible: D1K, N4S, L5Q, A8E, L11K, T12L, and L15E;

X3 comprises a third helical domain;

X4 comprises a fourth helical domain comprising the amino acid sequence having at least 50%, 55%, 60%, 65%, 70%, 75%, 80%, 85%, 90%, 91%, 92%, 93%, 94%, 95%, 96%, 97%, 98%, 99%, or 100% identity to the full length of RAVILAIM (SEQ ID NO:21), wherein 1, 2, 3, or all 4 of the following changes from SEQ ID NO:21 are not permissible: R1E, I4K, I7C, and M8E;

X5 comprises a fifth helical domain;

X6 comprises a sixth helical domain comprising the amino acid sequence having at least 50%, 55%, 60%, 65%, 70%, 75%, 80%, 85%, 90%, 91%, 92%, 93%, 94%, 95%, 96%, 97%, 98%, 99%, or 100% identity to the full length of RAIWLAAE (SEQ ID NO:22), wherein 1, 2, 3, or all 4 of the following changes from SEQ ID NO:22 are not permissible: R1L, I3C, W4E, and A7Q; and

X7 comprises seventh and eighth helical domains.

The polypeptides of this aspect are grazoprevir/NS3a complex reader (GNCR) polypeptides, defined as a protein that selectively binds the grazoprevir/NS3a complex over the apo NS3a protein, where NS3a is any variant of the HCV protease NS3/4a (any genotype and catalytically active or dead), as described in detail herein. The functional part of GNCR is the interface with grazoprevir/NS3a, which includes portions of helices 2, 4, and 6, as defined herein. This interface can be grafted onto any protein backbone that supported the arrangement of these helices and still serve as a grazoprevir/NS3a complex reader. Additionally, there is flexibility in the sequence of these interface helices, with exemplary mutational trends discussed in the examples herein.

In one embodiment, acceptable substitutions in X2 relative to SEQ ID NO:20 are selected from the group consisting of those shown in Table 6

TABLE 6

| | Residue at that | |
|---|---|---|
| Residue # in | position in | Allowed physiochemical |
| SEQ ID NO: 20 | SEQ ID NO: 20 | classes |
| 1 | D | Any |
| 2 | L | aliphatic |
| 3 | A | small |
| 4 | N | Any |
| 5 | L | polar, aliphatic |
| 6 | A | small |
| 7 | V | Any |
| 8 | A | small |
| 9 | A | small, aliphatic |
| 10 | V | aliphatic |
| 11 | L | aliphatic |
| 12 | T | small, aliphatic |
| 13 | A | small |
| 14 | C | small, aliphatic |
| 15 | L | small, aliphatic |

In another embodiment, acceptable substitutions in X2 relative to SEQ ID NO:20 are selected from the group shown in Table 7.

TABLE 7

| | Residue at that | |
|---|---|---|
| Residue # in | position in | |
| SEQ ID NO: 20 | SEQ ID NO: 20 | Substitutions |
| 1 | D | E, V, A, F, or W |
| 2 | L | |
| 3 | A | |
| 4 | N | Q, A, W, R, I, or N |
| 5 | L | E, Q, or I |
| 6 | A | |
| 7 | V | I, W, E, Y, F, M, or V |
| 8 | A | |
| 9 | A | V |
| 10 | V | |
| 11 | L | I |
| 12 | T | A, L, M, or S |
| 13 | A | |
| 14 | C | |
| 15 | L | L or S |

In a further embodiment, acceptable substitutions in X4 relative to SEQ ID NO:21 are selected from the group shown in Table 8

TABLE 8

| | Residue at that | |
|---|---|---|
| Residue # in | position in | Allowed physiochemical |
| SEQ ID NO: 21 | SEQ ID NO: 21 | classes |
| 1 | R | polar, aliphatic |
| 2 | A | small |
| 3 | V | small, aliphatic |
| 4 | I | small, aliphatic |
| 5 | L | aliphatic |
| 6 | A | small |
| 7 | I | aliphatic |
| 8 | M | small, aliphatic |

In another embodiment, acceptable substitutions in X4 relative to SEQ ID NO:21 are selected from the group consisting those shown in Table 9.

TABLE 9

| | Residue at that | |
|---|---|---|
| Residue # in | position in | |
| SEQ ID NO: 21 | SEQ ID NO: 21 | Substitutions |
| 1 | R | I or L |
| 2 | A | |
| 3 | V | |
| 4 | I | L, M, or A |
| 5 | L | |
| 6 | A | |
| 7 | I | |
| 8 | M | A |

In one embodiment, acceptable substitutions in X6 relative to SEQ ID NO:22 are selected from the group consisting of those shown in Table 10

TABLE 10

| | Residue at that | |
|---|---|---|
| Residue # in | position in | Allowed physiochemical |
| SEQ ID NO: 22 | SEQ ID NO: 22 | classes |
| 1 | R | aliphatic, polar |
| 2 | A | small |
| 3 | I | aliphatic |
| 4 | W | aliphatic, aromatic |
| 5 | L | aliphatic |
| 6 | A | small |
| 7 | A | small |
| 8 | E | polar, aliphatic |

In a further embodiment, acceptable substitutions in X6 relative to SEQ ID NO:22 are selected from those shown in Table 11.

TABLE 11

| | Residue at that | |
|---|---|---|
| Residue # in | position in | |
| SEQ ID NO: 22 | SEQ ID NO: 22 | Substitutions |
| 1 | R | V or L |
| 2 | A | |
| 3 | I | |
| 4 | W | L |
| 5 | L | |
| 6 | A | |
| 7 | A | I |
| 8 | E | L, M, or K |

In various embodiments,
X2 comprises a second helical domain comprising the amino acid sequence having at least 60% identity to the full length of

(SEQ ID NO: 20)
DLANLAVAAVLTACL,

wherein 1, 2, 3, 4, 5, 6, or all 7 of the following changes from SEQ ID NO: 20 are not permissible: D1K, N4S, L5Q, A8E, L11K, T12L, and L15E; X4 comprises a fourth helical domain comprising the amino acid sequence having at least 60% identity to the full length of RAVILAIM (SEQ ID NO:21), wherein 1, 2, 3, or all 4 of the following changes from SEQ ID NO:21 are not permissible: R1E, I4K, I7C, and M8E; and X6 comprises a sixth helical domain comprising the amino acid sequence having at least 60% identity to the full length of RAIWLAAE (SEQ ID NO:22), wherein 1, 2, 3, or all 4 of the following changes from SEQ ID NO:22 are not permissible: R1L, I3C, W4E, and A7Q;
X2 comprises a second helical domain comprising the amino acid sequence having at least 70% identity to the full length of

(SEQ ID NO: 20)
DLANLAVAAVLTACL,

wherein 1, 2, 3, 4, 5, 6, or all 7 of the following changes from SEQ ID NO:20 are not permissible: D1K, N4S, L5Q, A8E, L11K, T12L, and L15E; X4 comprises a fourth helical domain comprising the amino acid sequence having at least 70% identity to the full length of RAVILAIM (SEQ ID NO:21), wherein 1, 2, 3, or all 4 of the following changes from SEQ ID NO:21 are not permissible: R1E, I4K, I7C, and M8E; and X6 comprises a sixth helical domain comprising the amino acid sequence having at least 70% identity to the full length of RAIWLAAE (SEQ ID NO:22), wherein 1, 2, 3, or all 4 of the following changes from SEQ ID NO:22 are not permissible: R1L, I3C, W4E, and A7Q;
X2 comprises a second helical domain comprising the amino acid sequence having at least 80% identity to the full length of

(SEQ ID NO: 20)
DLANLAVAAVLTACL,

wherein 1, 2, 3, 4, 5, 6, or all 7 of the following changes from SEQ ID NO:20 are not permissible: D1K, N4S, L5Q, A8E, L11K, T12L, and L15E; X4 comprises a fourth helical domain comprising the amino acid sequence having at least 80% identity to the full length of RAVILAIM (SEQ ID NO:21), wherein 1, 2, 3, or all 4 of the following changes from SEQ ID NO:21 are not permissible: R1E, I4K, I7C, and M8E; and X6 comprises a sixth helical domain comprising the amino acid sequence having at least 80% identity to the full length of RAIWLAAE (SEQ ID NO:22), wherein 1, 2, 3, or all 4 of the following changes from SEQ ID NO:22 are not permissible: R1L, I3C, W4E, and A7Q;
X2 comprises a second helical domain comprising the amino acid sequence having at least 90% identity to the full length of

(SEQ ID NO: 20)
DLANLAVAAVLTACL,

wherein 1, 2, 3, 4, 5, 6, or all 7 of the following changes from SEQ ID NO:20 are not permissible: D1K, N4S, L5Q, A8E, L11K, T12L, and L15E; X4 comprises a fourth helical domain comprising the amino acid sequence having at least 90% identity to the full length of RAVILAIM (SEQ ID NO:21), wherein 1, 2, 3, or all 4 of the following changes from SEQ ID NO:21 are not permissible: R1E, I4K, I7C, and M8E; and X6 comprises a sixth helical domain comprising the amino acid sequence having at least 90% identity to the full length of RAIWLAAE (SEQ ID NO:22), wherein 1, 2, 3, or all 4 of the following changes from SEQ ID NO:22 are not permissible: R1L, I3C, W4E, and A7Q; or
X2 comprises a second helical domain comprising the amino acid sequence having 100% identity to the full length of

(SEQ ID NO: 20)
DLANLAVAAVLTACL,

wherein 1, 2, 3, 4, 5, 6, or all 7 of the following changes from SEQ ID NO:20 are not permissible: D1K, N4S, L5Q, A8E, L11K, T12L, and L15E; X4 comprises a fourth helical domain comprising the amino acid sequence having 100% identity to the full length of RAVILAIM (SEQ ID NO:21), wherein 1, 2, 3, or all 4 of the following changes from SEQ ID NO:21 are not permissible: R1E, I4K, I7C, and M8E; and X6 comprises a sixth helical domain comprising the amino acid sequence having 100% identity to the full length of RAIWLAAE (SEQ ID NO:22), wherein 1, 2, 3, or all 4 of the following changes from SEQ ID NO:22 are not permissible: R1L, 13C, W4E, and A7Q.

In another embodiment, X2 comprises the amino acid sequence having at least 25%, 30%, 35%0, 40%, 45%, 50%, 55%, 60%, 65%, 70%, 75%, 80%, 85%, 90%, 91%, 92%, 93%, 94%, 95%, 96%, 97%, 98%, 99%, or 100% identity to the full length of

(SEQ ID NO: 23)
QAAEDAEDLANLAVAAVLTACLLAQEH.

In a further embodiment, X4 comprises the amino acid sequence having at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identity to the full length of

(SEQ ID NO: 24)
QAARDAIKLASQAARAVILAIMLAA.

In one embodiment, X6 comprises the amino acid sequence having at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identity to the full length of

(SEQ ID NO: 25)
QAARDAIKLASQAAEAVERAIWLAAE.

In another embodiment, X1 comprises the amino acid sequence having at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identity to the full length of IEKLCKKAEEEAKEAQEKADELRQRH (SEQ ID NO:26). In a further embodiment, X3 comprises the amino acid sequence having at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identity to the full length of DIAKLCIKAASEAAEAASKAAELAQR (SEQ ID NO: 27). In one embodiment, X5 comprises the amino acid sequence having at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identity to the full length of DIAKLCIKAASEAAEAASKAAELAQR (SEQ ID NO:28). In another embodiment, X7 comprises the amino acid sequence having at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identity to the full length of

(SEQ ID NO: 29)
DIAKKCIKAASEAAEEASKAAEEAQRHPDSQKARDEIKEASQKAEEVKER.

In various embodiments
X2 comprises the amino acid sequence having at least 60% identity to the full length of

(SEQ ID NO: 23)
QAAEDAEDLANLAVAAVLTACLLAQEH,

X4 comprises the amino acid sequence having at least 60% identity to the full length of

(SEQ ID NO: 24)
QAARDAIKLASQAARAVILAIMLAA,

X6 comprises the amino acid sequence having at least 60% identity to the full length of

(SEQ ID NO: 25)
QAARDAIKLASQAAEAVERAIWLAAE,

X1 comprises the amino acid sequence having at least 60% identity to the full length of IEKLCKKAEEEAKEAQEKADELRQRH (SEQ ID NO:26), X3 comprises the amino acid sequence having at least 60% identity to the full length of DIAKLCIKAASEAAEAASKAAELAQR (SEQ ID NO: 27), X5 comprises the amino acid sequence having at least 60% identity to the full length of DIAKLCIKAASEAAEAASKAAELAQR (SEQ ID NO:28), and X7 comprises the amino acid sequence having at least 60% identity to the full length of

(SEQ ID NO: 29)
DIAKKCIKAASEAAEEASKAAEEAQRHPDSQKARDEIKEASQKAEEVKER;

X2 comprises the amino acid sequence having at least 70% identity to the full length of

(SEQ ID NO: 23)
QAAEDAEDLANLAVAAVLTACLLAQEH,

X4 comprises the amino acid sequence having at least 70% identity to the full length of

(SEQ ID NO: 24)
QAARDAIKLASQAARAVILAIMLAA,

X6 comprises the amino acid sequence having at least 70% identity to the full length of

(SEQ ID NO: 25)
QAARDAIKLASQAAEAVERAIWLAAE,

X1 comprises the amino acid sequence having at least 70% identity to the full length of IEKLCKKAEEEAKEAQEKADELRQRH (SEQ ID NO:26), X3 comprises the amino acid sequence having at least 70% identity to the full length of DIAKLCIKAASEAAEAASKAAELAQR (SEQ ID NO: 27), X5 comprises the amino acid sequence having at least 70% identity to the full length of DIAKLCIKAASEAAEAASKAAELAQR (SEQ ID NO:28), and X7 comprises the amino acid sequence having at least 70% identity to the full length of

(SEQ ID NO: 29)
DIAKKCIKAASEAAEEASKAAEEAQRHPDSQKARDEIKEASQKAEEVKER;

X2 comprises the amino acid sequence having at least 80% identity to the full length of

(SEQ ID NO: 23)
QAAEDAEDLANLAVAAVLTACLLAQEH,

X4 comprises the amino acid sequence having at least 80% identity to the full length of

(SEQ ID NO: 24)
QAARDAIKLASQAARAVILAIMLAA,

X6 comprises the amino acid sequence having at least 80% identity to the full length of

(SEQ ID NO: 25)
QAARDAIKLASQAAEAVERAIWLAAE,

X1 comprises the amino acid sequence having at least 80% identity to the full length of IEKLCKKAEEEAKEAQEKADELRQRH (SEQ ID NO:26), X3 comprises the amino acid sequence having at least 80% identity to the full length of DIAKLCIKAASEAAEAASKAAELAQR (SEQ ID NO: 27), X5 comprises the amino acid sequence having at least 80% identity to the full length of DIAKLCIKAASEAAEAASKAAELAQR (SEQ ID NO:28), and X7 comprises the amino acid sequence having at least 80% identity to the full length of

(SEQ ID NO: 29)
DIAKKCIKAASEAAEEASKAAEEAQRHPDSQKARDEIKEASQKAEEVKER;

X2 comprises the amino acid sequence having at least 90% identity to the full length of

(SEQ ID NO: 23)
QAAEDAEDLANLAVAAVLTACLLAQEH,

X4 comprises the amino acid sequence having at least 90% identity to the full length of

(SEQ ID NO: 24)
QAARDAIKLASQAARAVILAIMLAA,

X6 comprises the amino acid sequence having at least 90% identity to the full length of

(SEQ ID NO: 25)
QAARDAIKLASQAAEAVERAIWLAAE,

X1 comprises the amino acid sequence having at least 90% identity to the full length of IEKLCKKAEEEAKEAQEKADELRQRH (SEQ ID NO:26), X3 comprises the amino acid sequence having at least 90% identity to the full length of DIAKLCIKAASEAAEAASKAAELAQR (SEQ ID NO: 27), X5 comprises the amino acid sequence having at least 90% identity to the full length of DIAKLCIKAASEAAEAASKAAELAQR (SEQ ID NO:28), and X7 comprises the amino acid sequence having at least 90% identity to the full length of

(SEQ ID NO: 29)
DIAKKCIKAASEAAEEASKAAEEAQRHPDSQKARDEIKEASQKAEEVKER;

X2 comprises the amino acid sequence having at least 95% identity to the full length of

(SEQ ID NO: 23)
QAAEDAEDLANLAVAAVLTACLLAQEH,

X4 comprises the amino acid sequence having at least 95% identity to the full length of

(SEQ ID NO: 24)
QAARDAIKLASQAARAVILAIMLAA,

X6 comprises the amino acid sequence having at least 95% identity to the full length of

(SEQ ID NO: 25)
QAARDAIKLASQAAEAVERAIWLAAE,

X1 comprises the amino acid sequence having at least 95% identity to the full length of IEKLCKKAEEEAKEAQEKADELRQRH (SEQ ID NO:26), X3 comprises the amino acid sequence having at least 95% identity to the full length of DIAKLCIKAASEAAEAASKAAELAQR (SEQ ID NO: 27), X5 comprises the amino acid sequence having at least 95% identity to the full length of DIAKLCIKAASEAAEAASKAAELAQR (SEQ ID NO:28), and X7 comprises the amino acid sequence having at least 95% identity to the full length of

(SEQ ID NO: 29)
DIAKKCIKAASEAAEEASKAAEEAQRHPDSQKARDEIKEASQKAEE
VKER;
or

X2 comprises the amino acid sequence having 100% identity to the full length of

(SEQ ID NO: 23)
QAAEDAEDLANLAVAAVLTACLLAQEH,

X4 comprises the amino acid sequence having 100% identity to the full length of

(SEQ ID NO: 24)
QAARDAIKLASQAARAVILAIMLAA,

X6 comprises the amino acid sequence having 100% identity to the full length of

(SEQ ID NO: 25)
QAARDAIKLASQAAEAVERAIWLAAE ,

X1 comprises the amino acid sequence having 100% identity to the full length of IEKLCKKAEEEAKEAQEKADELRQRH (SEQ ID NO:26), X3 comprises the amino acid sequence having 100% identity to the full length of DIAKLCIKAASEAAEAASKAAELAQR (SEQ ID NO: 27), X5 comprises the amino acid sequence having 100% identity to the full length of DIAKLCIKAASEAAEAASKAAELAQR (SEQ ID NO:28), and X7 comprises the amino acid sequence having 100% identity to the full length of

(SEQ ID NO: 29)
DIAKKCIKAASEAAEEASKAAEEAQRHPDSQKARDEIKEASQKAEE
VKER.

In another embodiment, the polypeptide has at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identity to the full length of a polypeptide selected from the group consisting of SEQ ID NOS:11-12

(SEQ ID NO: 11)
DIEKLCKKAEEEAKEAQEKADELRQRHPDSQAAEDAEDLANLAVAAVLTACLLAQEHPNADI
AKLCIKAASEAAEAASKAAELAQRHPDSQAARDAIKLASQAARAVILAIMLAAENPNADIAK
LCIKAASEAAEAASKAAELAQRHPDSQAARDAIKLASQAAEAVERAIWLAAENPNADIAKKC
IKAASEAAEEASKAAEEAQRHPDSQKARDEIKEASQKAEEVKERCKS
(SEQ ID NO: 12)
DIEKLCKKAEEEAKEAQEKADELRQRHPDSQAAEDAEDLANEAEAAVLAACSLAQEHPNADI
AKLCIKAASEAAEAASKAAELAQRHPDSQAARDAIKLASQAARAVILAIMLAAENPNADIAK
LCIKAASEAAEAASKAAELAQRHPDSQAARDAIKLASQAAEAVERAIWLAAENPNADIAKKC
IKAASEAAEEASKAAEEAQRHPDSQKARDEIKEASQKAEEVKERCKS

The inventors have extensively characterized permitted variability in the sequence of the GNCR polypeptides disclosed herein. In one embodiment, acceptable substitutions relative to SEQ ID NO:11-12 are selected from the group shown in Table 12.

TABLE 12
GNCR permitted interface variation

| Position | GNCR1 | Favored | Allowed physicochemical |
|---|---|---|---|
| number* | residue | substitutions** | classes |
| 38 | D | E, V, A, F, or W | Any |
| 39 | L | | aliphatic |
| 40 | A | | small |
| 41 | N | Q, A, W, R, I, or N | Any |
| 42 | L | E, Q, or I | polar, aliphatic |
| 43 | A | | small |
| 44 | V | I, W, E, Y, F, M, | Any |
| | | or V | |
| 45 | A | | small |
| 46 | A | V | small, aliphatic |
| 47 | V | | aliphatic |
| 48 | L | I | aliphatic |
| 49 | T | A, L, M, or S | small, aliphatic |
| 50 | A | | small |
| 51 | C | | small, aliphatic |
| 52 | L | L or S | small, aliphatic |
| 105 | R | I or L | polar, aliphatic |
| 106 | A | | small |
| 107 | V | | small, aliphatic |
| 108 | I | L, M, or A | small, aliphatic |
| 109 | L | | aliphatic |
| 110 | A | | small |
| 111 | I | | aliphatic |
| 112 | M | A | small, aliphatic |
| 169 | R | V or L | aliphatic, polar |
| 170 | A | | small |
| 171 | I | | aliphatic |
| 172 | W | L | aliphatic, aromatic |
| 173 | L | | aliphatic |
| 174 | A | | small |
| 175 | A | I | small |
| 176 | E | L, M, or K | polar, aliphatic |

*Key residues of the GNCR1 interface are residues 38-52, 105-112, and 169-176. All residues outside these ranges can be replaced by any sequence that supports the positions of these helical fragments.
**Exemplary substitutions are based on computational predictions and experimental variation.

In another embodiment, amino acid substitutions relative to the reference peptides are conservative amino acid substitutions. As used herein, “conservative amino acid substitution” means a given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are known. Polypeptides comprising conservative amino acid substitutions can be tested in any one of the assays described herein to confirm that a desired activity, e.g. antigen-binding activity and specificity of a native or reference polypeptide is retained. Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into H is; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In all of the above embodiments of the DNCR and the GNCR polypeptides, the polypeptides may comprise amino acid linkers between one or more of the helical domains. Any suitable linkers can be used, having any amino acid composition and length as determined appropriate for an intended use. In various embodiments, the linkers may be flexible, for example being rich in glycine, serine, and/or threonine residues. In other embodiments, the linker may not include proline residues.

In one embodiment, the disclosure provides fusion protein comprising:

(a) the polypeptide of any embodiment or combination of embodiments disclosed herein; and

(b) a polypeptide localization domain at the N-terminus and/or the C-terminus of the fusion protein.

This embodiment permits localization to a cellular target. Any suitable localization domain can be used as deemed appropriate for an intended purpose. In non-limiting embodiments, the localization domain may target the fusion protein to the cell membrane, the nucleus, the mitochondria, Golgi apparatus, cell surface receptors, etc.

In another embodiment, the disclosure provides fusion protein comprising:

(a) the polypeptide of any embodiment or combination of embodiments disclosed herein; and

(b) a protein having one or more interaction surfaces.

This embodiment provide additional functionality to the polypeptides by regulating interactions with binding partners of the protein having one or more interaction surface. Any suitable protein can be used as deemed appropriate for an intended purpose. In non-limiting embodiments, the protein having one or more interaction surfaces comprises an enzymatic protein, protein-protein interaction domain, a nucleic acid-binding domain, etc. In various further embodiments, the protein having one or more interaction surfaces is selected from the group consisting of: Cas9 and related CRISPR proteins (catalytically active or dead), a DNA binding domain of a transcription factor (such as the Gal4 DNA binding domain), a pro-apoptotic domain (such as caspase 9), and a cell surface receptor (such as a chimeric antigen receptor).

In another aspect, the disclosure provides recombinant fusion proteins, comprising a polypeptide of the general formula X1-B1-X2-B2-X3, wherein

(a) one of X1 and X3 is selected from the group consisting of
(i) a peptide comprising the amino acid sequence having at least 75%, 80%, 85%, 90%, 91%, 92%, 93%, 94%, 95%, 96%, 97%, 98%, 99%, or 100% identity to the full length of the amino acid sequence selected from GELGRLVYLLDGPGYDPIHSD (SEQ ID NO:13), GELDELVYLLDGPGYDPIHSD (SEQ ID NO:14), GELGELVYLLDGPGYDPIHSD (SEQ ID NO: 15), or GELDRLVYLLDGPGYDPIHSD (SEQ ID NO:16), or GELDELVYLLDGPGYDPIHSDVVTRGGSHLFNF (SEQ ID NO:17) (“ANR peptide”);
(ii) the DNCR polypeptide of any embodiment or combination of embodiments disclosed herein; and
(iii) the GNCR polypeptide of any embodiment or combination of embodiments disclosed herein;

(b) the other of X1 and X3 is an NS3a peptide (either catalytically active or dead), wherein if X1 or X3 is the ANR peptide, then NS3a is one of the following variants of HCV protease NS3/4a: NS3a (SEQ ID NO:30), or engineered variants NS3a* (SEQ ID NO:31), NS3a-H1 (SEQ ID NO:32), -H2 (SEQ ID NO:33), -H3 (SEQ ID NO:34), -H4 (SEQ ID NO:35), -H5 (SEQ ID NO:36), or -H6 ((SEQ ID NO:37);

(c) X2 is a protein having one or more interaction surfaces; and

(d) B1 and B2 are optional amino acid linkers.

As described in detail in the examples that follow, the inventors have discovered that the recombinant fusion proteins of the disclosure may be used, for example, to disallow access to the X2 protein by occlusion of its interaction surface by an X1/X3 complex in the basal state (“intramolecular binding”). This complex can then be disrupted by any of the small molecule NS3a inhibitors, allowing access to the X2 protein, as described herein. Alternatively, when X1 or X3 is the DNCR or GNCR polypeptide, access to the X2 protein interaction surface is enabled in the basal state and occluded by interaction with NS3a when the appropriate small molecule NS3a inhibitor is present (danoprevir or grazoprevir, respectively).

In one embodiment, the NS3a peptide comprises the amino acid sequence having at least 80%, 85%, 90%, 91%, 92%, 93%, 94%, 95%, 96%, 97%, 98%, 99%, or 100% identity to the full length of the amino acid sequence selected from the group consisting of SEQ ID NO:30-38, wherein the bolded amino acid residue is the catalytic position, wherein the bolded “S” residue represents catalytically active NS3a peptides, and wherein the bolded ‘S” residue can be substituted with an alanine (or other) residue to render the NS3a peptide catalytically “dead” (which will also work in all applications):

NS3a Sequence:
(SEQ ID NO: 30)
MAKGSVVIVGRINLSGDTAYSQQTRGAAGTAATSATGRDKNQVDGEVQVLSTATQSFLATCVNGVCWT
VYHGAGSKTLAGPKGPITQMYTNVDQDLVGWPAPPGARSMTPCTCGSSDLYLVTRHADVIPVRRRGDS
RGSLLSPRPVSYLKGSSGGPLLCPSGHVVGIFRAAVCTRGVAKAVDFIPVESMETTMR
NS3a* Sequence
(SEQ ID NO: 31)
MKKKGSVVIVGRINLSGDTAYAQQTRGEEGCQETSQTGRDKNQVEGEVQIVSTATQTFLATSINGVLW
TVYHGAGTRTIASPKGPVTQMYTNVDKDLVGWQAPQGSRSLTPCTCGSSDLYLVTRHADVIPVRRRGD
SRGSLLSPRPISYLKGSSGGPLLCPAGHAVGIFRAAVSTRGVAKAVDFIPVESLETTMRSP
NS3a-H1 Sequence:
(SEQ ID NO: 32)
MKKKGSVVIVGRINLSGDTAYSQQTRGLEGCQETSQTGRDKNQVEGEVQVVSTATQSFLATSINGVLW
TVYHGAGTRTIASPKGPVTQMYTNVDKDLVGWQAPQGSRSLTPCTCGSSDLYLVTRHADVIPVRRRGD
SRGSLLSPRPISYLKGSSGGPLLCPAGHAVGIFRAAVSTRGVAKAVDFIPVESLETTMRSP
NS3a-H2 Sequence:
(SEQ ID NO: 33)
MKKKGSVVIVGRINLSGDTAYSQQTRGELGCQETSQTGRDKNQVEGEVQVVSTATQSFLATSINGVLW
TVYHGAGTRTIASPKGPVTQMYTNVDKDLVGWQAPQGSRSLTPCTCGSSDLYLVTRHADVIPVRRRGD
SRGSLLSPRPISYLKGSSGGPLLCPAGHAVGIFRAAVSTRGVAKAVDFIPVESLETTMRSP
NS3a-H3 Sequence:
(SEQ ID NO: 34)
MKKKGSVVIVGRINLSGDTAYSQQTRGLLGCQETSQTGRDKNQVEGEVQVVSTATQSFLATSINGVLW
TVYHGAGTRTIASPKGPVTQMYTNVDKDLVGWQAPQGSRSLTPCTCGSSDLYLVTRHADVIPVRRRGD
SRGSLLSPRPISYLKGSSGGPLLCPAGHAVGIFRAAVSTRGVAKAVDFIPVESLETTMRSP
NS3a-H4 Sequence:
(SEQ ID NO: 35)
MKKKGSVVIVGRINLSGDTAYSQQTRGLLGCIETSQTGRDKNQVEGEVQVVSTATQSFLATSINGVLW
TVYHGAGTRTIASPKGPVTQMYTNVDKDLVGWQAPQGSRSLTPCTCGSSDLYLVTRHADVIPVRRRGD
SRGSLLSPRPISYLKGSSGGPLLCPAGHAVGIFRAAVSTRGVAKAVDFIPVESLETTMRSP
NS3a-H5 Sequence:
(SEQ ID NO: 36)
MKKKGSVVIVGRINLSGDTAYSQQTRGLLGCIITSQTGRDKNQVEGEVQVVSTATQSFLATSINGVLW
TVYHGAGTRTIASPKGPVTQMYTNVDKDLVGWQAPQGSRSLTPCTCGSSDLYLVTRHADVIPVRRRGD
SRGSLLSPRPISYLKGSSGGPLLCPAGHAVGIFRAAVSTRGVAKAVDFIPVESLETTMRSP
NS3a-H6 Sequence:
(SEQ ID NO: 37)
MKKKGSVVIVGRINLSGDTAYSQQTRGLEGCIETSQTGRDKNQVEGEVQVVSTATQSFLATSINGVLW
TVYHGAGTRTIASPKGPVTQMYTNVDKDLVGWQAPQGSRSLTPCTCGSSDLYLVTRHADVIPVRRRGD
SRGSLLSPRPISYLKGSSGGPLLCPAGHAVGIFRAAVSTRGVAKAVDFIPVESLETTMRSP
NS3a-H7 Sequence:
(SEQ ID NO: 38)
MKKKGSVVIVGRINLSGDTAYSQQTRGEEGCQETSQTGRDKNQVEGEVQVVSTATQSFLATSINGVLW
TVYHGAGTRTIASPKGPVTQMYTNVDKDLVGWQAPQGSRSLTPCTCGSSDLYLVTRHADVIPVRRRGD
SRGSLLSPRPISYLKGSSGGPLLCPAGHAVGIFRAAVSTRGVAKAVDFIPVESLETTMRSP

In various embodiments, one or both of B1 and B2 are present, or both B1 and B2 are present. Any suitable linkers can be used, having any amino acid composition and length as determined appropriate for an intended use. As disclosed in the examples that follow, the inventors have provided extensive guidance on identifying the appropriate linkers in light of the protein having one or more interaction surfaces included in the fusion protein. In various embodiments, the linkers may be flexible, for example being rich in glycine, serine, and/or threonine residues. In other embodiments, the linker may not include proline residues.

In another embodiment, one of X1 and X3 is a peptide comprising the amino acid sequence having at least 75%, 80%, 85%, 90%, 91%, 92%, 93%, 94%, 95%, 96%, 97%, 98%, 99%, or 100% identity to the full length of the amino acid sequence selected from GELGRLVYLLDGPGYDPIHSD (SEQ ID NO:13), GELDELVYLLDGPGYDPIHSD (SEQ ID NO: 14), GELGELVYLLDGPGYDPIHSD (SEQ ID NO: 15), or GELDRLVYLLDGPGYDPIHSD (SEQ ID NO:16), or GELDELVYLLDGPGYDPIHSDVVTRGGSHLFNF (SEQ ID NO: 17) (“ANR peptide”). In this embodiment, the recombinant fusion proteins may be used, for example, to bring any protein domains that are genetically fused to ANR and NS3a together in the basal state. This complex can then be disrupted by any of the small molecule NS3a inhibitors as described herein.

Use of catalytically active vs. dead NS3a enables the creation of orthogonal ANR/NS3a systems, in which only the catalytically active NS3a/ANR complex can be disrupted by covalent inhibitors such as telaprevir or non-covalent inhibitors, while the catalytically dead NS3a/ANR complex can only be disrupted by non-covalent inhibitors such as asunaprevir. Catalytically active variants of NS3a contain the catalytic serine, bolded in “LKGSSGG” (SEQ ID NO:18) and in SEQ ID NOS:30-38, while catalytically dead versions have that serine mutated to an alanine. Exemplary embodiments of this system are described in the examples that follow, such as a demonstrated application for intramolecular gating of an enzyme or interaction domain, and a demonstrated application as an intermolecular off switch for transcription or signaling (demonstrated for transcriptional control for exogenous or endogenous promoters in mammalian cells).

In one embodiment, one of X1 and X3 is the DNCR polypeptide of any embodiment or combination of embodiments disclosed herein. In another embodiment, one of X1 and X3 is the GNCR polypeptide of any embodiment or combination of embodiments disclosed herein. In these embodiments, the recombinant fusion proteins may be used, for example, to turn off activity of the X2 protein. A possible application of this would be to have an enzymatic domain constitutively active in the basal, no drug-state, and inhibited upon NS3a inhibitor addition. Another possible application would be to allow constitutive transcription in the basal, no-drug state, where X2 is a transcription factor or catalytically dead Cas9 domain and have this transcription inactivated by formation of the complex or DNCR or GNCR with NS3a upon NS3a inhibitor addition.

The recombinant fusion protein may comprise any protein having one or more interaction surfaces as the X2 moiety, as deemed most suitable for an intended use, such as those described herein and in the attached appendices. Any suitable protein having one or more interaction surfaces can be used as deemed appropriate for an intended purpose. In non-limiting embodiments, the protein having one or more interaction surfaces comprises an enzymatic protein, protein-protein interaction domain, a nucleic acid-binding domain, etc. In various further embodiments, the protein having one or more interaction surfaces is selected from the group consisting of. Cas9 and related CRISPR proteins (catalytically active or dead), a DNA binding domain of a transcription factor (such as the Gal4 DNA binding domain), a pro-apoptotic domain (such as caspase 9), and a cell surface receptor (such as a chimeric antigen receptor). In another embodiment, X2 may be a protein including, but not limited to, a guanine nucleotide exchange factor GEF such as SOS, Cas9 and related CRISPR proteins (catalytically active or dead), a DNA binding domain of a transcription factor (such as the Gal4 DNA binding domain), a pro-apoptotic domain (such as caspase 9), and a cell surface receptor (such as a chimeric antigen receptor).

In another embodiment, the recombinant fusion protein further comprises a peptide localization tag at the N-terminus and/or the C-terminus of the fusion protein. Any suitable localization tag can be used as deemed appropriate for an intended purpose. In non-limiting embodiments, the localization tag may target the recombinant fusion protein to the cell membrane, the nucleus, the mitochondria, Golgi apparatus, cell surface receptors, etc. In one embodiment, the localization tag comprises a membrane localization or nuclear localization tag.

In non-limiting embodiments, the recombinant fusion protein comprises the amino acid sequence having at least 50%, 55%, 60%, 65%, 70%, 75%, 80%, 85%, 90%, 91%, 92%, 93%, 94%, 95%, 96%, 97%, 98%, 99%, or 100% identity to the full length of the amino acid sequence of:

NS3a-CDAR Sequence:
(SEQ ID NO: 39)
MGELDELVYLLDGPGYDPIHSDGSGTGSGTGSGTGSGTGDVYRFAEPDSEENIIFEENMQPKAGIPIIKAGTVIK
LIERLTYHMYADPNEVRTFLTTYRSFCKPQELLSLITERFEIPEPEPTEADRIAIENGDQPLSAELKRFRKEYIQ
PVQLRVLNVCRHWVEHHFYDFERDAYLLQRMEEFIGTVRGKAMKKWVESITKIIQRKKIARDNGPGHNITFQSSP
PTVEWHISRPGHIETFDLLTLHPIEIARQLTLLESDLYRAVQPSELVGSVWTKEDKEINSPNLLKMIRHTTNLTL
WFEKCIVETENLEERVAVVSRIIEILQVFQELNNFNGVLEVVSAMNSSPVYRLDHTFEQIPSRQKKILEEAHELS
EDHYKKYLAKLRSINPPCVPFFGIYLTNILKTEEGNPEVLKRHGKELINFSKRRKVAEILGEIQQYQNQPYCLRV
ESDIKRFFENLNPMGNSMEKEFTDYLFNKSLEIEPGSGTGSGMAKGSVVIVGRINLSGDTAYSQQTRGLLGIIIT
SLTGRDKNQVDGEVQVLSTATQSFLATCVNGVCWTVYHGAGSKTLAGPKGPITQMYTNVDQDLVGWPAPPGARSM
TPCTCGSSDLYLVTRHADVIPVRRRGDSRGSLLSPRPVSYLKGSSGGPLLCPSGHVVGIFRAAVCTRGVAKAVDF
IPVESMETTMRGSGTGSGGSGTGDYKDDDDKQHKLRKLNPPDESGPGCMSCKCVLS

In another aspect, the disclosure provides polypeptides comprising the amino acid sequence selected from the group consisting of SEQ ID NOS:31-38, wherein the bolded amino acid residue is the catalytic position, wherein the bolded “S” residue represents catalytically active NS3a peptides, and wherein the bolded ‘S” residue can be substituted with an alanine (or other) residue to render the NS3a peptide catalytically “dead” (which will also work in all applications):

NS3a* Sequence
(SEQ ID NO: 31)
MKKKGSVVIVGRINLSGDTAYAQQTRGEEGCQETSQTGRDKNQVEGEVQIVSTATQTFLATSINGVLW
TVYHGAGTRTIASPKGPVTQMYTNVDKDLVGWQAPQGSRSLTPCTCGSSDLYLVTRHADVIPVRRRGD
SRGSLLSPRPISYLKGSSGGPLLCPAGHAVGIFRAAVSTRGVAKAVDFIPVESLETTMRSP
NS3a-H1 Sequence:
(SEQ ID NO: 32)
MKKKGSVVIVGRINLSGDTAYSQQTRGLEGCQETSQTGRDKNQVEGEVQVVSTATQSFLATSINGVLW
TVYHGAGTRTIASPKGPVTQMYTNVDKDLVGWQAPQGSRSLTPCTCGSSDLYLVTRHADVIPVRRRGD
SRGSLLSPRPISYLKGSSGGPLLCPAGHAVGIFRAAVSTRGVAKAVDFIPVESLETTMRSP
NS3a-H2 Sequence:
(SEQ ID NO: 33)
MKKKGSVVIVGRINLSGDTAYSQQTRGELGCQETSQTGRDKNQVEGEVQVVSTATQSFLATSINGVLW
TVYHGAGTRTIASPKGPVTQMYTNVDKDLVGWQAPQGSRSLTPCTCGSSDLYLVTRHADVIPVRRRGD
SRGSLLSPRPISYLKGSSGGPLLCPAGHAVGIFRAAVSTRGVAKAVDFIPVESLETTMRSP
NS3a-H3 Sequence:
(SEQ ID NO: 34)
MKKKGSVVIVGRINLSGDTAYSQQTRGLLGCQETSQTGRDKNQVEGEVQVVSTATQSFLATSINGVLW
TVYHGAGTRTIASPKGPVTQMYTNVDKDLVGWQAPQGSRSLTPCTCGSSDLYLVTRHADVIPVRRRGD
SRGSLLSPRPISYLKGSSGGPLLCPAGHAVGIFRAAVSTRGVAKAVDFIPVESLETTMRSP
NS3a-H4 Sequence:
(SEQ ID NO: 35)
MKKKGSVVIVGRINLSGDTAYSQQTRGLLGCIETSQTGRDKNQVEGEVQVVSTATQSFLATSINGVLW
TVYHGAGTRTIASPKGPVTQMYTNVDKDLVGWQAPQGSRSLTPCTCGSSDLYLVTRHADVIPVRRRGD
SRGSLLSPRPISYLKGSSGGPLLCPAGHAVGIFRAAVSTRGVAKAVDFIPVESLETTMRSP
NS3a-H5 Sequence:
(SEQ ID NO: 36)
MKKKGSVVIVGRINLSGDTAYSQQTRGLLGCIITSQTGRDKNQVEGEVQVVSTATQSFLATSINGVLW
TVYHGAGTRTIASPKGPVTQMYTNVDKDLVGWQAPQGSRSLTPCTCGSSDLYLVTRHADVIPVRRRGD
SRGSLLSPRPISYLKGSSGGPLLCPAGHAVGIFRAAVSTRGVAKAVDFIPVESLETTMRSP
NS3a-H6 Sequence:
(SEQ ID NO: 37)
MKKKGSVVIVGRINLSGDTAYSQQTRGLEGCIETSQTGRDKNQVEGEVQVVSTATQSFLATSINGVLW
TVYHGAGTRTIASPKGPVTQMYTNVDKDLVGWQAPQGSRSLTPCTCGSSDLYLVTRHADVIPVRRRGD
SRGSLLSPRPISYLKGSSGGPLLCPAGHAVGIFRAAVSTRGVAKAVDFIPVESLETTMRSP
NS3a-H7 Sequence:
(SEQ ID NO: 38)
MKKKGSVVIVGRINLSGDTAYSQQTRGEEGCQETSQTGRDKNQVEGEVQVVSTATQSFLATSINGVLW
TVYHGAGTRTIASPKGPVTQMYTNVDKDLVGWQAPQGSRSLTPCTCGSSDLYLVTRHADVIPVRRRGD
SRGSLLSPRPISYLKGSSGGPLLCPAGHAVGIFRAAVSTRGVAKAVDFIPVESLETTMRSP

As disclosed herein, the polypeptides of this aspect of the disclosure reduce membrane binding of the Ns3A protein, and thus are particularly useful for the intermolecular binding aspects and embodiments disclosed herein. The polypeptides of this claim are engineered chimeras of natural genotype 1b HCV protease NS3/4a and a solubility optimized genotype 1a HCV protease NS3/4a (catalytically active or dead). These non-natural variants of NS3a allow binding to the peptide ANR while having reduced binding to cellular membranes.

In another aspect, the disclosure provides combinations, comprising:

(a) a first fusion protein comprising:
(i) a localization tag or a protein having one or more interaction surfaces; and
(ii) an NS3a peptide comprising the amino acid sequence having at least 80%, 85%, 90%, 91%, 92%, 93%, 94%, 95%, 96%, 97%, 98%, 99%, or 100% identity to the full length of the amino acid sequence selected from the group consisting of SEQ ID NOS:31-38, wherein the bolded amino acid residue is the catalytic position, wherein the bolded “S” residue represents catalytically active NS3a peptides, and wherein the bolded ‘S” residue can be substituted with an alanine (or other) residue to render the NS3a peptide catalytically “dead” (which will also work in all applications).

(b) one or more second fusion proteins comprising:
(i) a localization tag if the first fusion protein comprises a protein having one or more interaction surfaces; or a protein having one or more interaction surfaces if the first fusion protein comprises a localization tag; and
(ii) a polypeptide selected from the group consisting of selected from the group consisting of:
   (A) a polypeptide comprising the amino acid sequence having at least 50%, 55%, 60%, 65%, 70%, 75%, 80%, 85%, 90%9, 91%, 92%, 93%, 94%, 95%, 96%, 97%, 98%, 99%, or 100% identity to the full length of the amino acid sequence selected from GELGRLVYLLDGPGYDPIHSD (SEQ ID NO:13), GELDELVYLLDGPGYDPIHSD (SEQ ID NO:14), GELGELVYLLDGPGYDPIHSD (SEQ ID NO:15), GELDRLVYLLDGPGYDPIHSD (SEQ ID NO:16), or GELDELVYLLDGPGYDPIHSDVVTRGGSHLFNF (SEQ ID NO:17) (“ANR peptide”);
   (B) the DNCR polypeptide of any embodiment or combination of embodiments disclosed herein; and
   (C) the GNCR polypeptide of any embodiment or combination of embodiments disclosed herein.

These combinations can be used for intermolecular binding uses of any kind. Numerous exemplary embodiments are disclosed herein. The localization tags and proteins having one or more interaction surface can be any suitable ones, including but not limited to those disclosed herein and the attached examples. In one embodiment, the first fusion protein comprises the NS3a polypeptide comprising the amino acid sequence selected from the group consisting of SEQ ID NOS:31-38, wherein the bolded amino acid residue is the catalytic position, wherein the bolded “S” residue represents catalytically active NS3a peptides, and wherein the bolded ‘S” residue can be substituted with an alanine (or other) residue to render the NS3a peptide catalytically “dead”. In another embodiment, the second fusion protein comprises a polypeptide comprising the amino acid sequence having at least 50%, 55%, 60%, 65%, 70%, 75%, 80%, 85%, 90%, 91%, 92%, 93%, 94%, 95%, 96%, 97%, 98%, 99%, or 100% identity to the full length of the amino acid sequence selected from GELGRLVYLLDGPGYDPIHSD (SEQ ID NO:13), GELDELVYLLDGPGYDPIHSD (SEQ ID NO:14), GELGELVYLLDGPGYDPIHSD (SEQ ID NO:15), GELDRLVYLLDGPGYDPIHSD (SEQ ID NO:16), or GELDELVYLLDGPGYDPIHSDVVTRGGSHLFNF (SEQ ID NO:17) (“ANR peptide”).

In further embodiments, the second fusion protein comprises the DNCR polypeptide of any embodiment or combination of embodiments disclosed herein. In other embodiments, the second fusion protein comprises the GNCR polypeptide of any embodiment or combination of embodiments disclosed herein.

The polypeptides, fusion proteins, and recombinant fusion proteins described herein may be chemically synthesized or recombinantly expressed (when the polypeptide is genetically encodable), and may include additional residues at the N-terminus, C-terminus, or both that are not present in the polypeptides or peptide domains of the disclosure; these additional residues are not included in determining the percent identity of the polypeptides or peptide domains of the disclosure relative to the reference polypeptide. Such residues may be any residues suitable for an intended use, including but not limited to detection tags (i.e.: fluorescent proteins, antibody epitope tags, etc.), adaptors, ligands suitable for purposes of purification (His tags, etc.), other peptide domains that add functionality to the polypeptides, etc.

In a further aspect, the present disclosure provides nucleic acids encoding a polypeptide, fusion protein, and/or recombinant fusion proteins of the present invention that can be genetically encoded. The nucleic acid sequence may comprise RNA, DNA, and/or modified nucleic acids. Such nucleic acid sequences may comprise additional sequences useful for promoting expression and/or purification of the encoded protein, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides, fusion protein, and/or recombinant fusion proteins of the invention.

In another aspect, the present disclosure provides expression vectors comprising the nucleic acid of any embodiment or combination of embodiments disclosed herein operatively linked to a suitable control sequence. Expression vectors include vectors that operatively link a nucleic acid coding region or gene to any control sequences capable of effecting expression of the gene product. “Control sequences” operably linked to the nucleic acid sequences of the invention are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors include but not limited to, plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The expression vector must be replicable in the host organisms either as an episome or by integration into host chromosomal DNA. In various embodiments, the expression vector may comprise a plasmid, viral-based vector, or any other suitable expression vector.

In a further aspect, the present disclosure provides host cells that comprise the nucleic acid and/or expression vectors disclosed herein, wherein the host cells can be either prokaryotic or eukaryotic. The cells can be transiently or stably engineered to incorporate the expression vector of the invention, using standard techniques in the art, including but not limited to standard bacterial transformations, calcium phosphate co-precipitation, electroporation, or liposome mediated-, DEAE dextran mediated-, polycationic mediated-, or viral mediated transfection. (See, for example, Molecular Cloning: A Laboratory Manual (Sambrook, et al., 1989, Cold Spring Harbor Laboratory Press; Culture of Animal Cells: A Manual of Basic Technique, 2^{nd} Ed. (R.I. Freshney. 1987. Liss, Inc. New York, N.Y.). A method of producing a polypeptide according to the invention is an additional part of the disclosure. The method comprises the steps of (a) culturing a host according to this aspect of the invention under conditions conducive to the expression of the polypeptide, and (b) optionally, recovering the expressed polypeptide. The expressed polypeptide can be recovered from the cell free extract, but preferably they are recovered from the culture medium.

In another aspect, the disclosure provides use of the polypeptides, fusion proteins, recombinant fusion proteins, combinations, nucleic acids, expression vectors, and/or host cells of any embodiment or combination of embodiments disclosed herein, to carry out any methods, including but not limited to those disclosed herein. Numerous exemplary uses of the polypeptides, fusion proteins, recombinant fusion proteins, combinations, nucleic acids, expression vectors, and/or host cells are described in the examples that follow. In exemplary non-limiting embodiments, the methods may include:
1. Chemically-disrupted proximity system (CDP) based on the binding of a genetically-encoded inhibitor peptide, here called apo NS3a reader (ANR) to the HCV protease NS3/4a.
   a. Where NS3a is one of the following variants of HCV protease NS3/4a: engineered variants NS3a-H1, -H2, -H3, -H4, -H5, or -H6 (all either catalytically active or dead) (SEQ ID NOS:31-37).
   b. This CDP system can be used to bring any protein domains that are genetically fused to ANR and NS3a together in the basal state. This complex can then be disrupted by any of the small molecule NS3a inhibitors.
   c. Use of catalytically active vs. dead NS3a enables the creation of orthogonal ANR/NS3a systems, in which only the catalytically active NS3a/ANR complex can be disrupted by covalent inhibitors such as telaprevir or non-covalent inhibitors, while the catalytically dead NS3a/ANR complex can only be disrupted by non-covalent inhibitors such as asunaprevir.
   a. Demonstrated application of CDP system: Intramolecular gating of an enzyme domain (Example 1).
   b. Demonstrated application of CDP system: Intermolecular off switch for transcription or signaling (demonstrated for transcriptional control for exogenous or endogenous promoters in mammalian cells, Example 1).
2. PROCISiR: Pleotropic response outputs from a chemically-inducible single receiver
   a. A system in which a viral protease (HCV NS3a) functions as a receiver protein that binds multiple drug inputs wherein the viral protease is recognized by a set of selective, genetically-encoded protein readers to produce a plurality of divergent outputs.
   b. Where the readers are defined as ANR, DNCR, GNCR, or any other readers that are engineered to selectively recognize apo or inhibitor-bound states of NS3a.
   c. Use of this system to temporally or proportionally control three or more different cellular outputs based on the NS3a inhibitor applied:
      i. Three transcriptional outputs demonstrated in Example 2.
      ii. Two signaling outputs demonstrated in Example 2.
3. Reversible chemically-induced proximity (CIP) systems (DNCR/danoprevir/NS3a or GNCR/grazoprevir/NS3a).
   a. Enhanced reversibility of complexes formed by these CIP systems by treatment with a second small molecule inhibitor of NS3a that is not recognized by the employed reader.
   b. Demonstrated for DNCR2/danoprevir/NS3a in Example 2.
4. Tunable transcriptional or signaling output from CIPs through the use of combinations of inducer and competitor small molecules.
   a. Transcription tuning demonstrated in Example 2.
   b. Signaling (membrane association) tuning demonstrated in Example 2.
5. Proportional control of two outputs by combining DNCR and GNCR and treating with varying ratios of danoprevir and grazoprevir.
   a. Demonstrated for transcription in Example 2.
6. Use of the CIPs to induce (or repress) transcription from endogenous or exogenous promoters
   a. Transcription induced or repressed from endogenous promoters using association of the CIP components with any DNA binding domain that recognizes sequences in endogenous promoters (here, catalytically dead Cas9 (dCas9)) and transcriptional activation (here VPR) or repression domains (here KRAB or KRAB-MeCP2).
      i. Demonstrated in Example 2.
   b. Transcription induced from exogenous promoters using CIPs to bring together any exogenous DNA binding domain with a transcriptional activation domain.
      i. Demonstrated with the Gal4 DNA binding domain, DNCR2 and NS3a, and the VPR transcriptional activation domain in Example 2.
7. Use of CIPs to induce signaling at the plasma membrane in mammalian cells.
   a. Demonstrated in Example 2.

### EXAMPLES

### Example 1

Here, we describe a new chemically-controlled method for rapidly disrupting the interaction between two basally co-localized protein binding partners. Our chemically-disrupted proximity (CDP) system is based on the interaction between the hepatitis C virus protease (HCVp) NS3a and a genetically-encoded peptide inhibitor. Using clinically-approved antiviral inhibitors as chemical disrupters of the NS3a/peptide interaction, we demonstrate that our CDP system can be used to confer temporal control over diverse intracellular processes. This NS3a-based CDP system represents a new modality for engineering chemical control over intracellular protein function that is complementary to currently available technologies.

Rationally manipulating protein localization can provide fundamental insights into cellular processes and is a powerful tool for engineering cellular behaviors. Techniques that allow temporal regulation of protein localization are particularly valuable for interrogating and programming dynamic cellular processes, with light and small molecules serving as the most widely used means of user-defined control. A strategy for the chemical control of protein localization is the use of chemically-induced proximity (CIP), which allows two proteins to be colocalized upon addition of a bridging small molecule.

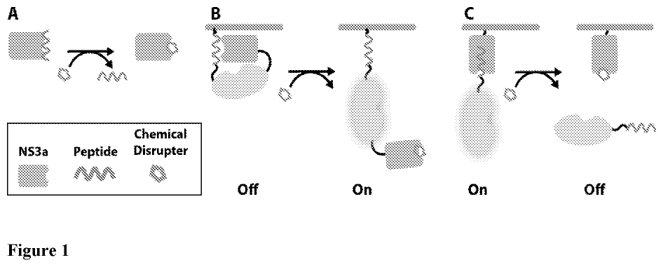

Systems that allow the interaction of two basally colocalized proteins to be rapidly disrupted with a small molecule provide a method for temporally controlling intracellular protein function (FIG. 1). Such chemically-disrupted proximity (CDP) systems can be used in numerous intramolecular and intermolecular cellular engineering applications. For example, we have demonstrated that a CDP system based on the interaction between the anti-apoptotic protein BCL-xL and a BH3 peptide can be used as a chemically-disruptable autoinhibitory switch for intramolecularly controlling the activities of various enzymes (FIG. 1). Intermolecular CDP systems that allow a basally localized activity to be chemically disrupted can be used as off-switches for a number of applications (FIG. 1C).

Here, we describe the development and use of a CDP system based on the hepatitis C virus protease (HCVp) NS3a and its interaction with a peptide inhibitor. Clinically-approved protease inhibitors that efficiently disrupt the NS3a/peptide interaction are available as bio-orthogonal inputs for this system. We first show that our NS3a-based CDP system can be used as a chemically-disruptable autoinhibitory switch for controlling the activity of an enzyme that activates RAS GTPase. We also demonstrate that the NS3a-based CDP system can be used to rapidly disrupt subcellular protein colocalization. Demonstrating the functional utility of chemically disrupting protein colocalization, we show that our NS3a-based CDP system can be used as a transcriptional off switch.

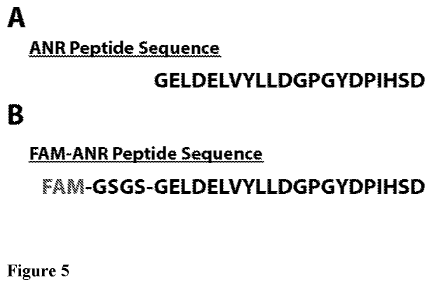

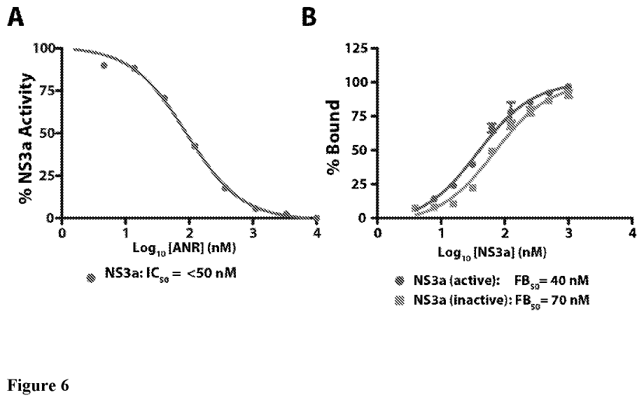

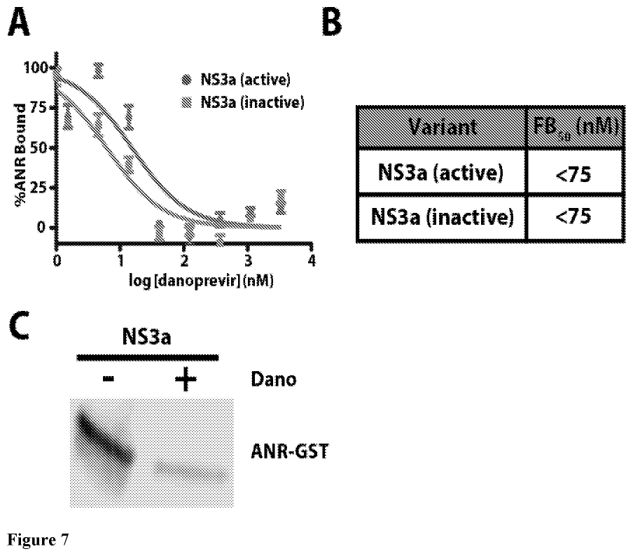

In order to use NS3a as a platform for a CDP system, a genetically-encoded binding partner that can be displaced with protease inhibitors was used. To provide this, we investigated the use of a peptide inhibitor of NS3a's serine protease activity (FIG. 5). We found that this peptide, which we call apo NS3a reader (ANR), binds tightly to NS3a (FIG. 6). Furthermore, we observed that the drug danoprevir was able to potently and dose-dependently displace ANR from NS3a (FIG. 7), demonstrating that this interaction can be used as the basis for a CDP system.

We first explored using the NS3a/ANR interaction as a chemically-disruptable autoinhibitory switch for intramolecularly controlling the guanine nucleotide exchange factor (GEF) activity of the RAS GTPase activator Son of sevenless (SOS).

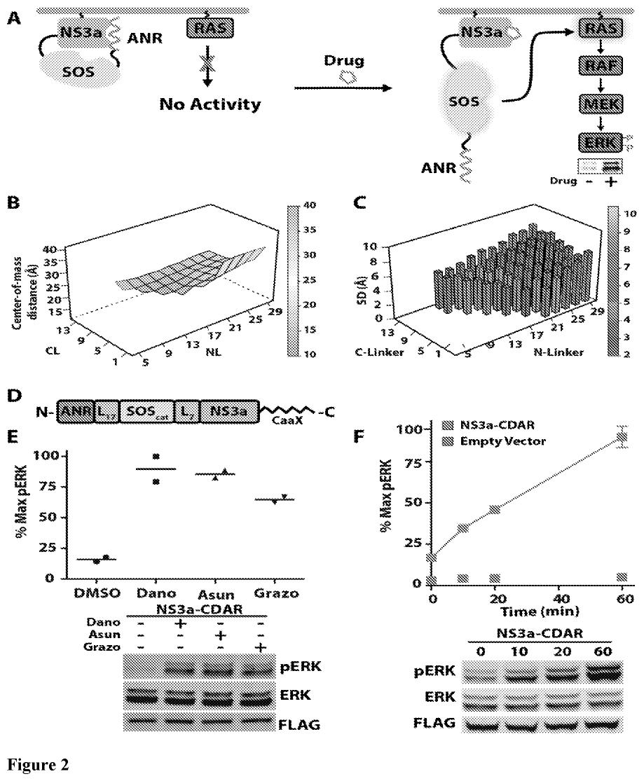

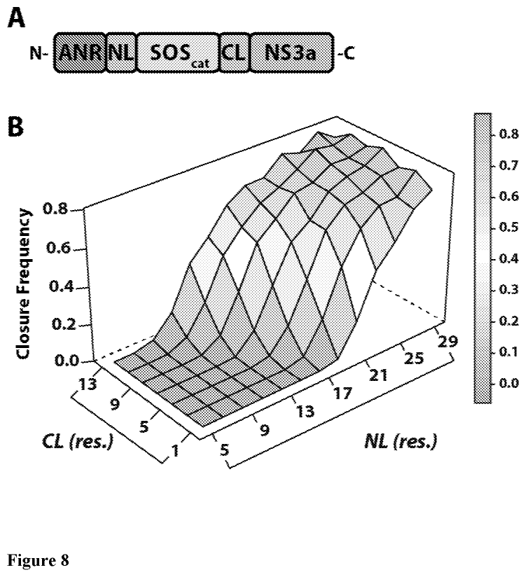

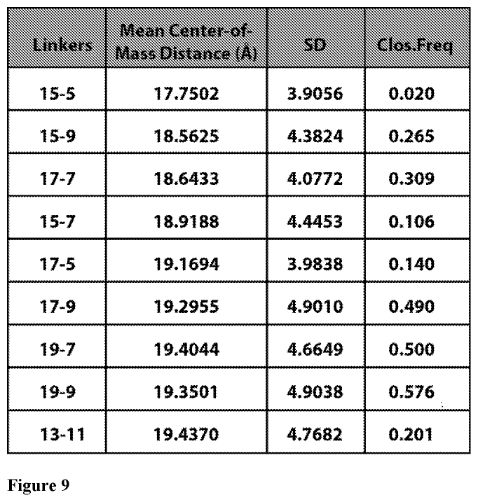

We used the computational modeling tool RosettaRemodel™ to guide the selection of flexible linker lengths with which to fuse ANR and NS3a to opposing termini of SOScat. Our goal was to identify linkers of sufficient length that NS3a and ANR can form an intramolecular complex but short enough that the complex is primarily centered over SOScat's active site, with an energetic penalty for adopting non-inhibitory conformations. To do this, we computationally treated variable linker length SOScat fusions with ANR at the N-terminus and NS3a at the C-terminus as a single loop closure problem (FIG. 8). An arbitrary break in one of the linkers of these fusion constructs was introduced, and subsequent chain closures were only permitted in geometrically allowed models. For each linker length combination, the percentage of successful chain closures was used to calculate the chain closure frequency (FIG. 8). For models that successfully closed, torsional angles within the linkers were allowed to further vary in order to sample the most energetically favorable conformations of the ANR/NS3a complex relative to SOScat. Using this algorithm, we determined how linker lengths ranging from 5-29 and 1-13 residues for the N- and C-terminal linkers, respectively, affects the frequency of closure and the overlap of the ANR/NS3a complex with SOScat's active site (FIG. 2B, 2C). We found that output PDBs showed the NS3a/ANR complex most tightly clustered over SOScat's active site-smallest center-of-mass distance and standard deviation-when the linkers connecting ANR to the N-terminus of SOScat was 17 amino acids and between the C-terminus of SOScat and NS3a was 7 amino acids (FIG. 9). Therefore, we next determined whether a construct with these linkers can function as a chemically disrupted activator of RAS (CDAR) in cells.

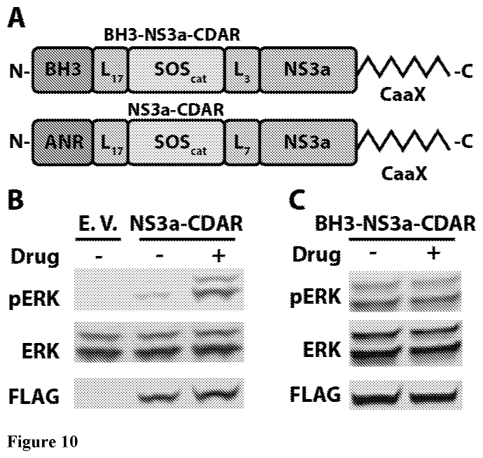

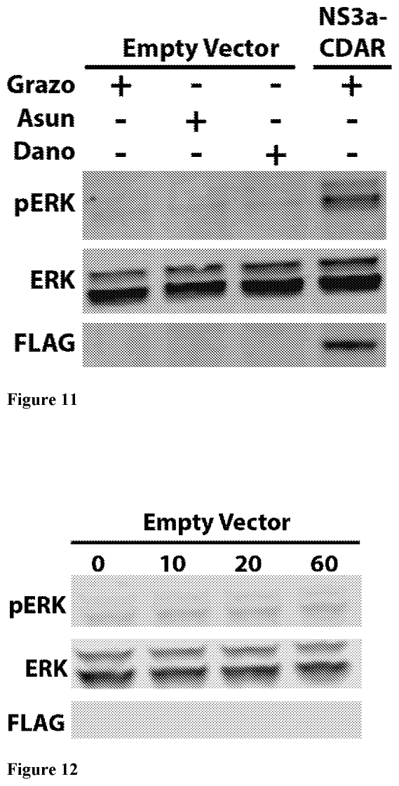

To demonstrate the utility of our NS3a-CDAR design for activating the RAS/ERK pathway, we transfected HEK293 cells with a membrane-targeted variant of our computationally-designed construct (FIG. 2D) and monitored downstream activation of ERK kinase (phospho-ERK) (FIG. 2E). In untreated cells expressing NS3a-CDAR, we found that phospho-ERK levels were low, which is consistent with the NS3a/ANR interaction providing significant autoinhibition of SOScat's GEF activity. In contrast, untreated cells expressing an NS3a-CDAR construct where ANR has been replaced with a peptide that has no affinity for NS3a demonstrated high basal phospho-ERK levels (FIG. 10). We observed a robust increase in phospho-ERK levels when danoprevir, asunaprevir, or grazoprevir were added to cells expressing NS3a-CDAR (FIG. 2E). However, these drugs did not lead to an increase in cellular phospho-ERK levels in the absence of the NS3a-CDAR construct (FIG. 11). We found that NS3a-CDAR rapidly activated RAS/ERK signaling(FIGS. 2F, 12). Thus, the NS3a/ANR interaction can serve as a drug-disruptable switch for rapidly activating RAS with clinically approved drugs that are orthogonal to mammalian systems.

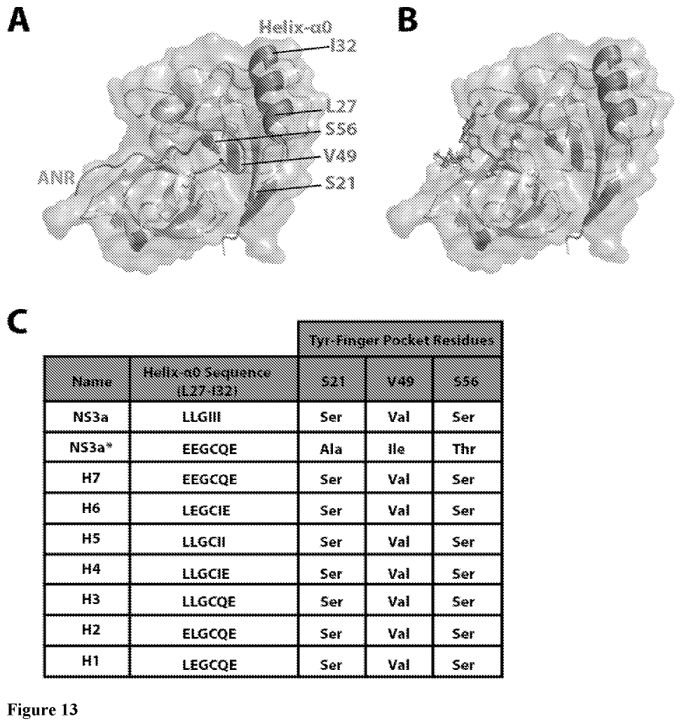

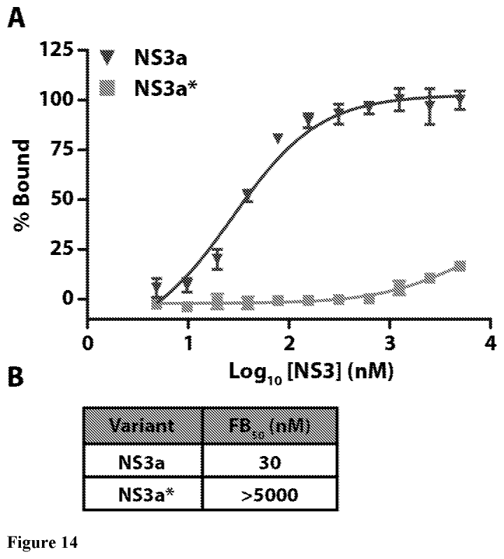

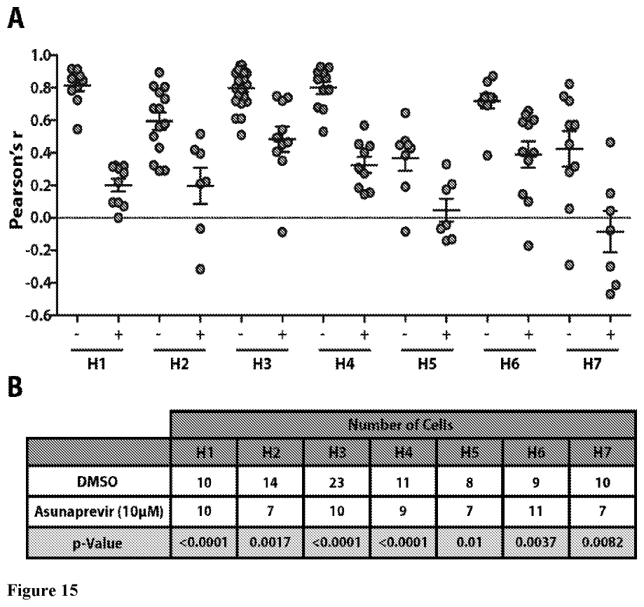

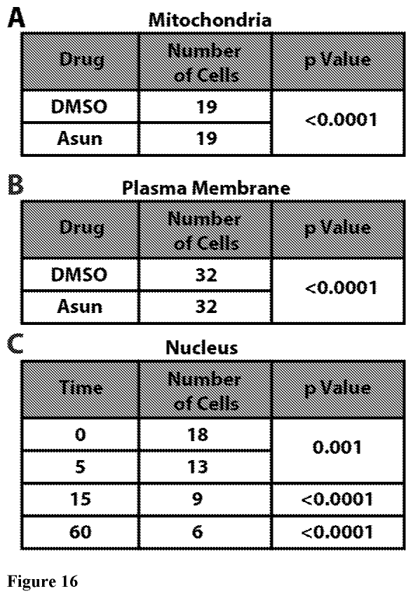

We next investigated the utility of the NS3a/ANR interaction as an intermolecular CDP system by determining whether it could provide temporal control over protein colocalization. An N-terminal amphipathic helix—helix α0—from the NS3a variant used in our NS3a-CDAR construct has previously been demonstrated to interact with membranes (FIG. 13), which we thought would be problematic for an intermolecular CDP system. Therefore, we determined whether a solubility-optimized NS3a variant-NS3a*-could be used with ANR as part of an intermolecular CDP system. Unfortunately, we observed that ANR has very low affinity for NS3a* (FIG. 14). Therefore, we generated and tested a series of NS3a/NS3a* chimeras for their ability to colocalize with ANR in cells (FIGS. 13, 15).

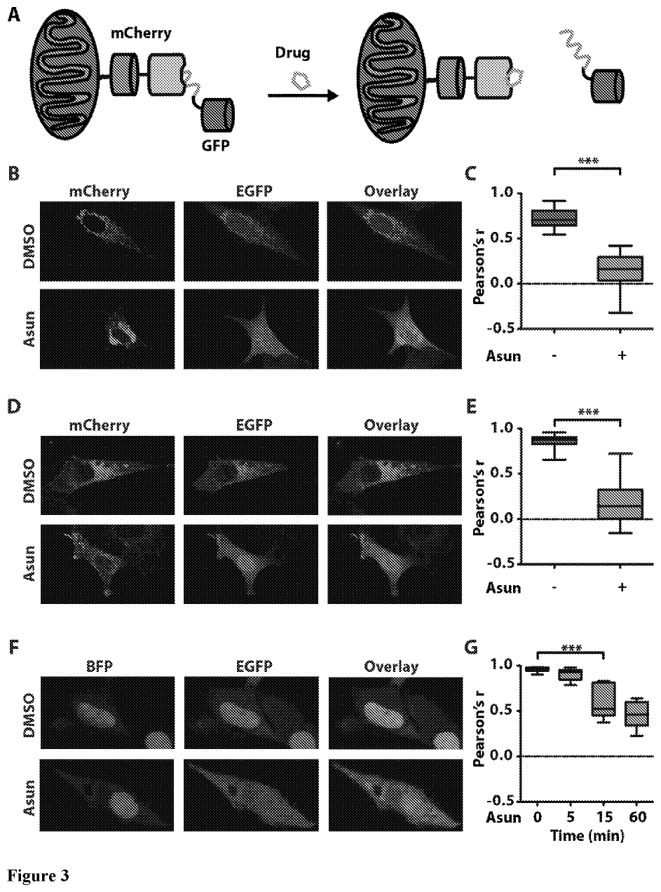

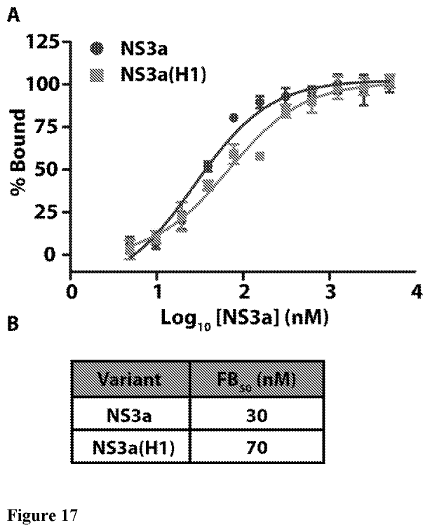

To functionally test our NS3a chimeras, we used a fluorescent protein colocalization assay (FIG. 3A). Each NS3a chimera was expressed as a mitochondrially-localized mCherry™ fusion and the amount of colocalization with an EGFP-ANR fusion protein was determined in cells treated with DMSO or asunaprevir (FIG. 15). We found that all NS3a chimeras were capable of localizing EGFP-ANR to mitochondria in the absence of drug but constructs lacking hydrophobic residues at the C-terminal end of helix α0 provided the highest degree of colocalization. Furthermore, we observed that these more polar chimeras—in particular NS3a(H1)—demonstrated the largest difference in colocalization between DMSO and asunaprevir-treated cells. Binding assays with purified NS3a(H1) showed that this chimera's affinity for ANR is similar to NS3a (FIG. 17). Therefore, we used the NS3a(H1) variant for all subsequent engineering efforts.

We next determined how rapidly the intracellular NS3a(H1)/ANR interaction can be disrupted. We found that the interaction between EGFP-ANR with mitochondrially-localized NS3a(H1) was completely disrupted within five minutes of asunaprevir addition (FIG. 3B, 3C). Furthermore, we observed similar disruption kinetics when EGFP-NS3a(H1) was colocalized to membranes with N-terminally myristoylated ANR (FIG. 3D, 3E). Robust, albeit slower, disruption of EGFP-NS3a(H1) nuclear localization was obtained when NLS-ANR-expressing cells were treated with asunaprevir. (FIG. 3F, 3G). Thus, the NS3a/ANR interaction can be used to colocalize proteins in diverse subcellular compartments, which chemical disruptors rapidly reverse.

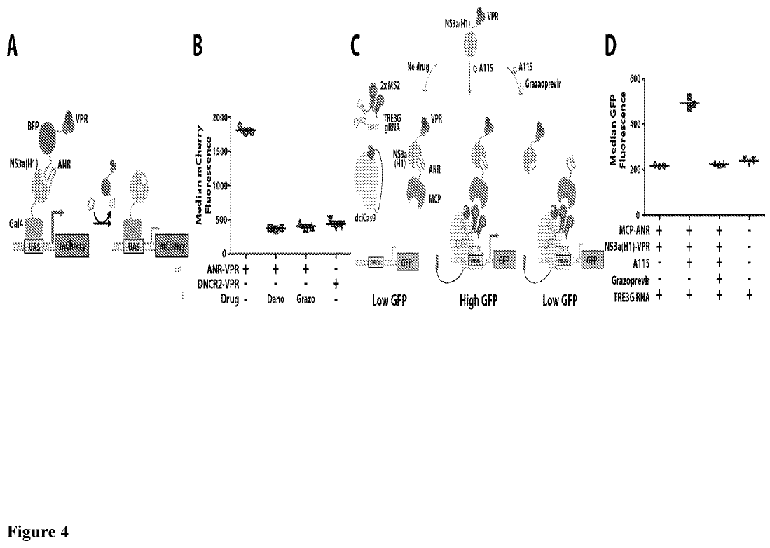

The localization of transcriptional activation domains upstream of genes can drive transcription and subsequent protein expression. We reasoned that the NS3a(H1)/ANR interaction could function as a chemically-disruptable off switch of transcription. To test this notion, we first determined whether ANR was capable of colocalizing the transcriptional activator VP64-p65-Rta (VPR) with a Gal4 DNA-binding domain-NS3a(H1) fusion bound upstream of an mCherry™ reporter gene (FIG. 4A). Consistent with the NS3a(H1)/ANR interaction promoting transcription, we observed a significant increase in mCherry™ expression in cells expressing a ANR-VPR fusion construct (FIG. 4B). We found that treatment of cells with danoprevir or grazoprevir decreased mCherry™ expression to background levels-defined by cells expressing a VPR fusion (DNCR2-VPR) that lacks ANR.

Finally, we explored whether our CDP system could be combined with chemical methods for activating transcription. To do this, we used a nuclease-null, chemically-inducible Cas9 (dciCas9) variant that is autoinhibited by the BCL-xL/BH3 interaction and can be activated with a chemical disrupter. An NS3a(H1)-VPR fusion was recruited upstream of a GFP reporter gene through its interaction with an MCP-ANR fusion bound to an MS2 stem loop of a scaffold RNA targeted to the Tet operator (FIG. 4C). Activation of dciCas9 with a drug—A115—that disrupts the autoinhibitory the BCL-xL/BH3 interaction led to an increase in GFP expression (FIG. 4D). We observed that this increase in expression was reversed when grazoprevir was co-administered with A115. Thus, the chemically-disruptable NS3a/ANR interaction can be combined with chemical systems for transcriptional activation to provide temporally-regulated on/off switches.

In sum, we have developed a CDP system based on the interaction between the viral protease NS3a and a genetically-encoded peptide inhibitor. We demonstrated that our NS3a-based CDP system can be used to engineer chemical control over a number of intracellular protein functions. The use of NS3a as a component of a CDP system further expands the utility of this protease as a chemically-controllable module. The reagents and chemically-controlled methods disclosed can be used to confer temporal control over intracellular protein function. Furthermore, the orthogonality of our CDP components to currently available CIP systems allows integration of these strategies.

### EXAMPLE 1 REFERENCES

(1) Haugh, J. M.; Lauffenburger, D. A. Physical modulation of intracellular signaling processes by locational regulation. Biophys. J. 1997, 72, 2014-31.
(2) Kholodenko, B. N.; Hoek, J. B.; Westerhoff, H. V. Why cytoplasmic signalling proteins should be recruited to cell membranes. Trends Cell Biol. 2000, 10, 173-8.
(3). Ptashne, M.; Gann, A. Transcriptional activation by recruitment. Nature 1997, 386, 569-77.
(4) Fegan, A.; White, B.; Carlson, J. C. T.; Wagner, C. R. Chemically controlled protein assembly: techniques and applications. Chem. Rev. 2010, 110, 3315-36.
(5) Putyrski, M.; Schultz, C. Protein translocation as a tool: The current rapamycin story. FEBS Lett. 2012, 586, 2097-105.
(6) Rakhit, R.; Navarro, R.; Wandless, T. J. Chemical biology strategies for posttranslational control of protein function. Chem. Biol. 2014, 21, 1238-52.
(7) Yazawa, M.; Sadaghiani, A. M.; Hsueh, B.; Dolmetsch, R. E. Induction of protein-protein interactions in live cells using light. Nat. Biotechnol. 2009, 27, 941-5.
(8) Stanton, B. Z.; Chory, E. J.; Crabtree, G. R. Chemically induced proximity in biology and medicine. Science 2018, 359, eaao5902.
(9) Goreshnik, I.; Maly, D. J. A small molecule-regulated guanine nucleotide exchange factor. J. Am. Chem. Soc. 2010, 132, 938-940.
(10) Rose, J. C.; Huang, P.-S.; Camp, N. D.; Ye, J.; Leidal, A. M.; Goreshnik, I.; Trevillian, B. M.; Dickinson, M. S.; Cunningham-Bryant, D.; Debnath, J.; Baker, D.; Wolf-Yadlin, A.; Maly, D. J. A computationally engineered RAS rheostat reveals RAS-ERK signaling dynamics. Nat. Chem. Biol. 2017, 13, 119-26.
(11) Rose, J. C.; Dieter, E. M.; Cunningham-Bryant, D.; Maly, D. J. Examining RAS pathway rewiring with a chemically inducible activator of RAS. Small GTPases 2018, in press.
(12) Rose, J. C.; Stephany, J. J.; Valente, W. J.; Trevillian, B. M.; Dang, H. V.; Bielas, J. H.; Maly, D. J.; Fowler, D. M. Rapidly inducible Cas9 and DSB-ddPCR to probe editing kinetics. Nat. Methods 2017, 14, 891-6.
(13) Rose, J. C.; Stephany, J. J.; Wei, C. T.; Fowler, D. M.; Maly, D. J. Rheostatic Control of Cas9-Mediated DNA Double Strand Break (DSB) Generation and Genome Editing. ACS Chem. Biol. 2018, 13, 438-42.
(14) McCauley, J. A.; Rudd, M. T. Hepatitis C virus NS3/4a protease inhibitors. Curr. Opin. Pharmacol. 2016, 30, 84-92.
(15) Kügler, J.; Schmelz, S.; Gentzsch, J.; Haid, S.; Pollmann, E.; van den Heuvel, J.; Franke, R.; Pietschmann, T.; Heinz, D. W.; Collins, J. High affinity peptide inhibitors of the hepatitis C virus NS3-4A protease refractory to common resistant mutants. J. Biol. Chem. 2012, 287, 39224-32.
(16) Huang, P.-S.; Ban, Y-E. A.; Richter, F.; Andre, I.; Vernon, R.; Schief, W. R.; Baker, D. RosettaRemodel: a generalized framework for flexible backbone protein design. PLoS ONE 2011, 6, e24109.
(17) Brass, V; Berke, J. M.; Montserret, R.; Blum, H. E.; Penin, F.; Moradpour, D. Structural determinants for membrane association and dynamic organization of the hepatitis C virus NS3-4A complex. Proc. Natl. Acad. Sci. U.S.A. 2008, 105, 14545-50.
(18) Wittekind, M.; Weinheirner, S.; Zhang, Y; Goldfarb, V. Modified forms of hepatitis C NS3 protease for facilitating inhibitor screening and structural studies of protease:inhibitor complexes. U.S. Pat. No. 6,333,186. 2004.
(19) Mali, P.; Aach, J.; Stranges, P. B.; Esvelt, K. M.; Moosburner, M.; Kosuri, S.; Yang, L.; Church, G. M. CAS9 transcriptional activators for target specificity screening and paired nickases for cooperative genome engineering. Nat. Biotechnol. 2013, 31, 833-8.
(20) Zalatan, J. G.; Lee, M. E.; Almeida, R.; Gilbert, L. A.; Whitehead, E. H.; La Russa, M.; Tsai, J. C.; Weissman, J. S.; Dueber, J. E.; Qi, L. S.; Lim, W. A. Engineering complex synthetic transcriptional programs with CRISPR RNA scaffolds. Cell 2015, 160, 339-50.
(21) Jacobs, C. L.; Badiee, R. K.; Lin, M. Z. StaPLs: versatile genetically encoded modules for engineering drug-inducible proteins. Nat. Methods 2018, 15, 523-6.
(22) Tague, E. P.; Dotson, H. L.; Tunney, S. N.; Sloas, D. C.; Ngo, J. T. Chemogenetic control of gene expression and cell signaling with antiviral drugs. Nat. Methods 2018, 15, 519-22.

### Methods

### 1. Computational Design of NS3a-CDAR

The NS3a-CDAR construct was modeled after a previously developed BCL-xL/BH3 autoinhibited SOScat fusion design wherein a BH3 peptide was fused to the N-terminus (residue 574) of SOScat and BCL-xL was fused to the C-terminus (residue 1020). Due to similarities in the topology between the BCL-xL/BH3 complex and the NS3a/ANR complex, we limited our computational modeling to a construct composed of SOScat (574-1029) containing ANR fused to the N-terminus and NS3a fused to the C-terminus. ANR and NS3a were fused to SOScat through flexible linkers.

The NS3a/ANR complex (PDB 4A1X) was modeled using the RosettaRemodel™ conformational sampling protocol described previously (Rose, J. C. et al. Nat. Chem. Biol. 2017, 13, 119-126.). Briefly, the NS3a/ANR autoinhibitory complex was treated as a single rigid-body between the N- and C-termini of SOScat (PDB 1XD2). To allow this setup, the SOScat structure was circularly permuted, with a chain break introduced arbitrarily, away from the termini. This scheme allows for treatment of the NS3a/ANR complex across the termini as a loop closure problem, wherein a break is randomly introduced into one of the linkers to be reconnected via both random fragment moves and chain-closure algorithms guided by the Rosetta™ energy function; trajectories that properly reconnected the chain were considered successful.

Linkers were assigned the identity of repeating glycine-serine/threonine residues. We tested N-terminal linkers between 1 and 13 residues in length at 2 residue increments, and C-terminal linkers between 5 and 29 residues in length at 2 residue increments, giving 91 different linker length combinations.

1,000 independent trajectories were sampled in 100 parallel runs that used the flags above. The lowest energy model from each successful trajectory was saved as a PDB file.

### 2. Plasmid Construction

### Bacterial Expression Constructs

Non-biotinylated NS3a variants and ANR-GST fusions were obtained as double stranded DNA G-Blocks (IDT) containing Gibson Assembly overhangs designed in NEBuilder™ (NEB). ANR was designed with an N-terminal hexahistidine tag and a C-terminal Glutathione S-Transferase domain. NS3a protease genes were sub-cloned into the pMCSG7 vector backbone by PCR linearization of the vector, then Gibson assembly of the vector with the gene insert (NEB, product number E2611L). All NS3a constructs contained an N-terminal hexahistidine tag. This NS3a fusion was used for all in vitro experiments with NS3a except for the protease assay shown in FIG. 6A and the pulldown experiments shown in FIG. 7C.

NS3a for biotinylation was cloned into the pDW363 vector. NS3a was N-terminally fused to AviTag™ biotin acceptor peptide followed by a hexahistidine tag. The pDW363 vector contains a bi-cistronic BirA biotin ligase. Avi-tagged NS3a was cloned into pDW363 via PCR-linearization of the vector, followed by Gibson assembly with the gene insert, obtained as double stranded DNA G-Blocks containing Gibson Assembly overhangs designed in NEBuilder™.

### Mammalian Expression Constructs

All constructs for NS3a-CDAR and sub-cellular colocalization microscopy experiments were obtained as codon-optimized, double-stranded DNA G-Blocks™ (Integrated DNA Technologies) containing Gibson Assembly overhangs designed in NEBuilder™ (NEB). Genes were sub-cloned into pcDNA5/FRT/TO vector (Thermo Fisher Scientific) by PCR linearization of the vector, then Gibson Assembly of the vector with the gene insert. ANR and NS3a sequence variants were obtained via Quikchange™ mutagenesis.

Plasmids containing single-guide RNAs (TRE3G) were generated by cloning into gRNA Cloning Vector (gifts from George Church (Addgene plasmid #41824)). DNA corresponding to the guide target was ordered as a single stranded oligonucleotide containing Gibson assembly overhangs complementary to the vector and assembled with AflII-digested gRNA vector. A scaffold RNA (scRNA) targeting TRE3G containing two MS2 hairpins was cloned into dual insert vectors derived from pSico™, expressing the scaffold RNA under a U6 promoter and the protein inserts under a CMV promoter: pJZC34 (MS2/MCP) (gift from Jesse Zalatan). All MS2 fusions were expressed as P2A-BFP fusions instead of the IRES-mCherry fusions in the original vectors.

The parental pLenti Gal4 reporter plasmid ‘G143’ (UAS-mCherry™/CMV-Gal4-ERT2-VP16-P2A-Puro) was a gift from Doug Fowler. The ERT2-VP16 and Puromycin resistance cassette was exchanged for NS3a(H1)-P2A-ANR—BFP-NLS-VPR. Fragments were obtained from the previously mentioned pcDNA5/FRT/TO expression systems by PCR and restriction digesting G143 with BamHI and SexAI. Fragments and digested vector were assembled using Gibson Assembly.

All PCR reactions (vector linearizations, Gibson assembly insert preparation, and Quikchanges) were performed with Q5 polymerase (New England Biolabs). All Gibson assembly reactions were performed with NEBuilder™ HiFi Assembly Master Mix (New England Biolabs). Oligonucleotides and Gene Blocks™ used for cloning were synthesized by Integrated DNA Technologies. Correct insertion of the genes and vector preparations were verified by whole gene sequencing (Genewiz). Protein sequences for all constructs used are provided in Table 13.

### 3. Protein Expression and Purification

### SNAPtag-NS3a

The SNAPtag™-NS3a-His₆ plasmid was transformed into BL21(DE3) E. coli cells. One colony was used to inoculate 5 mL of LB broth with ampicillin (100 μg/mL). 18 hours post inoculation, the entirety of the 5 mL culture was used to inoculate 500 mL of LB both with ampicillin (100 μg/mL). Cultures were grown at 37° C. to on OD₆₀₀ of 0.8, cooled to 18° C. and induced with 0.25 mM IPTG. Protein was expressed at 18° C. overnight. Cells were harvested by centrifugation and pellets stored at −80° C. For SNAPtag-NS3a purification, the pellets were thawed on ice and re-suspended in 10 mL of LS-His₆ Lysis Buffer (50 mM HEPES pH 7.8, 100 mM NaCl, 20% (w/v) glycerol, 20 mM imidazole, 5 mM DTT). The re-suspended cell pellet was lysed via sonication and the lysate was cleared by centrifugation. The cleared lysate was purified using Ni-NTA agarose (Qiagen) by rotating at 4° C. for 1 hour. The resin was subsequently washed with 10 mL of LS-Lysis Buffer and the protein was eluted in 3 mL of LS-Elution Buffer (50 mM HEPES pH 7.8, 100 mM NaCl, 20% (w/v) glycerol, 200 mM Imidazole, 5 mM DTT). Purified protein was dialyzed twice into 1000 mL LS-Storage Buffer (50 mM HEPES pH 7.8, 100 mM NaCl, 20% (w/v) glycerol, 5 mM DTT, 0.6 mM lauryldimethylamine-N-oxide). Protein was stored by snap-freezing aliquots and storing at −80° C.

### NS3a Variants

NS3a variant expressions were performed in BL21 (DE3) E. coli by growing cells at 37° C. to an O.D.600 of 0.5-1.0, then moved to 18° C. Immediately following transfer to 18° C., protein expression was induced with 0.5 mM IPTG overnight. For biotinylated constructs, 12.5 mg of D(+)-biotin/L was added simultaneously during inoculation with the overnight culture. Following 16-20 hours overnight growth, cultures were subsequently harvested, and cell pellets frozen at −80° C. Cell pellets were then re-suspended in 20 mM Tris pH 8.0, 500 mM NaCl, 5 mM imidazole, 1 mM DTT, 0.1% Tween-20. All buffers for NS3a variant purifications included 10% v/v glycerol. Cells were lysed by sonication, and the supernatant was incubated with Ni-NTA resin (Qiagen) for a minimum of 1 hour at 4° C. Ni-NTA resin was then washed with three volumes of “NS3a-Wash Buffer” (20 mM Tris pH 8.0, 500 mM NaCl, 20 mM imidazole, 10% glycerol), and proteins were eluted with “NS3a Elution Bufer” (20 mM Tris pH 8.0, 500 mM NaCl, 300 mM imidazole, 10% glycerol). Purified protein was dialyzed twice (3.5 kDa mwco Slide-A-Lyzer™ dialysis cassettes, Thermo Scientific) into 1000 mL NS3a-Storage Buffer (50 mM HEPES pH 7.8, 100 mM NaCl, 10% (w/v) glycerol, 5 mM DTT, 0.6 mM lauryldimethylamine-N-oxide). Protein was stored by snap-freezing aliquots in liquid nitrogen and storing at −80° C. Biotinylated constructs were then further purified by size exclusion chromatography on a Superdex-75 10/300 GL column (GE Healthcare) in a buffer of in 20 mM Tris pH 8.0, 300 mM NaCl, 1 mM DTT, 10% glycerol.

### ANR-GST

His₆-ANR-GST plasmid was expressed in BL21(DE3) E. coli cells. 18 hours post inoculation, the entirety of the 5 mL culture was used to inoculate 250 mL of LB both with ampicillin (100 μg/mL). Cultures were grown at 37° C. to on OD₆₀₀ of 0.8, cooled to 18° C. and induced with 0.5 mM IPTG. Protein was expressed at 18° C. overnight. Cells were harvested by centrifugation and pellets stored at −80° C. For ANR-GST purification, the pellet was thawed on ice and re-suspended in 10 mL of His₆ Lysis Buffer (50 mM HEPES pH 7.8, 100 mM NaCl, 20 mM imidazole, 5 mM DTT) supplemented with PMSF (1 mM). The re-suspended cell pellet was lysed via sonication and the lysate was cleared by centrifugation. The cleared lysate was purified using Ni-NTA agarose (Qiagen) by rotating at 4° C. for 1 hour. The resin was subsequently washed with 10 mL of Lysis Buffer and the protein was eluted in 3 mL of Elution Buffer (50 mM HEPES pH 7.8, 100 mM NaCl, 200 mM Imidazole, 5 mM DTT). Purified protein was dialyzed twice into 1000 mL Storage Buffer (50 mM HEPES pH 7.8, 100 mM NaCl, 5 mM DTT). Protein was stored by snap-freezing aliquots and storing at −80° C.

### Inhibitor Sources

Grazoprevir was purchased from MedChem Express (MK-5172, product #: HY-15298). Asunaprevir (BMS-650032, product #: A3195) and Danoprevir (RG7227, product #: A4024) were both purchased from ApexBio. A-115463 was purchased from ChemieTek (Product #: CT-A115).

### 4. Fluorescence Polarization Assays

### A. Determination of FB₅₀s

The affinities of the NS3a variants for ANR were determined using a fluorescence polarization assay. Fluorescently labeled ANR (FAM-ANR, FIG. 5B) was obtained as a crude mixture from GenScript™ and purified by HPLC. Titrations of recombinant NS3a variants (3-fold serial dilutions, starting at 5 μM) were diluted in FP-Buffer (50 mM HEPES, pH 7.8, 100 mM NaCl, 5 mM DTT, 1% Glycerol, 0.01% Tween, 5% v/v DMSO). These dilutions were added to a wells containing FAM-ANR (final concentration=10 nM). FAM-ANR/NS3a solutions were incubated at room temperature in the dark for 1 hour. Fluorescence polarization was measured on a Perkin Elmer EnVision™ fluorometer (excitation, 495 nm; emission 520 nm). All measurements were carried out in black 96-well plates (Corning, product #: 3720) and run in triplicate. Anisotropy values were obtained and a nonlinear regression model was used to determine binding constants in GraphPad™ Prism.

### B. Fluorescence Polarization Competition Assay

Fluorescence polarization competition assays were used to determine the ability of danoprevir to displace ANR. A 75 nM solution of NS3a in FP-Buffer was incubated with 50 nM FAM-ANR in a black 96-well plate for 1 hour in the dark. 3-fold serial dilutions of danoprevir were prepared in FP-Buffer such that, when added to the NS3a/FAM-ANR solution, the highest concentration of danoprevir tested was 10 μM. Plates were incubated for 1 hour in the dark. Fluorescence polarization was measured at 22° C. on a Perkin Elmer EnVision™ fluorometer (excitation, 495 nm; emission 520 nm). Each measurement was carried out in triplicate. Anisotropy values were obtained and a nonlinear regression model was used to fit curves with GraphPad Prism.

### 5. NS3a Protease Inhibition Assay

The potency of ANR against NS3a protease was determined via a FRET assay. Titrations of ANR-GST (3-fold serial dilutions starting at 10 μM) were added to a black 96-well plate (Corning, product number 3720) containing 50 nM SNAPtag-NS3a. Reactions were incubated with NS3a-SNAPtag at room temperature for 1 hour. To each well was simultaneously added substrate M-2235 (Bachem) to a final concentration of 5 μM and reactions were monitored by measuring the fluorescence intensity every minute for 30 minutes at 22° C. on a Perkin Elmer EnVision™ fluorometer (excitation, 360 nm; emission 460 nm). Each measurement was carried out in triplicate. Slopes of the fluorescence increase were compared to a no-protease control. A nonlinear regression model was used to fit curves using GraphPad™ Prism.

### 6. ANR-GST Pulldown

Pierce high-capacity streptavidin beads (Thermo-Fisher #P120359) were prepared by washing three times with Buffer PDA (TBS+0.05% tween+0.5 mg/mL BSA). For each condition and each replicate, beads were washed and incubated separately. The wash was performed by adding 200 μL Buffer PDA to 30 μL of a 50/50 bead slurry, inverting to mix, and spinning down (2500×g for 2 min). The supernatant was removed by pipetting, and the wash was repeated two more times to end with a 50/50 slurry of beads in wash buffer.

Purified biotinylated NS3a was prepared at a 50× final concentration and 10 μL were added to a 490 μL 50/50 slurry of streptavidin beads and Buffer PD for final NS3a concentration of 125 nM. Beads were incubated and rotated at 4° C. After one hour, beads were harvested and washed three times as described previously, ending in a 50/50 bead/buffer slurry. ANR was added to all samples at a final concentration of 5 μM. For the danoprevir treated samples, danoprevir was added to a final concentration of 10 μM. Buffer PD was added to a final volume of 500 μL, and the beads were incubated and rotated at 4° C. After 1 hour, beads were pelleted and washed three times in Buffer FDB (TBS buffer+0.05% Tween) with 5 minute incubations between washes on a rotator at 4° C. To obtain final bound protein, beads were pelleted and supernatant was aspirated, resulting in a final volume of beads of 20 μL. 10 μL 3×SDS loading dye was added directly to beads and boiled at 90° C. for 10 min. Bead mixture was pelleted and supernatants were loaded directly onto a polyacrylamide gel for Western Blot analysis (Mini-PROTEAN™ TGX Any kD, Bio-Rad #456-9036).

### 7. Mammalian Cell Culture

### A. NIH-3T3 Cell Culture and Transient Transfection Conditions

NIH-3T3 cells were maintained in DMEM (Gibco, product number 11065092) supplemented with 10% FBS (Gibco, product number A3160602). All transient transfections were done using LipoFectamine3000 (ThermoFisher, product number L3000015) at a ratio of 3:2:1 LipoFectamine3000:p3000Reagent:DNA (μg) prepared in OptiMem™ (Gibco, product number 11058021) 16-20 hours after plating of cells. Transfections were allowed to proceed for 24 hours before experiments were performed. Cells were tested and found free of mycoplasma monthly.

### B. Confocal Microscopy of Protein Colocalization

24 hours prior to transfection, 3×10⁴ 3T3 cells were plated onto 18 mm glass cover slips (Fisher, product number 12-546) in a standard 12-well plate. After co-transfection with the appropriate NS3a/ANR pairs (Tom20-mCherry™-NS3a(H #)/EGFP-ANR₂, Myr-mCherry™-ANR₂/EGFP-NS3a(H1), or NLS₃-BFP-ANR₂/EGFP-NS3a(H1)), cells were allowed to recover for 24 hours before treatment with 10 μM asunaprevir or DMSO (0.5% DMSO final concentration). Cells were incubated with drug for the stated time points before media was aspirated, then washed once with chilled PBS, and immediately fixed in 4% paraformaldehyde (Electron Microscopy Services, product number 15710). Paraformaldehyde solution was prepared in 1×PBS and cells were allowed to fix for 15 minutes. Paraformaldehyde was removed and cells were washed twice with chilled PBS. Slides were mounted onto glass cover slips using Fluoromount G (Southern Biotechnology, product number 0100-01) and sealed. Images were generated using a Leica SP8× Confocal Microscope. UV lasers at 405 nm was used for BFP. White lasers (488 nm and 587 nm) were used for EGFP and mCherry™, respectively. BFP fluorescence emissions were recorded using a PMT detector. EGFP and mCherry™ fluorescence emissions were recorded by separate HyD detectors. Images were acquired using a 63× oil objective at 512×512 resolution. Only images of cells exhibiting both mCherry™ and EGFP (or both BFP and EGFP for nuclear colocalization) were collected. The degree of colocalization was measured as Pearson's r-correlation coefficients. Pearson's r coefficients were determined using ImageJ™.

### Statistics

All P-values are from unpaired, two-sided t-tests, computed using Graphpad™ Prism 5.

### C. HEK293 and HEK293T Cell Culture and Transient Transfection Conditions

HEK293 and HEK293T cells were maintained in DMEM (Gibco, #11065092) supplemented with 10% FBS (Gibco, product number A3160602). Transient transfections for all experiments were carried out using TurboFectin8.0 (Origene) at a ratio of 3:1 TurboFectin™:DNA (μg) prepared in OptiMem™ (Gibco, #11058021) 16-20 hours after plating of cells. Transfections were allowed to proceed for 18-24 hours before experiments were performed or media was exchanged. Cells were tested and found free of mycoplasma monthly.

### Activation of NS3a-CDAR

18-24 hours prior to transfection, 3.0×10⁵ HEK293 cells were plated onto poly-D-lysine 12 well plates. Immediately prior to transfection, media was aspirated and cells were washed with 1 mL of pre-warmed (37° C.) PBS, then serum starved with FBS-free DMEM. Following serum starvation, cells were transfected with 1 μg of FLAG-tagged NS3a-CDAR, BH3-NS3a-CDAR, or an empty pCDNA5 vector. Transfected cells were allowed to serum stave for 18-20 hours prior to drug treatment. For drug treatment, serum-free media was prepared with DMSO or 10 μM of a drug. Media was aspirated, washed once with pre-warmed DPBS, then treated with drug/DMSO media for the requisite amount of time. Media was subsequently aspirated and the cells were washed twice with 1 mL chilled PBS, then lysed with 75 μL Mod. RIPA buffer (50 mM Tris, pH 7.8, 1% IGEPAL CA-630, 150 mM NaCl, 1 mM EDTA, 2 mM Na₃VO₄, 30 mM NaF, Pierce Protease Inhibitor Tablet). Cleared lysates were subjected to SDS-PAGE and transferred to nitrocellulose. Blocking and antibody incubations were done in TBS with 0.1% Tween-20 (v/v) and blocking buffer (Odyssey). Primary antibodies were all purchased from Cell Signaling Technologies and were diluted as follows: Total ERK (1:2500, #9107), phosphorylated ERK (1:2500, #4370), FLAG (1:2,500, #D6W5B). Blots were washed three times in TBS with 0.1% Tween-20. Antibody binding was detected by using near-infrared-dye-conjugated secondary antibodies and visualized on the LI-COR Odyssey scanner. Blots were quantified via densitometry with Image Studio (LI-COR).

### Chemically-Disruptable Gal4(DBD)-NS3a(H1)/ANR-VPR Transcriptional Regulation

18-24 hours prior to transfection, HEK293T cells were plated in a 12-well plate at a density of 1.25×10⁵ cells/mL. Cells were subsequently transfected with 1 μg of the Gal4 reporter plasmid (UAS-mCherry™/CMV-Gal4-NS3a(H1)-P2A-ANR-Myc-BFP-VPR-NLS) in OptiMem™. For the negative control experiment, 500 ng of a plasmid where ANR was replaced with the non-NS3a binding protein DNCR2 (UAS-mCherry™/CMV-Gal4-NS3a(H1)-P2A-DNCR2-Myc-VPR-NLS was co-transfected with 500 ng of a BFP expressing reporter plasmid in OptiMem™. 16 hours post transfection, cells were washed with 1 mL DPBS. Complete media containing 1 μM danoprevir, 1 μM grazoprevir, or DMSO was subsequently added to each well. 24 hours after drug treatment, media was removed and cells were washed with 1 mL DPBS, then detached with 200 μL Versene™ (Sigma-Aldrich, 15-040-066). Cells were then re-suspended with 500 μL DPBS, and pelleted at 2500 rpm for 3 min at room temperature. Supernatant was subsequently removed and the cells were re-suspended in 400 μL DPBS and analyzed on a FACS LSRII (BD Biosciences).

For Gal4/NS3a-CDP mediated transcriptional activation FACS experiments, 10,000 single cell events were collected for each of the samples run. Of these 10,00 single cell events, the median mCherry™ fluorescence signal is reported only for cells exhibiting BFP signal greater than that of non-transfected cells. The gathered FACS data were analyzed using FlowJo™ (v.10.1).

dciCas9-Mediated Transcription

GFP expression experiments were performed in a HEK293T cell line with GFP stably integrated downstream of a tetracycline-inducible landing pad (7x-TRE3G operator) created in a similar manner as a previously reported Tet-Bxb1-BFP HEK293T cell line (Matreyek et al. Nucleic Acids Res. 2017, 45, e102.). For the dciCas9-mediated transcriptional activation experiment, 6×10⁴ cells/well were plated in 12-well plates on day 1 and transfected with 1 μg total DNA on day 2 (0.3 μg dciCas9 vector, 0.3 μg NS3a(H1)-VPR vector, and 0.4 μg NLS-MCP-ANR₂/TRE3G scaffold RNA vector). 18 hours after transfection, media was replaced with complete DMEM containing DMSO, 10 μM A115, or 10 μM A115 and 10 μM grazoprevir. 48 hours post drug treatment, media was aspirated and cells were washed with 1 mL pre-warmed DPBS, then detached and analyzed as described in the chemically-disruptable Gal4(DBD)-NS3a(H1)/VPR-ANR/transcriptional regulation experiment.

For FACS analysis, 10,000 single cell events were collected for each of the samples run. Of these 10,00 single cell events, the median GFP fluorescence signal is reported only for cells exhibiting BFP signal greater than that of non-transfected cells. The gathered FACS data were analyzed using FlowJo (v.10.1).

### Statistics

All P-values are from unpaired, two-sided t-tests, computed using Graphpad™ Prism 5.

TABLE 13
Sequences of proteins and guide RNAs

| | | Sequence (NS3a sequences are shown in bold. |
|---|---|---|
| Sequence | | ANR sequences are shown in italics. |
| ID | Description | Regions of note are underlined or highlighted). |
| ANR-GST | His₆-ANR-GST | MHHHHHHGSGTGSGELGRLVYLLDGPGYDPIHSDGTGSSPILGYWKIKGLVQPT |
| | (E. coli) | RLLLEYLEEKYEEHLYERDEGDKWRNKKFELGLEFPNLPYYIDGDVKLTQSMA |
| | | IIRYIADKHNMLGGCPKERAEISMLEGAVLDIRYGVSRIAYSKDFETLKVDFLSK |
| | | LPEMLKMFEDRLCHKTYLNGDHVTHPDFMLYDALDVVLYMDPMCLDAFPKL |
| | | VCFKKRIEAIPQIDKYLKSSKYIAWPLQGWQATFGGGDHPPKSDLVPR |
| | | (SEQ ID NO: 50) |
| SNAP_{tag}- | SNAP_{tag}-NS3a-His₆ | MDKDCEMKRTTLDSPLGKLELSGCEQGLHEIKLLGKGTSAADAVEVPAPAAVL |
| NS3a | (E. coli) | GGPEPLMQATAWLNAYFHQPEAIEEFPVPALHHPVFQQESFTRQVLWKLLKVV |
| | | KFGEVISYQQLAALAGNPAATAAVKTALSGNPVPILIPCHRVVSSSGAVGGYEG |
| | | GLAVKEWLLAHEGHRLGKPGLGGTGTAKGSVVIVGRINLSGDTAYSQQTRG |
| | | LLGIIITSATGRDKNQVDGEVQVLSTATQSFLATCVNGVCWTVYHGAGSKT |
| | | LAGPKGPITQMYTNVDQDLVGWPAPPGARSMTPCTCGSSDLYLVTRHADV |
| | | IPVRRRGDSRGSLLSPRPVSYLKGSSGGPLLCPSGHVVGIFRAAVCTRGVAK |
| | | AVDFIPVESMETTMRGSHHHHHH (SEQ ID NO: 51) |
| NS3a | Avi-His₆-NS3a | MAGGLNDIFEAQKIEWHEDTGGSSHHHHHHGSGSGSMAKGSVVIVGRINLSG |
| (inactive) | solubility optimized | DTAYSQQTRGLLGCIITSATGRDKNQVDGEVQVLSTATQSFLATCVNGVC |
| | S139A | WTVYHGAGSKTLAGPKGPITQMYTNVDQDLVGWPAPPGARSMTPCTCGS |
| | (E. coli) | SDLYLVTRHADVIPVRRRGDSRGSLLSPRPVSYLKGSAGGPLLCPSGHVVGI |
| | | FRAAVCTRGVAKAVDFIPVESMETTMR (SEQ ID NO: 52) |
| NS3a | Avi-His₆-NS3a | MAGGLNDIFEAQKIEWHEDTGGSSHHHHHHGSGSGSMAKGSVVIVGRINLSG |
| (active) | solubility optimized | DTAYSQQTRGLLGCIITSATGRDKNQVDGEVQVLSTATQSFLATCVNGVC |
| | catalytically active | WTVYHGAGSKTLAGPKGPITQMYTNVDQDLVGWPAPPGARSMTPCTCGS |
| | (E. coli) | SDLYLVTRHADVIPVRRRGDSRGSLLSPRPVSYLKGSSGGPLLCPSGHVVGI |
| | | FRAAVCTRGVAKAVDFIPVESMETTMR (SEQ ID NO: 53) |
| NS3a* | His₆-NS3a | MAGGSSHHHHHHGSGSGSMKKKGSVVIVGRINLSGDTAYAQQTRGEEGCQ |
| (inactive) | solubility optimized | ETSQTGRDKNQVEGEVQIVSTATQTFLATSINGVLWTVYHGAGTRTIASPK |
| | S139A | GPVTQMYTNVDKDLVGWQAPQGSRSLTPCTCGSSDLYLVTRHADVIPVRR |
| | (E. coli) | RGDSRGSLLSPRPISYLKGSAGGPLLCPAGHAVGIFRAAVSTRGVAKAVDFI |
| | | PVESLETTMRSP (SEQ ID NO: 54) |
| NS3a* | His₆-NS3a | MAGGSSHHHHHHGSGSGSMKKKGSVVIVGRINLSGDTAYAQQTRGEEGCQ |
| (active) | solubility optimized- | ETSQTGRDKNQVEGEVQIVSTATQTFLATSINGVLWTVYHGAGTRTIASPK |
| | catalytically active | GPVTQMYTNVDKDLVGWQAPQGSRSLTPCTCGSSDLYLVTRHADVIPVRR |
| | (E. coli) | RGDSRGSLESPRPISYLKGSSGGPLLCPAGHAVGIFRAAVSTRGVAKAVDFI |
| | | PVESLETTMRSP (SEQ ID NO: 55) |
| NS3a(H1) | His₆-NS3a(H1) | MGGSSHHHHHHGSGSGSMKKKGSVVIVGRINLSGDTAYSQQTRGLEGCQE |
| (active) | Chimera- | TSQTGRDKNQVEGEVQVVSTATQSFLATSINGVLWTVYHGAGTRTIASPK |
| | catalytically active | GPVTQMYTNVDKDLVGWQAPQGSRSLTPCTCGSSDLYLVTRHADVIPVRR |
| | (E. coli) | RGDSRGSLLSPRPISYLKGSSGGPLLCPAGHAVGIFRAAVSTRGVAKAVDFI |
| | | PVESLETTMRSP (SEQ ID NO: 56) |
| Tom20- | Mitochondrial- | MVGRNSAIAAGVCGALFIGYCIYFDRKRRSDPNFGSGGSMVSKGEEDNMAIIKE |
| mCherry- | localized | FMRFKVHMEGSVNGHEFEIEGEGEGRPYEGTQTAKLKVTKGGPLPFAWDILSP |
| NS3a(H1) | NS3a/NS3a* | QFMYGSKAYVKHPADIPDYLKLSFPEGFKWERVMNFEDGGVVTVTQDSSLQD |
| | chimera H1- | GEFIYKVKLRGTNFPSDGPVMQKKTMGWEASSERMYPEDGALKGEIKQRLKL |
| | catalytically active, | KDGGHYDAEVKTTYKAKKPVQLPGAYNVNIKLDITSHNEDYTIVEQYERAEGR |
| | pcDNA5/FRT/TO | HSTGGMDELYKGSGSTGTSGSGSGTGSGSGTGMKKKGSVVIVGRINLSGDTA |
| | | YSQQTRGLEGCQETSQTGRDKNQVEGEVQVVSTATQSFLATSINGVLWTV |
| | | YHGAGTRTIASPKGPVTQMYTNVDKDLVGWQAPQGSRSLTPCTCGSSDLY |
| | | LVTRHADVIPVRRRGDSRGSLLSPRPISYLKGSSGGPLLCPAGHAVGIFRAA |
| | | VSTRGVAKAVDFIPVESLETTMRSPGSGTGSGTSGSTGTGSTGDYKDDDDK |
| | | (SEQ ID NO: 57) |
| Tom20- | Mitochondrial- | MVGRNSAIAAGVCGALFIGYCIYFDRKRRSDPNFGSGGSMVSKGEEDNMAIIKE |
| mCherry- | localized | FMRFKVHMEGSVNGHEFEIEGEGEGRPYEGTQTAKLKVTKGGPLPFAWDILSP |
| NS3a(H2) | NS3a/NS3a* | QFMYGSKAYVKHPADIPDYLKLSFPEGFKWERVMNFEDGGVVTVTQDSSLQD |
| | chimera H2- | GEFIYKVKLRGTNFPSDGPVMQKKTMGWEASSERMYPEDGALKGEIKQRLKL |
| | catalytically active, | KDGGHYDAEVKTTYKAKKPVQLPGAYNVNIKLDITSHNEDYTIVEQYERAEGR |
| | pcDNA5/FRT/TO | HSTGGMDELYKGSGSTGTSGSGSGTGSGSGTGMKKKGSVVIVGRINLSGDTA |
| | | YSQQTRGELGCQETSQTGRDKNQVEGEVQVVSTATQSFLATSINGVLWTV |
| | | YHGAGTRTIASPKGPVTQMYTNVDKDLVGWQAPQGSRSLTPCTCGSSDLY |
| | | LVTRHADVIPVRRRGDSRGSLLSPRPISYLKGSSGGPLLCPAGHAVGIFRAA |
| | | VSTRGVAKAVDFIPVESLETTMRSPGSGTGSGTSGSTGTGSTGDYKDDDDK |
| | | (SEQ ID NO: 58) |
| Tom20- | Mitochondrial- | MVGRNSAIAAGVCGALFIGYCIYFDRKRRSDPNFGSGGSMVSKGEEDNMAIIKE |
| mCherry- | localized | FMRFKVHMEGSVNGHEFEIEGEGEGRPYEGTQTAKLKVTKGGPLPFAWDILSP |
| NS3a(H3) | NS3a/NS3a* | QFMYGSKAYVKHPADIPDYLKLSFPEGFKWERVMNFEDGGVVTVTQDSSLQD |
| | chimera H3- | GEFIYKVKLRGTNFPSDGPVMQKKTMGWEASSERMYPEDGALKGEIKQRLKL |
| | catalytically active, | KDGGHYDAEVKTTYKAKKPVQLPGAYNVNIKLDITSHNEDYTIVEQYERAEGR |
| | pcDNA5/FRT/TO | HSTGGMDELYKGSGSTGTSGSGSGTGSGSGTGMKKKGSVVIVGRINLSGDTA |
| | | YSQQTRGLLGCQETSQTGRDKNQVEGEVQVVSTATQSFLATSINGVLWTV |
| | | YHGAGTRTIASPKGPVTQMYTNVDKDLVGWQAPQGSRSLTPCTCGSSDLY |
| | | LVTRHADVIPVRRRGDSRGSLLSPRPISYLKGSSGGPLLCPAGHAVGIFRAA |
| | | VSTRGVAKAVDFIPVESLETTMRSPGSGTGSGTSGSTGTGSTGDYKDDDDK |
| | | (SEQ ID NO: 59) |
| Tom20- | Mitochondrial- | MVGRNSAIAAGVCGALFIGYCIYFDRKRRSDPNFGSGGSMVSKGEEDNMAIIKE |
| mCherry- | localized | FMRFKVHMEGSVNGHEFEIEGEGEGRPYEGTQTAKLKVTKGGPLPFAWDILSP |
| NS3a(H4) | NS3a/NS3a* | QFMYGSKAYVKHPADIPDYLKLSFPEGFKWERVMNFEDGGVVTVTQDSSLQD |
| | chimera H4- | GEFIYKVKLRGTNFPSDGPVMQKKTMGWEASSERMYPEDGALKGEIKQRLKL |
| | catalytically active, | KDGGHYDAEVKTTYKAKKPVQLPGAYNVNIKLDITSHNEDYTIVEQYERAEGR |
| | pcDNA5/FRT/TO | HSTGGMDELYKGSGSTGTSGSGSGTGSGSGTGMKKKGSVVIVGRINLSGDTA |
| | | YSQQTRGLLGCIETSQTGRDKNQVEGEVQVVSTATQSFLATSINGVLWTV |
| | | YHGAGTRTIASPKGPVTQMYTNVDKDLVGWQAPQGSRSLTPCTCGSSDLY |
| | | LVTRHADVIPVRRRGDSRGSLLSPRPISYLKGSSGGPLLCPAGHAVGIFRAA |
| | | VSTRGVAKAVDFIPVESLETTMRSPGSGTGSGTSGSTGTGSTGDYKDDDDK |
| | | (SEQ ID NO: 60) |
| Tom20- | Mitochondrial- | MVGRNSAIAAGVCGALFIGYCIYFDRKRRSDPNFGSGGSMVSKGEEDNMAIIKE |
| mCherry- | localized | FMRFKVHMEGSVNGHEFEIEGEGEGRPYEGTQTAKLKVTKGGPLPFAWDILSP |
| NS3a(H5) | NS3a/NS3a* | QFMYGSKAYVKHPADIPDYLKLSFPEGFKWERVMNFEDGGVVTVTQDSSLQD |
| | chimera H5- | GEFIYKVKLRGTNFPSDGPVMQKKTMGWEASSERMYPEDGALKGEIKQRLKL |
| | catalytically active, | KDGGHYDAEVKTTYKAKKPVQLPGAYNVNIKLDITSHNEDYTIVEQYERAEGR |
| | pcDNA5/FRT/TO | HSTGGMDELYKGSGSTGTSGSGSGTGSGSGTGMKKKGSVVIVGRINLSGDTA |
| | | YSQQTRGLLGCIITSQTGRDKNQVEGEVQVVSTATQSFLATSINGVLWTVY |
| | | HGAGTRTIASPKGPVTQMYTNVDKDLVGWQAPQGSRSLTPCTCGSSDLYL |
| | | VTRHADVIPVRRRGDSRGSLLSPRPISYLKGSSGGPLLCPAGHAVGIFRAAV |
| | | STRGVAKAVDFIPVESLETTMRSPGSGTGSGTSGSTGTGSTGDYKDDDDK |
| | | (SEQ ID NO: 61) |
| Tom20- | Mitochondrial- | MVGRNSAIAAGVCGALFIGYCIYFDRKRRSDPNFGSGGSMVSKGEEDNMAIIKE |
| mCherry- | localized | FMRFKVHMEGSVNGHEFEIEGEGEGRPYEGTQTAKLKVTKGGPLPFAWDILSP |
| NS3a(H6) | NS3a/NS3a* | QFMYGSKAYVKHPADIPDYLKLSFPEGFKWERVMNFEDGGVVTVTQDSSLQD |
| | chimera H6- | GEFIYKVKLRGTNFPSDGPVMQKKTMGWEASSERMYPEDGALKGEIKQRLKL |
| | catalytically active, | KDGGHYDAEVKTTYKAKKPVQLPGAYNVNIKLDITSHNEDYTIVEQYERAEGR |
| | pcDNA5/FRT/TO | HSTGGMDELYKGSGSTGTSGSGSGTGSGSGTGMKKKGSVVIVGRINLSGDTA |
| | | YSQQTRGLEGCIETSQTGRDKNQVEGEVQVVSTATQSFLATSINGVLWTV |
| | | YHGAGTRTIASPKGPVTQMYTNVDKDLVGWQAPQGSRSLTPCTCGSSDLY |
| | | LVTRHADVIPVRRRGDSRGSLLSPRPISYLKGSSGGPLLCPAGHAVGIFRAA |
| | | VSTRGVAKAVDFIPVESLETTMRSPGSGTGSGTSGSTGTGSTGDYKDDDDK |
| | | (SEQ ID NO: 62) |
| Tom20- | Mitochondrial- | MVGRNSAIAAGVCGALFIGYCIYFDRKRRSDPNFGSGGSMVSKGEEDNMAIIKE |
| mCherry- | localized | FMRFKVHMEGSVNGHEFEIEGEGEGRPYEGTQTAKLKVTKGGPLPFAWDILSP |
| NS3a(H7) | NS3a/NS3a* | QFMYGSKAYVKHPADIPDYLKLSFPEGFKWERVMNFEDGGVVTVTQDSSLQD |
| | chimera H7- | GEFIYKVKLRGTNFPSDGPVMQKKTMGWEASSERMYPEDGALKGEIKQRLKL |
| | catalytically active, | KDGGHYDAEVKTTYKAKKPVQLPGAYNVNIKLDITSHNEDYTIVEQYERAEGR |
| | pcDNA5/FRT/TO | HSTGGMDELYKGSGSTGTSGSGSGTGSGSGTGMKKKGSVVIVGRINLSGDTA |
| | | YSQQTRGEEGCQETSQTGRDKNQVEGEVQVVSTATQSFLATSINGVLWTV |
| | | YHGAGTRTIASPKGPVTQMYTNVDKDLVGWQAPQGSRSLTPCTCGSSDLY |
| | | LVTRHADVIPVRRRGDSRGSLLSPRPISYLKGSSGGPLLCPAGHAVGIFRAA |
| | | VSTRGVAKAVDFIPVESLETTMRSPGSGTGSGTSGSTGTGSTGDYKDDDDK |
| | | (SEQ ID NO: 63) |
| EGFP- | EGFP-(ANR- | MVSKGEEDNMAIIKEFMRFKVHMEGSVNGHEFEIEGEGEGRPYEGTQTAKLKV |
| NS3a(H1) | binding-restored) | TKGGPLPFAWDILSPQFMYGSKAYVKHPADIPDYLKLSFPEGFKWERVMNFED |
| | NS3a/NS3a* | GGVVTVTQDSSLQDGEFIYKVKLRGTNFPSDGPVMQKKTMGWEASSERMYPE |
| | chimera H1- | DGALKGEIKQRLKLKDGGHYDAEVKTTYKAKKPVQLPGAYNVNIKLDITSHNE |
| | catalytically active | DYTIVEQYERAEGRHSTGGMDELYKGSGTGDYKDDDDKKKKGSVVIVGRIN |
| | pcDNA5/FRT/TO | LSGDTAYAQQTRGLEGCQETSQTGRDKNQVEGEVQIVSTATQTFLATSING |
| | | VLWTVYHGAGTRTIASPKGPVTQMYTNVDKDLVGWQAPQGSRSLTPCTC |
| | | GSSDLYLVTRHADVIPVRRRGDSRGSLLSPRPISYLKGSAGGPLLCPAGHAV |
| | | GIFRAAVSTRGVAKAVDFIPVESLETTMRSP (SEQ ID NO: 64) |
| EGFP-ANR₂ | EGFP-ANR-ANR | MVSKGEELFTGVVPILVELDGDVNGHKFSVSGEGEGDATYGKLTLKFICTTGK |
| | pcDNA5/FRT/TO | LPVPWPTLVTTLTYGVQCFSRYPDHMKQHDFFKSAMPEGYVQERTIFFKDDGN |
| | | YKTRAEVKFEGDTLVNRIELKGIDFKEDGNILGHKLEYNYNSHNVYIMADKQK |
| | | NGIKVNFKIRHNIEDGSVQLADHYQQNTPIGDGPVLLPDNHYLSTQSALSKDPN |
| | | EKRDHMVLLEFVTAAGITLGMDELYKSGSGEQKLISEEDLGSGTGSGTGSGTGT |
| | | TSGTGTGGSTGGELDELVYLLDGPGYDPIHSDGSGTGSGTGSGTGTTSGTGTGGS |
| | | TGGELDELVYLLDGPGYDPIHSD (SEQ ID NO: 65) |
| Myr- | Plasma membrane | MGCGCSSHPEDDGTGSGTGSMVSKGEEDNMAIIKEFMRFKVHMEGSVNGHEF |
| mCherry- | localized Myr-ANR- | EIEGEGEGRPYEGTQTAKLKVTKGGPLPFAWDILSPQFMYGSKAYVKHPADIPD |
| ANR₂ | ANR-mCherry | YLKLSFPEGFKWERVMNFEDGGVVTVTQDSSLQDGEFIYKVKLRGTNFPSDGP |
| | pcDNA5/FRT/TO | VMQKKTMGWEASSERMYPEDGALKGEIKQRLKLKDGGHYDAEVKTTYKAKK |
| | | PVQLPGAYNVNIKLDITSHNEDYTIVEQYERAEGRHSTGGMDELYKGSGSEQK |
| | | LISEEDLGSGTGSGTGSGTGTTSGTGTGGSTGGELDELVYLLDGPGYDPIHSDGS |
| | | GTGSGTGSGTGTTSGTGTGGSTGGELDELVYLLDGPGYDPIHSD |
| | | (SEQ ID NO: 66) |
| NLS₃-BFP- | Nuclear localized | MDPKKKRKVDPKKKRKVDPKKKRKVGSGSELIKENMHMKLYMEGTVDNHHF |
| ANR₂ | 3xNLS-BFP-ANR- | KCTSEGEGKPYEGTQTMRIKVVEGGPLPFAFDILATSFLYGSKTFINHTQGIPDFF |
| | ANR | KQSFPEGFTWERVTTYEDGGVLTATQDTSLQDGCLIYNVKIRGVNFTSNGPVM |
| | pcDNA5/FRT/TO | QKKTLGWEAFTETLYPADGGLEGRNDMALKLVGGSHLIANIKTTYRSKKPAK |
| | | NLKMPGVYYVDYRLERIKEANNETYVEQHEVAVARYCDLPSKLGHKLNSGSG |
| | | EQKLISEEDLGSGTGSGTGSGTGTTSGTGTGGSTGGELDELVYLLDGPGYDPIHS |
| | | DGSGTGSGTGSGTGTTSGTGTGGSTGGELDELVYLLDGPGYDPIHSD |
| | | (SEQ ID NO: 67) |
| NS3a-CDAR | 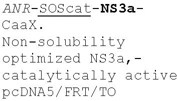 | 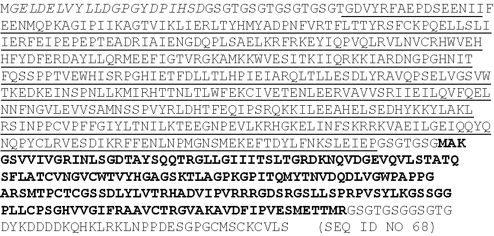 |
| BH3-NS3a- CDAR | 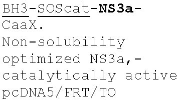 | 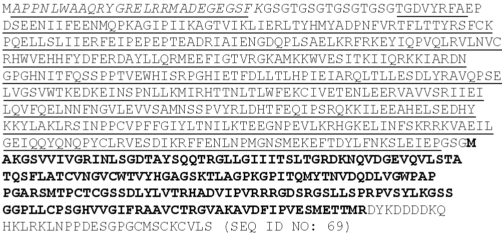 |
| dciCas9 | Catalytically | MDYKDDDDKDKKYSIGLAIGTNSVGWAVITDEYKVPSKKFKVLGNTDRHSIK |
| | inactive ciCas9 | KNLIGALLFDSGETAEATRLKRTARRRYTRRKNRICYLQEIFSNEMAKVDDSFF |
| | (D10A, H840A) | HRLEESFLVEEDKKHERHPIFGNIVDEVAYHEKYPTIYHLRKKLVDSTDKADLR |
| | pcDNA5/FRT/TO | LIYLALAHMIKFRGHFLIEGDLNPDNSSNRELVVDFLSYKLSQKGYSWSQFSDV |
| | | EENRTEAPEGTESEMETPSAINGNPSWHLADSPAVNGATGHSSSLDAREVIPMA |
| | | AVKQALREAGDEFELRYRRAFSDLTSQLHITPGTAYQSFEQVVNELFRDGVNW |
| | | GRIVAFFSFGGALCVESVDKEMQVLVSRIAAWMATYLNDHLEPWIQENGGWD |
| | | TFVELYGNNGSGTASGTGSGTGSATGSGTVNTEITKAPLSASMIKRYDEHHQDL |
| | | TLLKALVRQQLPEKYKEIFFDQSKNGYAGYIDGGASQEEFYKFIKPILEKMDGT |
| | | EELLVKLNREDLLRKQRTFDNGSIPHQIHLGELHAILRRQEDFYPFLKDNREKIE |
| | | KILTFRIPYYVGPLARGNSRFAWMTRKSEETITPWNFEEVVDKGASAQSFIERM |
| | | TNFDKNLPNEKVLPKHSLLYEYFTVYNELTKVKYVTEGMRKPAFLSGEQKKAI |
| | | VDLLFKTNRKVTVKQLKEDYFKKIECFDSVEISGVEDRFNASLGTYHDLLKIIK |
| | | DKDFLDNEENEDILEDIVLTLTLFEDREMIEERLKTYAHLFDDKVMKQLKRRRY |
| | | TGWGRLSRKLINGIRDKQSGKTILDFLKSDGFANRNFMQLIHDDSLTFKEDIQK |
| | | AQVSGQGDSLHEHIANLAGSPAIKKGILQTVKVVDELVKVMGRHKPENIVIEM |
| | | ARENQTTQKGQKNSRERMKRIEEGIKELGSQILKEHPVENTQLQNEKLYLYYL |
| | | QNGRDMYVDQELDINRLSDYDVDAIVPQSFLKDDSIDNKVLTRSDKNRGKSDN |
| | | VPSEEVVKKMKNYWRQLLNAKLITQRKFDNLTKAERGGLSELDKAGFIKRQL |
| | | VETRQITKHVAQILDSRMNTKYDENDKLIREVKVITLKSKLVSDFRKDFQFYKV |
| | | REINNYHHAHDAYLNAVVGTALIKKYPKLESEFVYGDYKVYDVRKMIAKSEQ |
| | | EIGKATAKYFFYSNIMNFFKTEITLANGEIRKRPLIETNGETGEIVWDKGRDFAT |
| | | VRKVLSMPQVNIVKKTEVQTGGFSKESILPKRNSDKLIARKKDWDPKKYGGFD |
| | | SPTVAYSVLVVAKVEKGKSKKLKSVKELLGITMERSSFEKNPIDFLEAKGYKE |
| | | VKKDLIIKLPKYSLFELENGRKRMLASAGELQKGNELALPSKYVNFLYLASHYE |
| | | KLKGSPEDNEQKQLFVEQHKHYLDEIIEQISEFSKRVILADANLDKVLSAYNKH |
| | | RDKPIREQAENIIHLFTLTNLGAPAAFKYFDTTIDRKRYTSTKEVLDATLIHQSIT |
| | | GLYETRIDLSQLGGDSRADPKKKRKVTGSGTAPPNLWAAQRYGRELRRMADE |
| | | GEGSFK (SEQ ID NO: 70) |
| NS3a(H1)- VPR | (ANR-binding- restored) NS3a/NS3a* chimera H1-VPR- catalytically active, pcDNA5/FRT/TO | 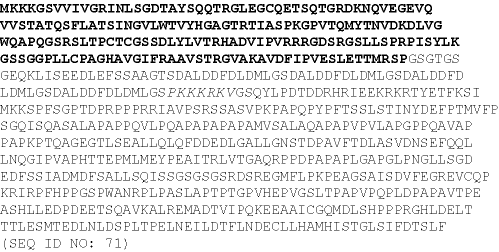 |
| NLS-MCP- ANR₂ | Nuclear localized MCP-ANR-ANR- P2A-BFP Expressed with TRE3G-2xMS2 | 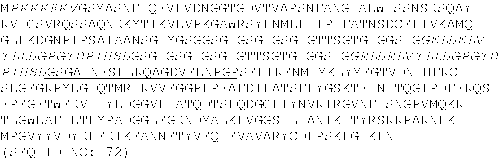 |
| Gal4(DBD)- | Gal4-(ANR-binding | MKLLSSIEQACDICRLKKLKCSKEKPKCAKCLKNNWECRYSPKTKRSPLTRAH |
| NS3a(H1)- | restored) | LTEVESRLERLEQLFLLIFPREDLDMILKMDSLQDIKALLGTPAAASTAGSGGM |
| P2A-ANR- | NS3a/NS3a* | AKGSVVIVGRINLSGDTAYSQQTRGLEGCQETSQTGRDKNQVEGEVQVVS |
| BFP-VPR- | chimera H1-P2A- | TATQSFLATSINGVLWTVYHGAGTRTIASPKGPVTQMYTNVDKDLVGWQ |
| NLS | ANR-Myc-BFP- | APQGSRSLTPCTCGSSDLYLVTRHADVIPVRRRGDSRGSLLSPRPISYLKGS |
| | VPR (internal NLS)- | AGGPLLCPAGHAVGIFRAAVSTRGVAKAVDFIPVESLETTMRSPGSGATNFS |
| | catalytically active | LLKQAGDVEENPGPGALSGMGELDELVYLLDGPGYDPIHSDGVLSGSGTGSGTG |
| | pcDNA5/FRT/TO | SGTGTTSGTGTGGSTGEQKLISEEDLGSGSSELIKENMHMKLYMEGTVDNHHF |
| | | KCTSEGEGKPYEGTQTMRIKVVEGGPLPFAFDILATSFLYGSKTFINHTQGIPDFF |
| | | KQSFPEGFTWERVTTYEDGGVLTATQDTSLQDGCLIYNVKIRGVNFTSNGPVM |
| | | QKKTLGWEAFTETLYPADGGLEGRNDMALKLVGGSHLIANIKTTYRSKKPAK |
| | | NLKMPGVYYVDYRLERIKEANNETYVEQHEVAVARYCDLPSKLGHKLNGSGS |
| | | DALDDFDLDMLGSDALDDFDLDMLGSDALDDFDLDMLGSDALDDFDLDMLG |
| | | SPKKKRKVGSQYLPDTDDRHRIEEKRKRTYETFKSIMKKSPFSGPTDPRPPPRRI |
| | | AVPSRSSASVPKPAPQPYPFTSSLSTINYDEFPTMVFPSGQISQASALAPAPPQVL |
| | | PQAPAPAPAPAMVSALAQAPAPVPVLAPGPPQAVAPPAPKPTQAGEGTLSEALL |
| | | QLQFDDEDLGALLGNSTDPAVFTDLASVDNSEFQQLLNQGIPVAPHTTEPMLM |
| | | EYPEAITRLVTGAQRPPDPAPAPLGAPGLPNGLLSGDEDFSSIADMDFSALLSQIS |
| | | SGSGSGSRDSREGMFLPKPEAGSAISDVFEGREVCQPKRIRPFHPPGSPWANRPL |
| | | PASLAPTPTGPVHEPVGSLTPAPVPQPLDPAPAVTPEASHLLEDPDEETSQAVKA |
| | | LREMADTVIPQKEEAAICGQMDLSHPPPRGHLDELTTTLESMTEDLNLDSPLTP |
| | | ELNEILDTFLNDECLLHAMHISTGLSIFDTSLF (SEQ ID NO: 73) |
| Gal4(DBD)- | Gal4-NS3a*-P2A- | MKLLSSIEQACDICRLKKLKCSKEKPKCAKCLKNNWECRYSPKTKRSPLTRAH |
| NS3a(H1)- | DNCR2-VPR | LTEVESRLERLEQLFLLIFPREDLDMILKMDSLQDIKALLGTPAAASTLEGGGSA |
| P2A-DNCR2- | (internal NLS)- | GSGGKKKGSVVIVGRINLSGDTAYAQQTRGEEGCQETSQTGRDKNQVEGE |
| VPR-NLS | catalytically | VQIVSTATQTFLATSINGVLWTVYHGAGTRTIASPKGPVTQMYTNVDKDL |
| | inactive | VGWQAPQGSRSLTPCTCGSSDLYLVTRHADVIPVRRRGDSRGSLLSPRPISY |
| | pcDNA5/FRT/TO | LKGSAGGPLLCPAGHAVGIFRAAVSTRGVAKAVDFIPVESLETTMRSPGSG |
| | | ATNFSLLKQAGDVEENPGPMSSDEEEARELIERAKEAAERAQEAAERTGDPRVREL |
| | | ARELKRLAQEAAEEVKRDPSSSDVNEALKLIVEAIEAAVDALEAAERTGDPEVRELAR |
| | | ELVRLAVEAAEEVQRNPSSSDVNEALHSIVYAIEAAIFALEAAERTGDPEVRELARELV |
| | | RLAVEAAEEVQRNPSSRNVEHALMRIVLAIYLAEENLREAEESGDPEKREKARERVRE |
| | | AVERAEEVQRDPSGWLNHEQKLISEEDLDALDDFDLDMLGSDALDDFDLDMLG |
| | | SDALDDFDLDMLGSDALDDFDLDMLGSPKKKRKVGSQYLPDTDDRHRIEEKR |
| | | KRTYETFKSIMKKSPFSGPTDPRPPPRRIAVPSRSSASVPKPAPQPYPFTSSLSTIN |
| | | YDEFPTMVFPSGQISQASALAPAPPQVLPQAPAPAPAPAMVSALAQAPAPVPVL |
| | | APGPPQAVAPPAPKPTQAGEGTLSEALLQLQFDDEDLGALLGNSTDPAVFTDLA |
| | | SVDNSEFQQLLNQGIPVAPHTTEPMLMEYPEAITRLVTGAQRPPDPAPAPLGAP |
| | | GLPNGLLSGDEDFSSIADMDFSALLSQISSGSGSGSRDSREGMFLPKPEAGSAISD |
| | | VFEGREVCQPKRIRPFHPPGSPWANRPLPASLAPTPTGPVHEPVGSLTPAPVPQP |
| | | LDPAPAVTPEASHLLEDPDEETSQAVKALREMADTVIPQKEEAAICGQMDLSHP |
| | | PPRGHLDELTTTLESMTEDLNLDSPLTPELNEILDTFLNDECLLHAMHISTGLSIF |
| | | DTSLF (SEQ ID NO: 74) |
| BFP | BFP reporter | MSELIKENMHMKLYMEGTVDNHHFKCTSEGEGKPYEGTQTMRIKVVEGGPLP |
| | pcDNA5/FRT/TO | FAFDILATSFLYGSKTFINHTQGIPDFFKQSFPEGFTWERVTTYEDGGVLTATQD |
| | | TSLQDGCLIYNVKIRGVNFTSNGPVMQKKTLGWEAFTETLYPADGGLEGRND |
| | | MALKLVGGSHLIANIKTTYRSKKPAKNLKMPGVYYVDYRLERIKEANNETYVE |
| | | QHEVAVARYCDLPSKLGHKLN (SEQ ID NO: 75) |
| TRE3G- | scRNA, wt + f6 M52 | GTACGTTCTCTATCACTGATAGTTTAAGAGCTATGCTGGAAACAGCATAG |
| 2xMS2 | expressed with | CAAGTTTAAATAAGGCTAGTCCGTTATCAACTTGAAAAAGTGGCACCGAGT |
| | NLS-MCP-ANR- | CGGTGCGGGAGCACATGAGGATCACCCATGTGCGACTCCCACAGTCACTGG |
| | ANR-P2A-BFP | GGAGTCTTCCCTTTTTTTGTTTTTTATGTCT (SEQ ID NO: 76) |
| TRE3G | TRE3G guide in | GTACGTTCTCTATCACTGATA (SEQ ID NO: 77) |
| | gRNA_Cloning | |
| | Vector | |

### Example 2

Methods for post-translational, dynamic control over intracellular protein function are valuable tools for studying naturally-occurring biological systems and for engineering synthetic systems. Existing chemical and optogenetic systems for controlling protein function are largely restricted to providing single-input/single-output control schemes. To address this, we have created a system using the hepatitis C virus protease NS3a as a single receiver protein that binds multiple drug inputs and is recognized by a set of reader proteins to produce divergent outputs. The keys to the development of this multi-input/multi-output system, called Pleiotropic Response Outputs from a Chemically-Inducible Single Receiver (PROCISiR), are computationally designed reader proteins that can discriminate between different NS3a-drug complexes. The unique, responsive architecture of PROCISiR enables proportional and temporal control modes that are unobtainable with current systems. In signaling or transcriptional applications, we demonstrate output reversibility, switching, tunability, ratiometric control, and fine specification of intermediate levels of two outputs. Given the availability of multiple NS3a-targeting drugs and our ability to create protein readers of specific drug-bound NS3a complexes, PROCISiR can be scaled to provide unprecedented multi-state control over intracellular protein function. These complex control modalities can be readily applied to both in vitro studies of mammalian cellular processes and in vivo signaling and transcriptional control programs for engineered cell therapies.

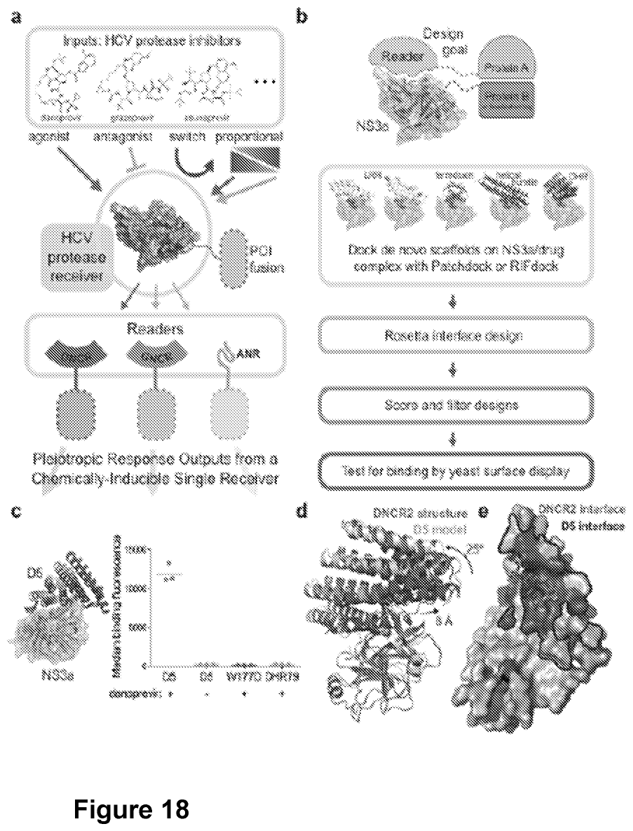

Mammalian cells are complex information processing systems that receive and transmit many signals through interconnected signaling networks to produce diverse arrays of responses. Multi-functional proteins, such as receptor tyrosine kinases and GPCRs, that can receive multiple inputs and provide variable outputs are central components of these networks, allowing flexible and complex control over cellular behavior. We identified HCV protease NS3a as an attractive central receiver protein that can serve as a control hub for a chemically-controlled multi-input/multi-output system called PROCISiR (FIG. 18a). NS3a has previously been integrated into engineered eukaryotic systems, and numerous drugs of varying geometries and affinities are available as inputs that are functionally silent in mammalian cells and well-tolerated in vivo. Furthermore, a genetically-encoded peptide inhibitor of NS3a, here called apo NS3a reader (ANR), serves as a “reader” of the apo NS3a state, forming a basal complex that is disruptable by small molecule NS3a inhibitors. We hypothesized that computational protein interface design could be used to generate protein “readers” capable of discriminating between NS3a's apo or inhibitor-bound states. The availability of numerous chemical inputs and ability to rationally engineer protein readers that discriminate between different NS3a drug-bound states provides a platform for generating diverse functional outputs emanating from a single receiver protein.

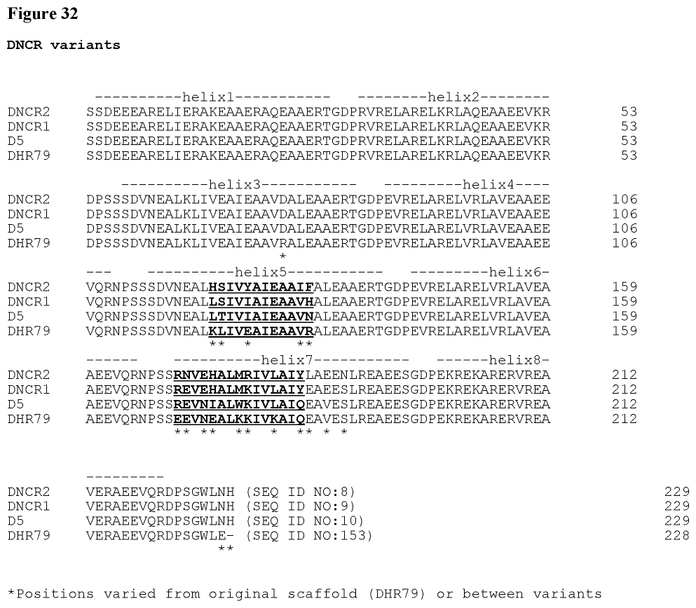

Rosetta™ interface design allowed us to develop protein readers that selectively recognize a binding surface centered on NS3a-bound inhibitors (FIG. 18b). First, we used a set of stable, de novo-designed proteins as scaffolds on which to design an interface with the danoprevir/NS3a complex. As a starting point, PatchDock™ was used to center each scaffold over danoprevir, followed by RosettaDesign™ on the scaffold surface that forms the binding interface.¹⁵ A design D5, one of 31 designs selected for testing via yeast surface display, showed modest, drug-dependent binding to NS3a (FIG. 18c). Validating our design, the parent designed helical repeat scaffold (DHR79) and D5 containing a predicted interface-disrupting mutation demonstrated undetectable binding to the NS3a/danoprevir complex (FIG. 18c). See FIG. 32 for exemplary alignments verses DHR79.

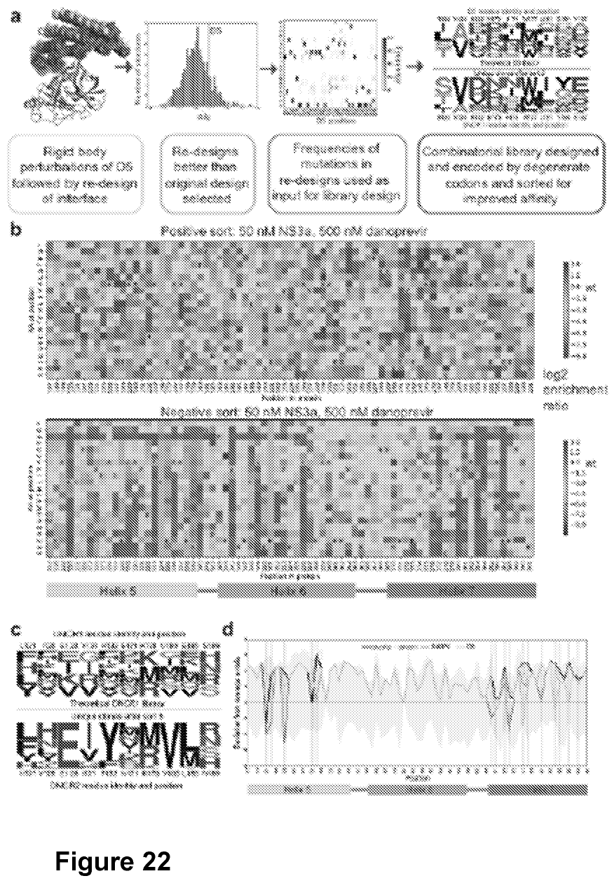

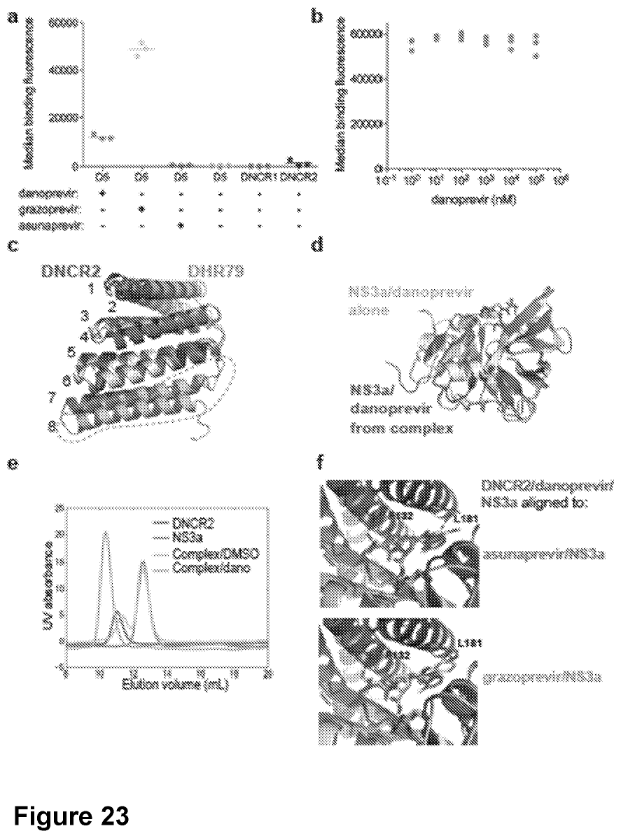

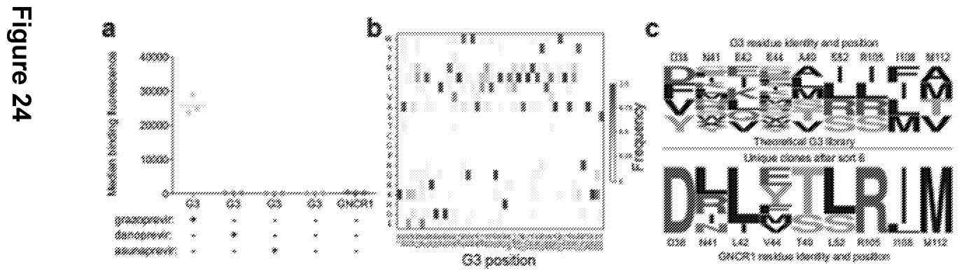

To improve D5's affinity for the NS3a/danoprevir complex, we used two sequential yeast surface display libraries (FIG. 22, Supplementary Note 1). Our final variant, DNCR2, with 14 mutations from D5, had an apparent affinity for the NS3a/danoprevir complex of 36 μM, no detectable binding to apo NS3a, and >20,000-fold specificity over NS3a bound to the drugs grazoprevir or asunaprevir (Extended Data Table 1, FIG. 23a). Further biochemical analysis confirmed that DNCR2 does not bind substantially to free danoprevir and that DNCR2/danoprevir/NS3a form a 1:1:1 complex (Supplementary Note 1, FIG. 23b,e). A 2.3 A resolution structure of the DNCR2/danoprevir/NS3a complex revealed a modest shift for DNCR2 relative to the D5 model with the interface formed via a conserved region of the DHR surface (FIG. 18d,e). The structural basis for the selective binding of DNCR2 to the NS3a/danoprevir complex, namely, clashes and non-ideal packing between DNCR2 and the small molecules, is clearly apparent when structures of asunaprevir- or grazoprevir-bound NS3a are aligned to the DNCR2/danoprevir/NS3a complex (FIG. 23f).

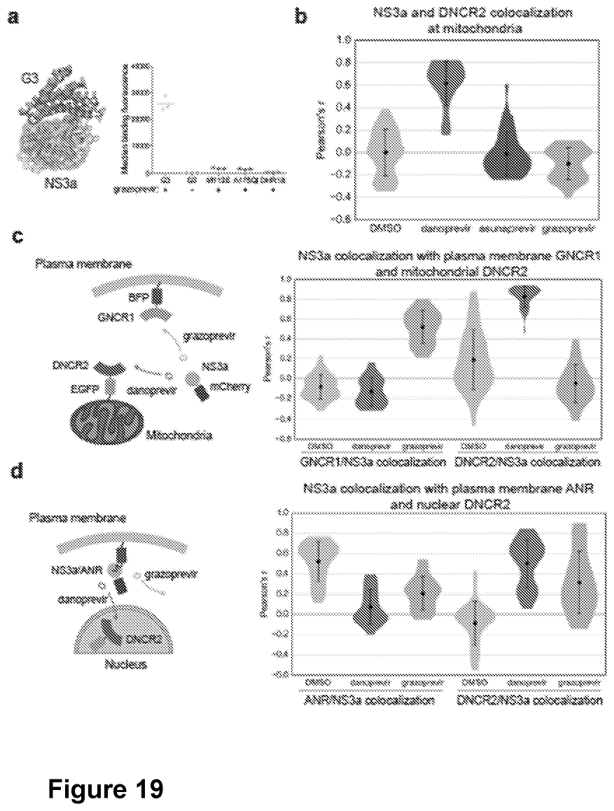

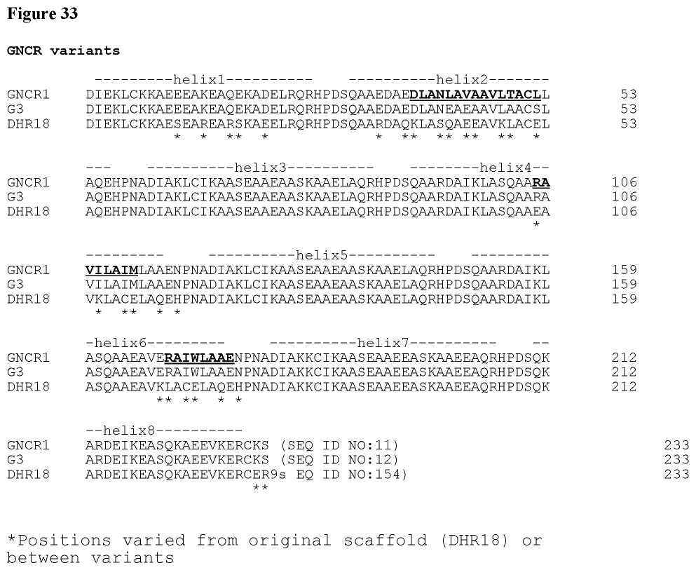

The high specificity of DNCR2 provided confidence that we could design additional readers that selectively recognize other NS3a/drug complexes. We computationally designed a reader of the grazoprevir/NS3a complex by applying a similar methodology. One design of the 29 tested, G3, showed modest, grazoprevir-dependent binding, which was not observed for the original scaffold, DHR18, or for G3 variants containing interface mutations (M112E and A175Q) (FIG. 19a). Screening a single library for improved affinity yielded grazoprevir/NS3a complex reader 1 (GNCR1) containing 4 mutations from G3. GNCR1 had an apparent affinity for the grazoprevir/NS3a complex of 140 nM and little-to-no affinity for apo, danoprevir-, or asunaprevir-bound NS3a (FIG. 24, Extended Data Table 1, and Supplementary Note 1). See FIG. 33 for alignments of exemplary variants of DHR18.

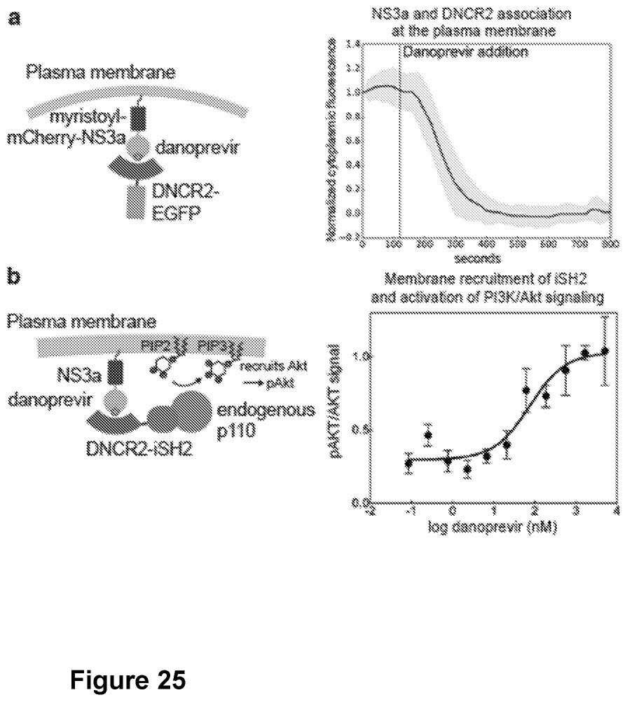

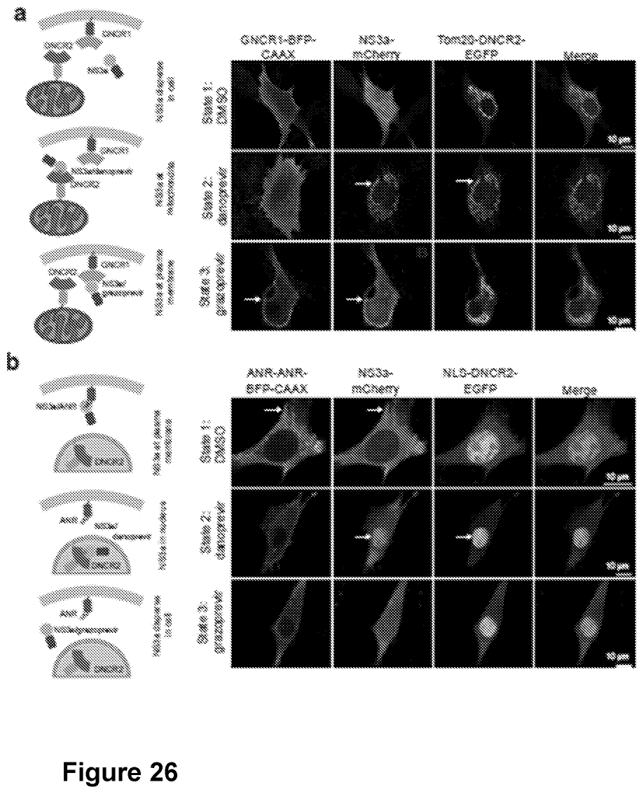

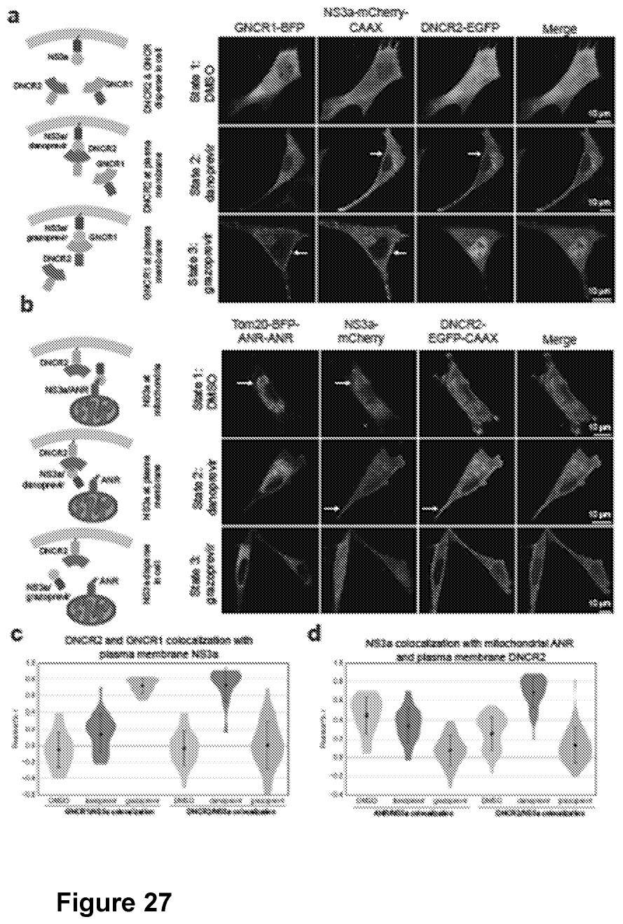

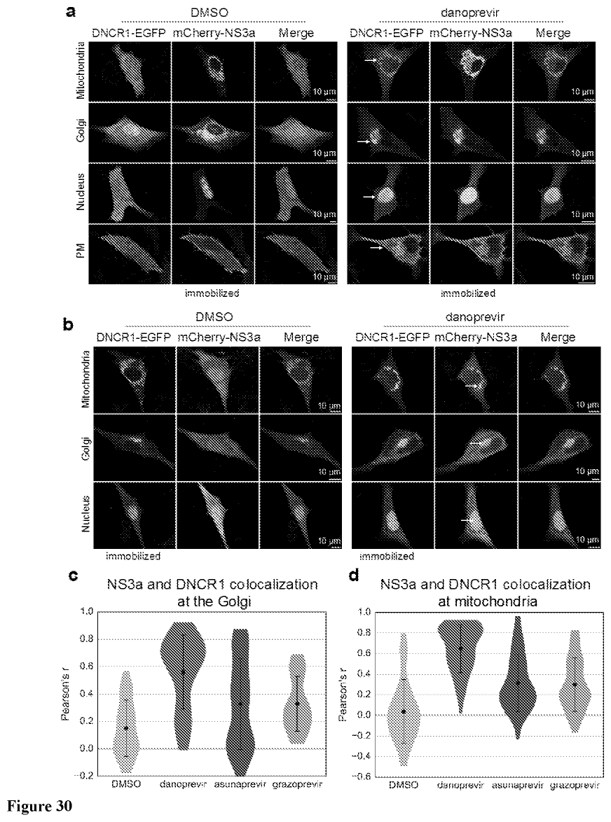

With our two drug/NS3a complex readers, DNCR2 and GNCR1, and the apo-NS3a reader (ANR), we now had three readers to combine with NS3a in our PROCISiR system (FIG. 18a). First, we verified the function of DNCR2 in mammalian cells using colocalization experiments, in which we demonstrated that DNCR2 rapidly colocalized with plasma membrane-localized NS3a after danoprevir addition (t_{1/2} of 76±27 sec (mean, standard deviation)) and that this membrane localization was capable of activating PI3K-Akt signaling when DNCR2 was fused to the inter-SH2 domain from the p85 regulatory subunit of PI3K (FIG. 25). The drug specificity of DNCR2 was maintained in cells, as neither grazoprevir nor asunaprevir induced DNCR2-EGFP colocalization with mitochondrial-localized Tom20-mCherry™-NS3a (FIG. 19b). We then combined DNCR2 with GNCR1 or ANR to control the localization of mCherry™-NS3a to two different subcellular locations. We observed that grazoprevir exclusively colocalized NS3a-mCherry™ to plasma membrane-targeted GNCR1-BFP-CAAX while only danoprevir led to colocalization with mitochondria-targeted Tom20-DNCR2-EGFP (FIG. 19c, FIG. 26a). Likewise, ANR—BFP-CAAX pre-localized NS3a-mCherry™ to the plasma membrane, while danoprevir treatment recruited NS3a to the nucleus with NLS-DNCR2-EGFP (FIG. 19d, FIG. 26b). These and additional colocalization experiments (Supplementary Note 2, FIG. 30, FIG. 27) validated that the three readers DNCR2, GNCR1, and ANR were selective for their targeted state of NS3a and could be used in concert.

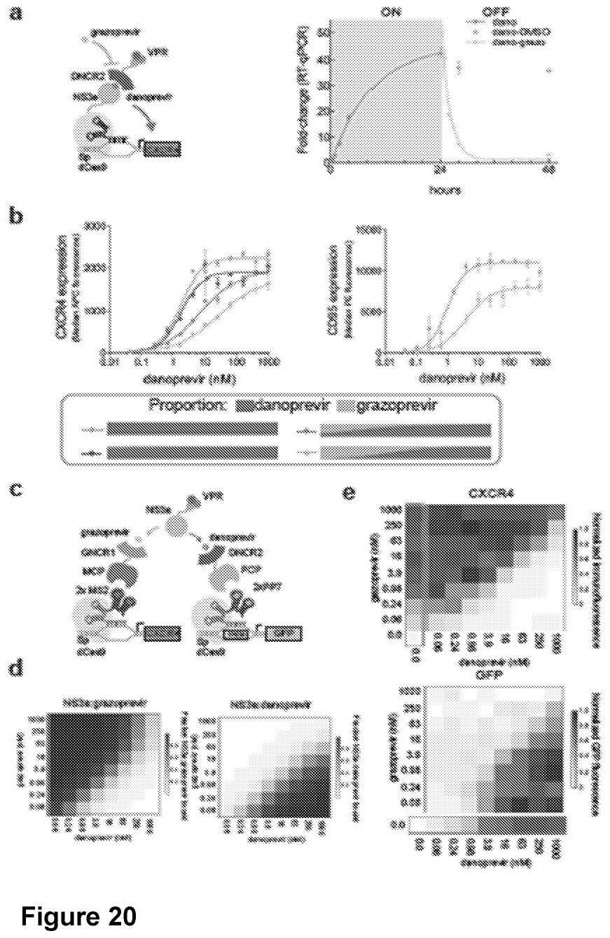

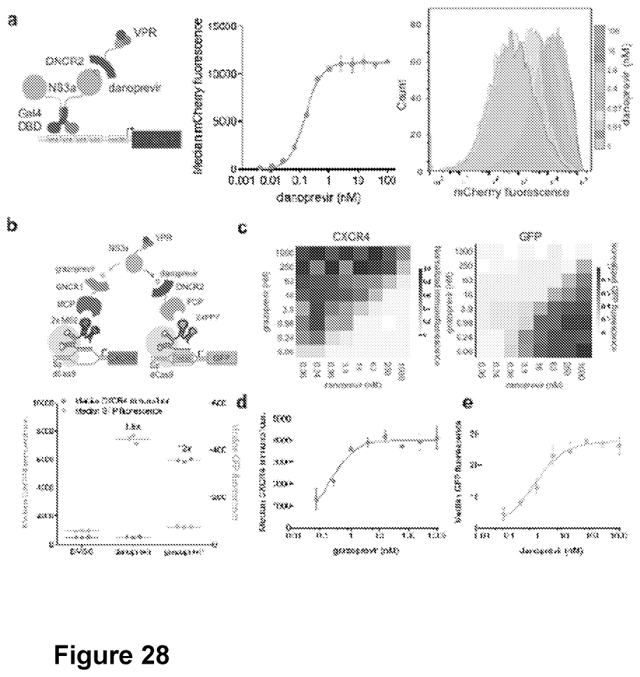

The ability of our readers to discriminate between different states of NS3a allows complex control modes to be achieved by combining inputs and/or readers, a capability not shared by chemically inducible systems for which there is only one input and one protein complex. First, we used danoprevir as an agonist and grazoprevir as an antagonist to temporally and proportionally control transcription of one endogenous gene using DNCR2-VPR (a transcriptional activator) and an NS3a-dCas9 fusion (Streptococcus pyogenes). We used danoprevir to induce transcriptional activation of CXCR4 from its endogenous promoter, and then rapidly reversed CXCR4 expression by using grazoprevir as a competitive chaser (mRNA reversion t_{1/2} of 1.3 hours) (FIG. 20a). Next, we co-treated cells with varying danoprevir/grazoprevir ratios to precisely tune the concentration of DNCR2-binding competent NS3a (FIG. 20b). Increasing the proportion of grazoprevir added to a constant titration of danoprevir yielded more graded CXCR4 expression, stretching the dose-response curve to produce a linear output for 3 orders of magnitude of danoprevir input. This ability to finely titrate gene expression up from endogenous levels was validated on the endogenous promoter for a second gene, CD95 (FIG. 20b). The combination of inducer and competitor inputs allows precise tuning of gene expression on a single cell-level at inducer concentration ranges outside of the narrow linear response range of a bimolecular binding curve. We also demonstrated the ability to titrate gene expression on the single-cell level from an exogenous promoter, using DNCR2/danoprevir/NS3a to complex the Gal4 DNA-binding domain with VPR (FIG. 28a). Commonly used mammalian gene induction systems, such as the doxycycline-induced TetR, have poor ability to achieve intermediate levels of gene expression.

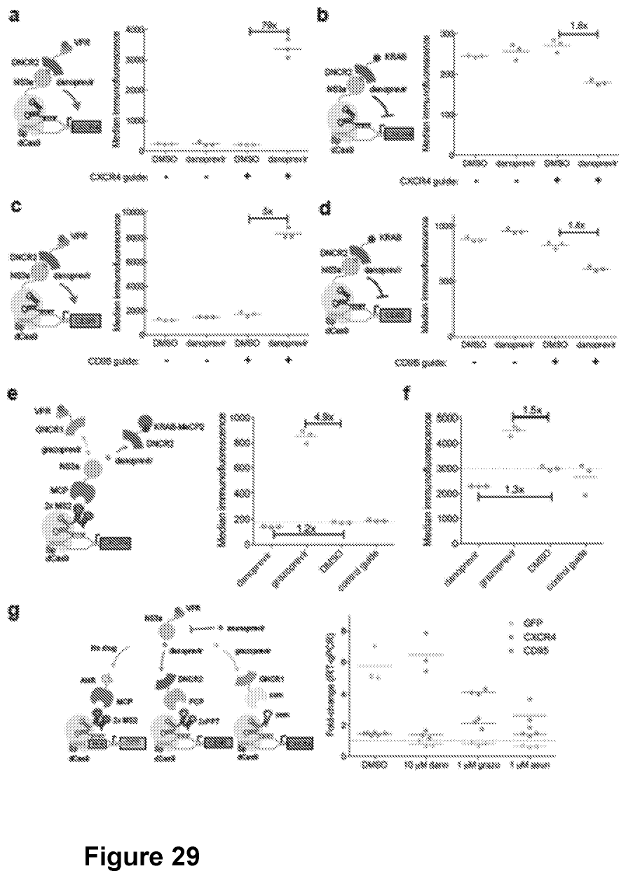

We then applied our PROCISiR method to provide orthogonal control of multi-gene transcription using dCas9 with scaffold RNAs (scRNAs) that contain loci-targeting, single guide RNAs and embedded stem loops recognized by RNA-binding proteins (RBPs). Using an MS2 scRNA targeting endogenous CXCR4 and a PP7 scRNA targeting the Tet operator of a GFP reporter, together with GNCR1-MCP and DNCR2-PCP RBP fusions, respectively, we directed NS3a-VPR to orthogonally induce transcription of each gene (FIG. 20c, FIG. 28b). Titration of each drug alone in this system demonstrated the high affinity of each reader with EC₅₀s of 0.16±0.03 nM and 0.79+0.15 nM (mean±standard deviation) for the grazoprevir/NS3a and danoprevir/NS3a readers, respectively, in close agreement with NS3a's Kᵢ value for each drug (FIG. 28d,e). This dependence of transcriptional output from each reader on their inducer's Kᵢ value allowed us to model the output from each reader/drug/NS3a complex in the presence of a range of mixed danoprevir and grazoprevir concentrations (FIG. 20d, Supplemental Note 3). Ratiometric expression output of CXCR4 and GFP across a matrix of danoprevir and grazoprevir concentrations demonstrated close concordance with the predicted NS3a: drug complexes (FIG. 20e, FIG. 28c). See Supplementary Note 4 FIG. 29 for a description of other transcriptional control modes demonstrated, including 3-gene control and switchable repression/overexpression. The responsive nature of the PROCISiR architecture enables diverse modes of temporal, proportional, and multi-state transcriptional control.

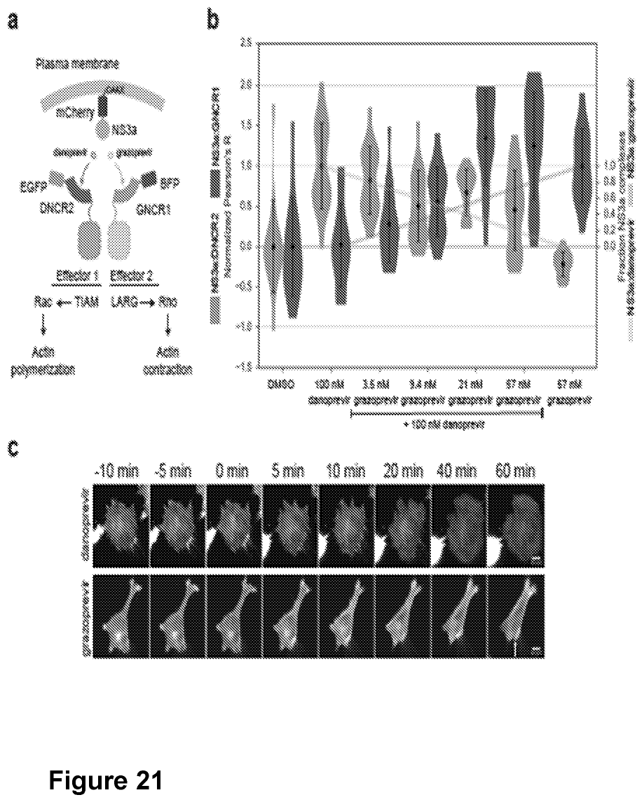

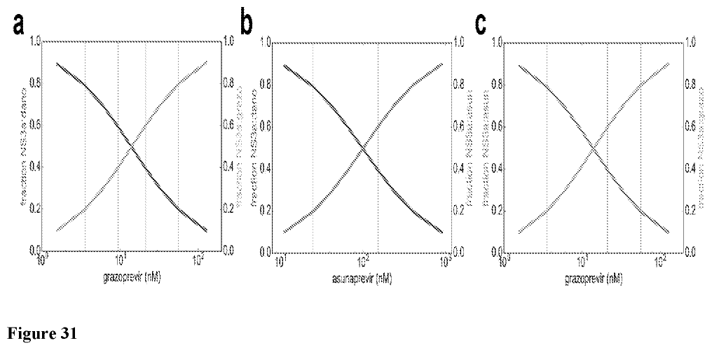

Finally, we applied PROCISiR to directly control the relative activation of two signaling pathways through localization of DNCR2 and GNCR1 to the plasma membrane via NS3a-CAAX (FIG. 21a). Modeling of drug concentrations predicted to yield a range of NS3a:danoprevir and NS3a:grazoprevir complexes showed relatively good concordance with a semi-quantitative fluorescent protein colocalization dataset; therefore, we went on to use these concentration regimes to control localization of pairs of signaling effectors at the plasma membrane (FIG. 21b Supplementary Note 3 and FIG. 31). The first combination of signaling effector domains we used were EGFP-DNCR2-TIAM (Rac GEF) and BFP-GNCR1-LARG (Rho GEF). When these constructs and NS3a-CAAX were transfected in HeLa cells, danoprevir treatment caused cell expansion, and grazoprevir treatment caused cell contraction (FIG. 21c). Thus, switching between treatment with danoprevir and grazoprevir can be used to switch between cell signaling pathways, allowing temporal and proportional control of signaling pathways.

Here, we present two new readers with de novo designed interfaces that selectively recognize highly similar protein-small molecule complexes. The ability to discriminate between such closely-related binding surfaces highlights the power of computational protein design and suggests that it will be possible to exploit the wealth of additional NS3a inhibitors available to rapidly expand the number of protein readers, and subsequent outputs, available for the PROCISiR system. Furthermore, a similar strategy can be applied to alternative protein-small molecule complexes. Our designed readers have several characteristics that will make them useful replacements for the existing chemically induced dimerizers, in particular, the high potency, reversibility, favorable pharmacokinetics, and bio-orthogonal nature of the NS3a inhibitors. These characteristics are in demand for in vivo applications such as drug-based control of cellular therapeutics.

The architecture of the PROCISiR system with its multiple inputs, three readers, and single receiver protein enables many unique, fine-scale modulations for in vitro mammalian cell biology. Use of PROCISiR as a post-translational controller allows simulation of a wide range of signaling and transcription states in a quantitative and targeted manner. Our ability to use a combination of inputs and readers to finely modulate gene expression allows temporal induction of the small-scale changes of gene expression observed during development and cancer progression, a capability not matched by the binary, and often non-physiological levels achievable with existing gene induction systems. We extended this fine proportional control of two outputs to concurrently modulate the levels of activity of two signaling pathways, demonstrating the ability to tune levels of individual pathway activity and their crosstalk. Because the danoprevir/grazoprevir ratios are manifested in the fractions of total NS3a bound to each drug, these proportional response regimes are not limited to the narrow drug concentrations of a bimolecular binding interaction, as they are for individual chemically induced dimerizers. The integrated nature of our system enables these more nuanced input-output response structures, which allows researchers to simulate and study the subtle perturbations to signaling and transcription that occur between normal and diseased cell states.

### REFERENCES

1. Ross, B., Mehta, S. & Zhang, J. Molecular tools for acute spatiotemporal manipulation of signal transduction. Curr Opin Chem Biol 34, 135-142 (2016).
2. Spencer, D. M., Wandless, T. J., Schreiber, S. L. & Crabtree, G. R. Controlling signal transduction with synthetic ligands. Science 262, 1019-1024 (1993).
3. Miyamoto, T. et al. Rapid and orthogonal logic gating with a gibberellin-induced dimerization system. Nat Chem Biol 8, 465-470 (2012).
4. Guntas, G. et al. Engineering an improved light-induced dimer (iLID) for controlling the localization and activity of signaling proteins. Proc Natl Acad Sci USA 112, 112-117 (2015).
5. Toettcher, J. E., Gong, D., Lim, W. A. & Weiner, O. D. Light-based feedback for controlling intracellular signaling dynamics. Nat Methods 8, 837-839 (2011).
6. Lemmon, M. A. & Schlessinger, J. Cell Signaling by Receptor Tyrosine Kinases. Cell 141, 1117-1134 (2010).
7. Grünberg, R. & Serrano, L. Strategies for protein synthetic biology. Nucleic Acids Res 38, 2663-2675 (2010).
8. De Luca, A., Bianco, C. & Rossetti, B. Treatment of HCV infection with the novel NS3/4A protease inhibitors. Curr Opin Pharmacol 18, 9-17 (2014).
9. Lin, M. Z., Glenn, J. S. & Tsien, R. Y. A drug-controllable tag for visualizing newly synthesized proteins in cells and whole animals. Proc Natl Acad Sci USA 105, 7744-7749 (2008).
10. Kügler, J. et al. High affinity peptide inhibitors of the hepatitis C virus NS3-4A protease refractory to common resistant mutants. J Biol Chem 287, 39224-39232 (2012).
11. Fleishman, S. J. et al. RosettaScripts: a scripting language interface to the Rosetta macromolecular modeling suite. PLoS ONE 6, e20161 (2011).
12. Park, K. et al. Control of repeat-protein curvature by computational protein design. Nat Struct Mol Biol 22, 167-174 (2015).
13. Brunette, T. J. et al. Exploring the repeat protein universe through computational protein design. Nature 528, 580-584 (2015).
14. King, I. C. et al. Precise assembly of complex beta sheet topologies from de novo designed building blocks. Elife 4, e11012 (2015).
15. Schneidman-Duhovny, D., Inbar, Y., Nussinov, R. & Wolfson, H. J. PatchDock and SymmDock: servers for rigid and symmetric docking. Nucleic Acids Res 33, 7 (2005).
16. Romano, K. P. et al. The molecular basis of drug resistance against hepatitis C virus NS3/4A protease inhibitors. PLoS Pathog 8, e1002832 (2012).
17. Soumana, D. I., Ali, A. & Schiffer, C. A. Structural analysis of asunaprevir resistance in HCV NS3/4A protease. ACS Chem Biol 9, 2485-2490 (2014).
18. Suh, B.-C., Inoue, T., Meyer, T. & Hille, B. Rapid Chemically Induced Changes of PtdIns(4,5)P2 Gate KCNQ Ion Channels. Science 314, 1454-1457 (2006).
19. Loew, R., Heinz, N., Hampf, M., Bujard, H. & Gossen, M. Improved Tet-responsive promoters with minimized background expression. BMC Biotechnol. 10, (2010).
20. Zalatan, J. G. et al. Engineering complex synthetic transcriptional programs with CRISPR RNA scaffolds. Cell 160, 339-350 (2015).
21. Qi, L. S. et al. Repurposing CRISPR as an RNA-guided platform for sequence-specific control of gene expression. Cell 152, 1173-1183 (2013).
22. Chavez, A. et al. Highly efficient Cas9-mediated transcriptional programming. Nat Methods 12, 326-328 (2015).
23. Hill, Z. B., Martinko, A. J., Nguyen, D. P. & Wells, J. A. Human antibody-based chemically induced dimerizers for cell therapeutic applications. Nat Chem Biol (2017). doi:10.1038/nchembio.2529
24. Wu, C.-Y., Roybal, K. T., Puchner, E. M., Onuffer, J. & Lim, W. A. Remote control of therapeutic T cells through a small molecule-gated chimeric receptor. Science 350, aab4077-aab4077 (2015).
25. Jacobs, C. L., Badiee, R. K. & Lin, M. Z. StaPLs: versatile genetically encoded modules for engineering drug-inducible proteins. Nat Methods 15, 523-526 (2018).
26. Tague, E. P., Dotson, H. L., Tunney, S. N., Sloas, D. C. & Ngo, J. T. Chemogenetic control of gene expression and cell signaling with antiviral drugs. Nat Methods 15, 519-522 (2018).
27. O'Boyle, N. M. et al. Open Babel: An open chemical toolbox. J Cheminform 3, 33 (2011).
28. Wittekind, M., Weinheimer, S. & Zhang, Y. Modified forms of hepatitis C NS3 protease for facilitating inhibitor screening and structural studies of protease:inhibitor complexes. US Patent (2004).
29. Tsao, K.-L., Debarbieri, B., Michel, H. & Waugh, D. S. A versatile plasmid expression vector for the production of biotinylated proteins by site-specific, enzymatic modification in Escherichia coli. Gene 169, 59-64 (1996).
30. Fleishman, S. J. et al. Computational design of proteins targeting the conserved stem region of influenza hemagglutinin. Science 332, 816-821 (2011).
31. Otwinowski, Z. & Minor, W. [20] Processing of X-ray diffraction data collected in oscillation mode. Meth Enzymol 276, 307-326 (1997).
32. Romano, K. P., Ali, A., Royer, W. E. & Schiffer, C. A. Drug resistance against HCV NS3/4A inhibitors is defined by the balance of substrate recognition versus inhibitor binding. Proc Natl Acad Sci USA 107, 20986-20991 (2010).
33. Emsley, P., Lohkamp, B., Scott, W. G. & Cowtan, K. Features and development of Coot. Acta Crystallogr D Biol Crystallogr 66, 486-501 (2010).
34. Collaborative Computational Project, Number 4. The CCP4 suite: programs for protein crystallography. Acta Crystallogr D Biol Crystallogr 50, 760-763 (1994).
35. Chen, T. S., Palacios, H. & Keating, A. E. Structure-based redesign of the binding specificity of anti-apoptotic Bcl-x(L). Journal of Molecular Biology 425, 171-185 (2013).
36. Dutta, S., Chen, T. S. & Keating, A. E. Peptide ligands for pro-survival protein Bfl-1 from computationally guided library screening. ACS Chem Biol 8, 778-788 (2013).
37. Foight, G. W., Chen, T. S., Richman, D. & Keating, A. E. Enriching Peptide Libraries for Binding Affinity and Specificity Through Computationally Directed Library Design. Methods Mol Biol 1561, 213-232 (2017).
38. Procko, E. et al. Computational design of a protein-based enzyme inhibitor. Journal of Molecular Biology 425, 3563-3575 (2013).
39. Berger, S. et al. Computationally designed high specificity inhibitors delineate the roles of BCL2 family proteins in cancer. Elife 5, e20352 (2016).
40. Fowler, D. M., Araya, C. L., Gerard, W. & Fields, S. Enrich: software for analysis of protein function by enrichment and depletion of variants. Bioinformatics 27, 3430-3431 (2011).
41. Costes, S. V. et al. Automatic and Quantitative Measurement of Protein-Protein Colocalization in Live Cells. Biophys J 86, 3993-4003 (2004).
42. Gao, Y. et al. Complex transcriptional modulation with orthogonal and inducible dCas9 regulators. Nat Methods 13, 1043 EP 1049 (2016).
43. Matreyek, K. A., Stephany, J. J. & Fowler, D. M. A platform for functional assessment of large variant libraries in mammalian cells. Nucleic Acids Res e102 (2017). doi:10.1093/nar/gkx183
44. Untergasser, A. et al. Primer3—new capabilities and interfaces. Nucleic Acids Res 40, e115-e115 (2012).
45. Livak, K. J. & Schmittgen, T. D. Analysis of Relative Gene Expression Data Using Real-Time Quantitative PCR and the 2-ΔΔCT Method. Methods 25, 402-408 (2001).

### Methods

### Protein Design

Briefly, small molecule parameters were generated with OpenBabel™ and scaffolds were docked to NS3a/drug complexes with PatchDock™ or RIFdock™ (grazoprevir/NS3a reader). The interface of the scaffold was designed with a custom RosettaScript™, and designs to test were manually selected after filtering by several design metrics.

### Constructs

Note that there were three variants of the NS3a protein sequence used in this study. A solubility optimized NS3a/4a (either catalytically active or catalytically dead, S139A) derived from HCV genotype 1a was used for the majority of the work with the designed readers. Genotype 1a NS3a/4a does not interact with the peptide ANR, which was selected to interact with genotype 1b NS3a; therefore, we engineered a hybrid NS3a/4a, NS3aH1, which is the solubility optimized NS3a/4a with four mutations needed for interaction with ANR: A7S, E13L, I35V, and T42S. NS3aH1 (catalytically active) was used for the majority of the microscopy colocalization and transcription-control constructs. NS3a/4a solubility optimized S139A was used for membrane signaling constructs with DNCR2 and GNCR1. The NS3a/4a fusion is referred to as NS3a throughout the paper. The NS3a variant used is described for each experiment below and in Table 14.

Bacterial expression constructs: Biotinylated proteins were expressed from the pDW363 vector, which encodes a bi-cistronic BirA biotin ligase. Proteins were N-terminally tagged with the biotin acceptor peptide, followed by a His₆ tag. Constructs were cloned into pDW363 via PCR-linearization of the vector, followed by Gibson assembly with the gene insert. Untagged proteins were expressed from the pCDB24 vector (gift of Christopher Bahl, Baker lab), which encodes proteins with an N-terminal His₁₀-Smt3 tag, which is scarlessly removed by ULP1. Linear gene inserts with overhangs and a stop codon added were inserted via Gibson assembly into pCDB24 that had been linearized with XhoI (New England Biolabs).

Yeast surface expression constructs: Danoprevir/NS3a reader designs were synthesized as linear genes by Gen9. All yeast constructs were cloned by homologous recombination in yeast with linearized pETCON™ vector (NdeI-/XhoI-cut, New England BioLabs). pETCON™ encodes Aga-2, the inserted gene, and a C-terminal c-myc tag for expression detection. Grazoprevir/NS3a reader designs were synthesized and constructed in complete pETCON™ plasmids by Genscript.

Mammalian expression constructs: All constructs were made in pcDNA5/FRT/TO (Thermo Fisher Scientific) unless otherwise noted. pcDNA5/FRT/TO was either linearized via PCR, or cut by BamHI and EcoRV, and inserts and vector were assembled by Gibson assembly. Dual expression constructs of DNCR2-VPR/KRAB and NS3aH1-dCas9 were made in PiggyBac™ vectors (pSLQ2818 pPB: CAG-PYL1-KRAB-IRES-Puro-WPRE-SV40PA-PGK-ABI-tagBFP-SpdCas9 and pSLQ2817 pPB: CAG-PYL1-VPR-IRES-Puro-WPRE-SV40PA-PGK-ABI-tagBFP-SpdCas9, gifts from Stanley Qi (Addgene plasmids #84241 and 84239)). The PiggyBac vectors were linearized by restriction enzyme digest, and PCR amplified inserts and digested vector were assembled by Gibson assembly. pCDNA5/FRT/TO-MCP-NS3a-P2a-DNCR2-KRAB-MeCP2-P2a-GNCR1-VPR-IRES-BFP was assembled with fragments PCR amplified from the following sources: MCP from pJZC34 (see below), KRAB-MeCP2 was a gift from Alejandro Chavez & George Church (Addgene 110821), VPR from one of the above-mentioned pPB vectors, and DNCR2, GNCR1, and NS3a (solubility optimized S139A) from gBlocks.

Single-guide RNAs (CXCR4, CD95, TRE3G) were cloned into the gRNA Cloning Vector, a gift from George Church (Addgene plasmid #41824). DNA corresponding to the guide target was ordered as a single stranded oligo with overlap to the vector and assembled with AflII-digested gRNA vector by Gibson Assembly. Scaffold RNAs (targeting CXCR4, CD95, or TRE3G with com, PP7, or MS2, respectively) were cloned into dual insert vectors derived from pSico™, expressing the scaffold RNA under a U6 promoter and the protein inserts under a CMV promoter: pJZC33 or 34 (MS2/MCP), pJZC43 (PP7/PCP), pJZC48 (com/com), gifts from Jesse Zalatan. All RNA-binding protein-reader fusions were expressed with P2a-tagBFP in place of the IRES-mCherry™ in the original vectors. This vector was also the basis of the scRNA-only vectors, which were used when all readers/RBPs were expressed separately. These vectors expressed only a tagBFP downstream of the CMV, and the guide plus 2×MS2 (wt+f6 sequences) under the U6 promoter.

pCDNA5/FRT/TO-Lifeact-mCherry™ was created from mCherry™-Lifeact-7, a gift from Michael Davidson (Addgene plasmid #54491). pEF5-FRT-mCherry-NS3a-CAAX-IRES-EGFP-DNCR2-P2a-BFP-GNCR1 was created by assembling readers and fluorescent proteins from other constructs in a pEF5-FRT backbone obtained by digestion of Addgene plasmid #61684, a gift from Maxence Nachury. pPB-NS3a-CAAX-IRES-EGFP-DNCR2-TIAM-BFP-GNCR1-LARG and pPB-NS3a-CAAX-IRES-EGFP-DNCR2-ITSN-BFP-GNCR1-iSH2 and were assembled with NS3a, reader, and fluorescent protein fragments from the previously mentioned construct, with addition of signaling effector domains from the following sources: human TIAM DH-domain residues 1033-1240 from Maly lab source, human ITSN DH-domain residues 1228-1429 from Maly lab source, LARG DH-domain was a gift from Michael Glotzer (Addgene plasmid #80408), iSH2 residues 420-615 aa from human p85 from Maly lab source. The PiggyBac vector used for these two constructs was linearized by digesting the multiple cloning site of PB501B (Systems Biosciences).

pLenti-UAS-minCMV-mCherry™/CMV-Gal4DBD-NS3a-P2a-DNCR2-VPR was based on a pLenti-UAS-minCMV-mCherry™/CMV-Gal4DBD-ERT2VP16 vector, a gift from Kenneth Matreyek, (from which the Gal4-UAS-minCMV was from Addgene plasmid #79130, a gift from Wendell Lim) which was digested with BamHI-HF and SexA1 to insert the NS3a-P2a-DNCR2-VPR fragment.

All cloning PCR reactions were performed with Q5 polymerase (New England BioLabs), and all Gibson assembly reactions were performed with NEBuilder HiFi™ Assembly Master Mix (New England BioLabs). Oligonucleotides and gBlocks were synthesized by Integrated DNA Technologies. The complete insert was verified by sequencing for each construct (Genewiz). Select mammalian expression vectors constructed in this study are available on Addgene, and bacterial or yeast expression vectors are available upon request. See Table 14 for all sequences.

### Inhibitor Sources

Grazoprevir was purchased from MedChem Express (MK-5172, product number HY-15298). Asunaprevir (BMS-650032, product number A3195) and danoprevir (RG7227, product number A4024) were purchased from ApexBio.

### Protein Expression and Purification

Proteins were expressed in BL21 (DE3) E. coli at 37° C. to an O.D.600 of 0.5-1.0, then moved to 18° C. and induced to 0.5 mM IPTG overnight. For biotinylated constructs, 12.5 mg D(+)-biotin/L culture was added upon inoculation with overnight culture. After 16-20 hours of overnight growth, cultures were harvested, and cell pellets frozen at −80° C. Cell pellets were resuspended in 20 mM Tris pH 8.0, 500 mM NaCl, 5 mM imidazole, 1 mM DTT, 0.1% v/v Tween-20. All buffers for NS3a purifications additionally included 10% v/v glycerol. Cells were lysed by sonication, and supernatant was incubated with NiNTA resin (Qiagen) for at least 1 h at 4° C. Resin was washed with 20 mM Tris pH 8.0, 500 mM NaCl, 20 mM imidazole, and proteins were eluted with 20 mM Tris pH 8.0, 500 mM NaCl, 300 mM imidazole. Biotinylated constructs were then further purified by size exclusion chromatography on a Superdex 75 10/300 GL column (GE Healthcare) in 20 mM Tris pH 8.0, 300 mM NaCl, 1 mM DTT, 10% v/v glycerol. Proteins were stored in this buffer at −80° C. For proteins tagged with His₁₀-Smt3, the tag was removed by overnight cleavage at room temperature using His-tagged ULP1 protease (purified in-house) at a ratio of 1 mg ULP1:250 mg protein. Cleavage was performed concurrent with dialysis (3.5 kDa mwco Slide-A-Lyzer™ dialysis cassettes, Thermo Scientific) in 20 mM Tris pH 8.0, 300 mM NaCl, 1 mM DTT, 10% v/v glycerol. Cleaved protein was then put through a second NiNTA purification, with the desired protein collected in the flowthrough and wash (20 mM Tris pH 8.0, 500 mM NaCl, 20 mM imidazole, 10% v/v glycerol). NS3a S139A and DNCR2 for crystallization were further purified via ion exchange chromatography on a HiTrap™ SP column (GE Healthcare) and HiTrap Q column (GE Healthcare), respectively, followed by size exclusion chromatography on a Superdex™ 75 10/300 GL column (GE Healthcare) in 20 mM Tris pH 8.0, 100 mM NaCl, 2 mM DTT. 60 μM NS3a and 100 μM DNCR2 were mixed with 500 μM danoprevir and incubated at 4° C. overnight. The NS3a S139A/DNCR2/danoprevir complex was further purified by size exclusion chromatography on a Superdex 75 10/300 GL column (GE Healthcare) in 20 mM Tris pH 8.0, 50 mM NaCl, 2 mM DTT. The protein complex peak fractions were pooled and subsequently concentrated to 7 mg/mL for crystallization.

### Crystallization of the DNCR2, NS3a and Danoprevir

Crystals were obtained using the hanging drop method by adding 1 μl of the above NS3a/DNCR2/danoprevir complex to 1 μl of a well solution containing 100 mM Bis-Tris, pH 6.5, 200 mM LiSO₄ and 22% w/v PEG 3350. Crystals formed in 24-36 h at room temperature. Crystals were flash-frozen with liquid nitrogen in a cryoprotectant with 20% v/v glycerol.

### X-Ray Data Collection and Structure Determination

Data collection was performed at the ALS beamlines 8.2.1 and 8.2.2. The diffraction data was processed by the HKL2000 package in the space group P2₁. The structure was determined, at 2.3 Å resolution, using one data set collected at a wavelength of 1.00 Å, which was also used for refinement (Extended Data Table 2). The initial phases were determined by molecular replacement with the program Phaser, using the crystal structure of NS3a (PDB code: 3M5L) as the initial search model. Two NS3/4a were found in one asymmetric unit, and the experimental electron density map clearly showed the presence of two molecules of DNCR2 with two molecules of danoprevir in one asymmetric unit. The complex model was improved using iterative cycles of manual rebuilding with the program COOT and refinement with Refmac5 of the CCP4 program suite. There were no Ramachandran outliers (98.3% most favored, 1.7% allowed).

### Analytical Size Exclusion Chromatography

5 nmoles of each protein or drug were mixed in 300 μL total volume (16.7 μM final concentration), in a buffer of 20 mM Tris pH 8.0, 300 mM NaCl, 10% glycerol, 1 mM DTT. Complexes were incubated on ice for 1 h before injection of 250 μL into a 500 μL loop and onto a Superdex™-75 10/300 GL column (GE Healthcare) at 4° C. Untagged NS3a S139A (solubility optimized) and untagged DNCR2 were used for SEC.

### Combinatorial Library Design

Library design to improve the affinity of the original designs proceeded through three stages: 1) Redesign of the D5 or G3 interface using RosettaDesign™, 2) selection of positions to vary in the library, and 3) optimization of degenerate codon choices to encode the library using a previously described integer linear programming approach.

Redesign of the interfaces was done using the RosettaScript™ cid_roll_design.xml (Supplemental Methods). ˜1000 redesigns were generated for D5/G3. Unique sequences from designs that had a ROSETTA™ ddg score below that of the original design (700-800 sequences) were used to assemble a position specific scoring matrix (PSSM).

To select positions to vary in the library, this PSSM was visually examined with reference to the original design and the redesign models. Positions with significant changes in the redesigns that were proximal to the interface were chosen to vary in the library. Additionally, to enable construction of each library from two oligonucleotides, the positions varied were constrained to two helices (helices 5 and 7 for D5, and helices 2 and 4 for G3).

The library design scripts require two inputs: a short list of residues required to be varied in the library, and a longer list of preferred residues and/or a PSSM.³⁷ Required residue lists generally included the original residue from the design, with a further hand-selected set of residues highly preferred in the redesigns. Preferred residue lists included all amino acids occurring in the redesigns. The D5 library was designed by optimizing degenerate codon choice to encode as many preferred residues as possible within a DNA library size constraint of 10⁷. The resulting library encoded 4.1×10⁶ protein variants). The G3 library was designed by optimizing the sum of the PSSM scores from the redesigns within a DNA library size constraint of 10⁷. The resulting G3 library encoded 7.1×10⁶ protein variants.

DNCR1 combinatorial library design used the same library optimization approach as above, but used experimentally determined mutational preferences as the input, rather than design-determined preferences. The enrichment values from the DNCR1 SSM library (see below) were standardized (Z-value) for each positive sort (performed at 50 nM or 500 nM NS3a). The Z-values for the two sorts were then averaged. These average standardized enrichment values were used as a PSSM input to the library design script. Positions to vary were hand-chosen based on their proximity to the designed interface (based on the original D5 model), as well as the presence of multiple enriched mutations in the SSM results. The mutations that were required to be included in the library design were also hand-picked from the most enriched mutations (top 10% of enrichment values), while the inclusion of additional mutations was optimized by maximizing the sum of the enrichment scores. Some large codon choices were removed to enforce a modest number of mutations at each position. Additionally, chemical diversity classes were defined to prioritize inclusion of certain classes of residues. The library DNA size was constrained to be <10⁸ variants, and final size in protein sequences was 2.76×10⁷.

### Yeast Display Library Construction

Combinatorial libraries were assembled from two ultramer oligonucleotides (Integrated DNA Technologies), which contained a short, overlapping region corresponding to part of the constant helix between the two varied helices (helix 6 for the D5 libraries, and helix 3 for the G3 library). Linear, double stranded fragments were generated in the first PCR by pairing each varied primer with a constant primer that annealed 5′ or 3′ to the end of the full gene. These fragments were excised and extracted from an agarose gel. A second round of PCR was performed to overlap these fragments, with further amplification by addition of the outside primers in the 10^{th} cycle (out of 35). The correct-sized product was gel extracted and used as the template for 1-2 more rounds of PCR with the outside primers to yield sufficient DNA. The DNCR1 SSM library was assembled using a pair of primers (Integrated DNA Technologies) for each of the 75 protein positions varied, where the forward primer contained the NNK site in a central position, and the reverse primer overlapped with the 5′ end of the forward primer.³⁸ Linear fragments corresponding to each primer pair were overlapped in a second round of PCR to yield the full gene insert. Combinatorial library PCRs were performed with Q5 polymerase (New England BioLabs), and the SSM library PCRs were performed with Phusion™ polymerase (Thermo Fisher Scientific). For all libraries, the linear library DNA was combined with NdeI- and XhoI-digested pETCON™ at a ratio of 4 μg insert:1 μg vector and electroporated into freshly-prepared electrocompetent EBY100 S. cerevisiae.

### Yeast Surface Display Analysis and Sorting

Yeast were grown overnight at 30° C. in yeast minimal media (-ura for strain selection, -trp for pETCON™ selection) supplemented with 2% w/v glucose. Overnights were used to inoculate SGCAA cultures (2% w/v galactose, 0.67% w/v yeast nitrogen base, 0.5% w/v casamino acids, and 0.1 M sodium phosphate, pH 6.6) to an O.D. 600 of 1.0-2.0 and protein expression was induced overnight at 30° C. Before sorting or analysis, cells were pelleted and resuspended in PBS supplemented with 0.5% w/v bovine serum albumin (PBSA). Protein solutions of biotinylated NS3a with danoprevir or grazoprevir were made in PBSA and incubated with the yeast for 30 min-1 h at 22° C. For analysis and sorting of initial, low-affinity designs, NS3a was pre-tetramerized by incubation with streptavidin-phycoerythrin (SAPE, Invitrogen) at a molar ratio of 1 SAPE:4 NS3a for at least 10 minutes prior to incubation with yeast; these sorts are denoted as “avid” below. Cells were washed in cold PBSA and incubated for 15 min on ice with SAPE and fluorescein isothiocyanate-conjugated chicken anti-c-myc (Immunology Consultants Laboratory), both diluted 1:100 in PBSA. After the labeling incubation, cells were washed again in cold PBSA and analyzed on a C6 flow cytometer (Accuri) or a FACSCanto™ cytometer (BD Biosciences), or sorted on a SH800 (Sony Biotechnology) cell sorter or a FACSAria III (BD Biosciences) cell sorter. All FACS data were analyzed using FlowJo (v.10.1). See FIG. 30a for yeast gating strategies. Sorted yeast recovered for 1-2 days at 30° C. in yeast minimal media plus 2% w/v glucose.

Titration curves for NS3a/drug complexes on yeast-displayed designs used construct NS3a_3 (solubility-optimized, catalytically active). Drug concentrations were at a fixed molar ratio of 10 drug:1 NS3a, with the exception of the DNCR2-danoprevir titration, for which a fixed concentration of 50 nM danoprevir was used for all points to stay above the NS3a/danoprevir Kᵢ. Curves were fit using Graphpad Prism 5 to a one-site specific binding model with Hill coefficient.

For the first D5 library, the following sequential sorts were performed using catalytically active NS3a (NS3a_3): 1 μM NS3a/10 μM danoprevir, 0.5 μM NS3a avid/5 μM danoprevir, 0.5 μM NS3a avid/5 μM danoprevir, 0.25 μM NS3a avid/2.5 μM danoprevir, 2 μM NS3a/20 μM danoprevir, 20 nM NS3a/200 nM danoprevir. The highest 1-3% PE/FITC-positive events were collected for each sort, with the gate set along the binding/expression diagonal. For the DNCR1 combinatorial library, the following sequential sorts were performed using catalytically inactive NS3a (NS3a_2): 100 nM NS3a/1 μM danoprevir, 100 nM NS3a/1 μM danoprevir, 50 nM NS3a/500 nM danoprevir, 5 nM NS3a/50 nM danoprevir, 500 μM NS3a/50 nM danoprevir, 20 μM NS3a/50 nM danoprevir. The top 0.5-9% were collected in each sort. For the G3 library, the following sequential sorts were performed using catalytically inactive NS3a (NS3a_2): 500 nM NS3a avid/5 μM grazoprevir, 50 nM NS3a avid/500 nM grazoprevir, 500 nM NS3a/5 μM grazoprevir, 500 nM NS3a/5 μM grazoprevir, 250 nM NS3a/2.5 μM grazoprevir, 100 nM NS3a/1 μM grazoprevir, 30 nM NS3a/300 nM grazoprevir.

The most-enriched clones were assessed by colony PCR and sequencing (Genewiz) of ˜50 colonies from the final 2-3 pools of each library. Titrations of NS3a/drug were performed on several of the most enriched clones to verify that the most-enriched clones (DNCR1 and GNCR1) exhibited the tightest binding. DNCR2 was selected from multiple very high-affinity clones based on its superior expression on yeast.

For the DNCR1 site saturation mutagenesis (SSM) library, two sorts were performed on the same day at 50 nM NS3a (NS3a_2)/500 nM danoprevir and 500 nM NS3a (NS3a_2)/5 μM danoprevir. For both conditions, a positive-sort gate was set to collect the top 1% of binders, and a negative-sort gate was set to collect the bottom 6% of binders. All gates were set along the binding/expression diagonal. The naïve population for sequencing analysis was saved from the same day of growth.

DNCR1 SSM library sequencing

At least 20 million cells were harvested for each selected library pool and the naïve library, and DNA was extracted and prepared for Illumina sequencing The first round of qPCR, to amplify the 150 bp varied region, was performed for 25-35 cycles using Phusion polymerase. After gel extraction, a second round of PCR was performed to add on barcodes and Illumina adaptors. Sequencing was performed with a 600-cycle reagent kit (Illumina) on a MiSeq™ sequencer (Ilumina). Enrich was used to align and filter the paired-end reads.⁴⁰ An average quality for each read was required to be greater than 20, no N's were allowed, and the maximum number of nucleotide mutations allowed per sequence was 3. The sequence counts output by Enrich were processed by an in-house Python script to calculate the enrichment value (enrichment ratio for each mutant, normalized by the wild-type enrichment ratio): log 2 (Fv,sel/Fv,inp)/(Fwt,sel/Fwt,inp), where Fv is the frequency of the variant in the selected or input (naïve library) pool, and Fwt is the frequency of the wild-type residue. Only single mutants that had at least 15 counts in the naïve library were included in the analysis.

### Mammalian Cell Culture

All cells were cultured in high-glucose DMEM, 4 mM L-glutamine, 10% fetal bovine serum (FBS, Life Technologies) at 37° C., 5% CO₂. Cells were tested and found free of mycoplasma monthly.

### Confocal Microscopy for Colocalization Analysis

A Leica SP8× system was used for confocal microscopy. A UV laser at 405 nm was used to excite tagBFP. White light lasers of 488 and 587 nm were used for EGFP and mCherry™, respectively. TagBFP emission was recorded on a PMT detector, and EGFP and mCherry™ were detected by separate HyD™ detectors. All images were taken using a 63× objective with oil, at 512×512 resolution.

Colocalization experiments were performed in NIH3T3 cells (Flp-In-3T3, Thermo Fisher Scientific). For fixed-cell experiments, cells were plated at 3×10⁴ cells/mL on sterile glass coverslides placed in 12-well culture plates. Cells were transfected 24 hours after plating with Lipofectamine™ 2000 or 3000 (Thermo Fisher Scientific) at a ratio of 3 μL reagent:1 μg DNA, according to manufacturer's instructions. 3-vector transfections were performed with 0.3 μg NS3a and 0.35 μg each ANR/DNCR2/GNBP vectors, while 2-vector transfections were performed with 0.3 μg free component and 0.7 μg of the immobilized component. One day after transfection, cells were treated with drug or DMSO and fixed. Drug additions were performed by exchanging the media for DMEM+10% v/v FBS plus drug. To fix, cells were washed once with DPBS (Thermo Fisher Scientific), then incubated with 4% v/v paraformaldehyde in DPBS for 15 minutes. After washing twice with DPBS, coverslides with cells were removed from the plate and mounted on glass slides using Fluoromount-G (SouthernBiotech).

For the live cell experiment assaying DNCR2 membrane association time, cells were plated at 3×10⁴ cells/mL in 35 mm glass-bottomed dishes (Matek), that were coated with poly-D-lysine. Experiments were performed in FluorBrite™ DMEM (Thermo Fisher Scientific) media supplemented with GlutaMax™ (Thermo Fisher Scientific) and 10% v/v FBS. Cells were imaged with dishes open on a heated stage (˜55° C., which resulted in the media at the center of the plate remaining at ˜30° C.). 5 μM drug additions were performed by removing 1 mL media from the dish, mixing with drug, and returning to the dish after 2 minutes of imaging. All cells were imaged within 30 minutes of removal from incubator, and no environmental controls were used beyond heating. The constructs used for live cell membrane localization kinetics were myristoyl-tag-mCherry™-NS3a and DNCR2-EGFP.

Colocalization of NS3a and DNCR1 at the plasma membrane, nucleus, mitochondria and Golgi was performed with two sets of constructs, with either NS3a or DNCR1 as the immobilized component. mCherry™-NS3a was used with Tom20-DNCR1-EGFP, DNCR1-EGFP-Giantin, and 3×NLS-DNCR1-EGFP. DNCR1-EGFP was used with Tom20-mCherry™-NS3a, mCherry-NS3a-Giantin, 3×NLS-mCherry™-NS3a, and myristoyl-tag-mCherry™-NS3a. Drug specificity of DNCR1 was analyzed with mCherry™-NS3a and Tom20-DNCR1-EGFP or DNCR1-EGFP-Giantin, and drug specificity of DNCR2 and NS3a with DNCR2-EGFP and Tom20-mCherry™-NS3a. Colocalization was analyzed after 1 h of 10 μM drug or equal volume DMSO treatment.

Colocalization of NS3a, ANR, and DNCR2 was performed with NS3aH1-mCherry™ in combination with 2 separate vectors encoding 3×NLS-DNCR2-EGFP and ANR-ANR—BFP-CAAX (0.3 μg, 0.35 μg, 0.35 μg, respectively) or one vector encoding Tom20-BFP-ANR-ANR-P2a-DNCR2-EGFP-CAAX (0.3 μg NS3a, 0.75 μg ANR/DNCR2). Colocalization of NS3a, DNCR2 and GNCR1 was performed with NS3aH1-mCherry™, Tom20-DNCR2-EGFP, and GNCR1-BFP-CAAX (2-location; 0.3 μg, 0.35 μg, 0.35 μg, respectively), or with DNCR2-EGFP, GNCR1-BFP, and NS3aH1-mCherry™-CAAX (1-location; 0.25 μg, 0.25 μg, 0.5 μg, respectively). For all 3-color experiments, 15-minute drug treatments with 5 μM danoprevir or grazoprevir or equal volume DMSO were performed prior to fixing.

For the colocalization experiment shown in FIG. 21b, a single pEF5 vector expressing mCherry™-NS3a(S139A)-CAAX-IRES-EGFP-DNCR2-P2a-BFP-GNCR1 was transiently transfected into NIH3T3 cells as previously described. Cells were treated with combinations of danoprevir and grazoprevir or equal volume DMSO for 1 hour before fixing.

All images were analyzed using ImageJ. Pearson's r values reported are Rcolocalization values generated using an automatic thresholding program (Colocalization Threshold plugin).⁴ For DNCR2 membrane associate kinetics analysis, a square ROI was set to include only cytoplasm. EGFP fluorescence was quantified in the ROI over the timecourse. 15 min timecourses (2 min pre-drug addition, 13 min post-drug) were collected for 18 cells from 4 independent plates. The cytoplasmic fluorescence was normalized to the value in the first and last frame for each cell. Because the cells were imaged at different time points (every ˜20-30 seconds), we used an in-house Python script to fit a 1-D interpolation to each timecourse and plotted the average and standard deviation value of the 1-D functions at 20 second intervals. Time points after drug addition were fit to an exponential decay model to calculate a t_{1/2} using Graphpad Prism 5 (y=(y₀−b)*e^{-kx}+b, where b was constrained to 0, but y₀ was left unconstrained to account for minor variability in drug addition and mixing times).

### Widefield Microscopy for Signaling Phenotype Analysis

Widefield images were collected in an environmental chamber with humidity control, 37° C., and 5% CO₂ on a Leica DMi8 automated fluorescence microscope. Cells were plated on glass-bottomed 96-well plates (Cellvis). Plates were treated with 10 μg/mL bovine fibronectin (Sigma Aldrich) for 1 hour and washed once with PBS.

The cell line used was TRex™-HeLa (ThermoFisher Scientific), into which Lifeact-mCherry™ was stably integrated into the doxycycline-regulated Flp-In site by co-transfection of the pCDNA5-FRT/TO-Lifeact-mCherry™ vector with the Flp recombinase plasmid pOG44 (ThermoFisher Scientific) according to manufacturer's protocols. Lifeact-mCherry™ was induced by addition of 1 μg/mL doxycycline to culture media. For expression of signaling effector proteins, 1 day prior to imaging, 5×10⁶ cells were transiently transfected with 10 μg DNA in a 100 μL electroporation tip using a Neon transfection system (ThermoFisher Scientific) according to manufacturer's recommendations for HeLa cells. 5×10³ cells were plated in each well of the 96-well plate used for imaging. Cells recovered in complete DMEM with 10% FBS overnight. The following day, media was aspirated, cells were washed once with PBS, and cells were serum starved for 3-8 hours before imaging in 100 μL FluorBrite™ DMEM (Thermo Fisher Scientific) media supplemented with GlutaMax™ (Thermo Fisher Scientific) (“imaging media”). For Rac/Rho regulation, the construct PB-NS3a-CAAX-IRES-EGFP-DNCR2-TIAM-P2a-BFP-GNCR1-LARG was used, with images collected for the mCherry™ (Lifeact) and EGFP (DNCR2-TIAM) channels. Cells were imaged for 10 minutes prior to drug addition, and drug was added by pipetting 100 μL 2× drug in prewarmed imaging media, after which cells were imaged for a further 60 minutes.

### AKT Western Blots

COS-7 cells (ATCC), were plated in 24-well plates at 2×10⁵ cells/mL (0.5 mL volume). One day later, cells were transfected using TurboFectin™ 8.0 (OriGene) according to the manufacturer's instructions with 0.75 μg myristoyl-tag-mCherry™-NS3a and 0.25 μg DNCR2-iSH2 vectors. One day after transfection, cells were washed once with DPBS, and media was replaced with serum-free DMEM. After serum-starving for 22 hours, cells were exposed to a 15-min drug treatment using 12, 3-fold dilutions of danoprevir from 5 μM to 0 μM, in triplicate. After drug treatment, cells were washed once in DPBS, then lysed in 50 μL modified RIPA buffer (50 mM Tris-HCl, pH 7.8, 1% v/v IGEPAL CA-630, 150 mM NaCl, 1 mM EDTA, 1× Pierce Protease Inhibitor Tablet) for 30 minutes on ice. Cell debris was cleared by centrifugation at 17 kg for 10 min at 4° C. Lysate was mixed with protein loading dye and denatured at 95° C. for 7 minutes then run on an SDS-PAGE gel (Criterion, Bio-Rad) and transferred to nitrocellulose. Blocking and primary antibody incubations were done in a 1:1 mix of TBS plus 0.1% v/v Tween-20 (TBST) and blocking buffer (Odyssey). Primary antibodies used were pSER473 AKT (1:2000, Cell Signaling Technologies #4060), and pan-AKT (1:2000, Cell Signaling Technologies #2920). Blots were washed with TBST, then incubated with secondary antibodies diluted 1:10,000 in TBST (goat anti-rabbit-IRDye™ 800 CW (926-32211) and goat anti-mouse-IRDye™ 680LT (926-68020), LI-COR), washed, and imaged on a LI-COR^{T}M Odyssey scanner. pAKT signal was divided by AKT signal for each lane, and the titration curve was fit to a three-parameter dose-response curve (fitting top, bottom, and EC50) in Graphpad™ Prism 5.

dCas9 Transcription Control

CXCR4 and CD95 induction experiments with DNCR2-VPR and NS3aH1-dCas9 were performed in HEK293T cells (293T/17, ATCC) following the protocol and using the same materials as detailed in Gao et al. Antibodies used were: APC anti-human CD184 (CXCR4) [12G5] (BioLegend 306510), PE anti-human CD95 (Fas) [DX2] (BioLegend 305607), PE Mouse IgG1, κ Isotype Ctrl [MOPC-21] (BioLegend 400111), APC Mouse IgG2b, K Isotype Ctrl [MPC-11] (BioLegend 400322). No binding of isotype controls was observed to HEK293T cells; therefore, no background adjustments were made for isotype binding. Briefly, cells were plated in 12-well plates at 6×10⁴ cells/mL on day 1 and transfected with TurboFectin™ 8.0 (OriGene) according to the manufacturer's instructions on day 2. 1 μg total DNA was transfected per well (0.5 μg pB-DNCR2-VPR/NS3a-dCas9, 0.5 μg equal mix of 3 CD95 or CXCR4 guide RNA vectors (or unrelated guide for “No guide” controls)). 10 μM danoprevir was added on day 3, and cells were harvested on day 5 (VPR), incubated with antibodies for 1 hr, and analyzed on a FACSLSRII™ (BD Biosciences). For gene repression experiments with KRAB, cells were passaged on day 5, incubated with fresh drug, and analyzed on day 7. For all mammalian FACS experiments (unless otherwise noted), 10,000 single cell events were collected for each sample, and the median fluorescence signal of cells with BFP signals greater than that of untransfected cells were reported. All FACS data were analyzed using FlowJo™ (v.10.1). See FIG. 30b for mammalian cell gating strategies.

Danoprevir/grazoprevir titrations to linearize CXCR4 or CD95 expression were performed with DNCR2-VPR and NS3a-dCas9 following the protocol detailed above for gene induction with VPR, but in 24-well plates with 0.5 μg total DNA. Danoprevir was titrated in 12 concentrations in 2.5-fold dilutions starting from 1000 nM. Grazoprevir dilutions were added to the danoprevir titration, all starting from 10 nM grazoprevir, and decreasing across 12 concentration points in 2-, 1.5-, or 1.25-fold dilutions. Data were fit to four-parameter log dose-response curves (fitting EC50, upper and lower baselines, and Hill coefficient) in Graphpad Prism 5.

Induction and reversion timecourses of CXCR4 expression that were analyzed by qPCR were performed in a similar manner, with 10 μM danoprevir replaced by 10 μM grazoprevir or equal DMSO after 24 hours of danoprevir treatment. Wells (in triplicate for each condition) were harvested at each time point by aspirating, washing with 1 mL DPBS, adding 300 μL Versene (ThermoFisher Scientific) and incubating for 5 minutes at 37° C., then pelleting at 3.5 krpm for 2 minutes at 4° C., aspirating, and freezing the pellets at −80° C.

GFP expression experiments were performed in a HEK293T cell line with GFP stably integrated in a single tetracycline-inducible landing pad (7×TRE3G operator with rTA) created in a similar manner as a previously published TetBxblBFP-rTA HEK293T cell line (gift from Doug Fowler). Combined CXCR4 and GFP induction was performed in this line transfected with 0.3 μg pCDNA5-FRT/TO-dCas9, 0.3 μg pCDNA5/FRT/TO-NS3aH1-VPR, 0.2 μg CXCR4-2×MS2/MCP-GNCR1-P2a-BFP (equal mix of 3 scRNAs), and 0.2 μg TRE3G-2×PP7/PCP-DNCR2-P2a-BFP. Drug treatment (48 hours) with 10 μM danoprevir or 10 μM grazoprevir or danoprevir/grazoprevir matrix, harvesting, CXCR4 antibody incubation and FACS analysis were performed as described above for immunofluorescence analysis.

The 3-gene experiment was performed in the GFP reporter HEK293T cell line transfected with 0.25 μg pCDNA5-FRT/TO-dCas9, 0.25 μg pCDNA5/FRT/TO NS3aH1-VPR, 0.166 μg TRE3G-2×MS2(wt+f6)/MCP-ANR-ANR-P2a-BFP, 0.166 μg CXCR4-com/com-GNCR1-P2a-BFP (equal mix of 3 scRNAs), and 0.166 μg CD95-2×PP7/PCP-DNCR2-P2a-BFP (equal mix of 3 scRNAs). Cells were plated in 12-well plates at 6×10⁴ cells/mL on day 1 and transfected with TurboFectin™ 8.0 (OriGene) according to the manufacturer's instructions on day 2 and 1 μM or 10 μM drug was added on day 3. Cells were harvested on day 5 as described above for other samples to be analyzed to qPCR.

For RT-qPCR analysis, RNA was extracted with the Arum™ Total RNA Mini Kit (Bio-Rad). Integrity of the total RNA was confirmed by running on an agarose gel. Reverse transcription was performed on 1 μg total RNA using the iScript™ Reverse Transcription Kit (Bio-Rad), according to manufacturer's instructions. A no-RT control was performed on several samples per experiment to confirm that there was no significant genomic DNA contamination. qPCR was performed on 50 ng cDNA (1 μL of RT reaction) in a 10 μL reaction volume using SsoAdvanced Universal SYBR™ Green Supermix (Bio-Rad). For each biological sample, technical duplicates of the qPCR were performed and averaged. qPCR primers for GAPDH (reference gene), CXCR4, CD95, and GFP are listed in Table 14. CXCR4 and GAPDH primers are from Zalatan et al., and CD95 and GFP primers were designed to amplify a 94 bp product using Primer3 (v. 0.4.0).^{20,44} A thermocycle of 95° C. for 2 min, (95° C. 10 sec, 58° C. 30 sec)×40 cycles, 65° C.-95° C. at 0.5° C. increments 5 sec/step was performed on a Bio-Rad CFX Connect Real-Time System. For the CXCR4 reversibility experiment, fold-change in CXCR4 expression was calculated relative to a 0 hr timepoint using the 2^{-ΔΔCT} method.⁴⁵ For analysis of the 3-gene experiment, fold-change was calculated relative to untransfected TRE3G-GFP HEK293Ts.

The switchable gene expression/repression experiment on CXCR4 and CD95 was performed in TReX™-HEK293 cell (ThermoFisher Scientific), into which Sp dCas9 was stably integrated using vector pCDNA5/FRT/TO-nFLAG-dCas9 and the Flp recombinase vector pOG44, according to manufacturer's protocols. This experiment followed our general dCas9 transcription experiment workflow described above. Briefly, cells were plated on day 1, transfected and induced with doxycycline on day 2, had 100 nM danoprevir or grazoprevir or equal volume DMSO added on day 3, and harvested for FACS analysis on day 5. All readers were transfected in via one plasmid, pCDNA5/FRT/TO-MCP-NS3a-P2a-DNCR2-KRAB-MeCP2-P2a-GNCR1-VPR-IRES-BFP. A mix of 3 guides each for CXCR4 or CD95 were transfected, or a gal4-4 control guide, all in a pU6-guide-2×MS2(wt+f6)/CMV-BFP vector. 0.5 μg reader and 0.5 μg guide plasmids were co-transfected in each well. Cells were incubated with antibodies and analyzed as described above, with 20,000 single-cell events collected per sample, and the median fluorescence plotted for cells with the top ˜30% BFP expression signal.

### Inducible Gal4 Transcription Factor

HEK293T/17 cells (ATCC) were plated at 7×10⁴ cells/mL in 0.5 mL in 24-well plates. One day later, they were transfected with 0.35 μg pLenti-UAS-mCherry™/CMV-Gal4DBD-NS3a-P2a-DNCR2-VPR and 0.15 μg of a BFP-expressing vector to use for gating on transfection-positive cells. The next day, a 12-point dilution series of danoprevir was added with 2.5-fold dilutions starting at 100 nM danoprevir. Two days later, cells were removed from the plate with Versene (Gibco), and analyzed for mCherry™ and BFP fluorescence on an FACSLSRII (BD Biosciences). 20,000 single-cell events were collected, and median mCherry™ fluorescence was reported for the cells with the top ˜50% of BFP signal for each sample.

### Statistics

All P-values are from unpaired, two-sided t-tests, computed using Graphpad™ Prism 5.

EXTENDED DATA TABLE 1
Apparent dissociation constants for yeast-displayed
design variants to NS3a/drug complexes

| | | K_{D} (nM) ± standard | | |
|---|---|---|---|---|
| | | deviation | | |
| Clone | Drug | or relative binding | | Fold drug specificity |
| D5 | danoprevir | ++ | | — |
| | grazoprevir | +++ | | none |
| | asunaprevir | + | | modest |
| DNCR1 | danoprevir | 190 ± 10 | nM | — |
| | grazoprevir | 2900 ± 100 | nM | 15 |
| | asunaprevir | 6300 ± 2600 | nM | 33 |
| DNCR2 | danoprevir | 0.036 ± 0.0002 | nM | — |
| | grazoprevir | 2000 ± 100 | nM | 56,000 |
| | asunaprevir | 770 ± 50 | nM | 21,000 |
| G3 | grazoprevir | ++ | | — |
| | danoprevir | no binding | | high |
| | asunaprevir | + | | modest |
| GNCR1 | grazoprevir | 140 ± 20 | nM | — |
| | danoprevir | >10,000 | nM | >71 |
| | asunaprevir | >10,000 | nM | >71 |

Data are from three technical replicates. Relative binding of different NS3a/inhibitor complexes is given for conditions where binding was too weak to achieve full titration curves on yeast, indicating binding in the low-to-mid micromolar-range, or weaker. See Supplementary FIG. 2a, f for single concentration point data for D5 and G3.

EXTENDED DATA TABLE 2
Crystallography data collection and refinement statistics

| | DNCR2/NS3a/danoprevir |
|---|---|
| Data collection | |
| Space group | P2₁ |
| Cell dimensions | |
| a, b, c (Å) | 70.84, 69.26, 99.34 |
| α, β, γ (°) | 90, 108.59, 90 |
| Resolution (Å) | 50.0-2.30 (2.37-2.30) * |
| R_{sym} | 0.102 (0.340) |
| I/σI | 14.4 (2.6) |
| Completeness (%) | 99.3 (93.6) |
| Redundancy | 4.1 (3.0) |
| Refinement | |
| Resolution (Å) | 50.0-2.30 |
| No. reflections | 38559 |
| R_{work}/R_{free} | 0.203/0.240 |
| No. atoms | |
| Protein | 5818 |
| Ligand/ion | 134 |
| Water | 209 |
| B-factors | |
| Protein | 29.9 |
| Ligand/ion | 33.4 |
| Water | 37.4 |
| R.m.s. deviations | |
| Bond lengths (Å) | 0.012 |
| Bond angles (°) | 1.545 |

This diffraction data set was collected from a single crystal.
*Values in parentheses are for highest-resolution shell.

### Supplementary Note 1

### Protein Engineering Details and Biochemical Characterization of DNCR and GNCR

The danoprevir/NS3a complex reader design process started with docking, using PatchDock™, a set of highly stable, de novo designed proteins on a danoprevir/NS3a structure: leucine-rich repeat proteins, designed helical repeat proteins (DHRs), ferredoxins, and helical bundles.¹⁻³ One design, D5, based on a DHR, showed danoprevir-dependent binding to NS3a when assayed via yeast surface display.

To improve D5's affinity for the NS3a/danoprevir complex, we used two sequential yeast surface display libraries (FIG. 22). First, a combinatorial library was designed based on the frequencies of mutations present in re-designs of the D5 interface (FIG. 22a). These Rosetta™ re-designs were obtained after small rigid-body perturbations of D5 relative to the danoprevir/NS3a complex. Sorting this library with increasingly stringent conditions led to a variant, danoprevir/NS3a complex reader 1 (DNCR1), that specifically bound the NS3a/danoprevir complex with high nanomolar affinity (Extended Data Table 1). Next, we characterized a single-site saturation mutagenesis (SSM) library of DNCR1's two designed primary interface helices (5 and 7) and the non-interface helix 6. Enrichment ratios, calculated after sorting for both NS3a/danoprevir complex binders and non-binders, supported the overall designed binding mode (FIG. 22b). Interestingly, the negative sort, which enriched for non-binders, gave us further structural insight into the binding mode of DNCR1. The surface residues of helix 6, which faces away from the interface, were very permissive of substitution. Likewise, a region from the C-terminus of helix 6 to the N-terminus of helix 7 was permissive of mutation to nearly any residue, including proline. The helices in this region were found to have unfolded in the DNCR2/danoprevir/NS3a structure, and the shift of DNCR2 results in this region of the DHR not participating in the interface (FIG. 23c). The trends seen in the negative sort SSM library enrichment ratios support the hypothesis that DNCR1 likely binds similarly to DNCR2. A second combinatorial library was designed based on the positive sort enrichment ratios, and enrichment of this library for NS3a/danoprevir binding resulted in multiple high affinity clones, of which one, DNCR2, was chosen for further characterization, based on its superior expression on the surface of yeast (FIG. 22c). The progression of improved binding from the original scaffold DHR79, to the design D5, and through two libraries resulting in DNCR1, and finally DNCR2, are illustrated by the DNCR1 SSM enrichment ratios in FIG. 22d.

We performed a detailed biochemical analysis of the DNCR2/danoprevir/NS3a complex to confirm that it had the expected properties of a chemically-induced heterodimer. DNCR2 does not appear to bind substantially to danoprevir alone based on the inability of a high concentration (100 μM) of the free drug to disrupt the DNCR2/danoprevir/NS3a complex on yeast (FIG. 23b). Size exclusion chromatography demonstrated that DNCR2 and NS3a behave as expected, forming a 1:1 complex only in the presence of danoprevir (FIG. 23e). This behavior, along with the drug specificity described in the main text (FIG. 23a,f), indicated that we had successfully designed and engineered a chemically-induced heterodimer that was only inducible by danoprevir.

For our drug/NS3a complex reader, we targeted the NS3a/grazoprevir complex. Grazoprevir is an FDA-approved drug with picomolar affinity to NS3a (Kᵢ of 140 μM).⁶ For this round of design, we exclusively used DHR scaffolds, as our first-generation design had indicated that they were more suitable scaffolds for our design goal. We assembled a DHR scaffold set of many curvatures and sizes from published DHR crystal structures, as well as an in-house set of models (available upon request). We used both PatchDock™ and a new rotamer interaction field docking protocol (RIFDock™) to center the DHR scaffolds over grazoprevir, followed by the same design approach that was used for the danoprevir CID design. We ordered and tested 29 designs by yeast surface display. Five designs based on DHR models showed very weak, but grazoprevir-dependent binding (data not shown). One design, G3, based on the crystal structure of DHR18, showed modest binding, similar to the first-generation danoprevir reader design, D5 (FIG. 24a).

We computationally characterized the mutational preferences of the G3 interface via a similar Rosetta™-based approach used to predict the mutational preferences of D5. The predicted mutational preferences at the G3 interface are shown in FIG. 24b. These mutational frequencies were used to design a combinatorial library varying 9 positions of G3, which was sorted for sequences with increased affinity to NS3a/grazoprevir (FIG. 24c). Both G3 and the final high-affinity clone, grazoprevir/NS3a complex reader 1 (GNCR1), showed high specificity for binding grazoprevir/NS3a over complexes of NS3a with danoprevir or asunaprevir, or apo NS3a (FIG. 24a, Extended Data Table 1). GNCR1 had a similar affinity for the grazoprevir/NS3a complex as DNCR1 had for the danoprevir/NS3a complex (<200 nM). Because this affinity was demonstrated to be perfectly adequate to function as a chemically-inducible dimerizer in mammalian cells, we did not engineer GNCR1 further.

### Supplementary Note 2

### Validation of DNCR1 and NS3a Ability to Localize to Multiple Subcellular Locations

As an assay for colocalization of NS3a and DBP, we used confocal fluorescence microscopy of NIH3T3 cells transiently transfected with pairs of NS3a-mCherry™ and DNCR1-EGFP constructs. NS3a was localized to different subcellular compartments via N-terminal Tom20 (mitochondria), nuclear localization signal (NLS, nucleus), or myristoylation tags (plasma membrane), or a C-terminal Giantin tag (Golgi). DNCR1-EGFP was diffuse throughout the cell under DMSO treatment (FIG. 30a, left), and colocalized with NS3a-mCherry™ after treatment with 10 μM danoprevir (FIG. 30a, right). The intermediate affinity reader also exhibited colocalization when the orientation was switched and DNCR1 was fused to the localization tags, demonstrating that the CID components have good modularity, being robust to immobilization in both orientations and fusions on both termini (FIG. 30b). DNCR1 also demonstrated functional binding specificity for the danoprevir/NS3a complex, as quantification of EGFP/mCherry™ signal correlation for multiple cells showed much lower correlation in cells treated with 10 μM asunaprevir or grazoprevir (FIG. 30c,d).

Subcellular Localization Control with PROCISiR

In addition to the GNCR1/DNCR2 and DNCR2/ANR combinations used for subcellular location control of NS3a demonstrated in FIGS. 2c,d and Extended Data FIG. 5, we also demonstrated two other PROCISiR combinations for location control. Colocalization of untagged DNCR2-EGFP and GNCR1-BFP with NS3a-mCherry™-CAAX clearly exhibited 3 states: no colocalization with no drug, DNCR2/NS3a colocalization with danoprevir, and GNCR1/NS3a colocalization with grazoprevir (FIG. 27a,c). Likewise, NS3a-mCherry could be pre-localized to mitochondria with Tom20-BFP-ANR, and moved to the plasma membrane after treatment with danoprevir and binding to plasma membrane-immobilized DNCR2-EGFP-CAAX (FIG. 27b,d). Thus, the different readers can be combined to specifically respond to different drug conditions and provide multiple states of localization.

### Supplementary Note 3

### Modeling of NS3a:Drug Complex Binding

To predict drug concentration regimes that would yield intermediate levels of NS3a:DNCR2 and NS3a:GNCR1 complexes, we modeled the fraction of NS3a bound to different drugs. For this, we simply used NS3a:drug Kᵢ values and the Cheng-Prussoff approximations for equilibrium drug:receptor binding in the presence of a competitive inhibitor:⁸

$${f}_{{N}_{d}} = \frac{D}{( 1 + \frac{C}{{K}_{i , c}} ) {K}_{i , d} + D}$$

$${f}_{{N}_{c}} = \frac{C}{( 1 + \frac{D}{{K}_{i , d}} ) {K}_{i , c} + C}$$

where f_{Nd} is the fraction of NS3a bound to the target drug, and f_{Nc} is the fraction of NS3a bound to the competitor drug, D is the free concentration of target drug, C is the free concentration of competitor drug, K_{i,d} is the NS3a Kᵢ for the target drug, and K_{i,c} is the NS3a Kᵢ for the competitor drug. The following NS3a:drug Kᵢ values used are from published enzyme inhibition studies: danoprevir:NS3a, 1.0 nM, asunaprevir:NS3a 1.0 nM, grazoprevir:NS3a, 0.14 nM.^{6,9} There are several assumptions made in applying these equations that are unlikely to be valid in all cellular conditions. These include that the total drug concentrations is equal to the free drug concentration and the direct inverse relationship between f_{Nd} and f_{Nc}, which is unlikely to be true when NS3a concentrations are high. Additionally, in applying these equations to model the fractions of NS3a:drug:reader complexes, we make the further approximation that all NS3a:drug complexes will be fully bound by their corresponding reader.

Nevertheless, in comparing the predicted fraction NS3a bound to danoprevir or grazoprevir with transcriptional outputs coming from NS3a:danoprevir:DNCR2 or NS3a:grazoprevir:GNCR1, we see very good correspondence between the model and experimental results in FIG. 20c,d. In transcriptional applications, the number of relevant NS3a molecules (those occurring at promoters, from which we see output), are low, making the approximations fairly valid. We also used these equations to model the amount of DNCR2 and GNCR1 that would colocalize with membrane-bound NS3a (FIG. 31, and FIG. 21b). In that application, the number of relevant NS3a molecules is high, and we see some divergence in NS3a:DNCR2 and NS3a:GNCR1 colocalization from the model. Divergence occurs particularly at higher concentrations of danoprevir and grazoprevir, where we observe higher levels of NS3a:DNCR2 and NS3a:GNCR1 than predicted, indicative of the ability to get mixed populations of NS3a:DNCR2 and NS3a:GNCR1 complexes at higher concentrations of NS3a. However, in the absence of experimentally determined intracellular NS3a, DNCR2, and GNCR1 concentrations, these models provide a reasonable starting point for predicting the drug concentration regimes needed to get mixed, intermediate output levels.

### Supplementary Note 4

### Additional Transcriptional Control Modes

In FIG. 29a-d, we use a direct fusion of NS3a-dCas9 to direct assembly of a transcription activation complex with DNCR2-VPR or a transcriptional repression complex with DNCR2-KRAB. We use this system to control expression of two endogenous genes in HEK293 cells, CXCR4 and CD95. Detection of expression by immunofluorescence and FACS revealed expression induction of 79-fold (CXCR4) or 5-fold (CD95) over a DMSO-treated control for the DNCR2-VPR constructs, and repression induction of 1.8-fold (CXCR4) or 1.4-fold (CD95) for the DNCR2-KRAB constructs. Danoprevir had no effect on gene expression in the absence of the guide RNA. The gene induction for CXCR4 and CD95 from DNCR2-VPR surpasses that seen from similar direct-fusion chemically-induced dimerization systems using gibberellin and absisic acid.¹⁰ Inducible repression using dCas9 on endogenous promoters has not been previously demonstrated, to our knowledge.

To enable temporal switching or graded control of gene expression from repression to overexpression, we utilized a scaffold RNA/RNA-binding protein (RBP) system with NS3a fused to the RBP MS2, GNCR1 fused to VPR, and DNCR2 fused to KRAB-MeCP2, a repressor with enhanced activity over KRAB.¹¹ While more modest than the effect seen from the direct fusion system, this switchable system also demonstrated statistically significant overexpression (from grazoprevir treatment) or repression (from danoprevir treatment) of CXCR4 and CD95 (FIG. 29e,f). Notably, this was using guides that were previously published as optimal for inducing overexpression of these genes and that anneal 5′ to the transcription start site for each gene. Optimization of guide positions, or utilization of multiple guides that tile before and after the transcription start site could be explored in the future to improve the dynamic range of this switchable VPR/KRAB-MeCP2 system.

Finally, in a demonstration of the multi-state transcriptional outputs that can be achieved with PROCISiR, we combined GNCR1, DNCR2, and ANR with three orthogonal scRNA/RBP pairs (com/com, PP7/PCP, and MS2/MCP) to control the expression of CXCR4, CD95, and GFP, respectively (FIG. 29g). This system exhibited four distinct transcriptional output states under four input states: DMSO (GFP expression under control of ANR), 10 μM danoprevir (CD95 expression under control of DNCR2), 1 μM grazoprevir (CXCR4 expression under control of GNCR1), and 1 μM asunaprevir (no gene expression, as asunaprevir disrupts ANR but does not induce DNCR2 or GNCR1 complexes with NS3a-VPR). This demonstrates that all 3 readers can be used orthogonally to control different multiple output states.

TABLE 14
Sequences of constructs and primers

| Sequence | | Sequence (Reader/NS3a component in bold, |
|---|---|---|
| ID | Description | fusions/tags/linkers in regular font) |
| NS3a_1 | Tagless | MGHHHHHHHHHHGSLQDSEVNQEAKPEVKPEVKPETHINLKVSDGSSEIFFK |
| | NS3a/4a- | IKKTTPLRRLMEAFAKRQGKEMDSLRFLYDGIRIQADQAPEDLDMEDNDIIE |
| | solubility | AHREQIGGMKKKGSVVIVGRINLSGDTAYAQQTRGEEGCQETSQTGRDKNQV |
| | optimized- | EGEVQIVSTATQTFLATSINGVLWIVYHGAGIRTIASPKGPVTQMYTNVDKD |
| | S13 9A | LVGWQAPQGSRSLTPCTCGSSDLYLVTRHADVIPVRRRGDSRGSLLSPRPIS |
| | (His-SMT3 | YLKGSAGGPLLCPAGHAVGIFRAAVSTRGVAKAVDFIPVESLETTMRSP |
| | removed by | (SEQ ID NO: 78) |
| | ULP1 | |
| | cleavage) | |
| | (E. coli) | |
| DNCR2_1 | Tagless | MGHHHHHHHHHHGSLQDSEVNQEAKPEVKPEVKPETHINLKVSDGSSEIFFK |
| | DNCR2 | IKKTTPLRRLMEAFAKRQGKEMDSLRFLYDGIRIQADQAPEDLDMEDNDIIE |
| | (His-SMT3 | AHREQIGGSSDEEEARELIERAKEAAERAQEAAERTGDPRVRELARELKRLA |
| | removed by | QEAAEEVKRDPSSSDVNEALKLIVEAIEAAVDALEAAERTGDPEVRELAREL |
| | ULP1 | VRLAVEAAEEVQRNPSSSDVNEALHSIVYAIEAAIFALEAAERTGDPEVREL |
| | cleavage) | ARELVRLAVEAAEEVQRNPSSRNVEHALMRIVLAIYLAEENLREAEESGDPE |
| | | KREKARERVREAVERAEEVQRDPSGWLNH (SEQ ID NO: 79) |
| | (E. coli) | |
| DNCR1_1 | Tagless | MGHHHHHHHHHHGSLQDSEVNQEAKPEVKPEVKPETHINLKVSDGSSEIFFK |
| | DNCR1 | IKKTTPLRRLMEAFAKRQGKEMDSLRFLYDGIRIQADQAPEDLDMEDNDIIE |
| | (His-SMT3 | AHREQIGGSSDEEEARELIERAKEAAERAQEAAERTGDPRVRELARELKRLA |
| | removed by | QEAAEEVKRDPSSSDVNEALKLIVEAIEAAVDALEAAERTGDPEVRELAREL |
| | ULP1 | VRLAVEAAEEVQRNPSSSDVNEALLSIVIAIEAAVHALEAAERTGDPEVREL |
| | cleavage) | ARELVRLAVEAAEEVQRNPSSREVEHALMKIVLAIYEAEESLREAEESGDPE |
| | | KREKARERVREAVERAEEVQRDPSGWLNH (SEQ ID NO: 80) |
| | (E. coli) | |
| NS3a_2 | Avi-His₆- | MAGGLNDIFEAQKIEWHEDIGGSSHHHHHHGSGSGSMKKKGSVVIVGRINLS |
| | NS3a/4a- | GDTAYAQQTRGEEGCQETSQTGRDKNQVEGEVQIVSTATQTFLATSINGVLW |
| | solubility | TVYHGAGTRTIASPKGPVTQMYTNVDKDLVGWQAPQGSRSLTPCTCGSSDLY |
| | optimized | LVTRHADVIPVRRRGDSRGSLLSPRPISYLKGSAGGPLLCPAGHAVGIFRAA |
| | S139A | VSTRGVAKAVDFIPVESLETTMRSP (SEQ ID NO: 81) |
| | (E. coli) | |
| NS3a_3 | Avi-His₆- | MAGGLNDIFEAQKIEWHEDTGGSSHHHHHHGSGSGSMKKKGSVVIVGRINLS |
| | NS3a/4a- | GDTAYAQQTRGEEGCQETSQTGRDKNQVEGEVQIVSTATQTFLATSINGVLW |
| | solubility | TVYHGAGIRTIASPKGPVTQMYTNVDKDLVGWQAPQGSRSLTPCTCGSSDLY |
| | optimized | LVTRHADVIPVRRRGDSRGSLLSPRPISYLKGSSGGPLLCPAGHAVGIFRAA |
| | catalytica- | VSTRGVAKAVDFIPVESLETTMRSP (SEQ ID NO: 82) |
| | lly active | |
| | (E. coli) | |
| G3 | Yeast | ...KDNSSTIEGRYPYDVPDYALQASGGGGSGGGGSGGGGSASHMDIEKLCK |
| | surface | KAEEEAKEAQEKADELRQRHPDSQAAEDAEDLANEAEAAVLAACSLAQEHPN |
| | display | ADIAKLCIKAASEAAEAASKAAELAQRHPDSQAARDAIKLASQAARAVILAI |
| | G3, C- | MLAAENPNADIAKLCIKAASEAAEAASKAAELAQRHPDSQAARDAIKLASQA |
| | terminal | AEAVERAIWLAAENPNADIAKKCIKAASEAAEEASKAAEEAQRHPDSQKARD |
| | fusion to | EIKEASQKAEEVKERCKSLEGGGSEQKLISEEDL (SEQ ID NO: 83) |
| | Aga2 | |
| GNCR1 | Yeast | ...KDNSSTIEGRYPYDVPDYALQASGGGGSGGGGSGGGGSASHMDIEKLCK |
| | surface | KAEEEAKEAQEKADELRQRHPDSQAAEDAEDLANLAVAAVLTACLLAQEHPN |
| | display | ADIAKLCIKAASEAAEAASKAAELAQRHPDSQAARDAIKLASQAARAVILAI |
| | G3, C- | MLAAENPNADIAKLCIKAASEAAEAASKAAELAQRHPDSQAARDAIKLASQA |
| | terminal | AEAVERAIWLAAENPNADIAKKCIKAASEAAEEASKAAEEAQRHPDSQKARD |
| | fusion to | EIKEASQKAEEVKERCKSLEGGGSEQKLISEEDL (SEQ ID NO: 84) |
| | Aga2 | |
| D5 | Yeast | ...KDNSSTIEGRYPYDVPDYALQASGGGGSGGGGSGGGGSASHMSSDEEEA |
| | surface | RELIERAKEAAERAQEAAERTGDPRVRELARELKRLAQEAAEEVKRDPSSSD |
| | display | VNEALKLIVEAIEAAVDALEAAERTGDPEVRELARELVRLAVEAAEEVQRNP |
| | D5, C- | SSSDVNEALLTIVIAIEAAVNALEAAERTGDPEVRELARELVRLAVEAAEEV |
| | terminal | QRNPSSREVNIALWKIVLAIQEAVESLREAEESGDPEKREKARERVREAVER |
| | fusion to | AEEVQRDPSGWLNHLEGGGSEQKLISEEDL (SEQ ID NO: 85) |
| | Aga2 | |
| DNCR1 | Yeast | ...KDNSSTIEGRYPYDVPDYALQASGGGGSGGGGSGGGGSASHMSSDEEEA |
| | surface | RELIERAKEAAERAQEAAERTGDPRVRELARELKRLAQEAAEEVKRDPSSSD |
| | display | VNEALKLIVEAIEAAVDALEAAERTGDPEVRELARELVRLAVEAAEEVQRNP |
| | DNCR1, C- | SSSDVNEALLSIVIAIEAAVHALEAAERTGDPEVRELARELVRLAVEAAEEV |
| | terminal | QRNPSSREVEHALMKIVLAIYEAEESLREAEESGDPEKREKARERVREAVER |
| | fusion to | AEEVQRDPSGWLNHLEGGGSEQKLISEEDL (SEQ ID NO: 86) |
| | Aga2 | |
| DNCR2 | Yeast | ...KDNSSTIEGRYPYDVPDYALQASGGGGSGGGGSGGGGSASHMSSDEEEA |
| | surface | RELIERAKEAAERAQEAAERTGDPRVRELARELKRLAQEAAEEVKRDPSSSD |
| | display | VNEALKLIVEAIEAAVDALEAAERTGDPEVRELARELVRLAVEAAEEVQRNP |
| | DNCR2, C- | SSSDVNEALHSIVYAIEAAIFALEAAERTGDPEVRELARELVRLAVEAAEEV |
| | terminal | QRNPSSRNVEHALMRIVLAIYLAEENLREAEESGDPEKREKARERVREAVER |
| | fusion to | AEEVQRDPSGWLNHLEGGGSEQKLISEEDL (SEQ ID NO: 87) |
| | Aga2 | |
| DNCR2-EGFP | DNCR2-EGFP | MSSDEEEARELIERAKEAAERAQEAAERTGDPRVRELARELKRLAQEAAEEV |
| | (in | KRDPSSSDVNEALKLIVEAIEAAVDALEAAERTGDPEVRELARELVRLAVEA |
| | pcDNA5/FRT/ | AEEVQRNPSSSDVNEALHSIVYAIEAAIFALEAAERTGDPEVRELARELVRL |
| | TO) | AVEAAEEVQRNPSSRNVEHALMRIVLAIYLAEENLREAEESGDPEKREKARE |
| | | RVREAVERAEEVQRDPSGWLNHEQKLISEEDLGSGIGSGTMVSKGEELFTGV |
| | | VPILVELDGDVNGHKFSVSGEGEGDATYGKLTLKFICTTGKLPVPWPTLVTT |
| | | LTYGVQCFSRYPDHMKQHDFFKSAMPEGYVQERTIFFKDDGNYKTRAEVKFE |
| | | GDILVNRIELKGIDFKEDGNILGHKLEYNYNSHNVYIMADKQKNGIKVNFKI |
| | | RHNIEDGSVQLADHYQQNTPIGDGPVLLPDNHYLSTQSALSKDPNEKRDHMV |
| | | LLEFVTAAGITLGMDELYK (SEQ ID NO: 88) |
| Tom20- | Tom20- | MVGRNSAIAAGVCGALFIGYCIYFDRKRRSDPNFSSDEEEARELIERAKEAA |
| DNCR1-EGFP | DNCR1-EGFP | ERAQEAAERTGDPRVRELARELKRLAQEAAEEVKRDPSSSDVNEALKLIVEA |
| | (in | IEAAVDALEAAERTGDPEVRELARELVRLAVEAAEEVQRNPSSSDVNEALLS |
| | pcDNA5/FRT/ | IVIAIEAAVHALEAAERTGDPEVRELARELVRLAVEAAEEVQRNPSSREVEH |
| | TO) | ALMKIVLAIYEAEESLREAEESGDPEKREKARERVREAVERAEEVQRDPSGW |
| | | LNHEQKLISEEDLGSGTGSGTMVSKGEELFTGVVPILVELDGDVNGHKFSVS |
| | | GEGEGDATYGKLTLKFICTTGKLPVPWPTLVTTLTYGVQCFSRYPDHMKQHD |
| | | FFKSAMPEGYVQERTIFFKDDGNYKTRAEVKFEGDTLVNRIELKGIDEKEDG |
| | | NILGHKLEYNYNSHNVYIMADKQKNGIKVNFKIRHNIEDGSVQLADHYQQNT |
| | | PIGDGPVLLPDNHYLSTQSALSKDPNEKRDHMVLLEFVTAAGITLGMDELYK |
| | | (SEQ ID NO: 89) |
| DNCR1- | (in | MSSDEEEARELIERAKEAAERAQEAAERTGDPRVRELARELKRLAQEAAEEV |
| EGFP- | pcDNA5/FRT/ | KRDPSSSDVNEALKLIVEAIEAAVDALEAAERTGDPEVRELARELVRLAVEA |
| Giantin | TO) | AEEVQRNPSSSDVNEALLSIVIAIEAAVHALEAAERTGDPEVRELARELVRL |
| | | AVEAAEEVQRNPSSREVEHALMKIVLAIYEAEESLREAEESGDPEKREKARE |
| | | RVREAVERAEEVQRDPSGWLNHEQKLISEEDLGSGTGSGTMVSKGEELFTGV |
| | | VPILVELDGDVNGHKFSVSGEGEGDATYGKLTLKFICTTGKLPVPWPTLVTT |
| | | LTYGVQCFSRYPDHMKQHDFFKSAMPEGYVQERTIFFKDDGNYKTRAEVKFE |
| | | GDTLVNRIELKGIDFKEDGNILGHKLEYNYNSHNVYIMADKQKNGIKVNFKI |
| | | RHNIEDGSVQLADHYQQNTPIGDGPVLLPDNHYLSTQSALSKDPNEKRDHMV |
| | | LLEFVTAAGITLGMDELYKGSGTGSGSGEPQQSFSEAQQQLCNTRQEVNELR |
| | | KLLEEERDQRVAAENALSVAEEQIRRLEHSEWDSSRTPIIGSCGTQEQALLI |
| | | DLTSNSCRRTRSGVGWKRVLRSLCHSRTRVPLLAAIYFLMIHVLLILCFTGH |
| | | L (SEQ ID NO: 90) |
| 3xNLS- | (in | MDPKKKRKVDPKKKRKVDPKKKRKVSSDEEEARELIERAKEAAERAQEAAER |
| DNCR1-EGFP | pcDNA5/FRT/ | TGDPRVRELARELKRLAQEAAEEVKRDPSSSDVNEALKLIVEATEAAVDALE |
| | TO) | AAERTGDPEVRELARELVRLAVEAAEEVQRNPSSSDVNEALLSIVIAIEAAV |
| | | HALEAAERTGDPEVRELARELVRLAVEAAEEVQRNPSSREVEHALMKIVLAI |
| | | YEAEESLREAEESGDPEKREKARERVREAVERAEEVQRDPSGWLNHEQKLIS |
| | | EEDLGSGTGSGTMVSKGEELFTGVVPILVELDGDVNGHKFSVSGEGEGDATY |
| | | GKLTLKFICTTGKLPVPWPTLVTTLTYGVQCFSRYPDHMKQHDFFKSAMPEG |
| | | YVQERTIFFKDDGNYKTRAEVKFEGDTLVNRIELKGIDFKEDGNILGHKLEY |
| | | NYNSHNVYIMADKQKNGIKVNFKIRHNIEDGSVQLADHYQQNTPIGDGPVLL |
| | | PDNHYLSTQSALSKDPNEKRDHMVLLEFVTAAGITLGMDELYK (SEQ ID |
| | | NO: 91) |
| DNCR1-EGFP | (in | MSSDEEEARELIERAKEAAERAQEAAERTGDPRVRELARELKRLAQEAAEEV |
| | pcDNA5/FRT/ | KRDPSSSDVNEALKLIVEAIEAAVDALEAAERTGDPEVRELARELVRLAVEA |
| | TO) | AEEVQRNPSSSDVNEALLSIVIAIEAAVHALEAAERTGDPEVRELARELVRL |
| | | AVEAAEEVQRNPSSREVEHALMKIVLAIYEAEESLREAEESGDPEKREKARE |
| | | RVREAVERAEEVQRDPSGWLNHEQKLISEEDLGSGTGSGTMVSKGEELFTGV |
| | | VPILVELDGDVNGHKFSVSGEGEGDATYGKLTLKFICTTGKLPVPWPTLVTT |
| | | LTYGVQCFSRYPDHMKQHDFFKSAMPEGYVQERTIFFKDDGNYKTRAEVKFE |
| | | GDTLVNRIELKGIDFKEDGNILGHKLEYNYNSHNVYIMADKQKNGIKVNFKI |
| | | RHNIEDGSVQLADHYQQNTPIGDGPVLLPDNHYLSTQSALSKDPNEKRDHMV |
| | | LLEFVTAAGITLGMDELYK (SEQ ID NO: 92) |
| mCherry- | NS3a | MVSKGEEDNMAIIKEFMRFKVHMEGSVNGHEFEIEGEGEGRPYEGTQTAKLK |
| NS3a | solubility | VTKGGPLPFAWDILSPQFMYGSKAYVKHPADIPDYLKLSEPEGFKWERVMNF |
| | optimized, | EDGGVVTVTQDSSLQDGEFIYKVKLRGTNFPSDGPVMQKKTMGWEASSERMY |
| | S139A | PEDGALKGEIKQRLKLKDGGHYDAEVKTTYKAKKPVQLPGAYNVNIKLDITS |
| | (in | HNEDYTIVEQYERAEGRHSTGGMDELYKGSGTGDYKDDDDKKKKGSVVIVGR |
| | pcDNA5/FRT/ | INLSGDTAYAQQTRGEEGCQETSQTGRDKNQVEGEVQIVSTATQTFLATSIN |
| | TO) | GVLWTVYHGAGTRTIASPKGPVTQMYTNVDKDLVGWQAPQGSRSLTPCTCGS |
| | | SDLYLVTRHADVIPVRRRGDSRGSLLSPRPISYLKGSAGGPLLCPAGHAVGI |
| | | FRAAVSTRGVAKAVDFIPVESLETTMRSP (SEQ ID NO: 93) |
| Tom20- | NS3a | MVGRNSAIAAGVCGALFIGYCIYFDRKRRSDPNFGSGMVSKGEEDNMAIIKE |
| mCherry- | solubility | FMREKVHMEGSVNGHEFEIEGEGEGRPYEGTQTAKLKVTKGGPLPFAWDILS |
| NS3a | optimized, | PQFMYGSKAYVKHPADIPDYLKLSFPEGFKWERVMNFEDGGVVTVTQDSSLQ |
| | S139A | DGEFIYKVKLRGTNFPSDGPVMQKKTMGWEASSERMYPEDGALKGEIKQRLK |
| | (in | LKDGGHYDAEVKTTYKAKKPVQLPGAYNVNIKLDITSHNEDYTIVEQYERAE |
| | pcDNA5/FRT/ | GRHSTGGMDELYKGSGTGDYKDDDDKKKKGSVVIVGRINLSGDTAYAQQTRG |
| | TO) | EEGCQETSQTGRDKNQVEGEVQIVSTATQTFLATSINGVLWTVYHGAGTRTI |
| | | ASPKGPVTQMYTNVDKDLVGWQAPQGSRSLTPCTCGSSDLYLVTRHADVIPV |
| | | RRRGDSRGSLLSPRPISYLKGSAGGPLLCPAGHAVGIFRAAVSTRGVAKAVD |
| | | FIPVESLETTMRSP (SEQ ID NO: 94) |
| mCherry- | NS3a | MVSKGEEDNMAIIKEFMRFKVHMEGSVNGHEFEIEGEGEGRPYEGTQTAKLK |
| NS3a- | solubility | VTKGGPLPFAWDILSPQFMYGSKAYVKHPADIPDYLKLSEPEGFKWERVMNF |
| Giantin | optimized, | EDGGVVTVTQDSSLQDGEFIYKVKLRGTNFPSDGPVMQKKTMGWEASSERMY |
| | S139A | PEDGALKGEIKQRLKLKDGGHYDAEVKTTYKAKKPVQLPGAYNVNIKLDITS |
| | (in | HNEDYTIVEQYERAEGRHSTGGMDELYKGSGTGDYKDDDDKKKKGSVVIVGR |
| | pcDNA5/FRT/ | INLSGDTAYAQQTRGEEGCQETSQTGRDKNQVEGEVQIVSTATQTFLATSIN |
| | TO) | GVLWTVYHGAGTRTIASPKGPVTQMYTNVDKDLVGWQAPQGSRSLTPCTCGS |
| | | SDLYLVTRHADVIPVRRRGDSRGSLLSPRPISYLKGSAGGPLLCPAGHAVGI |
| | | FRAAVSTRGVAKAVDFIPVESLETTMRSPGSGTGSGSGEPQQSFSEAQQQLC |
| | | NTRQEVNELRKLLEEERDQRVAAENALSVAEEQIRRLEHSEWDSSRTPIIGS |
| | | CGTQEQALLIDLTSNSCRRTRSGVGWKRVLRSLCHSRTRVPLLAAIYFLMIH |
| | | VLLILCFTGHL (SEQ ID NO: 95) |
| 3xNLS- | NS3a | MDPKKKRKVDPKKKRKVDPKKKRKVGSGMVSKGEEDNMAIIKEFMRFKVHME |
| mCherry- | solubility | GSVNGHEFEIEGEGEGRPYEGTQTAKLKVTKGGPLPFAWDILSPQFMYGSKA |
| NS3a | optimized, | YVKHPADIPDYLKLSFPEGFKWERVMNFEDGGVVTVTQDSSLQDGEFIYKVK |
| | S139A | LRGTNFPSDGPVMQKKTMGWEASSERMYPEDGALKGEIKQRLKLKDGGHYDA |
| | (in | EVKTTYKAKKPVQLPGAYNVNIKLDITSHNEDYTIVEQYERAEGRHSTGGMD |
| | pcDNA5/FRT/ | ELYKGSGTGDYKDDDDKKKKGSVVIVGRINLSGDTAYAQQTRGEEGCQETSQ |
| | TO) | TGRDKNQVEGEVQIVSTATQTFLATSINGVLWTVYHGAGTRTIASPKGPVTQ |
| | | MYTNVDKDLVGWQAPQGSRSLTPCTCGSSDLYLVTRHADVIPVRRRGDSRGS |
| | | LLSPRPISYLKGSAGGPLLCPAGHAVGIFRAAVSTRGVAKAVDFIPVESLET |
| | | TMRSP (SEQ ID NO: 96) |
| Myristoyl- | NS3a | MGCGCSSHPEDDGSGTGSGMVSKGEEDNMAIIKEFMRFKVHMEGSVNGHEFE |
| tag- | solubility | IEGEGEGRPYEGTQTAKLKVTKGGPLPFAWDILSPQFMYGSKAYVKHPADIP |
| mCherry- | optimized, | DYLKLSFPEGFKWERVMNFEDGGVVTVTQDSSLQDGEFIYKVKLRGTNFPSD |
| NS3a | S139A | GPVMQKKTMGWEASSERMYPEDGALKGEIKQRLKLKDGGHYDAEVKTTYKAK |
| | | KPVQLPGAYNVNIKLDITSHNEDYTIVEQYERAEGRHSTGGMDELYKGSGTG |
| | (in | DYKDDDDKKKKGSVVIVGRINLSGDTAYAQQTRGEEGCQETSQTGRDKNQVE |
| | pcDNA5/FRT/ | GEVQIVSTATQTFLATSINGVLWTVYHGAGTRTIASPKGPVTQMYTNVDKDL |
| | TO) | VGWQAPQGSRSLTPCTCGSSDLYLVTRHADVIPVRRRGDSRGSLLSPRPISY |
| | | LKGSAGGPLLCPAGHAVGIFRAAVSTRGVAKAVDFIPVESLETTMRSP |
| | | (SEQ ID NO: 97) |
| NS3aH1- | ANR- | MKKKGSVVIVGRINLSGDTAYSQQTRGLEGCQETSQTGRDKNQVEGEVQVVS |
| mCherry | binding- | TATQSFLATSINGVLWTVYHGAGTRTIASPKGPVTQMYTNVDKDLVGWQAPQ |
| | competent | GSRSLTPCTCGSSDLYLVTRHADVIPVRRRGDSRGSLLSPRPISYLKGSSGG |
| | NS3a, | PLLCPAGHAVGIFRAAVSTRGVAKAVDFIPVESLETTMRSPGSGTGSGMVSK |
| | catalytica- | GEEDNMAIIKEFMRFKVHMEGSVNGHEFEIEGEGEGRPYEGTQTAKLKVTKG |
| | lly active | GPLPFAWDILSPQFMYGSKAYVKHPADIPDYLKLSEPEGFKWERVMNFEDGG |
| | (in | VVTVTQDSSLQDGEFIYKVKLRGTNFPSDGPVMQKKTMGWEASSERMYPEDG |
| | pcDNA5/FRT/ | ALKGEIKQRLKLKDGGHYDAEVKTTYKAKKPVQLPGAYNVNIKLDITSHNED |
| | TO) | YTIVEQYERAEGRHSTGGMDELYKGSGTGDYKDDDDK (SEQ ID NO: 98) |
| 3xNLS- | (in | MDPKKKRKVDPKKKRKVDPKKKRKVGSGSSDEEEARELIERAKEAAERAQEA |
| DNCR2-EGFP | pcDNA5/FRT/ | AERTGDPRVRELARELKRLAQEAAEEVKRDPSSSDVNEALKLIVEAIEAAVD |
| | TO) | ALEAAERTGDPEVRELARELVRLAVEAAEEVQRNPSSSDVNEALHSIVYAIE |
| | | AAIFALEAAERTGDPEVRELARELVRLAVEAAEEVQRNPSSRNVEHALMRIV |
| | | LAIYLAEENLREAEESGDPEKREKARERVREAVERAEEVQRDPSGWLNHEQK |
| | | LISEEDLGSGTGSGTMVSKGEELFTGVVPILVELDGDVNGHKFSVSGEGEGD |
| | | ATYGKLTLKFICTTGKLPVPWPTLVTTLTYGVQCFSRYPDHMKQHDFFKSAM |
| | | PEGYVQERTIFFKDDGNYKTRAEVKFEGDTLVNRIELKGIDEKEDGNILGHK |
| | | LEYNYNSHNVYIMADKQKNGIKVNFKIRHNIEDGSVQLADHYQQNTPIGDGP |
| | | VLLPDNHYLSTQSALSKDPNEKRDHMVLLEFVTAAGITLGMDELYK (SEQ |
| | | ID NO: 99) |
| ANR-ANR- | (in | MGELDELVYLLDGPGYDPIHSDGSGTGSGTGSGTGTTSGTGTGGSTGGELDE |
| BFP-CAAX | pcDNA5/FRT/ | LVYLLDGPGYDPIHSDGSGTGSGTGSGTGTTSGTGTGGSTGEQKLISEEDLG |
| | TO) | SGSSELIKENMHMKLYMEGTVDNHHFKCTSEGEGKPYEGTQTMRIKVVEGGP |
| | | LPFAFDILATSFLYGSKTFINHTQGIPDFFKQSFPEGFTWERVTTYEDGGVL |
| | | TATQDTSLQDGCLIYNVKIRGVNFTSNGPVMQKKTLGWEAFTETLYPADGGL |
| | | EGRNDMALKLVGGSHLIANIKTTYRSKKPAKNLKMPGVYYVDYRLERIKEAN |
| | | NETYVEQHEVAVARYCDLPSKLGHKLNRKHKEKMSKDGKKKKKKSKTKCVIM |
| | | (SEQ ID NO: 100) |
| Tom20-BFP- | (in | MVGRNSAIAAGVCGALFIGYCIYFDRKRRSDPNFMSELIKENMHMKLYMEGT |
| ANR-ANR- | pcDNA5/FRT/ | VDNHHFKCTSEGEGKPYEGTQTMRIKVVEGGPLPFAFDILATSFLYGSKTFI |
| P2a-DNCR2- | TO) | NHTQGIPDFFKQSFPEGFTWERVTTYEDGGVLTATQDTSLQDGCLIYNVKIR |
| EGFP-CAAX | | GVNFTSNGPVMQKKTLGWEAFTETLYPADGGLEGRNDMALKLVGGSHLIANI |
| | | KTTYRSKKPAKNLKMPGVYYVDYRLERIKEANNETYVEQHEVAVARYCDLPS |
| | | KLGHKLNSGSGEQKLISEEDLGSGTGSGTGSGTGTTSGTGTGGSTGGELDEL |
| | | VYLLDGPGYDPIHSDGSGTGSGTGSGTGTTSGTGTGGSTGGELDELVYLLDG |
| | | PGYDPIHSDGSGATNFSLLKQAGDVEENPGPMSSDEEEARELIERAKEAAER |
| | | AQEAAERTGDPRVRELARELKRLAQEAAEEVKRDPSSSDVNEALKLIVEAIE |
| | | AAVDALEAAERTGDPEVRELARELVRLAVEAAEEVQRNPSSSDVNEALHSIV |
| | | YAIEAAIFALEAAERTGDPEVRELARELVRLAVEAAEEVQRNPSSRNVEHAL |
| | | MRIVLAIYLAEENLREAEESGDPEKREKARERVREAVERAEEVQRDPSGWLN |
| | | HEQKLISEEDLGSGTGSGTMVSKGEELFTGVVPILVELDGDVNGHKFSVSGE |
| | | GEGDATYGKLTLKFICTTGKLPVPWPTLVTTLTYGVQCFSRYPDHMKQHDFF |
| | | KSAMPEGYVQERTIFFKDDGNYKTRAEVKFEGDTLVNRIELKGIDEKEDGNI |
| | | LGHKLEYNYNSHNVYIMADKQKNGIKVNFKIRHNIEDGSVQLADHYQQNTPI |
| | | GDGPVLLPDNHYLSTQSALSKDPNEKRDHMVLLEFVTAAGITLGMDELYKRK |
| | | HKEKMSKDGKKKKKKSKTKCVIM (SEQ ID NO: 101) |
| Tom20- | (in | MVGRNSAIAAGVCGALFIGYCIYFDRKRRSDPNFSSDEEEARELIERAKEAA |
| DNCR2-EGFP | pcDNA5/FRT/ | ERAQEAAERTGDPRVRELARELKRLAQEAAEEVKRDPSSSDVNEALKLIVEA |
| | TO) | IEAAVDALEAAERTGDPEVRELARELVRLAVEAAEEVQRNPSSSDVNEALHS |
| | | IVYAIEAAIFALEAAERTGDPEVRELARELVRLAVEAAEEVQRNPSSRNVEH |
| | | ALMRIVLAIYLAEENLREAFESGDPEKREKARERVREAVERAEEVQRDPSGW |
| | | LNHEQKLISEEDLGSGTGSGTMVSKGEELFTGVVPILVELDGDVNGHKFSVS |
| | | GEGEGDATYGKLTLKFICTTGKLPVPWPTLVTTLTYGVQCFSRYPDHMKQHD |
| | | FFKSAMPEGYVQERTIFFKDDGNYKTRAEVKFEGDTLVNRIELKGIDEKEDG |
| | | NILGHKLEYNYNSHNVYIMADKQKNGIKVNFKIRHNIEDGSVQLADHYQQNT |
| | | PIGDGPVLLPDNHYLSTQSALSKDPNEKRDHMVLLEFVTAAGITLGMDELYK |
| | | (SEQ ID NO: 102) |
| GNCR1-BFP- | (in | MDIEKLCKKAEEEAKEAQEKADELRQRHPDSQAAEDAEDLANLAVAAVLTAC |
| CAAX | pcDNA5/FRT/ | LLAQEHPNADIAKLCIKAASEAAEAASKAAELAQRHPDSQAARDAIKLASQA |
| | TO) | ARAVILAIMLAAENPNADIAKLCIKAASEAAEAASKAAELAQRHPDSQAARD |
| | | AIKLASQAAEAVERAIWLAAENPNADIAKKCIKAASEAAEEASKAAEEAQRH |
| | | PDSQKARDEIKEASQKAEEVKERCKSEQKLISEEDLGSGSSELIKENMHMKL |
| | | YMEGTVDNHHFKCTSEGEGKPYEGTQTMRIKVVEGGPLPFAFDILATSFLYG |
| | | SKTFINHTQGIPDFFKQSFPEGFTWERVTTYEDGGVLTATQDTSLQDGCLIY |
| | | NVKIRGVNFTSNGPVMQKKTLGWEAFTETLYPADGGLEGRNDMALKLVGGSH |
| | | LIANIKTTYRSKKPAKNLKMPGVYYVDYRLERIKEANNETYVEQHEVAVARY |
| | | CDLPSKLGHKLNRKHKEKMSKDGKKKKKKSKTKCVIM (SEQ ID |
| | | NO: 103) |
| NS3aH1- | ANR- | MKKKGSVVIVGRINLSGDTAYSQQTRGLEGCQETSQTGRDKNQVEGEVQVVS |
| mCherry- | binding- | TATQSFLATSINGVLWTVYHGAGTRTIASPKGPVTQMYTNVDKDLVGWQAPQ |
| CAAX | competent | GSRSLTPCTCGSSDLYLVTRHADVIPVRRRGDSRGSLLSPRPISYLKGSSGG |
| | NS3a, | PLLCPAGHAVGIFRAAVSTRGVAKAVDFIPVESLETTMRSPGSGTGSGMVSK |
| | catalytica- | GEEDNMAIIKEFMRFKVHMEGSVNGHEFEIEGEGEGRPYEGTQTAKLKVTKG |
| | lly active | GPLPFAWDILSPQFMYGSKAYVKHPADIPDYLKLSFPEGFKWERVMNFEDGG |
| | (in | VVTVTQDSSLQDGEFIYKVKLRGTNFPSDGPVMQKKTMGWEASSERMYPEDG |
| | pcDNA5/FRT/ | ALKGEIKQRLKLKDGGHYDAEVKTTYKAKKPVQLPGAYNVNIKLDITSHNED |
| | TO) | YTIVEQYERAEGRHSTGGMDELYKGSGTGDYKDDDDKQHKLRKLNPPDESGP |
| | | GCMSCKCVLS (SEQ ID NO: 104) |
| GNCR1-BFP | (in | MDIEKLCKKAEEEAKEAQEKADELRQRHPDSQAAEDAEDLANLAVAAVLTAC |
| | pcDNA5/FRT/ | LLAQEHPNADIAKLCIKAASEAAEAASKAAELAQRHPDSQAARDAIKLASQA |
| | TO) | ARAVILAIMLAAENPNADIAKLCIKAASEAAEAASKAAELAQRHPDSQAARD |
| | | AIKLASQAAEAVERAIWLAAENPNADIAKKCIKAASEAAEEASKAAEEAQRH |
| | | PDSQKARDEIKEASQKAEEVKERCKSEQKLISEEDLGSGSSELIKENMHMKL |
| | | YMEGTVDNHHFKCTSEGEGKPYEGTQTMRIKVVEGGPLPFAFDILATSFLYG |
| | | SKTFINHTQGIPDFFKQSFPEGFTWERVTTYEDGGVLTATQDTSLQDGCLIY |
| | | NVKIRGVNFTSNGPVMQKKTLGWEAFTETLYPADGGLEGRNDMALKLVGGSH |
| | | LIANIKTTYRSKKPAKNLKMPGVYYVDYRLERIKEANNETYVEQHEVAVARY |
| | | CDLPSKLGHKLN (SEQ ID NO: 105) |
| DNCR2-iSH2 | Inter-SH2 | MSSDEEEARELIERAKEAAERAQEAAERTGDPRVRELARELKRLAQEAAEEV |
| | domain of | KRDPSSSDVNEALKLIVEAIEAAVDALEAAERTGDPEVRELARELVRLAVEA |
| | human | AEEVQRNPSSSDVNEALHSIVYAIEAAIFALEAAERTGDPEVRELARELVRL |
| | PIP3K | AVEAAEEVQRNPSSRNVEHALMRIVLAIYLAEENLREAEESGDPEKREKARE |
| | fused to | RVREAVERAEEVQRDPSGWLNHEQKLISEEDLGSGTGSGTRLLYPVSKYQQD |
| | C-term of | QIVKEDSVEAVGAQLKVYHQQYQDKSREYDQLYEEYTRTSQELQMKRTAIEA |
| | DNCR2 | FNETIKIFEEQGQTQEKCSKEYLERFRREGNEKEMQRILLNSERLKSRIAEI |
| | (in | HESRTKLEQQLRAQASDNREIDKRMNSLKPDLMQLRKIRDQYLVWLTQKGAR |
| | pcDNA5/FRT/ | QKKINEWLGIKNETEDQYALMEDEDDLP (SEQ ID NO: 106) |
| | TO) | |
| DNCR2-VPR | In pB-CAG- | MSSDEEEARELIERAKEAAERAQEAAERTGDPRVRELARELKRLAQEAAEEV |
| | DNCR2-VPR- | KRDPSSSDVNEALKLIVEAIEAAVDALEAAERTGDPEVRELARELVRLAVEA |
| | IRES-Puro- | AEEVQRNPSSSDVNEALHSIVYAIEAAIFALEAAERTGDPEVRELARELVRL |
| | WPRE- | AVEAAEEVQRNPSSRNVEHALMRIVLAIYLAEENLREAEESGDPEKREKARE |
| | SV40PA- | RVREAVERAEEVQRDPSGWLNHEQKLISEEDLEFSSAAGTSDALDDFDLDML |
| | PGK- | GSDALDDFDLDMLGSDALDDFDLDMLGSDALDDFDLDMLGSPKKKRKVGSQY |
| | NS3aH1- | LPDTDDRHRIEEKRKRTYETFKSIMKKSPFSGPTDPRPPPRRIAVPSRSSAS |
| | tagBFP- | VPKPAPQPYPFTSSLSTINYDEFPTMVFPSGQISQASALAPAPPQVLPQAPA |
| | SpdCas9 | PAPAPAMVSALAQAPAPVPVLAPGPPQAVAPPAPKPTQAGEGTLSEALLQLQ |
| | | FDDEDLGALLGNSTDPAVFTDLASVDNSEFQQLLNQGIPVAPHTTEPMLMEY |
| | | PEAITRLVTGAQRPPDPAPAPLGAPGLPNGLLSGDEDFSSIADMDFSALLSQ |
| | | ISSGSGSGSRDSREGMFLPKPEAGSAISDVFEGREVCQPKRIRPFHPPGSPW |
| | | ANRPLPASLAPTPTGPVHEPVGSLTPAPVPQPLDPAPAVTPEASHLLEDPDE |
| | | ETSQAVKALREMADTVIPQKEEAAICGQMDLSHPPPRGHLDELTTTLESMTE |
| | | DLNLDSPLTPELNEILDTFLNDECLLHAMHISTGLSIFDTSLF (SEQ ID |
| | | NO: 107) |
| DNCR2-KRAB | In pB-CAG- | MSSDEEEARELIERAKEAAERAQEAAERTGDPRVRELARELKRLAQEAAEEV |
| | DNCR2- | KRDPSSSDVNEALKLIVEAIEAAVDALEAAERTGDPEVRELARELVRLAVEA |
| | KRAB-IRES- | AEEVQRNPSSSDVNEALHSIVYAIEAAIFALEAAERTGDPEVRELARELVRL |
| | Puro-WPRE- | AVEAAEEVQRNPSSRNVEHALMRIVLAIYLAEENLREAEESGDPEKREKARE |
| | SV40PA- | RVREAVERAEEVQRDPSGWLNHEQKLISEEDLEFSSAAGTSGGGGGMDAKSL |
| | PGK- | TAWSRTLVTFKDVFVDFTREEWKLLDTAQQIVYRNVMLENYKNLVSLGYQLT |
| | NS3aH1- | KPDVILRLEKGEEP (SEQ ID NO: 108) |
| | tagBFP- | |
| | SpdCas9 | |
| NS3aH1- | ANR- | MKKKGSVVIVGRINLSGDTAYSQQTRGLEGCQETSQTGRDKNQVEGEVQVVS |
| tagBFP- | binding- | TATQSFLATSINGVLWTVYHGAGTRTIASPKGPVTQMYTNVDKDLVGWQAPQ |
| SpdCas9 | competent | GSRSLTPCTCGSSDLYLVTRHADVIPVRRRGDSRGSLLSPRPISYLKGSSGG |
| | NS3a, | PLLCPAGHAVGIFRAAVSTRGVAKAVDFIPVESLETTMRSPHMSSAAGATMS |
| | catalytica- | ELIKENMHMKLYMEGTVDNHHFKCTSEGEGKPYEGTQTMRIKVVEGGPLPFA |
| | lly | FDILATSFLYGSKTFINHTQGIPDFFKQSFPEGFTWERVTTYEDGGVLTATQ |
| | active, | DTSLQDGCLIYNVKIRGVNFTSNGPVMQKKTLGWEAFTETLYPADGGLEGRN |
| | In pB-CAG- | DMALKLVGGSHLIANIKTTYRSKKPAKNLKMPGVYYVDYRLERIKEANNETY |
| | DNCR2- | VEQHEVAVARYCDLPSKLGHKLNSSAAGATMDKKYSIGLAIGTNSVGWAVIT |
| | VPR/KRAB- | DEYKVPSKKFKVLGNTDRHSIKKNLIGALLFDSGETAEATRLKRTARRRYTR |
| | IRES-Puro- | RKNRICYLQEIFSNEMAKVDDSFFHRLEESFLVEEDKKHERHPIFGNIVDEV |
| | WPRE- | AYHEKYPTIYHLRKKLVDSTDKADLRLIYLALAHMIKERGHFLIEGDLNPDN |
| | SV40PA- | SDVDKLFIQLVQTYNQLFEENPINASGVDAKAILSARLSKSRRLENLIAQLP |
| | PGK- | GEKKNGLFGNLIALSLGLTPNFKSNFDLAEDAKLQLSKDTYDDDLDNLLAQI |
| | NS3aH1- | GDQYADLFLAAKNLSDAILLSDILRVNTEITKAPLSASMIKRYDEHHQDLTL |
| | tagBFP- | LKALVRQQLPEKYKEIFFDQSKNGYAGYIDGGASQEEFYKFIKPILEKMDGT |
| | SpdCas9 | EELLVKLNREDLLRKQRTFDNGSIPHQIHLGELHAILRRQEDFYPFLKDNRE |
| | | KIEKILTFRIPYYVGPLARGNSRFAWMTRKSEETITPWNFEEVVDKGASAQS |
| | | FIERMTNFDKNLPNEKVLPKHSLLYEYFTVYNELTKVKYVTEGMRKPAFLSG |
| | | EQKKAIVDLLFKTNRKVTVKQLKEDYFKKIECEDSVEISGVEDRFNASLGTY |
| | | HDLLKIIKDKDFLDNEENEDILEDIVLTLTLFEDREMIEERLKTYAHLFDDK |
| | | VMKQLKRRRYTGWGRLSRKLINGIRDKQSGKTILDFLKSDGFANRNFMQLIH |
| | | DDSLTEKEDIQKAQVSGQGDSLHEHIANLAGSPAIKKGILQTVKVVDELVKV |
| | | MGRHKPENIVIEMARENQTTQKGQKNSRERMKRIEEGIKELGSQILKEHPVE |
| | | NTQLQNEKLYLYYLQNGRDMYVDQELDINRLSDYDVDAIVPQSFLKDDSIDN |
| | | KVLTRSDKNRGKSDNVPSEEVVKKMKNYWRQLLNAKLITQRKEDNLTKAERG |
| | | GLSELDKAGFIKRQLVETRQITKHVAQILDSRMNTKYDENDKLIREVKVITL |
| | | KSKLVSDFRKDFQFYKVREINNYHHAHDAYLNAVVGTALIKKYPKLESEFVY |
| | | GDYKVYDVRKMIAKSEQEIGKATAKYFFYSNIMNFEKTEITLANGEIRKRPL |
| | | IETNGETGEIVWDKGRDFATVRKVLSMPQVNIVKKTEVQTGGESKESILPKR |
| | | NSDKLIARKKDWDPKKYGGFDSPTVAYSVLVVAKVEKGKSKKLKSVKELLGI |
| | | TIMERSSFEKNPIDFLEAKGYKEVKKDLIIKLPKYSLFELENGRKRMLASAG |
| | | ELQKGNELALPSKYVNFLYLASHYEKLKGSPEDNEQKQLFVEQHKHYLDEII |
| | | EQISEFSKRVILADANLDKVLSAYNKHRDKPIREQAENIIHLFTLTNLGAPA |
| | | AFKYFDTTIDRKRYTSTKEVLDATLIHQSITGLYETRIDLSQLGGDAYPYDV |
| | | PDYASLGSGSPKKKRKVEDPKKKRKVDGIGSGSNG (SEQ ID NO: 109) |
| CXCR4-C1 | C1 guide | GCGGGTGGTCGGTAGTGAGTC (SEQ ID NO: 110) |
| | in | |
| | gRNA_Cloning | |
| | Vector | |
| CXCR4-C2 | C2 guide | GCAGACGCGAGGAAGGAGGGCGC (SEQ ID NO: 111) |
| | in | |
| | gRNA_Cloning | |
| | Vector | |
| CXCR4-C3 | C3 guide | GCCTCTGGGAGGTCCTGTCCGGCTC (SEQ ID NO: 112) |
| | in | |
| | gRNA_Cloning | |
| | Vector | |
| CD95-1 | CD95-1 | GTACAGCAGAAGCCTTTAGAA (SEQ ID NO: 113) |
| | guide in | |
| | gRNA_Cloning | |
| | Vector | |
| CD95-2 | CD95-2 | GTGGCATGCTCACTTCAGGTG (SEQ ID NO: 114) |
| | guide in | |
| | gRNA_Cloning | |
| | Vector | |
| CD95-3 | CD95-3 | GAAGCCTCGCTGGGGAACGCC (SEQ ID NO: 115) |
| | guide in | |
| | gRNA_Cloning | |
| | Vector | |
| TRE3G | TRE3G | GTACGTTCTCTATCACTGATA (SEQ ID NO: 116) |
| | guide in | |
| | gRNA_Cloning | |
| | Vector | |
| CXCR4-C1- | scRNA, wt + | GCGGGTGGTCGGTAGTGAGTCGTTTAAGAGCTATGCTGGAAACAGCATAGCA |
| 2xMS2 | f6 MS2 | AGTTTAAATAAGGCTAGTCCGTTATCAACTTGAAAAAGTGGCACCGAGTCGG |
| | expressed | TGCGGGAGCACATGAGGATCACCCATGTGCGACTCCCACAGTCACTGGGGAG |
| | with NLS- | TCTTCCCTTTTTTTGTTTTTTATGTCT (SEQ ID NO: 117) |
| | MCP-GNCR1- | |
| | P2a-BFP | |
| CXCR4-C2- | scRNA, 2x | GCAGACGCGAGGAAGGAGGGCGCGTTTAAGAGCTATGCTGGAAACAGCATAG |
| 2xMS2 | wt MS2 | CAAGTTTAAATAAGGCTAGTCCGTTATCAACTTGAAAAAGTGGCACCGAGTC |
| | expressed | GGTGCGGGAGCACATGAGGATCACCCATGTGCCACGAGCGACATGAGGATCA |
| | with NLS- | CCCATGTCGCTCGTGTTCCCTTTTTTTGTTTTTTATGTCT |
| | MCP-GNCR1- | (SEQ ID NO: 118) |
| | P2a-BFP | |
| CXCR4-C3- | scRNA, wt + | GCCTCTGGGAGGTCCTGTCCGGCTCGTTTAAGAGCTATGCTGGAAACAGCAT |
| 2xMS2 | f6 | AGCAAGTTTAAATAAGGCTAGTCCGTTATCAACTTGAAAAAGTGGCACCGAG |
| | expressed | TCGGTGCGGGAGCACATGAGGATCACCCATGTGCGACTCCCACAGTCACTGG |
| | with NLS- | GGAGTCTTCCCTTTTTTTGTTTTTTATGTCT (SEQ ID NO: 119) |
| | MCP-GNCR1- | |
| | P2a-BFP | |
| NLS-MCP- | Expressed | MPKKKRKVGSMASNFTQFVLVDNGGTGDVTVAPSNFANGIAEWISSNSRSQA |
| GNCR1-P2a- | with | YKVTCSVRQSSAQNRKYTIKVEVPKGAWRSYLNMELTIPIFATNSDCELIVK |
| BFP | CXCR4- | AMQGLLKDGNPIPSAIAANSGIYGSGGSGDIEKLCKKAEEEAKEAQEKADEL |
| | 2xMS2 | RQRHPDSQAAEDAEDLANLAVAAVLTACLLAQEHPNADIAKLCIKAASEAAE |
| | scRNAs | AASKAAELAQRHPDSQAARDAIKLASQAARAVILAIMLAAENPNADIAKLCI |
| | | KAASEAAEAASKAAELAQRHPDSQAARDAIKLASQAAEAVERAIWLAAENPN |
| | | ADIAKKCIKAASEAAEEASKAAEEAQRHPDSQKARDEIKEASQKAEEVKERC |
| | | KSEQKLISEEDLGSGATNFSLLKQAGDVEENPGPSELIKENMHMKLYMEGTV |
| | | DNHHFKCTSEGEGKPYEGTQTMRIKVVEGGPLPFAFDILATSFLYGSKTFIN |
| | | HTQGIPDFFKQSFPEGFTWERVTTYEDGGVLTATQDTSLQDGCLIYNVKIRG |
| | | VNFTSNGPVMQKKTLGWEAFTETLYPADGGLEGRNDMALKLVGGSHLIANIK |
| | | TTYRSKKPAKNLKMPGVYYVDYRLERIKEANNETYVEQHEVAVARYCDLPSK |
| | | LGHKLN (SEQ ID NO: 120) |
| CXCR4-C1- | scRNA | GCGGGTGGTCGGTAGTGAGTCGTTTAAGAGCTATGCTGGAAACAGCATAGCA |
| com | expressed | AGTTTAAATAAGGCTAGTCCGTTATCAACTTGAAAAAGTGGCACCGAGTCGG |
| | with NLS- | TGCCTGAATGCCTGCGAGCATCTTTTTTTGTTTTTTATGTCT (SEQ ID |
| | com-GNCR1- | NO: 121) |
| | P2a-BFP | |
| CXCR4-C2- | scRNA | GCAGACGCGAGGAAGGAGGGCGCGTTTAAGAGCTATGCTGGAAACAGCATAG |
| com | expressed | CAAGTTTAAATAAGGCTAGTCCGTTATCAACTTGAAAAAGTGGCACCGAGTC |
| | with NLS- | GGTGCCTGAATGCCTGCGAGCATCTTTTTTTGTTTTTTATGTCT (SEQ |
| | com-GNCR1- | ID NO: 122) |
| | P2a-BFP | |
| CXCR4-C3- | scRNA | GCCTCTGGGAGGTCCTGTCCGGCTCGTTTAAGAGCTATGCTGGAAACAGCAT |
| com | expressed | AGCAAGTTTAAATAAGGCTAGTCCGTTATCAACTTGAAAAAGTGGCACCGAG |
| | with NLS- | TCGGTGCCTGAATGCCTGCGAGCATCTTTTTTTGTTTTTTATGTCT (SEQ |
| | com-GNCR1- | ID NO: 123) |
| | P2a-BFP | |
| NLS-com- | Expressed | MPKKKRKVGSMKSIRCKNCNKLLFKADSFDHIEIRCPRCKRHIIMLNACEHP |
| GNCR1-P2a- | with | TEKHCGKREKITHSDETVRYGSGSGSGDIEKLCKKAEEEAKEAQEKADELRQ |
| BFP | CXCR4-com | RHPDSQAAEDAEDLANLAVAAVLTACLLAQEHPNADIAKLCIKAASEAAEAA |
| | scRNAs | SKAAELAQRHPDSQAARDAIKLASQAARAVILAIMLAAENPNADIAKLCIKA |
| | | ASEAAEAASKAAELAQRHPDSQAARDAIKLASQAAEAVERAIWLAAENPNAD |
| | | IAKKCIKAASEAAEEASKAAEEAQRHPDSQKARDEIKEASQKAEEVKERCKS |
| | | EQKLISEEDLGSGATNFSLLKQAGDVEENPGPSELIKENMHMKLYMEGTVDN |
| | | HHFKCTSEGEGKPYEGTQTMRIKVVEGGPLPFAFDILATSFLYGSKTFINHT |
| | | QGIPDFFKQSFPEGFTWERVTTYEDGGVLTATQDTSLQDGCLIYNVKIRGVN |
| | | FTSNGPVMQKKTLGWEAFTETLYPADGGLEGRNDMALKLVGGSHLIANIKTT |
| | | YRSKKPAKNLKMPGVYYVDYRLERIKEANNETYVEQHEVAVARYCDLPSKLG |
| | | HKLN (SEQ ID NO: 124) |
| CD95-1- | scRNA | GTACAGCAGAAGCCTTTAGAAGTTTAAGAGCTATGCTGGAAACAGCATAGCA |
| 2xPP7 | expressed | AGTTTAAATAAGGCTAGTCCGTTATCAACTTGAAAAAGTGGCACCGAGTCGG |
| | with NLS- | TGCGGGAGCTAAGGAGTTTATATGGAAACCCTTAGCCTGCTGCGTAAGGAGT |
| | PCP-DNCR2- | TTATATGGAAACCCTTACGCAGCAGTTCCCTTTTTTTGTTTTTTATGTCT |
| | P2a-BFP | (SEQ ID NO: 125) |
| CD95-2- | scRNA | GTGGCATGCTCACTTCAGGTGGTTTAAGAGCTATGCTGGAAACAGCATAGCA |
| 2xPP7 | expressed | AGTTTAAATAAGGCTAGTCCGTTATCAACTTGAAAAAGTGGCACCGAGTCGG |
| | with NLS- | TGCGGGAGCTAAGGAGTTTATATGGAAACCCTTAGCCTGCTGCGTAAGGAGT |
| | PCP-DNCR2- | TTATATGGAAACCCTTACGCAGCAGTTCCCTTTTTTTGTTTTTTATGTCT |
| | P2a-BFP | (SEQ ID NO: 126) |
| CD95-3- | scRNA | GAAGCCTCGCTGGGGAACGCCGTTTAAGAGCTATGCTGGAAACAGCATAGCA |
| 2xPP7 | expressed | AGTTTAAATAAGGCTAGTCCGTTATCAACTTGAAAAAGTGGCACCGAGTCGG |
| | with NLS- | TGCGGGAGCTAAGGAGTTTATATGGAAACCCTTAGCCTGCTGCGTAAGGAGT |
| | PCP-DNCR2- | TTATATGGAAACCCTTACGCAGCAGTTCCCTTTTTTTGTTTTTTATGTCT |
| | P2a-BFP | (SEQ ID NO: 127) |
| TRE3G- | scRNA | GTACGTTCTCTATCACTGATAGTTTAAGAGCTATGCTGGAAACAGCATAGCA |
| 2xPP7 | expressed | AGTTTAAATAAGGCTAGTCCGTTATCAACTTGAAAAAGTGGCACCGAGTCGG |
| | with NLS- | TGCGGGAGCTAAGGAGTTTATATGGAAACCCTTAGCCTGCTGCGTAAGGAGT |
| | PCP-DNCR2- | TTATATGGAAACCCTTACGCAGCAGTTCCCTTTTTTTGTTTTTTATGTCT |
| | P2a-BFP | (SEQ ID NO: 128) |
| NLS-PCP- | Expressed | MPKKKRKVGSMSKTIVLSVGEATRTLTEIQSTADRQIFEEKVGPLVGRLRLT |
| DNCR2-P2a- | with | ASLRQNGAKTAYRVNLKLDQADVVDSGLPKVRYTQVWSHDVTIVANSTEASR |
| BFP | TRE3G- | KSLYDLTKSLVATSQVEDLVVNLVPLGRGSGSGSSDEEEARELIERAKEAAE |
| | 2xPP7 or | RAQEAAERTGDPRVRELARELKRLAQEAAEEVKRDPSSSDVNEALKLIVEAI |
| | CD95-2xPP7 | EAAVDALEAAERTGDPEVRELARELVRLAVEAAEEVQRNPSSSDVNEALHSI |
| | scRNAs | VYAIEAAIFALEAAERTGDPEVRELARELVRLAVEAAEEVQRNPSSRNVEHA |
| | | LMRIVLAIYLAEENLREAEESGDPEKREKARERVREAVERAEEVQRDPSGWL |
| | | NHEQKLISEEDLGSGATNFSLLKQAGDVEENPGPSELIKENMHMKLYMEGTV |
| | | DNHHFKCTSEGEGKPYEGTQTMRIKVVEGGPLPFAFDILATSFLYGSKTFIN |
| | | HTQGIPDFFKQSFPEGFTWERVTTYEDGGVLTATQDTSLQDGCLIYNVKIRG |
| | | VNFTSNGPVMQKKTLGWEAFTETLYPADGGLEGRNDMALKLVGGSHLIANIK |
| | | TTYRSKKPAKNLKMPGVYYVDYRLERIKEANNETYVEQHEVAVARYCDLPSK |
| | | LGHKLN (SEQ ID NO: 129) |
| TRE3G- | scRNA, | GTACGTTCTCTATCACTGATAGTTTAAGAGCTATGCTGGAAACAGCATAGCA |
| 2xMS2 | wt + f6 MS2 | AGTTTAAATAAGGCTAGTCCGTTATCAACTTGAAAAAGTGGCACCGAGTCGG |
| | expressed | TGCGGGAGCACATGAGGATCACCCATGTGCGACTCCCACAGTCACTGGGGAG |
| | with NLS- | TCTTCCCTTTTTTTGTTTTTTATGTCT (SEQ ID NO: 130) |
| | MCP-ANR- | |
| | ANR-P2a- | |
| | BFP | |
| NLS-MCP- | Expressed | MPKKKRKVGSMASNFTQFVLVDNGGTGDVTVAPSNFANGIAEWISSNSRSQA |
| ANR-ANR- | with | YKVTCSVRQSSAQNRKYTIKVEVPKGAWRSYLNMELTIPIFATNSDCELIVK |
| P2a-BFP | TRE3G- | AMQGLLKDGNPIPSAIAANSGIYGSGGSGTGSGTGSGTGTTSGTGTGGSTGG |
| | 2xMS2 | ELDELVYLLDGPGYDPIHSDGSGTGSGTGSGTGTTSGTGTGGSTGGELDELV |
| | | YLLDGPGYDPIHSDGSGATNESLLKQAGDVEENPGPSELIKENMHMKLYMEG |
| | | TVDNHHFKCTSEGEGKPYEGTQTMRIKVVEGGPLPFAFDILATSFLYGSKTF |
| | | INHTQGIPDFFKQSFPEGFTWERVTTYEDGGVLTATQDTSLQDGCLIYNVKI |
| | | RGVNFTSNGPVMQKKTLGWEAFTETLYPADGGLEGRNDMALKLVGGSHLIAN |
| | | IKTTYRSKKPAKNLKMPGVYYVDYRLERIKEANNETYVEQHEVAVARYCDLP |
| | | SKLGHKLN (SEQ ID NO: 131) |
| NS3aH1-VPR | ANR- | MKKKGSVVIVGRINLSGDTAYSQQTRGLEGCQETSQTGRDKNQVEGEVQVVS |
| | binding- | TATQSFLATSINGVLWTVYHGAGTRTIASPKGPVTQMYTNVDKDLVGWQAPQ |
| | competent | GSRSLTPCTCGSSDLYLVTRHADVIPVRRRGDSRGSLLSPRPISYLKGSSGG |
| | NS3a, | PLLCPAGHAVGIFRAAVSTRGVAKAVDFIPVESLETTMRSPGSGTGSGEQKL |
| | catalytica- | ISEEDLEFSSAAGTSDALDDFDLDMLGSDALDDFDLDMLGSDALDDFDLDML |
| | lly | GSDALDDFDLDMLGSPKKKRKVGSQYLPDTDDRHRIEEKRKRTYETFKSIMK |
| | active, | KSPFSGPTDPRPPPRRIAVPSRSSASVPKPAPQPYPFTSSLSTINYDEFPTM |
| | In | VFPSGQISQASALAPAPPQVLPQAPAPAPAPAMVSALAQAPAPVPVLAPGPP |
| | pcDNA5/FRT/ | QAVAPPAPKPTQAGEGTLSEALLQLQFDDEDLGALLGNSTDPAVFTDLASVD |
| | TO | NSEFQQLLNQGIPVAPHTTEPMLMEYPEAITRLVTGAQRPPDPAPAPLGAPG |
| | | LPNGLLSGDEDFSSIADMDFSALLSQISSGSGSGSRDSREGMFLPKPEAGSA |
| | | ISDVFEGREVCQPKRIRPFHPPGSPWANRPLPASLAPTPTGPVHEPVGSLTP |
| | | APVPQPLDPAPAVTPEASHLLEDPDEETSQAVKALREMADTVIPQKEEAAIC |
| | | GQMDLSHPPPRGHLDELTTTLESMTEDLNLDSPLTPELNEILDTFLNDECLL |
| | | HAMHISTGLSIFDTSLF (SEQ ID NO: 132) |
| dCas9 | N-terminal | MDYKDDDDKDKKYSIGLAIGTNSVGWAVITDEYKVPSKKFKVLGNTDRHSIK |
| | FLAG-Sp- | KNLIGALLFDSGETAEATRLKRTARRRYTRRKNRICYLQEIFSNEMAKVDDS |
| | dCas9- | FFHRLEESFLVEEDKKHERHPIFGNIVDEVAYHEKYPTIYHLRKKLVDSTDK |
| | SV40-NLS | ADLRLIYLALAHMIKFRGHFLIEGDLNPDNSDVDKLFIQLVQTYNQLFEENP |
| | in | INASGVDAKAILSARLSKSRRLENLIAQLPGEKKNGLFGNLIALSLGLTPNF |
| | pCDNA5/FRT/ | KSNFDLAEDAKLQLSKDTYDDDLDNLLAQIGDQYADLFLAAKNLSDAILLSD |
| | TO | ILRVNTEITKAPLSASMIKRYDEHHQDLTLLKALVRQQLPEKYKEIFFDQSK |
| | | NGYAGYIDGGASQEEFYKFIKPILEKMDGTEELLVKLNREDLLRKQRTFDNG |
| | | SIPHQIHLGELHAILRRQEDFYPFLKDNREKIEKILTFRIPYYVGPLARGNS |
| | | RFAWMTRKSEETITPWNFEEVVDKGASAQSFIERMTNFDKNLPNEKVLPKHS |
| | | LLYEYFTVYNELTKVKYVTEGMRKPAFLSGEQKKAIVDLLFKTNRKVTVKQL |
| | | KEDYFKKIECFDSVEISGVEDRFNASLGTYHDLLKIIKDKDFLDNEENEDIL |
| | | EDIVLTLTLFEDREMIEERLKTYAHLFDDKVMKQLKRRRYTGWGRLSRKLIN |
| | | GIRDKQSGKTILDFLKSDGFANRNFMQLIHDDSLTEKEDIQKAQVSGQGDSL |
| | | HEHIANLAGSPAIKKGILQTVKVVDELVKVMGRHKPENIVIEMARENQTTQK |
| | | GQKNSRERMKRIEEGIKELGSQILKEHPVENTQLQNEKLYLYYLQNGRDMYV |
| | | DQELDINRLSDYDVDAIVPQSFLKDDSIDNKVLTRSDKNRGKSDNVPSEEVV |
| | | KKMKNYWRQLLNAKLITQRKEDNLTKAERGGLSELDKAGFIKRQLVETRQIT |
| | | KHVAQILDSRMNTKYDENDKLIREVKVITLKSKLVSDFRKDFQFYKVREINN |
| | | YHHAHDAYLNAVVGTALIKKYPKLESEFVYGDYKVYDVRKMIAKSEQEIGKA |
| | | TAKYFFYSNIMNFEKTEITLANGEIRKRPLIETNGETGEIVWDKGRDFATVR |
| | | KVLSMPQVNIVKKTEVQTGGESKESILPKRNSDKLIARKKDWDPKKYGGFDS |
| | | PTVAYSVLVVAKVEKGKSKKLKSVKELLGITIMERSSFEKNPIDFLEAKGYK |
| | | EVKKDLIIKLPKYSLFELENGRKRMLASAGELQKGNELALPSKYVNFLYLAS |
| | | HYEKLKGSPEDNEQKQLFVEQHKHYLDEIIEQISEFSKRVILADANLDKVLS |
| | | AYNKHRDKPIREQAENIIHLFTLTNLGAPAAFKYFDTTIDRKRYTSTKEVLD |
| | | ATLIHQSITGLYETRIDLSQLGGDSRADPKKKRKV (SEQ ID NO: 133) |
| CXCR4-C1- | pU6-guide- | GCGGGTGGTCGGTAGTGAGTCGTTTAAGAGCTATGCTGGAAACAGCATAGCA |
| 2xMS2 | 2xMS2(wt + f | AGTTTAAATAAGGCTAGTCCGTTATCAACTTGAAAAAGTGGCACCGAGTCGG |
| | 6)/CMV-BFP | TGCGGGAGCACATGAGGATCACCCATGTGCGACTCCCACAGTCACTGGGGAG |
| | | TCTTCCCTTTTTTTGTTTTTTATGTCT (SEQ ID NO: 134) |
| CXCR4-C2- | pU6-guide- | GCAGACGCGAGGAAGGAGGGCGCGTTTAAGAGCTATGCTGGAAACAGCATAG |
| 2xMS2 | 2xMS2(wt + f | CAAGTTTAAATAAGGCTAGTCCGTTATCAACTTGAAAAAGTGGCACCGAGTC |
| | 6)/CMV-BFP | GGTGCGGGAGCACATGAGGATCACCCATGTGCGACTCCCACAGTCACTGGGG |
| | | AGTCTTCCCTTTTTTTGTTTTTTATGTCT (SEQ ID NO: 135) |
| CXCR4-C3- | pU6-guide- | GCCTCTGGGAGGTCCTGTCCGGCTCGTTTAAGAGCTATGCTGGAAACAGCAT |
| 2xMS2 | 2xMS2(wt + f | AGCAAGTTTAAATAAGGCTAGTCCGTTATCAACTTGAAAAAGTGGCACCGAG |
| | 6)/CMV-BFP | TCGGTGCGGGAGCACATGAGGATCACCCATGTGCGACTCCCACAGTCACTGG |
| | | GGAGTCTTCCCTTTTTTTGTTTTTTATGTCT (SEQ ID NO: 136) |
| CD95-1- | pU6-guide- | GTACAGCAGAAGCCTTTAGAAGTTTAAGAGCTATGCTGGAAACAGCATAGCA |
| 2xMS2 | 2xMS2(wt + f | AGTTTAAATAAGGCTAGTCCGTTATCAACTTGAAAAAGTGGCACCGAGTCGG |
| | 6)/CMV-BFP | TGCGGGAGCACATGAGGATCACCCATGTGCGACTCCCACAGTCACTGGGGAG |
| | | TCTTCCCTTTTTTTGTTTTTTATGTCT (SEQ ID NO: 137) |
| CD95-2- | pU6-guide- | GTGGCATGCTCACTTCAGGTGGTTTAAGAGCTATGCTGGAAACAGCATAGCA |
| 2xMS2 | 2xMS2(wt + f | AGTTTAAATAAGGCTAGTCCGTTATCAACTTGAAAAAGTGGCACCGAGTCGG |
| | 6)/CMV-BFP | TGCGGGAGCACATGAGGATCACCCATGTGCGACTCCCACAGTCACTGGGGAG |
| | | TCTTCCCTTTTTTTGTTTTTTATGTCT (SEQ ID NO: 138) |
| CD95-3- | pU6-guide- | GAAGCCTCGCTGGGGAACGCCGTTTAAGAGCTATGCTGGAAACAGCATAGCA |
| 2xMS2 | 2xMS2(wt + f | AGTTTAAATAAGGCTAGTCCGTTATCAACTTGAAAAAGTGGCACCGAGTCGG |
| | 6)/CMV-BFP | TGCGGGAGCACATGAGGATCACCCATGTGCGACTCCCACAGTCACTGGGGAG |
| | | TCTTCCCTTTTTTTGTTTTTTATGTCT (SEQ ID NO: 139) |
| MCP-NS3a- | NS3a | MPKKKRKVGSMASNFTQFVLVDNGGTGDVTVAPSNFANGIAEWISSNSRSQA |
| P2a-DNCR2- | solubility | YKVTCSVRQSSAQNRKYTIKVEVPKGAWRSYLNMELTIPIFATNSDCELIVK |
| KRAB- | optimized | AMQGLLKDGNPIPSAIAANSGIYGSGGSGTGEQKLISEEDLGGKKKGSVVIV |
| MeCP2-P2a- | S139A, | GRINLSGDTAYAQQTRGEEGCQETSQTGRDKNQVEGEVQIVSTATQTFLATS |
| GNCR1-VPR- | DNCR2, and | INGVLWTVYHGAGTRTIASPKGPVTQMYTNVDKDLVGWQAPQGSRSLTPCTC |
| (IRES-BFP) | GNCR1 all | GSSDLYLVTRHADVIPVRRRGDSRGSLLSPRPISYLKGSAGGPLLCPAGHAV |
| | in bold | GIFRAAVSTRGVAKAVDFIPVESLETTMRSPGSGATNFSLLKQAGDVEENPG |
| | (in | PMSSDEEEARELIERAKEAAERAQEAAERTGDPRVRELARELKRLAQEAAEE |
| | pCDNA5/FRT/ | VKRDPSSSDVNEALKLIVEAIEAAVDALEAAERTGDPEVRELARELVRLAVE |
| | TO) | AAEEVQRNPSSSDVNEALHSIVYAIEAAIFALEAAERTGDPEVRELARELVR |
| | | LAVEAAEEVQRNPSSRNVEHALMRIVLAIYLAEENLREAEESGDPEKREKAR |
| | | ERVREAVERAEEVQRDPSGWLNHEQKLISEEDLSGGGSGGSGSMDAKSLTAW |
| | | SRTLVTFKDVFVDFTREEWKLLDTAQQIVYRNVMLENYKNLVSLGYQLTKPD |
| | | VILRLEKGEEPWLVSGGGSGGSGSSPKKKRKVEASVQVKRVLEKSPGKLLVK |
| | | MPFQASPGGKGEGGGATTSAQVMVIKRPGRKRKAEADPQAIPKKRGRKPGSV |
| | | VAAAAAEAKKKAVKESSIRSVQETVLPIKKRKTRETVSIEVKEVVKPLLVST |
| | | LGEKSGKGLKTCKSPGRKSKESSPKGRSSSASSPPKKEHHHHHHHAESPKAP |
| | | MPLLPPPPPPEPQSSEDPISPPEPQDLSSSICKEEKMPRAGSLESDGCPKEP |
| | | AKTQPMVAAAATTTTTTTTTVAEKYKHRGEGERKDIVSSSMPRPNREEPVDS |
| | | RTPVTERVSGSGATNFSLLKQAGDVEENPGPDIEKLCKKAEEEAKEAQEKAD |
| | | ELRQRHPDSQAAEDAEDLANLAVAAVLTACLLAQEHPNADIAKLCIKAASEA |
| | | AEAASKAAELAQRHPDSQAARDAIKLASQAARAVILAIMLAAENPNADIAKL |
| | | CIKAASEAAEAASKAAELAQRHPDSQAARDAIKLASQAAEAVERAIWLAAEN |
| | | PNADIAKKCIKAASEAAEEASKAAEEAQRHPDSQKARDEIKEASQKAEEVEE |
| | | RCKSEQKLISEEDLEFSSAAGTSDALDDFDLDMLGSDALDDFDLDMLGSDAL |
| | | DDFDLDMLGSDALDDFDLDMLGSPKKKRKVGSQYLPDTDDRHRIEEKRKRTY |
| | | ETFKSIMKKSPFSGPTDPRPPPRRIAVPSRSSASVPKPAPQPYPFTSSLSTI |
| | | NYDEEPTMVEPSGQISQASALAPAPPQVLPQAPAPAPAPAMVSALAQAPAPV |
| | | PVLAPGPPQAVAPPAPKPTQAGEGTLSEALLQLQFDDEDLGALLGNSTDPAV |
| | | FTDLASVDNSEFQQLLNQGIPVAPHTTEPMLMEYPEAITRLVTGAQRPPDPA |
| | | PAPLGAPGLPNGLLSGDEDFSSIADMDFSALLSQISSGSGSGSRDSREGMFL |
| | | PKPEAGSAISDVFEGREVCQPKRIRPFHPPGSPWANRPLPASLAPTPTGPVH |
| | | EPVGSLTPAPVPQPLDPAPAVTPEASHLLEDPDEETSQAVKALREMADTVIP |
| | | QKEEAAICGQMDLSHPPPRGHLDELTTTLESMTEDLNLDSPLTPELNEILDT |
| | | FLNDECLLHAMHISTGLSIFDTSLF (SEQ ID NO: 140) |
| LifeAct- | (in | MGVADLIKKFESISKEEGDPPVATMVSKGEEDNMAIIKEFMREKVHMEGSVN |
| mCherry | pCDNA5/FRT/ | GHEFEIEGEGEGRPYEGTQTAKLKVTKGGPLPFAWDILSPQFMYGSKAYVKH |
| | TO) | PADIPDYLKLSFPEGFKWERVMNFEDGGVVTVTQDSSLQDGEFIYKVKLRGT |
| | | NFPSDGPVMQKKTMGWEASSERMYPEDGALKGEIKQRLKLKDGGHYDAEVKT |
| | | TYKAKKPVQLPGAYNVNIKLDITSHNEDYTIVEQYERAEGRHSTGGMDELYK |
| | | (SEQ ID NO: 141) |
| mCherry- | NS3a | MVSKGEEDNMAIIKEFMRFKVHMEGSVNGHEFEIEGEGEGRPYEGTQTAKLK |
| NS3a-CAAX- | solubility | VTKGGPLPFAWDILSPQFMYGSKAYVKHPADIPDYLKLSEPEGFKWERVMNF |
| (IRES)- | optimized | EDGGVVTVTQDSSLQDGEFIYKVKLRGTNFPSDGPVMQKKTMGWEASSERMY |
| EGFP- | S139A. | PEDGALKGEIKQRLKLKDGGHYDAEVKTTYKAKKPVQLPGAYNVNIKLDITS |
| DCNR2-P2a- | CAAX from | HNEDYTIVEQYERAEGRHSTGGMDELYKGSGTGEQKLISEEDLGGKKKGSVV |
| BFP-GNCR1 | KRAS4b. | IVGRINLSGDTAYAQQTRGEEGCQETSQTGRDKNQVEGEVQIVSTATQTFLA |
| | IRES not | TSINGVLWTVYHGAGTRTIASPKGPVTQMYTNVDKDLVGWQAPQGSRSLTPC |
| | shown. | TCGSSDLYLVTRHADVIPVRRRGDSRGSLLSPRPISYLKGSAGGPLLCPAGH |
| | NS3a, | AVGIFRAAVSTRGVAKAVDFIPVESLETTMRSPSAGGSAGGEKMSKDGKKKK |
| | DNCR1, and | KKSKTKCVIM - (IRES) - |
| | GNCR1 in | MVSKGEELFTGVVPILVELDGDVNGHKFSVSGEGEGDATYGKLTLKFICTTG |
| | bold (in | KLPVPWPTLVTTLTYGVQCFSRYPDHMKQHDFFKSAMPEGYVQERTIFFKDD |
| | pEF5-FRT) | GNYKTRAEVKFEGDTLVNRIELKGIDFKEDGNILGHKLEYNYNSHNVYIMAD |
| | | KQKNGIKVNFKIRHNIEDGSVQLADHYQQNTPIGDGPVLLPDNHYLSTQSAL |
| | | SKDPNEKRDHMVLLEFVTAAGITLGMDELYKSGSGEQKLISEEDLGSGSSDE |
| | | EEARELIERAKEAAERAQEAAERTGDPRVRELARELKRLAQEAAEEVKRDPS |
| | | SSDVNEALKLIVEAIEAAVDALEAAERTGDPEVRELARELVRLAVEAAEEVQ |
| | | RNPSSSDVNEALHSIVYAIEAAIFALEAAERTGDPEVRELARELVRLAVEAA |
| | | EEVQRNPSSRNVEHALMRIVLAIYLAEENLREAEESGDPEKREKARERVREA |
| | | VERAEEVQRDPSGWLNHSAGGSAGGSAGGSAGGSGASGSGATNFSLLKQAGD |
| | | VEENPGPSELIKENMHMKLYMEGTVDNHHFKCTSEGEGKPYEGTQTMRIKVV |
| | | EGGPLPFAFDILATSFLYGSKTFINHTQGIPDFFKQSFPEGFTWERVTTYED |
| | | GGVLTATQDTSLQDGCLIYNVKIRGVNFTSNGPVMQKKTLGWEAFTETLYPA |
| | | DGGLEGRNDMALKLVGGSHLIANIKTTYRSKKPAKNLKMPGVYYVDYRLERI |
| | | KEANNETYVEQHEVAVARYCDLPSKLGHKLNSGSGEQKLISEEDLGSGTGSG |
| | | TGSGTGTTSGTGTGGSTGMDIEKLCKKAEEEAKEAQEKADELRQRHPDSQAA |
| | | EDAEDLANLAVAAVLTACLLAQEHPNADIAKLCIKAASEAAEAASKAAELAQ |
| | | RHPDSQAARDAIKLASQAARAVILAIMLAAENPNADIAKLCIKAASEAAEAA |
| | | SKAAELAQRHPDSQAARDAIKLASQAAEAVERAIWLAAENPNADIAKKCIKA |
| | | ASEAAEEASKAAEEAQRHPDSQKARDEIKEASQKAEEVKERCKSSAGGSAGG |
| | | SAGGSAGGSAG (SEQ ID NO: 142) |
| NS3a-CAAX- | NS3a | MEQKLISEEDLGGKKKGSVVIVGRINLSGDTAYAQQTRGEEGCQETSQTGRD |
| (IRES)- | solubility | KNQVEGEVQIVSTATQTFLATSINGVLWTVYHGAGTRTIASPKGPVTQMYTN |
| EGFP- | optimized | VDKDLVGWQAPQGSRSLTPCTCGSSDLYLVTRHADVIPVRRRGDSRGSLLSP |
| DCNR2- | S139A. | RPISYLKGSAGGPLLCPAGHAVGIFRAAVSTRGVAKAVDFIPVESLETTMRS |
| TIAM-P2a- | CAAX from | PSAGGSAGGEKMSKDGKKKKKKSKTKCVIM - (IRES) - |
| BFP-GNCR1- | KRAS4b. | MVSKGEELFTGVVPILVELDGDVNGHKFSVSGEGEGDATYGKLTLKFICTTG |
| LARG | IRES not | KLPVPWPTLVTTLTYGVQCFSRYPDHMKQHDFFKSAMPEGYVQERTIFFKDD |
| | shown. | GNYKTRAEVKFEGDTLVNRIELKGIDFKEDGNILGHKLEYNYNSHNVYIMAD |
| | NS3a, | KQKNGIKVNFKIRHNIEDGSVQLADHYQQNTPIGDGPVLLPDNHYLSTQSAL |
| | DNCR1, and | SKDPNEKRDHMVLLEFVTAAGITLGMDELYKSGSGEQKLISEEDLGSGSSDE |
| | GNCR1 in | EEARELIERAKEAAERAQEAAERTGDPRVRELARELKRLAQEAAEEVKRDPS |
| | bold. TIAM | SSDVNEALKLIVEATEAAVDALEAAERTGDPEVRELARELVRLAVEAAEEVQ |
| | and LARG | RNPSSSDVNEALHSIVYAIEAAIFALEAAERTGDPEVRELARELVRLAVEAA |
| | underlined | EEVQRNPSSRNVEHALMRIVLAIYLAEENLREAEESGDPEKREKARERVREA |
| | (in | VERAEEVQRDPSGWLNHSAGGSAGGSAGGSAGGSGASRQLSDADKLRKVICE |
| | PB501B) | LLETERTYVKDLNCLMERYLKPLQKETFLTQDELDVLFGNLTEMVEFQVEFL |
| | | KTLEDGVRLVPDLEKLEKVDQFKKVLFSLGGSFLYYADRFKLYSAFCASHTK |
| | | VPKVLVKAKTDTAFKAFLDAQNPKQQHSSTLESYLIKPIQRILKYPLLLREL |
| | | FALTDAESEEHYHLDVAIKTMNKVASHINEMQKIHEEGSGATNESLLKQAGD |
| | | VEENPGPSELIKENMHMKLYMEGTVDNHHFKCTSEGEGKPYEGTQTMRIKVV |
| | | EGGPLPFAFDILATSFLYGSKTFINHTQGIPDFFKQSFPEGFTWERVTTYED |
| | | GGVLTATQDTSLQDGCLIYNVKIRGVNFTSNGPVMQKKTLGWEAFTETLYPA |
| | | DGGLEGRNDMALKLVGGSHLIANIKTTYRSKKPAKNLKMPGVYYVDYRLERI |
| | | KEANNETYVEQHEVAVARYCDLPSKLGHKLNSGSGEQKLISEEDLGSGTGSG |
| | | TGSGTGTTSGTGTGGSTGMDIEKLCKKAEEEAKEAQEKADELRQRHPDSQAA |
| | | EDAEDLANLAVAAVLTACLLAQEHPNADIAKLCIKAASEAAEAASKAAELAQ |
| | | RHPDSQAARDAIKLASQAARAVILAIMLAAENPNADIAKLCIKAASEAAEAA |
| | | SKAAELAQRHPDSQAARDAIKLASQAAEAVERAIWLAAENPNADIAKKCIKA |
| | | ASEAAEEASKAAEEAQRHPDSQKARDEIKEASQKAEEVKERCKSSAGGSAGG |
| | | SAGGSAGGSAGTPPNWQQLVSREVLLGLKPCEIKRQEVINELFYTERAHVRT |
| | | LKVLDQVFYQRVSREGILSPSELRKIFSNLEDILQLHIGLNEQMKAVRKRNE |
| | | TSVIDQIGEDLLTWFSGPGEEKLKHAAATFCSNQPFALEMIKSRQKKDSRFQ |
| | | TFVQDAESNPLCRRLQLKDIIPTQMQRLTKYPLLLDNIAKYTEWPTEREKVK |
| | | KAADHCRQILNYVNQAVKEAENKQR (SEQ ID NO: 143) |
| Gal4DBD- | From | MKLLSSIEQACDICRLKKLKCSKEKPKCAKCLKNNWECRYSPKTKRSPLTRA |
| NS3a-P2a- | pLenti- | HLTEVESRLERLEQLFLLIFPREDLDMILKMDSLQDIKALLGTPAAASTLEG |
| DNCR2-VPR | UAS- | GGSAGSGGKKKGSVVIVGRINLSGDTAYAQQTRGEEGCQETSQTGRDKNQVE |
| | minCMV- | GEVQIVSTATQTFLATSINGVLWTVYHGAGTRTIASPKGPVTQMYTNVDKDL |
| | mCherry/CM | VGWQAPQGSRSLTPCTCGSSDLYLVTRHADVIPVRRRGDSRGSLLSPRPISY |
| | V-Gal4DBD- | LKGSAGGPLLCPAGHAVGIFRAAVSTRGVAKAVDFIPVESLETTMRSPGSGA |
| | NS3a-P2a- | TNESLLKQAGDVEENPGPMSSDEEEARELIERAKEAAERAQEAAERTGDPRV |
| | DNCR2-VPR, | RELARELKRLAQEAAEEVKRDPSSSDVNEALKLIVEAIEAAVDALEAAERTG |
| | with NS3a | DPEVRELARELVRLAVEAAEEVQRNPSSSDVNEALHSIVYAIEAAIFALEAA |
| | and DNCR2 | ERTGDPEVRELARELVRLAVEAAEEVQRNPSSRNVEHALMRIVLAIYLAEEN |
| | in bold | LREAEESGDPEKREKARERVREAVERAEEVQRDPSGWLNHEQKLISEEDLDA |
| | | LDDFDLDMLGSDALDDFDLDMLGSDALDDFDLDMLGSDALDDFDLDMLGSPK |
| | | KKRKVGSQYLPDTDDRHRIEEKRKRTYETFKSIMKKSPFSGPTDPRPPPRRI |
| | | AVPSRSSASVPKPAPQPYPFTSSLSTINYDEFPTMVFPSGQISQASALAPAP |
| | | PQVLPQAPAPAPAPAMVSALAQAPAPVPVLAPGPPQAVAPPAPKPTQAGEGT |
| | | LSEALLQLQFDDEDLGALLGNSTDPAVFTDLASVDNSEFQQLLNQGIPVAPH |
| | | TTEPMLMEYPEAITRLVTGAQRPPDPAPAPLGAPGLPNGLLSGDEDFSSIAD |
| | | MDFSALLSQISSGSGSGSRDSREGMFLPKPEAGSAISDVFEGREVCQPKRIR |
| | | PFHPPGSPWANRPLPASLAPTPTGPVHEPVGSLTPAPVPQPLDPAPAVTPEA |
| | | SHLLEDPDEETSQAVKALREMADTVIPQKEEAAICGQMDLSHPPPRGHLDEL |
| | | TTTLESMTEDLNLDSPLTPELNEILDTFLNDECLLHAMHISTGLSIFDTSLF |
| | | (SEQ ID NO: 144) |
| CXCR4_fwd | qPCR | GAAGCTGTTGGCTGAAAAGG (SEQ ID NO: 145) |
| | primer | |
| CXCR4_rev | qPCR | CTCACTGACGTTGGCAAAGA (SEQ ID NO: 146) |
| | primer | |
| GAPDH_fwd | qPCR | ACAGTCAGCCGCATCTTCTT (SEQ ID NO: 147) |
| | primer | |
| GAPDH_rev | qPCR | ACGACCAAATCCGTTGACTC (SEQ ID NO: 148) |
| | primer | |
| GFP_fwd | qPCR | AGCAGAAGAACGGCATCAAG (SEQ ID NO: 149) |
| | primer | |
| GFP_rev | qPCR | GGGGTGTTCTGCTGGTAGTG (SEQ ID NO: 150) |
| | primer | |
| CD95_fwd | qPCR | ATGGTGTCAATGAAGCCAAA (SEQ ID NO: 151) |
| | primer | |
| CD95_rev | qPCR | TGATGCCAATTACGAAGCAG (SEQ ID NO: 152) |
| | primer | |

We claim:

1. A non-naturally occurring polypeptide comprising the general formula X1-X2-X3-X4-X5, wherein:
X1 optionally comprises first, second, third, and fourth helical domains;
X2 comprises a fifth helical domain comprising the amino acid sequence having at least 50%, 55%, 60%, 65%, 70%, 75%, 80%, 85%, 90%, 91%, 92%, 93%, 94%, 95%, 96%, 97%, 98%, 99%, or 100% identity to the full length of HSIVYAIEAAIF (SEQ ID NO:1), wherein 1, 2, 3, or all 4 of the following changes from SEQ ID NO:1 are not permissible: H1K, S2L, Y5E, and F12R
X3 comprises a sixth helical domain;
X4 comprises a seventh helical domain comprising the amino acid sequence having at least 50%, 55%, 60%, 65%, 70%, 75%, 80%, 85%, 90%, 91%, 92%, 93%, 94%, 95%, 96%, 97%, 98%, 99%, or 100% identity to the full length of RNVEHALMRIVLAIY (SEQ ID NO:2), wherein 1, 2, 3, or all 4 of the following changes from SEQ ID NO:2 are not permissible: R1E, H5E, M8K, and L12K; and
X5 comprises an eighth helical domain.

2. The polypeptide of claim 1, wherein acceptable substitutions in X2 relative to SEQ ID NO:1 are selected from the group shown in Table 1.

3. The polypeptide of claim 1, wherein acceptable substitutions in X2 relative to SEQ ID NO:1 are selected from the group shown in Table 2.

4. The polypeptide of any one of claims 1-3, wherein acceptable substitutions in X4 relative to SEQ ID NO:2 are selected from the group shown in Table 3.

5. The polypeptide of any one of claims 1-3, wherein acceptable substitutions in X4 relative to SEQ ID NO:2 are selected from the group shown in Table 4.

6. The polypeptide of any one of claims 1-5, wherein X2 comprises the amino acid sequence having at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identity to the full length of
(SEQ ID NO: 3)
SDVNEALHSIVYAIEAAIFALEAAERT.

7. The polypeptide of any one of claims 1-6, wherein X4 comprises the amino acid sequence having at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identity to the full length of
(SEQ ID NO: 4)
RNVEHALMRIVLAIYLAEENLREAEES.

8. The polypeptide of any one of claims 1-7, wherein X3 comprises the amino acid sequence having at least 25%, 30%, 35%, 40%, 45%, 50^{%}, 55%, 60%, 65%, 70%, 75%, 80%, 85%, 90%, 91%, 92%, 93%, 94%, 95%, 96%, 97%, 98%, 99%, or 100% identity to the full length of EVRELARELVRLAVEAAEEVQR (SEQ ID NO:5).

9. The polypeptide of any one of claims 1-8, wherein X5 comprises the amino acid sequence having at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identity to the full length of EKREKARERVREAVERAEEVQR (SEQ ID NO:6).

10. The polypeptide of any one of claims 1-9, wherein X1, when present, comprises the amino acid sequence having at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identity to the full length of:
(SEQ ID NO: 7)
SDEEEARELIERAKEAAERAQEAAERTGDPRVRELARELKRLAQEAAEEV
KRDPSSSDVNEALKLIVEAIEAAVDALEAAERTGDPEVRELARELVRLAV
EAAEEVQR.

11. The polypeptide of any one of claims 1-10, having at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identity to the full length of SEQ ID NO:8, SEQ ID NO:9, or SEQ ID NO:10.

12. The polypeptide of claim 11, wherein acceptable substitutions relative to SEQ ID NO:8-10 are selected from the group shown in Table 5.

13. The polypeptide of any one of claims 1-12, wherein
X2 comprises a fifth helical domain comprising the amino acid sequence having at least 60% identity to the full length of HSIVYAIEAAIF (SEQ ID NO:1), wherein 1, 2, 3, or all 4 of the following changes from SEQ ID NO:1 are not permissible: H1K, S2L, Y5E, and F12R, and X4 comprises a seventh helical domain comprising the amino acid sequence having at least 60% identity to the full length of RNVEHALMRIVLAIY (SEQ ID NO:2), wherein 1, 2, 3, or all 4 of the following changes from SEQ ID NO:2 are not permissible: R1E, H5E, M8K, and L12K;
X2 comprises a fifth helical domain comprising the amino acid sequence having at least 70% identity to the full length of HSIVYAIEAAIF (SEQ ID NO:1), wherein 1, 2, 3, or all 4 of the following changes from SEQ ID NO:1 are not permissible: H1K, S2L, Y5E, and F12R, and X4 comprises a seventh helical domain comprising the amino acid sequence having at least 70% identity to the full length of RNVEHALMRIVLAIY (SEQ ID NO:2), wherein 1, 2, 3, or all 4 of the following changes from SEQ ID NO:2 are not permissible: R1E, H5E, M8K, and L12K;
X2 comprises a fifth helical domain comprising the amino acid sequence having at least 80% identity to the full length of HSIVYAIEAAIF (SEQ ID NO:1), wherein 1, 2, 3, or all 4 of the following changes from SEQ ID NO:1 are not permissible: H1K, S2L, Y5E, and F12R, and X4 comprises a seventh helical domain comprising the amino acid sequence having at least 80% identity to the full length of RNVEHALMRIVLAIY (SEQ ID NO:2), wherein 1, 2, 3, or all 4 of the following changes from SEQ ID NO:2 are not permissible: R1E, H5E, M8K, and L12K;
X2 comprises a fifth helical domain comprising the amino acid sequence having at least 85% identity to the full length of HSIVYAIEAAIF (SEQ ID NO:1), wherein 1, 2, 3, or all 4 of the following changes from SEQ ID NO:1 are not permissible: H1K, S2L, Y5E, and F12R, and X4 comprises a seventh helical domain comprising the amino acid sequence having at least 85% identity to the full length of RNVEHALMRIVLAIY (SEQ ID NO:2), wherein 1, 2, 3, or all 4 of the following changes from SEQ ID NO:2 are not permissible: R1E, H5E, M8K, and L12K;
X2 comprises a fifth helical domain comprising the amino acid sequence having at least 90% identity to the full length of HSIVYAIEAAIF (SEQ ID NO:1), wherein 1, 2, 3, or all 4 of the following changes from SEQ ID NO:1 are not permissible: H1K, S2L, Y5E, and F12R, and X4 comprises a seventh helical domain comprising the amino acid sequence having at least 90% identity to the full length of RNVEHALMRIVLAIY (SEQ ID NO:2), wherein 1, 2, 3, or all 4 of the following changes from SEQ ID NO:2 are not permissible: R1E, H5E, M8K, and L12K;
X2 comprises a fifth helical domain comprising the amino acid sequence having at least 95% identity to the full length of HSIVYAIEAAIF (SEQ ID NO:1), wherein 1, 2, 3, or all 4 of the following changes from SEQ ID NO:1 are not permissible: H1K, S2L, Y5E, and F12R, and X4 comprises a seventh helical domain comprising the amino acid sequence having at least 95% identity to the full length of RNVEHALMRIVLAIY (SEQ ID NO:2), wherein 1, 2, 3, or all 4 of the following changes from SEQ ID NO:2 are not permissible: R1E, H5E, M8K, and L12K;
X2 comprises a fifth helical domain comprising the amino acid sequence having 100% identity to the full length of HSIVYAIEAAIF (SEQ ID NO:1), and X4 comprises a seventh helical domain comprising the amino acid sequence having 100% identity to the full length of RNVEHALMRIVLAIY (SEQ ID NO:2);

14. The polypeptide of any one of claims 1-13, wherein
X2 comprises the amino acid sequence having at least 60% identity to the full length of
(SEQ ID NO: 3)
SDVNEALHSIVYAIFAAIFALEAAERT,
X4 comprises the amino acid sequence having at least 60% identity to the full length of
(SEQ ID NO: 4)
RNVEHALMRIVLAIYLAEENLREAEES,
X3 comprises the amino acid sequence having at least 60% identity to the full length of EVRELARELVRLAVEAAEEVQR (SEQ ID NO:5), X5 comprises the amino acid sequence having at least 60% identity to the full length of EKREKARERVREAVERAEEVQR (SEQ ID NO:6), and X1, when present, comprises the amino acid sequence having at least 60% identity to the full length of SEQ ID NO:7;
X2 comprises the amino acid sequence having at least 70% identity to the full length of
(SEQ ID NO: 3)
SDVNEALHSIVYAIEAAIFALEAAERT,
X4 comprises the amino acid sequence having at least 70% identity to the full length of
(SEQ ID NO: 4)
RNVEHALMRIVLAIYLAEENLREAEES,
X3 comprises the amino acid sequence having at least 70% identity to the full length of EVRELARELVRLAVEAAEEVQR (SEQ ID NO:5), X5 comprises the amino acid sequence having at least 70% identity to the full length of EKREKARERVREAVERAEEVQR (SEQ ID NO:6), and X1, when present, comprises the amino acid sequence having at least 70% identity to the full length of SEQ ID NO:7;
X2 comprises the amino acid sequence having at least 80% identity to the full length of
(SEQ ID NO: 3)
SDVNEALHSIVYAIEAAIFALEAAERT,
X4 comprises the amino acid sequence having at least 80% identity to the full length of
(SEQ ID NO: 4)
RNVEHALMRIVLAIYLAEENLREAEES,
X3 comprises the amino acid sequence having at least 80% identity to the full length of EVRELARELVRLAVEAAEEVQR (SEQ ID NO:5), X5 comprises the amino acid sequence having at least 80% identity to the full length of EKREKARERVREAVERAEEVQR (SEQ ID NO:6), and X1, when present, comprises the amino acid sequence having at least 80% identity to the full length of SEQ ID NO:7;
X2 comprises the amino acid sequence having at least 80% identity to the full length of
(SEQ ID NO: 3)
SDVNEALHSIVYAIEAAIFALEAAERT,
X4 comprises the amino acid sequence having at least 80% identity to the full length of
(SEQ ID NO: 4)
RNVEHALMRIVLAIYLAEENLREAEES,
X3 comprises the amino acid sequence having at least 80% identity to the full length of EVRELARELVRLAVEAAEEVQR (SEQ ID NO:5), X5 comprises the amino acid sequence having at least 80% identity to the full length of EKREKARERVREAVERAEEVQR (SEQ ID NO:6), and X1, when present, comprises the amino acid sequence having at least 80% identity to the full length of SEQ ID NO:7;
X2 comprises the amino acid sequence having at least 90% identity to the full length of
(SEQ ID NO: 3)
SDVNEALHSIVYAIEAAIFALEAAERT,
X4 comprises the amino acid sequence having at least 90% identity to the full length of
(SEQ ID NO: 4)
RNVEHALMRIVLAIYLAEENLREAEES,
X3 comprises the amino acid sequence having at least 90% identity to the full length of EVRELARELVRLAVEAAEEVQR (SEQ ID NO:5), X5 comprises the amino acid sequence having at least 90% identity to the full length of EKREKARERVREAVERAEEVQR (SEQ ID NO:6), and X1, when present, comprises the amino acid sequence having at least 90% identity to the full length of SEQ ID NO:7;
X2 comprises the amino acid sequence having at least 95% identity to the full length of
(SEQ ID NO: 3)
SDVNEALHSIVYAIEAAIFALEAAERT,
X4 comprises the amino acid sequence having at least 95% identity to the full length of
(SEQ ID NO: 4)
RNVEHALMRIVLAIYLAEENLREAEES,
X3 comprises the amino acid sequence having at least 95% identity to the full length of EVRELARELVRLAVEAAEEVQR (SEQ ID NO:5), X5 comprises the amino acid sequence having at least 95% identity to the full length of EKREKARERVREAVERAEEVQR (SEQ ID NO:6), and X1, when present, comprises the amino acid sequence having at least 95% identity to the full length of SEQ ID NO:7; or
X2 comprises the amino acid sequence having at least 100% identity to the full length of
(SEQ ID NO: 3)
SDVNEALHSIVYAIEAAIFALEAAERT,
X4 comprises the amino acid sequence having 100% identity to the full length of
(SEQ ID NO: 4)
RNVEHALMRIVLAIYLAEENLREAEES,
X3 comprises the amino acid sequence having 100% identity to the full length of EVRELARELVRLAVEAAEEVQR (SEQ ID NO:5), X5 comprises the amino acid sequence having 100% identity to the full length of EKREKARERVREAVERAEEVQR (SEQ ID NO:6), and X1, when present, comprises the amino acid sequence having 100% identity to the full length of SEQ ID NO:7.

15. A non-naturally occurring polypeptide comprising the general formula X1-X2-X3-X4-X5-X6-X7, wherein:
X1 comprises first helical domain;
X2 comprises a second helical domain comprising the amino acid sequence having at least 50%, 55%, 60%, 65%, 70%, 75%, 80%, 85%, 90%, 91%, 92%, 93%, 94%, 95%, 96%, 97%, 98%, 99%, or 100% identity to the full length of
(SEQ ID NO: 20)
DLANLAVAAVLTACL,
wherein 1, 2, 3, 4, 5, 6, or all 7 of the following changes from SEQ ID NO:20 are not permissible: D1K, N4S, L5Q, A8E, L11K, T12L, and L15E;
X3 comprises a third helical domain;
X4 comprises a fourth helical domain comprising the amino acid sequence having at least 50%, 55%, 60%, 65%, 70%, 75%, 80%, 85%, 90%, 91%, 92%, 93%, 94%, 95%, 96%, 97%, 98%, 99%, or 100% identity to the full length of RAVILAIM (SEQ ID NO:21), wherein 1, 2, 3, or all 4 of the following changes from SEQ ID NO:21 are not permissible: R1E, I4K, I7C, and M8E;
X5 comprises a fifth helical domain;
X6 comprises a sixth helical domain comprising the amino acid sequence having at least 50%, 55%, 60%, 65%, 70%, 75%, 80%, 85%, 90%, 91%, 92%, 93%, 94%, 95%, 96%, 97%, 98%, 99%, or 100% identity to the full length of RAIWLAAE (SEQ ID NO:22), wherein 1, 2, 3, or all 4 of the following changes from SEQ ID NO:22 are not permissible: R1L, I3C, W4E, and A7Q; and
X7 comprises seventh and eighth helical domains.

16. The polypeptide of claim 15, wherein acceptable substitutions in X2 relative to SEQ ID NO:20 are selected from those shown in Table 6.

17. The polypeptide of claim 15, wherein acceptable substitutions in X2 relative to SEQ ID NO:20 are selected from those shown in Table 7.

18. The polypeptide of any one of claims 15-17, wherein acceptable substitutions in X4 relative to SEQ ID NO:21 are selected from those shown in Table 8.

19. The polypeptide of any one of claims 15-17, wherein acceptable substitutions in X4 relative to SEQ ID NO:21 are selected from those shown in Table 9.

20. The polypeptide of any one of claims 15-19, wherein acceptable substitutions in X6 relative to SEQ ID NO:22 are selected from those shown in Table 10.

21. The polypeptide of any one of claims 15-19, wherein acceptable substitutions in X6 relative to SEQ ID NO:22 are selected from those shown in Table 11.

22. The polypeptide of any one of claims 15-21, wherein X2 comprises the amino acid sequence having at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identity to the full length of
(SEQ ID NO: 23)
QAAEDAEDLANLAVAAVLTACLLAQEH.

23. The polypeptide of any one of claims 15-22, wherein X4 comprises the amino acid sequence having at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identity to the full length of
(SEQ ID NO: 24)
QAARDAIKLASQAARAVILAIMLAA.

24. The polypeptide of any one of claims 15-23, wherein X6 comprises the amino acid sequence having at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identity to the full length of
(SEQ ID NO: 25)
QAARDAIKLASQAAEAVERAIWLAAE.

25. The polypeptide of any one of claims 15-24, wherein X1 comprises the amino acid sequence having at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identity to the full length of IEKLCKKAEEEAKEAQEKADELRQRH (SEQ ID NO:26).

26. The polypeptide of any one of claims 15-25, wherein X3 comprises the amino acid sequence having at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identity to the full length of DIAKLCIKAASEAAEAASKAAELAQR (SEQ ID NO: 27).

27. The polypeptide of any one of claims 15-26, wherein X5 comprises the amino acid sequence having at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identity to the full length of DIAKLCIKAASEAAEAASKAAELAQR (SEQ ID NO:28).

28. The polypeptide of any one of claims 15-27, wherein X7 comprises the amino acid sequence having at least 25%, 30%, 35%, 40%, 45%, 50^{%}, 55%, 60%, 65%, 70%, 75%, 80%, 85%, 90%, 91%, 92%, 93%, 94%, 95%, 96%, 97%, 98%, 99%, or 100% identity to the full length of DIAKKCIKAASEAAEEASKAAEEAQRHPDSQKARDEIKEASQKAEEVKER (SEQ ID NO:29).

29. The polypeptide of any one of claims 15-28, having at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identity to the full length of a polypeptide selected from the group consisting of SEQ ID NOS:11-12.

30. The polypeptide of claim 29, wherein acceptable substitutions relative to SEQ ID NO:11-12 are selected from the group shown in Table 12.

31. The polypeptide of any one of claims 15-30, wherein:
X2 comprises a second helical domain comprising the amino acid sequence having at least 60% identity to the full length of
(SEQ ID NO: 20)
DLANLAVAAVLTACL,
wherein 1, 2, 3, 4, 5, 6, or all 7 of the following changes from SEQ ID NO:20 are not permissible: D1K, N4S, L5Q, A8E, L11K, T12L, and L15E; X4 comprises a fourth helical domain comprising the amino acid sequence having at least 60% identity to the full length of RAVILAIM (SEQ ID NO:21), wherein 1, 2, 3, or all 4 of the following changes from SEQ ID NO:21 are not permissible: R1E, I4K, I7C, and M8E; and X6 comprises a sixth helical domain comprising the amino acid sequence having at least 60% identity to the full length of RAIWLAAE (SEQ ID NO:22), wherein 1, 2, 3, or all 4 of the following changes from SEQ ID NO:22 are not permissible: R1L, I3C, W4E, and A7Q;
X2 comprises a second helical domain comprising the amino acid sequence having at least 70% identity to the full length of
(SEQ ID NO: 20)
DLANLAVAAVLTACL,
wherein 1, 2, 3, 4, 5, 6, or all 7 of the following changes from SEQ ID NO:20 are not permissible: D1K, N4S, L5Q, A8E, L11K, T12L, and L15E; X4 comprises a fourth helical domain comprising the amino acid sequence having at least 70% identity to the full length of RAVILAIM (SEQ ID NO:21), wherein 1, 2, 3, or all 4 of the following changes from SEQ ID NO:21 are not permissible: R1E, I4K, I7C, and M8E; and X6 comprises a sixth helical domain comprising the amino acid sequence having at least 70% identity to the full length of RAIWLAAE (SEQ ID NO:22), wherein 1, 2, 3, or all 4 of the following changes from SEQ ID NO:22 are not permissible: R1L, I3C, W4E, and A7Q;
X2 comprises a second helical domain comprising the amino acid sequence having at least 80% identity to the full length of
(SEQ ID NO: 20)
DLANLAVAAVLTACL,
wherein 1, 2, 3, 4, 5, 6, or all 7 of the following changes from SEQ ID NO:20 are not permissible: D1K, N4S, L5Q, A8E, L11K, T12L, and L15E; X4 comprises a fourth helical domain comprising the amino acid sequence having at least 80% identity to the full length of RAVILAIM (SEQ ID NO:21), wherein 1, 2, 3, or all 4 of the following changes from SEQ ID NO:21 are not permissible: R1E, I4K, I7C, and M8E; and X6 comprises a sixth helical domain comprising the amino acid sequence having at least 80% identity to the full length of RAIWLAAE (SEQ ID NO:22), wherein 1, 2, 3, or all 4 of the following changes from SEQ ID NO:22 are not permissible: R1L, I3C, W4E, and A7Q;
X2 comprises a second helical domain comprising the amino acid sequence having at least 90% identity to the full length of
(SEQ ID NO: 20)
DLANLAVAAVLTACL,
wherein 1, 2, 3, 4, 5, 6, or all 7 of the following changes from SEQ ID NO:20 are not permissible: D1K, N4S, L5Q, A8E, L11K, T12L, and L15E; X4 comprises a fourth helical domain comprising the amino acid sequence having at least 90% identity to the full length of RAVILAIM (SEQ ID NO:21), wherein 1, 2, 3, or all 4 of the following changes from SEQ ID NO:21 are not permissible: R1E, I4K, I7C, and M8E; and X6 comprises a sixth helical domain comprising the amino acid sequence having at least 90% identity to the full length of RAIWLAAE (SEQ ID NO:22), wherein 1, 2, 3, or all 4 of the following changes from SEQ ID NO:22 are not permissible: R1L, I3C, W4E, and A7Q; or
X2 comprises a second helical domain comprising the amino acid sequence having 100% identity to the full length of
(SEQ ID NO: 20)
DLANLAVAAVLTACL,
wherein 1, 2, 3, 4, 5, 6, or all 7 of the following changes from SEQ ID NO:20 are not permissible: D1K, N4S, L5Q, A8E, L11K, T12L, and L15E; X4 comprises a fourth helical domain comprising the amino acid sequence having 100% identity to the full length of RAVILAIM (SEQ ID NO:21), wherein 1, 2, 3, or all 4 of the following changes from SEQ ID NO:21 are not permissible: R1E, I4K, I7C, and M8E; and X6 comprises a sixth helical domain comprising the amino acid sequence having 100% identity to the full length of RAIWLAAE (SEQ ID NO:22), wherein 1, 2, 3, or all 4 of the following changes from SEQ ID NO:22 are not permissible: R1L, 13C, W4E, and A7Q.

32. The polypeptide of any one of claims 15-31, wherein:
X2 comprises the amino acid sequence having at least 60% identity to the full length of
(SEQ ID NO: 23)
QAAEDAEDLANLAVAAVLTACLLAQEH,
X4 comprises the amino acid sequence having at least 60% identity to the full length of
(SEQ ID NO: 24)
QAARDAIKLASQAARAVILAIMLAA,
X6 comprises the amino acid sequence having at least 60% identity to the full length of
(SEQ ID NO: 25)
QAARDAIKLASQAAEAVERAIWLAAE,
X1 comprises the amino acid sequence having at least 60% identity to the full length of IEKLCKKAEEEAKEAQEKADELRQRH (SEQ ID NO:26), X3 comprises the amino acid sequence having at least 60% identity to the full length of DIAKLCIKAASEAAEAASKAAELAQR (SEQ ID NO: 27), X5 comprises the amino acid sequence having at least 60% identity to the full length of DIAKLCIKAASEAAEAASKAAELAQR (SEQ ID NO:28), and X7 comprises the amino acid sequence having at least 60% identity to the full length of
(SEQ ID NO: 29)
DIAKKCIKAASEAAEEASKAAEEAQRHPDSQKARDEIKEASQKAEEVKER;
X2 comprises the amino acid sequence having at least 70% identity to the full length of
(SEQ ID NO: 23)
QAAEDAEDLANLAVAAVLTACLLAQEH,
X4 comprises the amino acid sequence having at least 70% identity to the full length of
(SEQ ID NO: 24)
QAARDAIKLASQAARAVILAIMLAA,
X6 comprises the amino acid sequence having at least 70% identity to the full length of
(SEQ ID NO: 25)
QAARDAIKLASQAAEAVERAIWLAAE,
X1 comprises the amino acid sequence having at least 70% identity to the full length of IEKLCKKAEEEAKEAQEKADELRQRH (SEQ ID NO:26), X3 comprises the amino acid sequence having at least 70% identity to the full length of DIAKLCIKAASEAAEAASKAAELAQR (SEQ ID NO: 27), X5 comprises the amino acid sequence having at least 70% identity to the full length of DIAKLCIKAASEAAEAASKAAELAQR (SEQ ID NO:28), and X7 comprises the amino acid sequence having at least 70% identity to the full length of
(SEQ ID NO: 29)
DIAKKCIKAASEAAEEASKAAEEAQRHPDSQKARDEIKEASQKAEEVKER;
X2 comprises the amino acid sequence having at least 80% identity to the full length of
(SEQ ID NO: 23)
QAAEDAEDLANLAVAAVLTACLLAQEH,
X4 comprises the amino acid sequence having at least 80% identity to the full length of
(SEQ ID NO: 24)
QAARDAIKLASQAARAVILAIMLAA,
X6 comprises the amino acid sequence having at least 80% identity to the full length of
(SEQ ID NO: 25)
QAARDAIKLASQAAEAVERAIWLAAE,
X1 comprises the amino acid sequence having at least 80% identity to the full length of IEKLCKKAEEEAKEAQEKADELRQRH (SEQ ID NO:26), X3 comprises the amino acid sequence having at least 80% identity to the full length of DIAKLCIKAASEAAEAASKAAELAQR (SEQ ID NO: 27), X5 comprises the amino acid sequence having at least 80% identity to the full length of DIAKLCIKAASEAAEAASKAAELAQR (SEQ ID NO:28), and X7 comprises the amino acid sequence having at least 80% identity to the full length of
(SEQ ID NO: 29)
DIAKKCIKAASEAAEEASKAAEEAQRHPDSQKARDEIKEASQKAEEVKER;
X2 comprises the amino acid sequence having at least 90% identity to the full length of
(SEQ ID NO: 23)
QAAEDAEDLANLAVAAVLTACLLAQEH,
X4 comprises the amino acid sequence having at least 90% identity to the full length of
(SEQ ID NO: 24)
QAARDAIKLASQAARAVILAIMLAA,
X6 comprises the amino acid sequence having at least 90% identity to the full length of
(SEQ ID NO: 25)
QAARDAIKLASQAAEAVERAIWLAAE,
X1 comprises the amino acid sequence having at least 90% identity to the full length of IEKLCKKAEEEAKEAQEKADELRQRH (SEQ ID NO:26), X3 comprises the amino acid sequence having at least 90% identity to the full length of DIAKLCIKAASEAAEAASKAAELAQR (SEQ ID NO: 27), X5 comprises the amino acid sequence having at least 90% identity to the full length of DIAKLCIKAASEAAEAASKAAELAQR (SEQ ID NO:28), and X7 comprises the amino acid sequence having at least 90% identity to the full length of
(SEQ ID NO: 29)
DIAKKCIKAASEAAEEASKAAEEAQRHPDSQKARDEIKEASQKAEEVKER;
X2 comprises the amino acid sequence having at least 95% identity to the full length of
(SEQ ID NO: 23)
QAAEDAEDLANLAVAAVLTACLLAQEH,
X4 comprises the amino acid sequence having at least 95% identity to the full length of
(SEQ ID NO: 24)
QAARDAIKLASQAARAVILAIMLAA,
X6 comprises the amino acid sequence having at least 95% identity to the full length of
(SEQ ID NO: 25)
QAARDAIKLASQAAEAVERAIWLAAE,
X1 comprises the amino acid sequence having at least 95% identity to the full length of IEKLCKKAEEEAKEAQEKADELRQRH (SEQ ID NO:26), X3 comprises the amino acid sequence having at least 95% identity to the full length of DIAKLCIKAASEAAEAASKAAELAQR (SEQ ID NO: 27), X5 comprises the amino acid sequence having at least 95% identity to the full length of DIAKLCIKAASEAAEAASKAAELAQR (SEQ ID NO:28), and X7 comprises the amino acid sequence having at least 95% identity to the full length of
(SEQ ID NO: 29)
DIAKKCIKAASEAAEEASKAAEEAQRHPDSQKARDEIKEASQKAEEVKER;
or
X2 comprises the amino acid sequence having 100% identity to the full length of
(SEQ ID NO: 23)
QAAEDAEDLANLAVAAVLTACLLAQEH,
X4 comprises the amino acid sequence having 100% identity to the full length of
(SEQ ID NO: 24)
QAARDAIKLASQAARAVILAIMLAA,
X6 comprises the amino acid sequence having 100% identity to the full length of
(SEQ ID NO: 25)
QAARDAIKLASQAAEAVERAIWLAAE,
X1 comprises the amino acid sequence having 100% identity to the full length of IEKLCKKAEEEAKEAQEKADELRQRH (SEQ ID NO:26), X3 comprises the amino acid sequence having 100% identity to the full length of DIAKLCIKAASEAAEAASKAAELAQR (SEQ ID NO: 27), X5 comprises the amino acid sequence having 100% identity to the full length of DIAKLCIKAASEAAEAASKAAELAQR (SEQ ID NO:28), and X7 comprises the amino acid sequence having 100% identity to the full length of
(SEQ ID NO: 29)
DIAKKCIKAASEAAEEASKAAEEAQRHPDSQKARDEIKEASQKAEEVKER.

33. A fusion protein comprising:
(a) the polypeptide of any one of claims 1-32; and
(b) a polypeptide localization domain at the N-terminus and/or the C-terminus of the fusion protein.

34. A fusion protein comprising:
(a) the polypeptide of any one of claims 1-32; and
(b) a protein having one or more interaction surfaces.

35. The fusion of claim 34, wherein the protein having one or more interaction surfaces comprises an enzymatic protein, protein-protein interaction domain or a nucleic acid-binding domain.

36. The fusion protein of any one of claims 34-35, wherein the protein having one or more interaction surfaces is selected from the group consisting of: Cas9 and related CRISPR proteins (catalytically active or dead), a DNA binding domain of a transcription factor (such as the Gal4 DNA binding domain), a pro-apoptotic domain (such as caspase 9), and a cell surface receptor (such as a chimeric antigen receptor).

37. A recombinant fusion protein, comprising a polypeptide of the general formula X1-B1-X2-B2-X3, wherein
(a) one of X1 and X3 is selected from the group consisting of
(i) a peptide comprising the amino acid sequence having at least 75%, 80%, 85%, 90%, 91%, 92%, 93%, 94%, 95%, 96%, 97%, 98%, 99%, or 100% identity to the full length of the amino acid sequence selected from GELGRLVYLLDGPGYDPIHSD (SEQ ID NO:13), GELDELVYLLDGPGYDPIHSD (SEQ ID NO:14), GELGELVYLLDGPGYDPIHSD (SEQ ID NO: 15), or GELDRLVYLLDGPGYDPIHSD (SEQ ID NO:16), or GELDELVYLLDGPGYDPIHSDVVTRGGSHLFNF (SEQ ID NO:17) (“ANR peptide”).
(ii) the DNCR polypeptide of any one of claims 1-14; and
(iii) the GNCR polypeptide of any one of claims 15-32;
(b) the other of X1 and X3 is an NS3a peptide (either catalytically active or dead), wherein if X1 or X3 is the ANR peptide, then NS3a is one of SEQ ID NOS:30-38;
(c) X2 is a protein having one or more interaction surfaces; and
(d) B1 and B2 are optional amino acid linkers.

38. The recombinant fusion protein of claim 37, wherein the NS3a peptide comprises the amino acid sequence having at least 80%, 75%, 90%, 91%, 92%, 93%, 94%, 95%, 96%, 97%, 98%, 99%, or 100% identity to the full length of the amino acid sequence selected from the group consisting of SEQ ID NOS:30-38, wherein the bolded amino acid residue is the catalytic position, wherein the bolded “S” residue represents catalytically active NS3a peptides, and wherein the bolded ‘S” residue can be substituted with an alanine (or other) residue to render the NS3a peptide catalytically dead.

39. The recombinant fusion protein of any one of claims 37-38, wherein one or both of B1 and B2 are present.

40. The recombinant fusion protein of claim 39, wherein both B1 and B2 are present.

41. The recombinant fusion protein of any one of claims 37-40, wherein one of X1 and X3 is a peptide comprising the amino acid sequence having at least 75%, 80%, 85%, 90%, 91%, 92%, 93%, 94%, 95%, 96%, 97%, 98%, 99%, or 100% identity to the full length of the amino acid sequence selected from GELGRLVYLLDGPGYDPIHSD (SEQ ID NO:13), GELDELVYLLDGPGYDPIHSD (SEQ ID NO:14), GELGELVYLLDGPGYDPIHSD (SEQ ID NO:15), or GELDRLVYLLDGPGYDPIHSD (SEQ ID NO:16), or GELDELVYLLDGPGYDPIHSDVVTRGGSHLFNF (SEQ ID NO: 17).

42. The recombinant fusion protein of any one of claims 37-40, wherein one of X1 and X3 is the polypeptide of any one of claims 1-14.

43. The recombinant fusion protein of any one of claims 37-40, wherein one of X1 and X3 is the polypeptide of any one of claims 15-32.

44. The recombinant fusion protein of any one of claims 37-43, wherein X2 is an enzymatic protein, protein-protein interaction domain, or nucleic acid-binding domain.

45. The recombinant fusion protein of any one of claims 37-44, wherein X2 is a protein selected from the group consisting of a GEF such as SOS, Cas9 and related CRISPR proteins (catalytically active or dead), a DNA binding domain of a transcription factor (such as the Gal4 DNA binding domain), a pro-apoptotic domain (such as caspase 9), and a cell surface receptor (such as a chimeric antigen receptor).

46. The recombinant fusion protein of any one of claims 37-45, further comprising a peptide localization tag at the N-terminus and/or the C-terminus of the fusion protein, including but not limited to a membrane localization or nuclear localization tag.

47. The recombinant fusion protein of any one of claims 37-46, wherein the recombinant fusion protein comprises the comprises the amino acid sequence having at least 50%, 55%, 60%, 65%, 70%, 75%, 80%, 85%, 90%, 91%, 92%, 93%, 94%, 95%, 96%, 97%, 98%, 99%, or 100% identity to the full length of the amino acid sequence of SEQ ID NO:39.

48. A polypeptide comprising the amino acid sequence selected from the group consisting SEQ ID NO:31-38, wherein the bolded amino acid residue is the catalytic position, wherein the bolded “S” residue represents catalytically active NS3a peptides, and wherein the bolded ‘S” residue can be substituted with an alanine (or other) residue to render the NS3a peptide catalytically dead.

49. A combination, comprising:
(a) a first fusion protein comprising:
(i) a localization tag or a protein having one or more interaction surfaces; and
(ii) an NS3a peptide comprising the amino acid sequence having at least 80%, 85%, 90%, 91%, 92%, 93%, 94%, 95%, 96%, 97%, 98%, 99%, or 100% identity to the full length of the amino acid sequence selected from the group consisting of SEQ ID NOS:31-38, wherein the bolded amino acid residue is the catalytic position, wherein the bolded “S” residue represents catalytically active NS3a peptides, and wherein the bolded ‘S” residue can be substituted with an alanine (or other) residue to render the NS3a peptide catalytically dead; and
(b) one or more second fusion proteins comprising:
(i) a localization tag if the first fusion protein comprises a protein having one or more interaction surfaces; or a protein having one or more interaction surfaces if the first fusion protein comprises a localization tag; and
(ii) a polypeptide selected from the group consisting of selected from the group consisting of:
(A) a polypeptide comprising the amino acid sequence having at least 50%, 55%, 60%, 65%, 70%, 75%, 80%, 85%, 90%, 91%, 92%, 93%, 94%, 95%, 96%, 97%, 98%, 99%, or 100% identity to the full length of the amino acid sequence selected from GELGRLVYLLDGPGYDPIHSD (SEQ ID NO:13), GELDELVYLLDGPGYDPIHSD (SEQ ID NO:14), GELGELVYLLDGPGYDPIHSD (SEQ ID NO:15), GELDRLVYLLDGPGYDPIHSD (SEQ ID NO:16), or GELDELVYLLDGPGYDPIHSDVVTRGGSHLFNF (SEQ ID NO:17);
(B) the DNCR polypeptide of any one of claims 1-14; and
(C) the GNCR polypeptide of any one of claims 15-32.

50. The combination of claim 49, wherein the first fusion protein comprises the NS3a polypeptide of claim 48.

51. The combination of claim 48 or 49, wherein the second fusion protein comprises a polypeptide comprising the amino acid sequence having at least 50%, 55%, 60%, 65%, 70%, 75%, 80%, 85%, 90%, 91%, 92%, 93%, 94%, 95%, 96%, 97%, 98%, 99%, or 100% identity to the full length of the amino acid sequence selected from SEQ ID NO:13-17.

52. The combination of claim 48 or 49, wherein the second fusion protein comprises the DNCR polypeptide of any one of claims 1-14.

53. The combination of claim 48 or 49, wherein the second fusion protein comprises the GNCR polypeptide of any one of claims 15-32.

54. A nucleic acid encoding the polypeptide of any one of claims 1-32 or 48, the fusion protein of any one of claims 33-36, or the recombinant fusion protein of any one of claims 37-47.

55. An expression vector comprising the nucleic acid of claim 54 operatively linked to a promoter sequence.

56. A host cell comprising the nucleic acid of claim 54 and/or the expression vector of claim 55.

57. Use of the polypeptide, fusion protein, recombinant fusion protein, combination, nucleic acid, expression vector, or host cell or any embodiment disclosed herein to carry out any methods, including but not limited to those disclosed herein.
